SEQUENCE LISTING (187 sequences)

SEQ ID NO: 1 - protein, 107 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 2 - protein, 120 aa, Artificial Sequence
  source: /note=“Description of Artificial Sequence: Synthetic polypeptide”
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 11 aa, Artificial Sequence
  source: /note=“Description of Artificial Sequence: Synthetic peptide”
RASQDISKYLN

SEQ ID NO: 4 - protein, 7 aa, Artificial Sequence
  source: /note=“Description of Artificial Sequence: Synthetic peptide”
HTSRLHS

SEQ ID NO: 5 - protein, 9 aa, Artificial Sequence
  source: /note=“Description of Artificial Sequence: Synthetic peptide”
QQGNTLPYT

SEQ ID NO: 6 - protein, 5 aa, Artificial Sequence
  source: /note=“Description of Artificial Sequence: Synthetic peptide”
DYGVS

SEQ ID NO: 7 - protein, 16 aa, Artificial Sequence
  source: /note=“Description of Artificial Sequence: Synthetic peptide”
VIWGSETTYYNSALKS

SEQ ID NO: 8 - protein, 12 aa, Artificial Sequence
  source: /note=“Description of Artificial Sequence: Synthetic peptide”
HYYYGGSYAMDY

SEQ ID NO: 9 - protein, 18 aa, Artificial Sequence
  source: /note=“Description of Artificial Sequence: Synthetic peptide”
GSTSGSGKPGSGEGSTKG

SEQ ID NO: 10 - protein, 22 aa, Homo sapiens
MLLLVTSLLLCELPHPAFLLIP

SEQ ID NO: 11 - protein, 267 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 12 - protein, 245 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 13 - protein, 267 aa, Artificial Sequence
  source: /note=“Description of Artificial Sequence: Synthetic polypeptide”
MLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 14 - protein, 245 aa, Artificial Sequence
  source: /note=“Description of Artificial Sequence: Synthetic polypeptide”
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 15 - protein, 263 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 16 - protein, 242 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 17 - protein, 15 aa, Artificial Sequence
  source: /note=“Description of Artificial Sequence: Synthetic peptide”
GGGGSGGGGSGGGGS

SEQ ID NO: 18 - protein, 21 aa, Artificial Sequence
  source: /note=“Description of Artificial Sequence: Synthetic peptide”
MALPVTALLLPLALLLHAARP

SEQ ID NO: 19 - DNA, 321 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcacc

SEQ ID NO: 20 - DNA, 360 bp, Artificial Sequence
  source: /note=“Description of Artificial Sequence: Synthetic polynucleotide”
gaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagc

SEQ ID NO: 21 - DNA, 33 bp, Artificial Sequence
  source: /note=“Description of Artificial Sequence: Synthetic oligonucleotide”
cgggccagccaggacatcagcaagtacctgaac

SEQ ID NO: 22 - DNA, 21 bp, Artificial Sequence
  source: /note=“Description of Artificial Sequence: Synthetic oligonucleotide”
cacaccagccggctgcacagc

SEQ ID NO: 23 - DNA, 27 bp, Artificial Sequence
  source: /note=“Description of Artificial Sequence: Synthetic oligonucleotide”
cagcagggcaacacactgccctacacc

SEQ ID NO: 24 - DNA, 15 bp, Artificial Sequence
  source: /note=“Description of Artificial Sequence: Synthetic oligonucleotide”
gactacggcgtgtcc

SEQ ID NO: 25 - DNA, 48 bp, Artificial Sequence
  source: /note=“Description of Artificial Sequence: Synthetic oligonucleotide”
gtgatctggggcagcgagaccacctactacaacagcgccctgaagagc

SEQ ID NO: 26 - DNA, 36 bp, Artificial Sequence
  source: /note=“Description of Artificial Sequence: Synthetic oligonucleotide”
cactactactacggcggcagctacgccatggactac

SEQ ID NO: 27 - DNA, 54 bp, Artificial Sequence
  source: /note=“Description of Artificial Sequence: Synthetic oligonucleotide”
ggcagcacctccggcagcggcaagcctggcagcggcgagggcagcaccaagggc

SEQ ID NO: 28 - DNA, 66 bp, Homo sapiens
atgctgctgctggtgaccagcctgctgctgtgtgagctgccccaccccgcctttctgctgatcccc

SEQ ID NO: 29 - DNA, 801 bp, Artificial Sequence
  source: /note=“Description of Artificial Sequence: Synthetic polynucleotide”
atgctgctgctggtgaccagcctgctgctgtgtgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagc

SEQ ID NO: 30 - DNA, 735 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagc

SEQ ID NO: 31 - DNA, 801 bp, Artificial Sequence
  source: /note=“Description of Artificial Sequence: Synthetic polynucleotide”
atgctgctgctggtgaccagcctgctgctgtgtgagctgccccaccccgcctttctgctgatccccgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcacc

SEQ ID NO: 32 - DNA, 735 bp, Artificial Sequence
  source: /note=“Description of Artificial Sequence: Synthetic polynucleotide”
gaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcacc

SEQ ID NO: 33 - DNA, 789 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 34 - DNA, 726 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 35 - DNA, 45 bp, Artificial Sequence
  source: /note=“Description of Artificial Sequence: Synthetic oligonucleotide”
ggtggcggtggctcgggcggtggtgggtcgggtggcggcggatct

SEQ ID NO: 36 - DNA, 63 bp, Artificial Sequence
  source: /note=“Description of Artificial Sequence: Synthetic oligonucleotide”
atggccttaccagtgaccgccttgctcctgccgctggccttgctgctccacgccgccaggccg

SEQ ID NO: 37 - protein, 47 aa, Homo sapiens
KPTTTPAPRPPTPAPTIASQPLSLRPEACRPAAGGAVHTRGLDFACD

SEQ ID NO: 38 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 39 - protein, 42 aa, Homo sapiens
AAAIEVMYPPPYLDNEKSNGTIIHVKGKHLCPSPLFPGPSKP

SEQ ID NO: 40 - DNA, 141 bp, Homo sapiens
aagcccaccaccacccctgcccctagacctccaaccccagcccctacaatcgccagccagcccctgagcctgaggcccgaagcctgtagacctgccgctggcggagccgtgcacaccagaggcctggatttcgcctgcgac

SEQ ID NO: 41 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 42 - DNA, 126 bp, Homo sapiens
gcggccgcaattgaagttatgtatcctcctccttacctagacaatgagaagagcaatggaaccattatccatgtgaaagggaaacacctttgtccaagtcccctatttcccggaccttctaagccc

SEQ ID NO: 43 - protein, 28 aa, Homo sapiens
IYIWAPLAGTCGVLLLSLVITLYCNHRN

SEQ ID NO: 44 - protein, 24 aa, Artificial Sequence
  source: /note=“Description of Artificial Sequence: Synthetic peptide”
IYIWAPLAGTCGVLLLSLVITLYC

SEQ ID NO: 45 - protein, 27 aa, Homo sapiens
FWVLVVVGGVLACYSLLVTVAFIIFWV

SEQ ID NO: 46 - protein, 75 aa, Homo sapiens
KPTTTPAPRPPTPAPTIASQPLSLRPEACRPAAGGAVHTRGLDFACDIYIWAPLAGTCGVLLLSLVITLYCNHRN

SEQ ID NO: 47 - protein, 69 aa, Artificial Sequence
  source: /note=“Description of Artificial Sequence: Synthetic polypeptide”
TTTPAPRPPTPAPTIASQPLSLRPEACRPAAGGAVHTRGLDFACDIYIWAPLAGTCGVLLLSLVITLYC

SEQ ID NO: 48 - protein, 69 aa, Homo sapiens
AAAIEVMYPPPYLDNEKSNGTIIHVKGKHLCPSPLFPGPSKPFWVLVVVGGVLACYSLLVTVAFIIFWV

SEQ ID NO: 49 - DNA, 84 bp, Homo sapiens
atctacatctgggcccctctggccggcacctgtggcgtgctgctgctgagcctggtcatcaccctgtactgcaaccaccggaat

SEQ ID NO: 50 - DNA, 84 bp, Homo sapiens
atctacatctgggcacctctggccggcacctgtggcgtgctgctgctgagcctggtcatcaccctgtactgcaaccaccggaat

SEQ ID NO: 51 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 52 - DNA, 81 bp, Homo sapiens
ttttgggtgctggtggtggttgggggagtcctggcttgctatagcttgctagtaacagtggcctttattattttctgggtg

SEQ ID NO: 53 - DNA, 225 bp, Homo sapiens
aagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaat

SEQ ID NO: 54 - DNA, 225 bp, Homo sapiens
aagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcacctctggccggcacctgtggcgtgctgctgctgagcctggtcatcaccctgtactgcaaccaccggaat

SEQ ID NO: 55 - DNA, 207 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 56 - DNA, 207 bp, Homo sapiens
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtg

SEQ ID NO: 57 - protein, 41 aa, Artificial Sequence
  source: /note=“Description of Artificial Sequence: Synthetic polypeptide”
RSKRSRGGHSDYMNMTPRRPGPTRKHYQPYAPPRDFAAYRS

SEQ ID NO: 58 - protein, 41 aa, Homo sapiens
RSKRSRLLHSDYMNMTPRRPGPTRKHYQPYAPPRDFAAYRS

SEQ ID NO: 59 - protein, 42 aa, Homo sapiens
KRGRKKLLYIFKQPFMRPVQTTQEEDGCSCRFPEEEEGGCEL

SEQ ID NO: 60 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 61 - protein, 153 aa, Artificial Sequence
  source: /note=“Description of Artificial Sequence: Synthetic polypeptide”
RSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - protein, 154 aa, Homo sapiens
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 63 - protein, 153 aa, Homo sapiens
RSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 64 - DNA, 123 bp, Artificial Sequence
  source: /note=“Description of Artificial Sequence: Synthetic polynucleotide”
aggagcaagcggagcagaggcggccacagcgactacatgaacatgaccccccggaggcctggccccacccggaagcactaccagccctacgcccctcccagggacttcgccgcctaccggagc

SEQ ID NO: 65 - DNA, 123 bp, Homo sapiens
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 66 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 67 - DNA, 336 bp, Homo sapiens
cgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 68 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 69 - DNA, 459 bp, Artificial Sequence
  source: /note=“Description of Artificial Sequence: Synthetic polynucleotide”
aggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 70 - DNA, 462 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 71 - DNA, 459 bp, Homo sapiens
aggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 72 - protein, 495 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 519 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGVKQTLNFDLLKLAGDVESNPG

SEQ ID NO: 74 - protein, 473 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 497 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGVKQTLNFDLLKLAGDVESNPG

SEQ ID NO: 76 - protein, 495 aa, Artificial Sequence
  source: /note=“Description of Artificial Sequence: Synthetic polypeptide”
MLLLVTSLLLCELPHPAFLLIPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 473 aa, Artificial Sequence
  source: /note=“Description of Artificial Sequence: Synthetic polypeptide”
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 465 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 489 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 467 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - DNA, 1485 bp, Artificial Sequence
  source: /note=“Description of Artificial Sequence: Synthetic polynucleotide”
atgctgctgctggtgaccagcctgctgctgtgtgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 83 - DNA, 1485 bp, Artificial Sequence
  source: /note=“Description of Artificial Sequence: Synthetic polynucleotide”
atgctgctgctggtgaccagcctgctgctgtgtgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcacctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 84 - DNA, 1557 bp, Artificial Sequence
  source: /note=“Description of Artificial Sequence: Synthetic polynucleotide”
atgctgctgctggtgaccagcctgctgctgtgtgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccagaggctccggagtgaagcagaccctgaatttcgacctgctgaagctggccggggacgtggagagcaaccctggc

SEQ ID NO: 85 - skipped

SEQ ID NO: 86 - DNA, 1419 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 87 - DNA, 1419 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcacctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 88 - DNA, 1491 bp, Artificial Sequence
  source: /note=“Description of Artificial Sequence: Synthetic polynucleotide”
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccagaggctccggagtgaagcagaccctgaatttcgacctgctgaagctggccggggacgtggagagcaaccctggc

SEQ ID NO: 89 - skipped

SEQ ID NO: 90 - DNA, 1485 bp, Artificial Sequence
  source: /note=“Description of Artificial Sequence: Synthetic polynucleotide”
atgctgctgctggtgaccagcctgctgctgtgtgagctgccccaccccgcctttctgctgatccccgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcacctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 91 - DNA, 1419 bp, Artificial Sequence
  source: /note=“Description of Artificial Sequence: Synthetic polynucleotide”
gaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcggcagcacctccggcagcggcaagcctggcagcggcgagggcagcaccaagggcgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcacctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 92 - DNA, 1458 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 93 - DNA, 1395 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 94 - DNA, 1467 bp, Artificial Sequence
  source: /note=“Description of Artificial Sequence: Synthetic polynucleotide”
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 95 - DNA, 1401 bp, Artificial Sequence
  source: /note=“Description of Artificial Sequence: Synthetic polynucleotide”
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 96 - protein, 264 aa, Artificial Sequence
  source: /note=“Description of Artificial Sequence: Synthetic polypeptide”
MRLPAQLLGLLMLWVPGSSGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGGSGGGGSGGGGSFWVLVVVGGVLACYSLLVTVAFIIFWVRSKRS

SEQ ID NO: 97 - protein, 244 aa, Artificial Sequence
  source: /note=“Description of Artificial Sequence: Synthetic polypeptide”
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGGSGGGGSGGGGSFWVLVVVGGVLACYSLLVTVAFIIFWVRSKRS

SEQ ID NO: 98 - protein, 171 aa, Homo sapiens
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQ

SEQ ID NO: 99 - protein, 141 aa, Homo sapiens
VCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPS

SEQ ID NO: 100 - protein, 21 aa, Homo sapiens
VCHALCSPEGCWGPEPRDCVS

SEQ ID NO: 101 - protein, 32 aa, Homo sapiens
FWVLVVVGGVLACYSLLVTVAFIIFWVRSKRS

SEQ ID NO: 102 - protein, 20 aa, Artificial Sequence
  source: /note=“Description of Artificial Sequence: Synthetic peptide”
GGGGSGGGGSGGGGSGGGGS

SEQ ID NO: 103 - protein, 355 aa, Artificial Sequence
  source: /note=“Description of Artificial Sequence: Synthetic polypeptide”
MRLPAQLLGLLMLWVPGSSGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 104 - protein, 335 aa, Artificial Sequence
  source: /note=“Description of Artificial Sequence: Synthetic polypeptide”
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 105 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 106 - protein, 263 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIE

SEQ ID NO: 107 - DNA, 792 bp, Artificial Sequence
  source: /note=“Description of Artificial Sequence: Synthetic polynucleotide”
atgaggctccctgctcagctcctggggctgctaatgctctgggtgccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttggtacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 108 - DNA, 732 bp, Artificial Sequence
  source: /note=“Description of Artificial Sequence: Synthetic polynucleotide”
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttggtacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 109 - DNA, 732 bp, Artificial Sequence
  source: /note=“Description of Artificial Sequence: Synthetic polynucleotide”
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 110 - DNA, 513 bp, Homo sapiens
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttggtacctccggtcagaaaaccaaaattataagcaacagaggtgaaaacagctgcaaggccacaggccag

SEQ ID NO: 111 - DNA, 423 bp, Homo sapiens
gtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtcc

SEQ ID NO: 112 - DNA, 63 bp, Homo sapiens
gtctgccatgccttgtgctcccccgagggctgctggggcccggagcccagggactgcgtctct

SEQ ID NO: 113 - DNA, 96 bp, Homo sapiens
ttttgggtgctggtggtggttggtggagtcctggcttgctatagcttgctagtaacagtggcctttattattttctgggtgaggagtaagaggagc

SEQ ID NO: 114 - DNA, 60 bp, Artificial Sequence
  source: /note=“Description of Artificial Sequence: Synthetic oligonucleotide”
ggtggcggtggctcgggcggtggtgggtcgggtggcggcggatctggtggcggtggctcg

SEQ ID NO: 115 - DNA, 1065 bp, Artificial Sequence
  source: /note=“Description of Artificial Sequence: Synthetic polynucleotide”
atgaggctccctgctcagctcctggggctgctaatgctctgggtgccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 116 - DNA, 1005 bp, Artificial Sequence
  source: /note=“Description of Artificial Sequence: Synthetic polynucleotide”
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatg

SEQ ID NO: 117 - DNA, 891 bp, Homo sapiens
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctcct

SEQ ID NO: 118 - DNA, 789 bp, Homo sapiens
atgaccacaccacggaactctgtgaatggcaccttcccagcagagccaatgaagggaccaatcgcaatgcagagcggacccaagcctctgtttcggagaatgagctccctggtgggcccaacccagtccttctttatgagagagtctaagacactgggcgccgtgcagatcatgaacggactgttccacatcgccctgggaggactgctgatgatcccagccggcatctacgcccctatctgcgtgaccgtgtggtaccctctgtggggcggcatcatgtatatcatctccggctctctgctggccgccacagagaagaacagcaggaagtgtctggtgaagggcaagatgatcatgaatagcctgtccctgtttgccgccatctctggcatgatcctgagcatcatggacatcctgaacatcaagatcagccacttcctgaagatggagagcctgaacttcatcagagcccacaccccttacatcaacatctataattgcgagcctgccaacccatccgagaagaattctccaagcacacagtactgttattccatccagtctctgttcctgggcatcctgtctgtgatgctgatctttgccttctttcaggagctggtcatcgccggcatcgtggagaacgagtggaagaggacctgcagccgccccaagtccaatatcgtgctgctgtccgccgaggagaagaaggagcagacaatcgagatcaaggaggaggtggtgggcctgaccgagacatctagccagcctaagaatgaggaggatatcgag

SEQ ID NO: 119 - protein, 395 aa, Artificial Sequence [sequence omitted]

SEQ ID NO: 120 - protein, 417 aa, Artificial Sequence
  source: /note=“Description of Artificial Sequence: Synthetic polypeptide”
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLLEGGGEGRGSLLTCGDVEENPG

SEQ ID NO: 121 - protein, 377 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 122 - protein, 399 aa, Artificial Sequence
  source: /note=“Description of Artificial Sequence: Synthetic polypeptide”
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLLEGGGEGRGSLLTCGDVEENPG

SEQ ID NO: 123 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 124 - protein, 237 aa, Homo sapiens
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 125 - protein, 26 aa, Artificial Sequence
  source: /note=“Description of Artificial Sequence: Synthetic peptide”
SGGGSGGGGSGGGGSGGGGSGGGSLQ

SEQ ID NO: 126 - DNA, 1185 bp, Artificial Sequence
  source: /note=“Description of Artificial Sequence: Synthetic polynucleotide”
atggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctg

SEQ ID NO: 127 - DNA, 1185 bp, Artificial Sequence
  source: /note=“Description of Artificial Sequence: Synthetic polynucleotide”
atggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaggacctggagaattgttctcaccacctg

SEQ ID NO: 128 - DNA, 1251 bp, Artificial Sequence
  source: /note=“Description of Artificial Sequence: Synthetic polynucleotide”
atggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctgctggagggcggcggagagggcagaggaagtcttctaacatgcggtgacgtggaggagaatcccggc

SEQ ID NO: 129 - DNA, 1254 bp, Artificial Sequence
  source: /note=“Description of Artificial Sequence: Synthetic polynucleotide”
cccatggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctgctggagggcggcggagagggcagaggaagtcttctaacatgcggtgacgtggaggagaatcccggc

SEQ ID NO: 130 - DNA, 1131 bp, Artificial Sequence
  source: /note=“Description of Artificial Sequence: Synthetic polynucleotide”
aactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctg

SEQ ID NO: 131 - DNA, 1131 bp, Artificial Sequence
  source: /note=“Description of Artificial Sequence: Synthetic polynucleotide”
aactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaggacctggagaattgttctcaccacctg

SEQ ID NO: 132 - DNA, 1197 bp, Artificial Sequence
  source: /note=“Description of Artificial Sequence: Synthetic polynucleotide”
aactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctgctggagggcggcggagagggcagaggaagtcttctaacatgcggtgacgtggaggagaatcccggc

SEQ ID NO: 133 - skipped

SEQ ID NO: 134 - DNA, 342 bp, Homo sapiens
aactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagc

SEQ ID NO: 135 - DNA, 711 bp, Homo sapiens
attacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctg

SEQ ID NO: 136 - DNA, 78 bp, Artificial Sequence
  source: /note=“Description of Artificial Sequence: Synthetic oligonucleotide”
tctggcggaggatctggaggaggcggatctggaggaggaggcagtggaggcggaggatctggcggaggatctctgcag

SEQ ID NO: 137 - protein, 22 aa, Foot-and-mouth disease virus
VKQTLNFDLLKLAGDVESNPGP

SEQ ID NO: 138 - protein, 25 aa, Artificial Sequence
  source: /note=“Description of Artificial Sequence: Synthetic polypeptide”
GSGVKQTLNFDLLKLAGDVESNPGP

SEQ ID NO: 139 - protein, 18 aa, Thosea asigna virus
EGRGSLLTCGDVEENPGP

SEQ ID NO: 140 - protein, 24 aa, Artificial Sequence
  source: /note=“Description of Artificial Sequence: Synthetic polypeptide”
LEGGGEGRGSLLTCGDVEENPGPR

SEQ ID NO: 141 - DNA, 66 bp, Foot-and-mouth disease virus
gtgaagcagaccctgaatttcgacctgctgaagctggccggggacgtggagagcaaccctggcccc

SEQ ID NO: 142 - DNA, 75 bp, Artificial Sequence
  source: /note=“Description of Artificial Sequence: Synthetic oligonucleotide”
ggctccggagtgaagcagaccctgaatttcgacctgctgaagctggccggggacgtggagagcaaccctggcccc

SEQ ID NO: 143 - DNA, 54 bp, Thosea asigna virus
gagggcagaggaagtcttctaacatgcggtgacgtggaggagaatcccggccct

SEQ ID NO: 144 - DNA, 72 bp, Artificial Sequence
  source: /note=“Description of Artificial Sequence: Synthetic oligonucleotide”
ctggagggcggcggagagggcagaggaagtcttctaacatgcggtgacgtggaggagaatcccggccctagg

SEQ ID NO: 145 - DNA, 72 bp, Artificial Sequence
  source: /note=“Description of Artificial Sequence: Synthetic oligonucleotide”
ctcgagggcggcggagagggcagaggaagtcttctaacatgcggtgacgtggaggagaatcccggccctagg

SEQ ID NO: 146 - DNA, 551 bp, Artificial Sequence
  source: /note=“Description of Artificial Sequence: Synthetic polynucleotide”
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagat

SEQ ID NO: 147 - skipped

SEQ ID NO: 148 - DNA, 231 bp, Artificial Sequence
  source: /note=“Description of Artificial Sequence: Synthetic polynucleotide”
gatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 149 - DNA, 3609 bp, Artificial Sequence
  source: /note=“Description of Artificial Sequence: Synthetic polynucleotide”
atgctgctgctggtgaccagcctgctgctgtgtgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcccctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccagaggctccggagtgaagcagaccctgaatttcgacctgctgaagctggccggggacgtggagagcaaccctggccccatggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctgctggagggcggcggagagggcagaggaagtcttctaacatgcggtgacgtggaggagaatcccggccctaggatgaggctccctgctcagctcctggggctgctaatgctctgggtgccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttggtacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 150 - DNA, 3609 bp, Artificial Sequence
  source: /note=“Description of Artificial Sequence: Synthetic polynucleotide”
atggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaggacctggagaattgttctcaccacctgctcgagggcggcggagagggcagaggaagtcttctaacatgcggtgacgtggaggagaatcccggccctaggatgaggctccctgctcagctcctggggctgctaatgctctgggtcccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagcggctccggagtgaagcagaccctgaatttcgacctgctgaagctggccggggacgtggagagcaaccctggccccatgctgctgctggtgaccagcctgctgctgtgtgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcacctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 151 - DNA, 3612 bp, Artificial Sequence
  source: /note=“Description of Artificial Sequence: Synthetic polynucleotide”
atgaggatgaggctccctgctcagctcctggggctgctaatgctctgggtcccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagcctcgagggcggcggagagggcagaggaagtcttctaacatgcggtgacgtggaggagaatcccggccctatggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaggacctggagaattgttctcaccacctgggctccggagtgaagcagaccctgaatttcgacctgctgaagctggccggggacgtggagagcaaccctggccccatgctgctgctggtgaccagcctgctgctgtgtgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggagcaggaggacatcgccacctacttttgccagcagggcaacacactgccctacacctttggcggcggaacaaagctggagatcaccggcagcacctccggcagcggcaagcctggcagcggcgagggcagcaccaagggcgaggtgaagctgcaggagagcggccctggcctggtggcccccagccagagcctgagcgtgacctgtaccgtgtccggcgtgtccctgcccgactacggcgtgtcctggatccggcagccccctaggaagggcctggagtggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgtgccaagcactactactacggcggcagctacgccatggactactggggccagggcaccagcgtgaccgtgtccagcaagcccaccaccacccctgcccctagacctccaaccccagcccctacaatcgccagccagcccctgagcctgaggcccgaagcctgtagacctgccgctggcggagccgtgcacaccagaggcctggatttcgcctgcgacatctacatctgggcacctctggccggcacctgtggcgtgctgctgctgagcctggtcatcaccctgtactgcaaccaccggaataggagcaagcggagcagaggcggccacagcgactacatgaacatgaccccccggaggcctggccccacccggaagcactaccagccctacgcccctcccagggacttcgccgcctaccggagccgggtgaagttcagccggagcgccgacgcccctgcctaccagcagggccagaaccagctgtacaacgagctgaacctgggccggagggaggagtacgacgtgctggacaagcggagaggccgggaccctgagatgggcggcaagccccggagaaagaaccctcaggagggcctgtataacgaactgcagaaagacaagatggccgaggcctacagcgagatcggcatgaagggcgagcggcggaggggcaagggccacgacggcctgtaccagggcctgagcaccgccaccaaggatacctacgacgccctgcacatgcaggccctgccccccaga

SEQ ID NO: 152 - protein, 1203 aa, Artificial Sequence
  source: /note=“Description of Artificial Sequence: Synthetic polypeptide”
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGVKQTLNFDLLKLAGDVESNPGPMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLLEGGGEGRGSLLTCGDVEENPGPRMRLPAQLLGLLMLWVPGSSGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGGSGGGGSGGGGSFWVLVVVGGVLACYSLLVTVAFIIFWVRSKRS

SEQ ID NO: 153 - protein, 1203 aa, Artificial Sequence
  source: /note=“Description of Artificial Sequence: Synthetic polypeptide”
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLLEGGGEGRGSLLTCGDVEENPGPRMRLPAQLLGLLMLWVPGSSGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGGSGGGGSGGGGSFWVLVVVGGVLACYSLLVTVAFIIFWVRSKRSGSGVKQTLNFDLLKLAGDVESNPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 1204 aa, Artificial Sequence
  source: /note=“Description of Artificial Sequence: Synthetic polypeptide”
MRMRLPAQLLGLLMLWVPGSSGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGGSGGGGSGGGGSFWVLVVVGGVLACYSLLVTVAFIIFWVRSKRSLEGGGEGRGSLLTCGDVEENPGPMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLGSGVKQTLNFDLLKLAGDVESNPGPMLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSKPTTTPAPRPPTPAPTIASQPLSLRPEACRPAAGGAVHTRGLDFACDIYIWAPLAGTCGVLLLSLVITLYCNHRNRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - DNA, 226 bp, Artificial Sequence
  source: /note=“Description of Artificial Sequence: Synthetic polynucleotide”
agttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaa

SEQ ID NO: 156 - DNA, 246 bp, Artificial Sequence
  source: /note=“Description of Artificial Sequence: Synthetic polynucleotide”
atatcaattgagttgaagtcggaagtttacatacacttaagttggagtcattaaaactcgtttttcaactacaccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaacaattgatat

SEQ ID NO: 157 - DNA, 228 bp, Artificial Sequence
  source: /note=“Description of Artificial Sequence: Synthetic polynucleotide”
ttgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactg

SEQ ID NO: 158 - skipped

SEQ ID NO: 159 - DNA, 248 bp, Artificial Sequence
  source: /note=“Description of Artificial Sequence: Synthetic polynucleotide”
atatctcgagttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaactctcgagatat

SEQ ID NO: 160 - protein, 340 aa, Artificial Sequence
  source: /note=“Description of Artificial Sequence: Synthetic polypeptide”
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFARAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQQDNDPKHTSKHVRKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 161 - DNA, 1020 bp, Artificial Sequence
  source: /note=“Description of Artificial Sequence: Synthetic polynucleotide”
atgggaaaatcaaaagaaatcagccaagacctcagaaaaaaaattgtagacctccacaagtctggttcatccttgggagcaatttccaaacgcctgaaagtaccacgttcatctgtacaaacaatagtacgcaagtataaacaccatgggaccacgcagccgtcataccgctcaggaaggagacgcgttctgtctcctagagatgaacgtactttggtgcgaaaagtgcaaatcaatcccagaacaacagcaaaggaccttgtgaagatgctggaggaaacaggtacaaaagtatctatatccacagtaaaacgagtcctatatcgacataacctgaaaggccgctcagcaaggaagaagccactgctccaaaaccgacataagaaagccagactacggtttgcaagagcacatggggacaaagatcgtactttttggagaaatgtcctctggtctgatgaaacaaaaatagaactgtttggccataatgaccatcgttatgtttggaggaagaagggggaggcttgcaagccgaagaacaccatcccaaccgtgaagcacgggggtggcagcatcatgttgtgggggtgctttgctgcaggagggactggtgcacttcacaaaatagatggcatcatgaggaaggaaaattatgtggatatattgaagcaacatctcaagacatcagtcaggaagttaaagcttggtcgcaaatgggtcttccaacaagacaatgaccccaagcatacttccaaacacgtgagaaaatggcttaaggacaacaaagtcaaggtattggagtggccatcacaaagccctgacctcaatcctatagaaaatttgtgggcagaactgaaaaagcgtgtgcgagcaaggaggcctacaaacctgactcagttacaccagctctgtcaggaggaatgggccaaaattcacccaacttattgtgggaagcttgtggaaggctacccgaaacgtttgacccaagttaaacaatttaaaggcaatgctaccaaatac

SEQ ID NO: 162 - DNA, 792 bp, Artificial Sequence
  source: /note=“Description of Artificial Sequence: Synthetic polynucleotide”
atgaggctccctgctcagctcctggggctgctaatgctctgggtcccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 163 - DNA, 711 bp, Homo sapiens
attacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaggacctggagaattgttctcaccacctg

SEQ ID NO: 164 - DNA, 513 bp, Homo sapiens
cgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccag

SEQ ID NO: 165 - DNA, 69 bp, Artificial Sequence
  source: /note=“Description of Artificial Sequence: Synthetic oligonucleotide”
ctcgagggcggcggagagggcagaggaagtcttctaacatgcggtgacgtggaggagaatcccggccct

SEQ ID NO: 166 - protein, 266 aa, Artificial Sequence
  source: /note=“Description of Artificial Sequence: Synthetic polypeptide”
MRMRLPAQLLGLLMLWVPGSSGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGGSGGGGSGGGGSFWVLVVVGGVLACYSLLVTVAFIIFWVRSKRS

SEQ ID NO: 167 - protein, 266 aa, Artificial Sequence
  source: /note=“Description of Artificial Sequence: Synthetic polypeptide”
PRMRLPAQLLGLLMLWVPGSSGRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSGGGGSGGGGSGGGGSGGGGSFWVLVVVGGVLACYSLLVTVAFIIFWVRSKRS

SEQ ID NO: 168 - skipped

SEQ ID NO: 169 - protein, 20 aa, Artificial Sequence
  source: /note=“Description of Artificial Sequence: Synthetic peptide”
MRLPAQLLGLLMLWVPGSSG

SEQ ID NO: 170 - protein, 22 aa, Artificial Sequence
  source: /note=“Description of Artificial Sequence: Synthetic peptide”
MRMRLPAQLLGLLMLWVPGSSG

SEQ ID NO: 171 - protein, 22 aa, Artificial Sequence
  source: /note=“Description of Artificial Sequence: Synthetic peptide”
PRMRLPAQLLGLLMLWVPGSSG

SEQ ID NO: 172 - skipped

SEQ ID NO: 173 - DNA, 798 bp, Artificial Sequence
  source: /note=“Description of Artificial Sequence: Synthetic polynucleotide”
atgaggatgaggctccctgctcagctcctggggctgctaatgctctgggtcccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 174 - DNA, 798 bp, Artificial Sequence
  source: /note=“Description of Artificial Sequence: Synthetic polynucleotide”
cctaggatgaggctccctgctcagctcctggggctgctaatgctctgggtgccaggatccagtgggcgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttggtacctccggtcagaaaaccaaaattataagcaacagaggtgaaaacagctgcaaggccacaggccaggtctgccatgccttgtgctcccccgagggctgctggggcccggagcccagggactgcgtctctggtggcggtggctcgggcggtggtgggtcgggtggcggcggatctggtggcggtggctcgttttgggtgctggtggtggttggtggagtcctggcttgctatagcttgctagtaacagtggcctttattattttctgggtgaggagtaagaggagc

SEQ ID NO: 175 - skipped

SEQ ID NO: 176 - protein, 18 aa, Artificial Sequence
  source: /note=“Description of Artificial Sequence: Synthetic peptide”
MDWTWILFLVAAATRVHS

SEQ ID NO: 177 - DNA, 54 bp, Artificial Sequence
  source: /note=“Description of Artificial Sequence: Synthetic oligonucleotide”
atggattggacctggattctgtttctggtggccgctgccacaagagtgcacagc

SEQ ID NO: 178 - DNA, 66 bp, Artificial Sequence
  source: /note=“Description of Artificial Sequence: Synthetic oligonucleotide”
atgaggatgaggctccctgctcagctcctggggctgctaatgctctgggtcccaggatccagtggg

SEQ ID NO: 179 - DNA, 66 bp, Artificial Sequence
  source: /note=“Description of Artificial Sequence: Synthetic oligonucleotide”
cctaggatgaggctccctgctcagctcctggggctgctaatgctctgggtgccaggatccagtggg

SEQ ID NO: 180 - protein, 396 aa, Artificial Sequence
  source: /note=“Description of Artificial Sequence: Synthetic polypeptide”
PMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 181 - DNA, 1188 bp, Artificial Sequence
  source: /note=“Description of Artificial Sequence: Synthetic polynucleotide”
cccatggattggacctggattctgtttctggtggccgctgccacaagagtgcacagcaactgggtgaatgtgatcagcgacctgaagaagatcgaggatctgatccagagcatgcacattgatgccaccctgtacacagaatctgatgtgcaccctagctgtaaagtgaccgccatgaagtgttttctgctggagctgcaggtgatttctctggaaagcggagatgcctctatccacgacacagtggagaatctgatcatcctggccaacaatagcctgagcagcaatggcaatgtgacagagtctggctgtaaggagtgtgaggagctggaggagaagaacatcaaggagtttctgcagagctttgtgcacatcgtgcagatgttcatcaatacaagctctggcggaggatctggaggaggcggatctggaggaggaggcagtggaggcggaggatctggcggaggatctctgcagattacatgccctcctccaatgtctgtggagcacgccgatatttgggtgaagtcctacagcctgtacagcagagagagatacatctgcaacagcggctttaagagaaaggccggcacctcttctctgacagagtgcgtgctgaataaggccacaaatgtggcccactggacaacacctagcctgaagtgcattagagatcctgccctggtccaccagaggcctgcccctccatctacagtgacaacagccggagtgacacctcagcctgaatctctgagcccttctggaaaagaacctgccgccagctctcctagctctaataataccgccgccacaacagccgccattgtgcctggatctcagctgatgcctagcaagtctcctagcacaggcacaacagagatcagcagccacgaatcttctcacggaacaccttctcagaccaccgccaagaattgggagctgacagcctctgcctctcaccagcctccaggagtgtatcctcagggccactctgatacaacagtggccatcagcacatctacagtgctgctgtgtggactgtctgccgtgtctctgctggcctgttacctgaagtctagacagacacctcctctggcctctgtggagatggaggccatggaagccctgcctgtgacatggggaacaagcagcagagatgaagacctggagaattgttctcaccacctg

SEQ ID NO: 182 - protein, 23 aa, Artificial Sequence
  source: /note=“Description of Artificial Sequence: Synthetic peptide”
LEGGGEGRGSLLTCGDVEENPGP

SEQ ID NO: 183 - protein, 418 aa, Artificial Sequence
  source: /note=“Description of Artificial Sequence: Synthetic polypeptide”
PMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGSGGGGSGGGGSGGGGSGGGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHLLEGGGEGRGSLLTCGDVEENPG

SEQ ID NO: 184 - DNA, 228 bp, Artificial Sequence
  source: /note=“Description of Artificial Sequence: Synthetic polynucleotide”
ttgagtgtatgttaacttctgacccactgggaatgtgatgaaagaaataaaagctgaaatgaatcattctctctactattattctgatatttcacattcttaaaataaagtggtgatcctaactgaccttaagacagggaatctttactcggattaaatgtcaggaattgtgaaaaagtgagtttaaatgtatttggctaaggtgtatgtaaacttccgacttcaact

SEQ ID NO: 185 - DNA, 60 bp, Artificial Sequence
  source: /note=“Description of Artificial Sequence: Synthetic oligonucleotide”
atgaggctccctgctcagctcctggggctgctaatgctctgggtgccaggatccagtggg

SEQ ID NO: 186 - DNA, 60 bp, Artificial Sequence
  source: /note=“Description of Artificial Sequence: Synthetic oligonucleotide”
atgaggctccctgctcagctcctggggctgctaatgctctgggtcccaggatccagtggg

SEQ ID NO: 187 - protein, 4 aa, Unknown
  source: /note=“Description of Unknown: Furin cleavage site”
RAKR

(19) United States
(12) Patent Application Publication
(10) Pub. No.: US 2024/0316199 A1
(43) Pub. Date: Sep. 26, 2024

(54) Recombinant Vectors Comprising Polycistronic Expression Cassettes and Methods of Use Thereof

(71) Applicant: Alaunos Therapeutics, Inc., Houston, TX (US)

(72) Inventors: Simon OLIVARES, Houston, TX (US); Harjet SINGH, Houston, TX (US); Laurence James Neil COOPER, Houston, TX (US); Lenka Victoria HURTON, Houston, TX (US)

(73) Assignee: Alaunos Therapeutics, Inc., Houston, TX (US)

(21) Appl. No.: 18/259,985

(22) PCT Filed: Dec. 29, 2021
(86) PCT No.: PCT/US2021/073145

§ 371 Date: Jun. 29, 2023

Related U.S. Application Data
(60) Provisional application No. 63/132,434, filed on Dec. 30, 2020.

(51) Int. Cl. A61K 39/00; A61P 35/00; C07K 14/47; C07K 14/54; C07K 14/725; C07K 16/28

(52) U.S. Cl. CPC A61K 39/464412; A61K 39/4611; A61K 39/4632; A61P 35/00; C07K 14/4748; C07K 14/5443; C07K 14/7051; C07K 16/2803; C07K 2317/622; C07K 2317/732; C07K 2319/92

(57) ABSTRACT

Provided herein are vectors comprising a polycistronic expression casstte comprising a polynucleotide that encodes a CD 19 specific chimeric antigen receptor, a polynucleotide that encodes a cytokine, and a polynucleotide that encodes a marker protein, wherein the polynucleotide that encodes the CD 19-specific chimeric antigen receptor and the polynucleotide that encodes the cytokine coding sequence are separated by a polynucleotide sequence that comprises an F2A element, and wherein the polynucleotide sequence that encodes the cytokine and the polynucleotide sequence that encodes the marker protein are separated by a polynucleotide sequence that comprises a T2A element.

### 1. FIELD

The instant disclosure relates to polycistronic vectors comprising at least three cistrons and methods of using the same.

### 2. BACKGROUND

Co-expression of multiple genes in each cell of a population is critical for a wide variety of biomedical applications, including adoptive cell therapy, e.g., chimeric antigen receptor T-cell (CAR T-cell) therapy. A standard strategy for multigene expression is to incorporate the transgenes into multiple vectors and introduce each vector into the cell. However, the use of multiple vectors often produces a substantially heterogeneous population of engineered cells, wherein not all cells express each of the transgenes or do not express each of the transgenes to a similar degree. Such heterogeneity leads to several problems, particularly for therapeutic applications, including e.g., diminished persistence of the desired engineered cell phenotype in vivo, complex manufacturing and purification requirements, and lot-to-lot variability of the engineered cell product.

Given the problems associated with the use of multiple vectors to co-express multiple genes in single cells, there is an unmet need for single polycistronic vectors capable of not only expressing a plurality of transgenes in a single cell, but also of expressing some or all transgenes to a similar degree across a cell population, resulting in an engineered cell population optimized for therapeutic use. 3. SUMMARY

The instant disclosure provides vectors comprising a polycistronic expression cassette, comprising a polynucleotide encoding an anti-CD19 chimeric antigen receptor (CAR), a polynucleotide encoding a fusion protein that comprises IL-15 and IL-15Rα, and a polynucleotide that encodes a marker protein, wherein the polynucleotide encoding the anti-CD19 CAR is separated from the polynucleotide encoding the fusion protein by a polynucleotide sequence that comprises an F2A element, and the polynucleotide encoding the fusion protein is separated from the polynucleotide sequence encoding the marker protein by a polynucleotide sequence that comprises a T2A element. Also provided are pharmaceutical compositions comprising cells, e.g., immune effector cells, engineered utilizing the vectors described herein, and methods of treating a subject using these pharmaceutical compositions. The recombinant vectors disclosed herein are particularly useful in modifying immune effector cells (e.g., T cells) for use in adoptive cell therapy.

Accordingly, in one aspect, the instant disclosure provides a recombinant vector comprising a polycistronic expression cassette, wherein said polycistronic expression cassette comprises a transcriptional regulatory element operably linked to a polynucleotide that comprises, from 5′ to 3′: a first polynucleotide sequence that encodes a chimeric antigen receptor (CAR) that comprises an extracellular antigen-binding domain that specifically binds to CD19, a transmembrane domain, and a cytoplasmic domain; a second polynucleotide sequence that comprises an F2A element; a third polynucleotide sequence that encodes a fusion protein that comprises IL-15, or a functional fragment or functional variant thereof, and IL-15Rα, or a functional fragment or functional variant thereof, a fourth polynucleotide sequence that comprises a T2A element; and a fifth polynucleotide sequence that encodes a marker protein.

In some embodiments, said F2A element comprises a polynucleotide sequence that encodes the amino acid sequence of SEQ ID NO: 137, or the amino acid sequence of SEQ ID NO: 137, comprising 1, 2, or 3 amino acid modifications. In some embodiments, said F2A element comprises a polynucleotide sequence at least 75%, 80%, 85%, 90%, 95%, 96%, 97%, 98%, 99%, or 100% identical to the polynucleotide sequence of SEQ ID NO: 141. In some embodiments, said F2A element comprises a polynucleotide sequence that encodes the amino acid sequence of SEQ ID NO: 138, or the amino acid sequence of SEQ ID NO: 138, comprising 1, 2, or 3 amino acid modifications. In some embodiments, said F2A element comprises a polynucleotide sequence at least 75%, 80%, 85%, 90%, 95%, 96%, 97%, 98%, 99%, or 100% identical to the polynucleotide sequence of SEQ ID NO: 142.

In some embodiments, said T2A element comprises a polynucleotide sequence that encodes the amino acid sequence of SEQ ID NO: 139, or the amino acid sequence of SEQ ID NO: 139, comprising 1, 2, or 3 amino acid modifications. In some embodiments, said T2A element comprises a polynucleotide sequence at least 75%, 80%, 85%, 90%, 95%, 96%, 97%, 98%, 99%, or 100% identical to the polynucleotide sequence SEQ ID NO: 143. In some embodiments, said T2A element comprises a polynucleotide sequence that encodes the amino acid sequence of SEQ ID NO: 140 or 182, or the amino acid sequence of SEQ ID NO: 140 or 182, comprising 1, 2, or 3 amino acid modifications. In some embodiments, said T2A element comprises a polynucleotide sequence at least 75%, 80%, 85%, 90%, 95%, 96%, 97%, 98%, 99%, or 100% identical to the polynucleotide sequence of SEQ ID NO: 144, 145, or 165.

In some embodiments, said antigen-binding domain comprises: a heavy chain variable region (VH) comprising complementarity determining regions VH CDR1, VH CDR2, and VH CDR3; and a light chain variable region (VL) comprising complementarity determining regions VL CDR1, VL CDR2, and VL CDR3. In some embodiments, said antigen-binding domain comprises an scFv that comprises said VH and said VL operably linked via a first peptide linker.

In some embodiments, said VH comprises the VH CDR1, VH CDR2, and VH CDR3 amino acid sequences set forth in SEQ ID NO: 2. In some embodiments, said VH CDR1 comprises the amino acid sequence of SEQ ID NO: 6; or the amino acid sequence of SEQ ID NO: 6, comprising 1, 2, or 3 amino acid modifications; said VH CDR2 comprises the amino acid sequence of SEQ ID NO: 7; or the amino acid sequence of SEQ ID NO: 7, comprising 1, 2, or 3 amino acid modifications; and said VH CDR3 comprises the amino acid sequence of SEQ ID NO: 8; or the amino acid sequence of SEQ ID NO: 8, comprising 1, 2, or 3 amino acid modifications.

In some embodiments, said VL comprises the VL CDR1, VL CDR2, and VL CDR3 amino acid sequences set forth in SEQ ID NO: 1. In some embodiments, said VL CDR1 comprises the amino acid sequence of SEQ ID NO: 3; or the amino acid sequence of SEQ ID NO: 3, comprising 1, 2, or 3 amino acid modifications; said VL CDR2 comprises the amino acid sequence of SEQ ID NO: 4; or the amino acid sequence of SEQ ID NO: 4, comprising 1, 2, or 3 amino acid modifications; and said VL CDR3 comprises the amino acid sequence of SEQ ID NO: 5; or the amino acid sequence of SEQ ID NO: 5, comprising 1, 2, or 3 amino acid modifications.

In some embodiments, said VH comprises an amino acid sequence at least 95%, 96%, 97%, 98%, 99%, or 100% identical to the amino acid sequence of SEQ ID NO: 2. In some embodiments, said VH is encoded by a polynucleotide sequence at least 75%, 80%, 85%, 90%, 95%, 96%, 97%, 98%, 99%, or 100% identical to the polynucleotide sequence of SEQ ID NO: 20.

In some embodiments, said VL comprises an amino acid sequence at least 95%, 96%, 97%, 98%, 99%, or 100% identical to the amino acid sequence of SEQ ID NO: 1. In some embodiments, said VL is encoded by a polynucleotide sequence at least 75%, 80%, 85%, 90%, 95%, 96%, 97%, 98%, 99%, or 100% identical to the polynucleotide sequence of SEQ ID NO: 19.

In some embodiments, said first peptide linker comprises the amino acid sequence of SEQ ID NO: 9 or SEQ ID NO: 17, or the amino acid sequence of SEQ ID NO: 9 or 17, comprising 1, 2, or 3 amino acid modifications. In some embodiments, said first peptide linker is encoded by a polynucleotide sequence at least 75%, 80%, 85%, 90%, 95%, 96%, 97%, 98%, 99%, or 100% identical to the polynucleotide sequence of SEQ ID NO: 27 or SEQ ID NO: 35. In some embodiments, said first peptide linker is encoded by a polynucleotide sequence at least 75%, 80%, 85%, 90%, 95%, 96%, 97%, 98%, 99%, or 100% identical to the polynucleotide sequence of SEQ ID NO: 27.

In some embodiments, said CAR further comprises a hinge region positioned between said antigen-binding domain and said transmembrane domain of said CAR. In some embodiments, said hinge region comprises the amino acid sequence of SEQ ID NO: 37, 38, or 39, or the amino acid sequence of SEQ ID NO: 37, 38, or 39, comprising 1, 2, or 3 amino acid modifications. In some embodiments, said hinge region is encoded by a polynucleotide sequence at least 75%, 80%, 85%, 90%, 95%, 96%, 97%, 98%, 99%, or 100% identical to the polynucleotide sequence of SEQ ID NO: 40, 41, or 42.

In some embodiments, said transmembrane domain of said CAR comprises the amino acid sequence of SEQ ID NO: 43, 44, or 45, or the amino acid sequence of SEQ ID NO: 43, 44, or 45, comprising 1, 2, or 3 amino acid modifications. In some embodiments, said transmembrane domain of said CAR is encoded by a polynucleotide sequence at least 75%, 80%, 85%, 90%, 95%, 96%, 97%, 98%, 99%, or 100% identical to the polynucleotide sequence of SEQ ID NO: 49, 50, 51, or 52.

In some embodiments, said hinge region and said transmembrane domain together comprise the amino acid sequence of SEQ ID NO: 46, 47, or 48, or the amino acid sequence of SEQ ID NO: 46, 47, or 48, comprising 1, 2, or 3 amino acid modifications. In some embodiments, said hinge region and said transmembrane domain together are encoded by a polynucleotide sequence at least 75%, 80%, 85%, 90%, 95%, 96%, 97%, 98%, 99%, or 100% identical to the polynucleotide sequence of SEQ ID NO: 53, 54, 55, or 56.

In some embodiments, said cytoplasmic domain comprises a primary signaling domain of human CD3ζ, or a functional fragment or functional variant thereof. In some embodiments, said cytoplasmic domain comprises an amino acid sequence at least 95%, 96%, 97%, 98%, 99%, or 100% identical to the amino acid sequence of SEQ ID NO: 60. In some embodiments, said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 67 or 68.

In some embodiments, said cytoplasmic domain comprises a co-stimulatory domain, or functional fragment or variant thereof, of a protein selected from the group consisting of CD28, 4-1BB, OX40, CD2, CD7, CD27, CD30, CD40, CDS, ICAM-1, LFA-1, B7-H3, and ICOS. In some embodiments, said protein is CD28 or 4-1BB.

In some embodiments, said protein is CD28. In some embodiments, said cytoplasmic domain comprises the amino acid sequence of SEQ ID NO: 57 or 58, or the amino acid sequence of SEQ ID NO: 57 or 58, comprising 1, 2, or 3 amino acid modifications. In some embodiments, said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 64 or 65.

In some embodiments, said protein is 4-1BB. In some embodiments, said cytoplasmic domain comprises the amino acid sequence of SEQ ID NO: 59, or the amino acid sequence of SEQ ID NO: 59, comprising 1, 2, or 3 amino acid modifications. In some embodiments, said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 66.

In some embodiments, said cytoplasmic domain comprises the amino acid sequence of SEQ ID NO: 61, 62, or 63, or the amino acid sequence of SEQ ID NO: 61, 62, or 63, comprising 1, 2, or 3 amino acid modifications. In some embodiments, said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 69, 70, or 71.

In some embodiments, said CAR comprises an amino acid sequence at least at least 95%, 96%, 97%, 98%, 99%, or 100% identical to the amino acid sequence of SEQ ID NO: 72, 74, 76, 77, 78, 79, 80, or 81. In some embodiments, said CAR is encoded by a polynucleotide sequence at least 75%, 80%, 85%, 90%, 95%, 96%, 97%, 98%, 99%, or 100% identical to the polynucleotide sequence of SEQ ID NO: 82, 83, 86, 87, 90, 91, 92, 93, 94, or 95.

In some embodiments, said IL-15, or said functional fragment or functional variant thereof, is operably linked to said IL-15Rα, or said functional fragment or functional variant thereof, via a second peptide linker. In some embodiments, said fusion protein comprises an amino acid sequence at least 95%, 96%, 97%, 98%, 99%, or 100% identical to the amino acid sequence of SEQ ID NO: 119, 121, or 180. In some embodiments, said fusion protein is encoded by a polynucleotide sequence at least 75%, 80%, 85%, 90%, 95%, 96%, 97%, 98%, 99%, or 100% identical to the polynucleotide sequence of SEQ ID NO: 126, 127, 130, 131, or 181.

In some embodiments, said marker protein comprises: domain III of HER1, or a functional fragment or functional variant thereof, an N-terminal portion of domain IV of HER1; and a transmembrane domain of CD28, or a functional fragment or functional variant thereof.

In some embodiments, said domain III of HER1, or a functional fragment or functional variant thereof, comprises an amino acid sequence at least 95%, 96%, 97%, 98%, 99%, or 100% identical to the amino acid sequence of SEQ ID NO: 98. In some embodiments, said domain III of HER1, or a functional fragment or functional variant thereof, is encoded by a polynucleotide sequence at least 75%, 80%, 85%, 90%, 95%, 96%, 97%, 98%, 99%, or 100% identical to the polynucleotide sequence of SEQ ID NO: 110 or 164.

In some embodiments, said N-terminal portion of domain IV of HER1 comprises amino acids 1-40, 1-39, 1-38, 1-37, 1-36, 1-35, 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, or 1-10 of SEQ ID NO: 99. In some embodiments, said N-terminal portion of domain IV of HER1 comprises amino acids 1-21 of SEQ ID NO: 99. In some embodiments, said N-terminal portion of domain IV of HER1 comprises the amino acid sequence of SEQ ID NO: 100, or the amino acid sequence of SEQ ID NO: 100, comprising 1, 2, or 3 amino acid modifications. In some embodiments, said N-terminal portion of domain IV of HER1 is encoded by a polynucleotide sequence at least 75%, 80%, 85%, 90%, 95%, 96%, 97%, 98%, 99%, or 100% identical to the polynucleotide sequence of SEQ ID NO: 112.

In some embodiments, said transmembrane region of CD28 comprises the amino acid sequence of SEQ ID NO: 101, or the amino acid sequence of SEQ ID NO: 101, comprising 1, 2, or 3 amino acid modifications. In some embodiments, said transmembrane region of CD28 is encoded by a polynucleotide sequence at least 75%, 80%, 85%, 90%, 95%, 96%, 97%, 98%, 99%, or 100% identical to the polynucleotide sequence of SEQ ID NO: 113.

In some embodiments, said marker protein comprises an amino acid sequence at least 95%, 96%, 97%, 98%, 99%, or 100% identical to the amino acid sequence of SEQ ID NO: 96, 97, 166, or 167. In some embodiments, said marker protein is encoded by a polynucleotide sequence at least 75%, 80%, 85%, 90%, 95%, 96%, 97%, 98%, 99%, or 100% identical to the polynucleotide sequence of SEQ ID NO: 107, 108, 109, 162, 173, or 174.

In some embodiments, said regulatory element comprises a promoter. In some embodiments, said promoter is a human elongation factor 1-alpha (hEF-1α) hybrid promoter. In some embodiments, said promoter comprises a polynucleotide sequence at least 75%, 80%, 85%, 90%, 95%, 96%, 97%, 98%, 99%, or 100% identical to the polynucleotide sequence of SEQ ID NO: 146.

In some embodiments, said vector further comprises a polyA sequence 3′ of said fifth polynucleotide sequence. In some embodiments, said polyA sequence comprises a polynucleotide sequence at least 75%, 80%, 85%, 90%, 95%, 96%, 97%, 98%, 99%, or 100% identical to the polynucleotide sequence of SEQ ID NO: 148.

In another aspect, the instant disclosure provides a recombinant vector comprising a polycistronic expression cassette, wherein said polycistronic expression cassette comprises a transcriptional regulatory element operably linked to a polynucleotide that comprises, from 5′ to 3′: a first polynucleotide sequence that encodes a CAR that comprises an amino acid sequence at least 95%, 96%, 97%, 98%, 99%, or 100% identical to the amino acid sequence of SEQ ID NO: 72 or 74; a second polynucleotide sequence that comprises an F2A element; a third polynucleotide sequence that encodes a fusion protein that comprises an amino acid sequence at least 95%, 96%, 97%, 98%, 99%, or 100% identical to the amino acid sequence of SEQ ID NO: 119, 121, or 180; a fourth polynucleotide sequence that comprises a T2A element; and a fifth polynucleotide sequence that encodes a marker protein that comprises an amino acid sequence at least 95%, 96%, 97%, 98%, 99%, or 100% identical to the amino acid sequence of SEQ ID NO: 96 or 97.

In some embodiments, said F2A element comprises a polynucleotide sequence that encodes the amino acid sequence of SEQ ID NO: 137, or the amino acid sequence of SEQ ID NO: 137, comprising 1, 2, or 3 amino acid modifications. In some embodiments, said F2A element comprises a polynucleotide sequence at least 75%, 80%, 85%, 90%, 95%, 96%, 97%, 98%, 99%, or 100% identical to the polynucleotide sequence of SEQ ID NO: 141. In some embodiments, said F2A element comprises a polynucleotide sequence that encodes the amino acid sequence of SEQ ID NO: 138, or the amino acid sequence of SEQ ID NO: 138, comprising 1, 2, or 3 amino acid modifications. In some embodiments, said F2A element comprises a polynucleotide sequence at least 75%, 80%, 85%, 90%, 95%, 96%, 97%, 98%, 99%, or 100% identical to the polynucleotide sequence of SEQ ID NO: 142.

In some embodiments, said T2A element comprises a polynucleotide sequence that encodes the amino acid sequence of SEQ ID NO: 139, or the amino acid sequence of SEQ ID NO: 139, comprising 1, 2, or 3 amino acid modifications. In some embodiments, said T2A element comprises a polynucleotide sequence at least 75%, 80%, 85%, 90%, 95%, 96%, 97%, 98%, 99%, or 100% identical to the polynucleotide sequence SEQ ID NO: 143. In some embodiments, said T2A element comprises a polynucleotide sequence that encodes the amino acid sequence of SEQ ID NO: 140 or 182, or the amino acid sequence of SEQ ID NO: 140 or 182, comprising 1, 2, or 3 amino acid modifications. In some embodiments, said T2A element comprises a polynucleotide sequence at least 75%, 80%, 85%, 90%, 95%, 96%, 97%, 98%, 99%, or 100% identical to the polynucleotide sequence of SEQ ID NO: 144, 145, or 165.

In another aspect, the instant disclosure provides a recombinant vector comprising a polycistronic expression cassette, wherein said polycistronic expression cassette comprises a transcriptional regulatory element operably linked to a polynucleotide that comprises, from 5′ to 3′: a first polynucleotide sequence at least 75%, 80%, 85%, 90%, 95%, 96%, 97%, 98%, 99%, or 100% identical to the polynucleotide sequence of SEQ ID NO: 82, 83, 86, or 87; a second polynucleotide sequence that comprises an F2A element; a third polynucleotide sequence at least 75%, 80%, 85%, 90%, 95%, 96%, 97%, 98%, 99%, or 100% identical to the polynucleotide sequence of SEQ ID NO: 126, 127, 130, 131, or 181; a fourth polynucleotide sequence that comprises a T2A element; and a fifth polynucleotide sequence at least 75%, 80%, 85%, 90%, 95%, 96%, 97%, 98%, 99%, or 100% identical to the polynucleotide sequence of SEQ ID NO: 107, 108, 109, or 162.

In some embodiments, said F2A element comprises a polynucleotide sequence that encodes the amino acid sequence of SEQ ID NO: 137, or the amino acid sequence of SEQ ID NO: 137, comprising 1, 2, or 3 amino acid modifications. In some embodiments, said F2A element comprises a polynucleotide sequence at least 75%, 80%, 85%, 90%, 95%, 96%, 97%, 98%, 99%, or 100% identical to the polynucleotide sequence of SEQ ID NO: 141. In some embodiments, said F2A element comprises a polynucleotide sequence that encodes the amino acid sequence of SEQ ID NO: 138, or the amino acid sequence of SEQ ID NO: 138, comprising 1, 2, or 3 amino acid modifications. In some embodiments, said F2A element comprises a polynucleotide sequence at least 75%, 80%, 85%, 90%, 95%, 96%, 97%, 98%, 99%, or 100% identical to the polynucleotide sequence of SEQ ID NO: 142.

In some embodiments, said T2A element comprises a polynucleotide sequence that encodes the amino acid sequence of SEQ ID NO: 139, or the amino acid sequence of SEQ ID NO: 139, comprising 1, 2, or 3 amino acid modifications. In some embodiments, said T2A element comprises a polynucleotide sequence at least 75%, 80%, 85%, 90%, 95%, 96%, 97%, 98%, 99%, or 100% identical to the polynucleotide sequence SEQ ID NO: 143. In some embodiments, said T2A element comprises a polynucleotide sequence that encodes the amino acid sequence of SEQ ID NO: 140 or 182, or the amino acid sequence of SEQ ID NO: 140 or 182, comprising 1, 2, or 3 amino acid modifications. In some embodiments, said T2A element comprises a polynucleotide sequence at least 75%, 80%, 85%, 90%, 95%, 96%, 97%, 98%, 99%, or 100% identical to the polynucleotide sequence of SEQ ID NO: 144, 145, or 165.

In some embodiments of the recombinant vectors described herein, the vector further comprises a Left inverted terminal repeat (ITR) and a Right ITR, wherein said Left ITR and said Right ITR flank said polycistronic expression cassette. In some embodiments, the recombinant vector comprises, from 5′ to 3′: said Left ITR; said transcriptional regulatory element; said first polynucleotide sequence; said second polynucleotide sequence; said third polynucleotide sequence; said fourth polynucleotide sequence; said fifth polynucleotide sequence; and said Right ITR.

In another aspect, the instant disclosure provides a recombinant vector comprising a polycistronic expression cassette, wherein said polycistronic expression cassette comprises a transcriptional regulatory element operably linked to a polynucleotide sequence at least 75%, 80%, 85%, 90%, 95%, 96%, 97%, 98%, 99%, or 100% identical to the polynucleotide sequence of SEQ ID NO: 149. In another aspect, the instant disclosure provides a recombinant vector comprising a polycistronic expression cassette, wherein said polycistronic expression cassette comprises a transcriptional regulatory element operably linked to a polynucleotide that encodes an amino acid sequence at least 95%, 96%, 97%, 98%, 99%, or 100% identical to the amino acid sequence of SEQ ID NO: 152.

In some embodiments, any of the recombinant vectors described herein further comprise a Left inverted terminal repeat (ITR) and a Right ITR, wherein said Left ITR and said Right ITR flank said polycistronic expression cassette. In some embodiments, said Left ITR and said Right ITR are ITRs of a DNA transposon selected from the group consisting of a Sleeping Beauty transposon, a piggyBac transposon, TcBuster transposon, and a Tol2 transposon. In some embodiments, said DNA transposon is said Sleeping Beauty transposon.

In some embodiments of the recombinant vectors described herein, said vector is a non-viral vector. In some embodiments, said non-viral vector is a plasmid. In some embodiments of the recombinant vectors described herein, said vector is a viral vector. In some embodiments of the recombinant vectors described herein, said vector is a polynucleotide.

In another aspect, the instant disclosure provides a polynucleotide encoding an amino acid sequence at least 95%, 96%, 97%, 98%, 99%, or 100% identical to the amino acid sequence of SEQ ID NO: 152.

In another aspect, the instant disclosure provides a population of cells that comprise the vector as described herein. In some embodiments, said vector is integrated into the genome of said population of cells.

In another aspect, the instant disclosure provides a population of cells that comprise a polynucleotide encoding an amino acid sequence at least 95%, 96%, 97%, 98%, 99%, or 100% identical to the amino acid sequence of SEQ ID NO: 152. In some embodiments, said polynucleotide is integrated into the genome of said population of cells.

In another aspect, the instant disclosure provides a population of cells that comprise a polypeptide comprising an amino acid sequence encoded by a polynucleotide encoding an amino acid sequence at least 95%, 96%, 97%, 98%, 99%, or 100% identical to the amino acid sequence of SEQ ID NO: 152.

In some embodiments of the populations of cells described herein, the cells comprise a CAR comprising the amino acid sequence of SEQ ID NO: 72, 73, 74, 75, 76, 77, 78, 79, 80, or 81; a fusion protein comprising the amino acid sequence of SEQ ID NO: 119, 120, 121, 122, 180, or 183; and a marker protein comprising the amino acid sequence of SEQ ID NO: 96, 97, 166, or 167. In some embodiments of the populations of cells described herein, the cells comprise a CAR comprising the amino acid sequence of SEQ ID NO: 74; a fusion protein comprising the amino acid sequence of SEQ ID NO: 121; and a marker protein comprising the amino acid sequence of SEQ ID NO: 97. In some embodiments of the populations of cells described herein, the cells comprise a CAR comprising the amino acid sequence of SEQ ID NO: 75; a fusion protein comprising the amino acid sequence of SEQ ID NO: 122; and a marker protein comprising the amino acid sequence of SEQ ID NO: 97.

In some embodiments of the populations of cells described herein, the cells are immune effector cells. In some embodiments, said immune effector cells are selected from the group consisting of T cells, natural killer (NK) cells, B cells, mast cells, and myeloid-derived phagocytes. In some embodiments, said immune effector cells are T cells. In some embodiments, the population of T cells comprise alpha/beta T cells, gamma/delta T cells, or natural killer T (NK-T) cells. In some embodiments, the population of T cells comprise CD4⁺ T cells, CD8⁺ T cells, or both CD4⁺ T cells and CD8⁺ T cells.

In some embodiments of the populations of cells described herein, the cells are ex vivo. In some embodiments of the populations of cells described herein, the cells are human.

In another aspect, the instant disclosure provides a method of producing a population of engineered cells, comprising: introducing into a population of cells a recombinant vector comprising a Left ITR and a Right ITR, wherein said Left ITR and said Right ITR flank said polycistronic expression cassette and culturing said population of cells under conditions wherein said transposase integrates the polycistronic expression cassette into the genome of said population of cells, thereby producing the population of engineered cells. In some embodiments, the recombinant vector comprises, from 5′ to 3′: said Left ITR; said transcriptional regulatory element; said first polynucleotide sequence; said second polynucleotide sequence; said third polynucleotide sequence; said fourth polynucleotide sequence; said fifth polynucleotide sequence; and said Right ITR.

In some embodiments, said Left ITR and said Right ITR are ITRs of a DNA transposon selected from the group consisting of a Sleeping Beauty transposon, a piggyBac transposon, a TcBuster transposon, and a Tol2 transposon. In some embodiments, said DNA transposon is said Sleeping Beauty transposon. In some embodiments, said transposase is a Sleeping Beauty transposase. In some embodiments, said Sleeping Beauty transposase is selected from the group consisting of SB11, SB100X, hSB110, and hSB81. In some embodiments, said Sleeping Beauty transposase is SB11. In some embodiments, said SB11 comprises an amino acid sequence at least 95%, 96%, 97%, 98%, 99%, or 100% identical to the amino acid sequence of SEQ ID NO: 160. In some embodiments, said SB11 is encoded by a polynucleotide sequence at least at least 75%, 80%, 85%, 90%, 95%, 96%, 97%, 98%, 99%, or 100% identical to the polynucleotide sequence of SEQ ID NO: 161. In some embodiments, said polynucleotide encoding said DNA transposase is a DNA vector or an RNA vector.

In some embodiments, said Left ITR comprises a polynucleotide sequence at least 75%, 80%, 85%, 90%, 95%, 96%, 97%, 98%, 99%, or 100% identical to the polynucleotide sequence of SEQ ID NO: 155 or 156; and said Right ITR comprises a polynucleotide sequence at least 75%, 80%, 85%, 90%, 95%, 96%, 97%, 98%, 99%, or 100% identical to the polynucleotide sequence of SEQ ID NO: 157, 159, or 184.

In some embodiments, said recombinant vector, and said DNA transposase or polynucleotide encoding said DNA transposase, are introduced to said population of cells using electro-transfer, calcium phosphate precipitation, lipofection, particle bombardment, microinjection, mechanical deformation by passage through a microfluidic device, or a colloidal dispersion system. In some embodiments, said recombinant vector, and said DNA transposase or polynucleotide encoding said DNA transposase, are introduced to said population of cells using electro-transfer. In some embodiments, said method is completed in less than two days. In some embodiments, said method is completed in 1-2 days. In some embodiments, said method is completed in more than two days.

In some embodiments, said population of cells is cryopreserved and thawed before introduction of said recombinant vector and said DNA transposase or polynucleotide encoding said DNA transposase. In some embodiments, said population of cells is rested before introduction of said recombinant vector and said DNA transposase or polynucleotide encoding said DNA transposase. In some embodiments, said population of cells comprises human ex vivo cells. In some embodiments, said population of cells is not activated ex vivo. In some embodiments, said population of cells comprises T cells.

In another aspect, the instant disclosure provides a method of treating cancer in a subject in need thereof comprising administering to the subject a therapeutically effective amount of a population of cells described herein, thereby treating the cancer.

In another aspect, the instant disclosure provides a method of treating cancer in a subject in need thereof comprising administering to the subject a therapeutically effective amount of a population of engineered cells produced by a method of producing a population of engineered cells described herein, thereby treating the cancer.

In another aspect, the instant disclosure provides a method of treating an autoimmune disease or disorder in a subject in need thereof comprising administering to the subject a therapeutically effective amount of a population of cells described herein, thereby treating the autoimmune disease or disorder.

In another aspect, the instant disclosure provides a method of treating an autoimmune disease or disorder in a subject in need thereof comprising administering to the subject a therapeutically effective amount of a population of engineered cells produced by a method of producing a population of engineered cells described herein, thereby treating the autoimmune disease or disorder.

In some embodiments, any of the polynucleotide sequences described herein (e.g., polynucleotide sequences set forth in Tables 1-7, 10, 11, and 13) may be followed by a stop codon (e.g., TAA, TAG, or TGA) at the 3′ end, with or without an intervening polynucleotide sequence.

### 4. BRIEF DESCRIPTION OF THE DRAWINGS

FIG. 1A is a schematic of the CD19-specific CAR CD19CAR, which incorporates, from N terminus to C terminus, an N-terminal signal sequence; anti-human CD19 VL; peptide linker; anti-human CD19 VH; human CD8α hinge domain; human CD8α transmembrane (TM) domain; human CD28 cytoplasmic domain; and human CD3ζ cytoplasmic domain. FIG. 1B is a schematic of the membrane-bound IL-15/IL-15Rα fusion protein mbIL15, which incorporates, from N terminus to C terminus, an N-terminal signal sequence; human IL-15; linker peptide; and human IL-15Rα. FIG. 1C is a schematic of the marker protein HER1t, which incorporates, from N terminus to C terminus, an N-terminal signal sequence; domain III of human HER1; truncated domain IV of human HER1; peptide linker; and human CD28 TM domain.

FIG. 2 is a schematic diagram depicting double transposition (dTp) and single transposition (sTp) approaches using an SB11 transposon/transposase system to generate CAR-T cells expressing CD19CAR, mbIL15, and HER1t.

FIGS. 3A-3E are graphs showing percent cell viability (FIG. 3A), CD3 frequency (FIG. 3B), CD19CAR expression (FIG. 3C), mbIL15 expression (FIG. 3D), and HER1t expression (FIG. 3E) on Day 1 post-electroporation of T cell-enriched cryopreserved cell product from three separate donors with no plasmid (Negative Control), 1:1 combination of Plasmids DP1 and DP2 (dTp Control), or Plasmids A-F.

FIGS. 4A-4F are sets of 2-parameter flow plots showing transgene co-expression as assessed on Day 1 and at the end of AaPC stimulation cycles (“Stims”) 1, 2, 3, and 4 for dTp Control-modified T cells from Donor A. Percent cells are shown in each quadrant. Specifically, FIG. 4A is a set of flow plots showing CD19CAR expression vs. CD3 expression. FIG. 4B is a set of flow plots showing HER1t expression vs. CD3 expression. FIG. 4C is a set of flow plots showing mbIL15 expression vs. CD3 expression. FIG. 4D is a set of flow plots showing CD19CAR expression vs. HER1t expression. FIG. 4E is a set of flow plots showing CD19CAR expression vs. mbIL15 expression. FIG. 4F is a set of flow plots showing HER1t expression vs. mbIL15 expression. The flow plots of FIGS. 4D-4F show transgene expression on CD3⁺-gated cells.

FIGS. 5A-5F are sets of 2-parameter flow plots showing transgene co-expression as assessed on Day 1 and at the end of Stims 1, 2, 3, and 4 for Plasmid A-modified T cells from Donor A. Percent cells are shown in each quadrant. Specifically, FIG. 5A is a set of flow plots showing CD19CAR expression vs. CD3 expression. FIG. 5B is a set of flow plots showing HER1t expression vs. CD3 expression. FIG. 5C is a set of flow plots showing mbIL15 expression vs. CD3 expression. FIG. 5D is a set of flow plots showing CD19CAR expression vs. HER1t expression. FIG. 5E is a set of flow plots showing CD19CAR expression vs. mbIL15 expression. FIG. 5F is a set of flow plots showing HER1t expression vs. mbIL15 expression. The flow plots of FIGS. 5D-5F show transgene expression on CD3⁺-gated cells.

FIGS. 6A-6F are sets of 2-parameter flow plots showing transgene co-expression as assessed on Day 1 and at the end of Stims 1, 2, 3, and 4 for Plasmid B-modified T cells from Donor A. Percent cells are shown in each quadrant. Specifically, FIG. 6A is a set of flow plots showing CD19CAR expression vs. CD3 expression. FIG. 6B is a set of flow plots showing HER1t expression vs. CD3 expression. FIG. 6C is a set of flow plots showing mbIL15 expression vs. CD3 expression. FIG. 6D is a set of flow plots showing CD19CAR expression vs. HER1t expression. FIG. 6E is a set of flow plots showing CD19CAR expression vs. mbIL15 expression. FIG. 6F is a set of flow plots showing HER1t expression vs. mbIL15 expression. The flow plots of FIGS. 6D-6F show transgene expression on CD3⁺-gated cells.

FIGS. 7A-7F are sets of 2-parameter flow plots showing transgene co-expression as assessed on Day 1 and at the end of Stims 1, 2, 3, and 4 for Plasmid C-modified T cells from Donor A. Percent cells are shown in each quadrant. Specifically, FIG. 7A is a set of flow plots showing CD19CAR expression vs. CD3 expression. FIG. 7B is a set of flow plots showing HER1t expression vs. CD3 expression. FIG. 7C is a set of flow plots showing mbIL15 expression vs. CD3 expression. FIG. 7D is a set of flow plots showing CD19CAR expression vs. HER1t expression. FIG. 7E is a set of flow plots showing CD19CAR expression vs. mbIL15 expression. FIG. 7F is a set of flow plots showing HER1t expression vs. mbIL15 expression. The flow plots of FIGS. 7D-7F show transgene expression on CD3⁺-gated cells.

FIGS. 8A-8F are sets of 2-parameter flow plots showing transgene co-expression as assessed on Day 1 and at the end of Stims 1, 2, 3, and 4 for Plasmid D-modified T cells from Donor A. Percent cells are shown in each quadrant. Specifically, FIG. 8A is a set of flow plots showing CD19CAR expression vs. CD3 expression. FIG. 8B is a set of flow plots showing HER1t expression vs. CD3 expression. FIG. 8C is a set of flow plots showing mbIL15 expression vs. CD3 expression. FIG. 8D is a set of flow plots showing CD19CAR expression vs. HER1t expression. FIG. 8E is a set of flow plots showing CD19CAR expression vs. mbIL15 expression. FIG. 8F is a set of flow plots showing HER1t expression vs. mbIL15 expression. The flow plots of FIGS. 8D-8F show transgene expression on CD3⁺-gated cells.

FIGS. 9A-9F are sets of 2-parameter flow plots showing transgene co-expression as assessed on Day 1 and at the end of Stims 1, 2, 3, and 4 for Plasmid E-modified T cells from Donor A. Percent cells are shown in each quadrant. Specifically, FIG. 9A is a set of flow plots showing CD19CAR expression vs. CD3 expression. FIG. 9B is a set of flow plots showing HER1t expression vs. CD3 expression. FIG. 9C is a set of flow plots showing mbIL15 expression vs. CD3 expression. FIG. 9D is a set of flow plots showing CD19CAR expression vs. HER1t expression. FIG. 9E is a set of flow plots showing CD19CAR expression vs. mbIL15 expression. FIG. 9F is a set of flow plots showing HER1t expression vs. mbIL15 expression. The flow plots of FIGS. 9D-9F show transgene expression on CD3⁺-gated cells.

FIGS. 10A-10F are sets of 2-parameter flow plots showing transgene co-expression as assessed on Day 1 and at the end of Stims 1, 2, 3, and 4 for Plasmid F-modified T cells from Donor A. Percent cells are shown in each quadrant. Specifically, FIG. 10A is a set of flow plots showing CD19CAR expression vs. CD3 expression. FIG. 10B is a set of flow plots showing HER1t expression vs. CD3 expression. FIG. 10C is a set of flow plots showing mbIL15 expression vs. CD3 expression. FIG. 10D is a set of flow plots showing CD19CAR expression vs. HER1t expression. FIG. 10E is a set of flow plots showing CD19CAR expression vs. mbIL15 expression. FIG. 10F is a set of flow plots showing HER1t expression vs. mbIL15 expression. The flow plots of FIGS. 10D-10F show transgene expression on CD3⁺-gated cells.

FIGS. 11A-11C are bar graphs showing transgene expression as assessed on Day 1 and at the end of Stims 1, 2, 3, and 4 for CD3-enriched T cells from Donor A transfected with dTp Control or Plasmids A-F. Bar graphs shows expression (CD3⁺-gated) of CD19CAR (FIG. 11A), mbIL15 (FIG. 11B), and HER1t (FIG. 11C).

FIGS. 12A-12C are images of Western blots confirming expression of CD19CAR (FIG. 12A), mbIL15 (FIG. 12B), and HER1t (FIG. 12C) in cell lysates from ex vivo expanded CD19CAR-mbIL15-CAR-T cells. Cells from one normal donor is shown, except where an additional donor was indicated (in samples labeled Z).

FIGS. 13A-13C are graphs showing inferred cell count as assessed on Day 1 and at the end of Stims 1, 2, 3, and 4 for T cell-enriched starting product from Donor A transfected with dTp Control or Plasmids A-F and ex vivo expanded. The inferred cell counts for CD3-gated CD19CAR⁺ (FIG. 13A), mbIL15⁺ (FIG. 13B), and HER1t⁺ (FIG. 13C) were plotted over time.

FIGS. 14A-14H are graphs showing cytotoxicity of ex vivo expanded CD19 specific T cells either not transfected (Negative Control) (FIG. 14A) or transfected with dTp Control (FIG. 14B), Plasmid A (FIG. 14C), Plasmid B (FIG. 14D), Plasmid C (FIG. 14E), Plasmid D (FIG. 14F), Plasmid E (FIG. 14G), and Plasmid F (FIG. 14H), as determined by a chromium release assay against CD19⁺ (Daudi β2M, NALM-6, and CD19 EL-4) and CD19^{neg} (parental EL-4) target cells at different effector-to-target (E:T) ratios by measuring lysis of radiolabeled (⁵¹Cr) target cells. Mean±standard deviation (SD) percent lysis of triplicate wells of several E:T is shown for cells derived from Donor A. Error bars represent the SD and may be obscured by the symbols.

FIG. 15 is a graph showing antibody-dependent cellular cytotoxicity (ADCC) of ex vivo expanded CD19CAR-mbIL15-HER1t T cells. The genetically modified T cells served as targets in a chromium release assay in the presence of cetuximab (EGFR-specific antibody) or rituximab (CD20-specific antibody; negative control) using Fc receptor-expressing NK cells as effectors. Mock transfected (No DNA) T cells were used as a negative control. Data for Donor A at a 40:1 E:T ratio are shown. Bars represent means values of lysis of gene-modified T cells normalized to maximum NK cell percent lysis.

FIG. 16 is a graph showing the transgene copy number of ex vivo expanded CD19CAR-mbIL15-HER1t T cells from Donor A transfected with the double-transposon control or test plasmids (dTp Control or Plasmids A-F, respectively), Mock transfected CD3 (no DNA negative control), CD19CAR⁺ Jurkat cells (positive control for CD19CAR), mbIL15⁺ Jurkat cells (positive control for mbIL15), or CD19CAR⁺HER1t⁺ T cells (positive control for HER1t). Copy number was assessed using ddPCR, in quintuplicate for each sample, and normalized to the human reference gene EIF2C1.

FIGS. 17A-17C are graphs showing inferred cell count as assessed on Day 1 and at the end of Stims 1, 2, 3, and 4 for T cell-enriched products electroporated with dTp Control (FIG. 17A), Plasmid A (FIG. 17B), and Plasmid D (FIG. 17C) that were ex vivo expanded via co-culture on irradiated Clone 9 AaPCs. Expansion of total cells, CD3⁺, CD3⁺-gated CD19CAR⁺, and CD3⁺-gated HER1t⁺ over time is plotted and shown as mean±SD of multiple donor samples pooled from multiple experiments. Error bars represent the SD and may be obscured by the symbols.

FIGS. 18A-18C are bar graphs showing percent transgene sub-population heterogeneity (CD19CAR⁺HER1t^{neg}, CD19CAR⁺HER1t⁺, CD19CAR^{neg}HER1t⁺, CD19CAR^{neg}HER1t^{neg}) plotted for 18-hour post-electroporation (Day 1) and Stim 4 timepoints for T cell-enriched products electroporated with dTp Control (FIG. 18A), Plasmid A (FIG. 18B), and Plasmid D (FIG. 18C) that were ex vivo expanded via co-culture on irradiated Clone 9 AaPCs. Data are shown as mean±SD of multiple donor samples pooled from multiple experiments.

FIGS. 19A-19C are graphs showing cytotoxicity of ex vivo expanded CD19 specific T cells transfected with dTp Control (FIG. 19A), Plasmid A (FIG. 19B), or Plasmid D (FIG. 19C), as determined by a chromium release assay against CD19+(Daudi β2M, NALM-6, and CD19 EL-4) and CD19^{neg} (parental EL-4) target cells at different effector-to-target (E:T) ratios by measuring lysis of radiolabeled (⁵¹Cr) target cells. Mean±SD percent lysis is shown for multiple donor samples pooled from multiple experiments. Error bars represent the SD and may be obscured by the symbols.

FIG. 20 is a graph showing antibody-dependent cellular cytotoxicity (ADCC) of ex vivo expanded CD19CAR-mbIL15-HER1t T cells. The genetically modified T cells served as targets in a chromium release assay in the presence of cetuximab (EGFR-specific antibody) or rituximab (CD20-specific antibody; negative control) using Fc receptor-expressing NK cells as effectors. Mean±SD percent lysis at a 40:1 E:T ratio are shown for multiple donors pooled from multiple experiments. Bars represent means values of lysis of gene-modified T cells normalized to maximum NK cell percent lysis.

FIG. 21 is a graph showing the transgene copy number of ex vivo expanded CD19CAR-mbIL15-HER1t T cells transfected with dTp Control, Plasmid A, or Plasmid D, or Mock transfected CD3 (no DNA negative control). Copy number was assessed using ddPCR, in triplicate for each sample, and normalized to the human reference gene EIF2C1. Data are shown as mean±SD transgene copies per cell and represent multiple donor samples pooled from multiple experiments.

FIGS. 22A-22C are sets of 2-parameter flow plots showing transgene co-expression as assessed for Mock PBMC, dTp Control (P, 5e6), Plasmid A (P, 5e6), and Plasmid A (T, 1e6)/Plasmid A (T, 0.5e6), as defined in Example 4. Percent cells are shown in each quadrant. Specifically, FIG. 22A is a set of flow plots showing CD19CAR expression vs. CD3 expression. FIG. 22B is a set of flow plots showing CD19CAR expression vs. HER1t expression. FIG. 22C is a set of flow plots showing HER1t expression vs. mbIL15 expression. Gating strategy: lymphocytes>singlets>viable>CD3⁺ events.

FIGS. 23A-23C are sets of 2-parameter flow plots showing transgene co-expression as assessed for cells resulting from ex vivo expansion of Mock PBMC, dTp Control (P, 5e6), and Plasmid A (P, 5e6). Percent cells are shown in each quadrant. Specifically, FIG. 23A is a set of flow plots showing CD19CAR expression vs. CD3 expression. FIG. 23B is a set of flow plots showing CD19CAR expression vs. HER1t expression. FIG. 23C is a set of flow plots showing HER1t expression vs. mbIL15 expression. Gating strategy: lymphocytes>singlets>viable>CD3⁺ events.

FIGS. 24A-24G are graphs showing tumor flux over time for NOD.Cg-Prkdc^{scid} Il2rg^{tmIWjl}/SzJ (NSG) mice that were intravenously injected with 1.5×10⁴ CD19⁺NALM-6 leukemia cells expressing firefly luciferase (fLUC) and subsequently either left untreated (Tumor Only; FIG. 24A) or treated with Mock PBMC (FIG. 24B), Mock CD3 (FIG. 24C), dTp Control (P, 5e6) (FIG. 24D), Plasmid A (P, 5e6) (FIG. 24E), Plasmid A (T, 1e6) (FIG. 24F), or Plasmid A (T, 0.5e6) (FIG. 24G) RPM T cells on Day 7. The tumor flux over time is presented for each treatment group, with each line representing an individual animal. Dotted line represents the “2×background” threshold for determining disease-free mice.

FIG. 25 is a scatterplot showing tumor flux of individual mice at the final BLI before mortality or euthanasia. Bars represent the geometric mean and SD; significance was determined by one-way ANOVA (Dunnett post-test). Error bars represent the SD and may be obscured by the symbols.

FIGS. 26A-26C are Kaplan-Meier survival curves showing overall survival (OS) for each mouse treatment group. Specifically, FIG. 26A is a survival curve for the Tumor Only treatment group (Group A). FIG. 26B is a survival curve for the Mock PBMC (Group B), dTp Control (Group D), and Plasmid A (P, 5e6) (Group E) treatment groups. FIG. 26C is a survival curve for the Mock CD3 (Group C), Plasmid A (T, 1e6) (Group F), and Plasmid A (T, 0.5e6) (Group G) treatment groups.

FIGS. 27A-27C are Kaplan-Meier survival curves showing xGvHD-free survival for each mouse treatment group. The xGvHD-free survival analysis censored mice that died with low tumor burden (i.e., total flux<1×10⁸ p/s) with mortality likely ascribed to xGvHD. Specifically, FIG. 27A is a survival curve for the Tumor Only treatment group (Group A). FIG. 27B is a survival curve for the Mock PBMC (Group B), dTp Control (Group D), and Plasmid A (P, 5e6) (Group E) treatment groups. FIG. 27C is a survival curve for the Mock CD3 (Group C), Plasmid A (T, 1e6) (Group F), and Plasmid A (T, 0.5e6) (Group G) treatment groups.

FIGS. 28A-28C are bar graphs showing CD3⁺ frequency as a percent of viable CD45+cells in peripheral blood (PB) (FIG. 28A), bone marrow (BM), (FIG. 28B), and spleen (FIG. 28C) for each of the Tumor Only (Group A), Mock PBMC (Group B), Mock CD3 (Group C), dTp Control (Group D), Plasmid A (P, 5e6) (Group E), Plasmid A (T, 1e6) (Group F), and Plasmid A (T, 0.5e6) (Group G) treatment groups. Cells were co-stained with antibodies including anti-CD45 and anti-CD3, followed by flow cytometric analysis. Circles represent individual mice, and bars depict mean and range.

FIG. 29A is a set of representative 2-parameter flow plots showing expression of CD19CAR vs. CD3 in cells from peripheral blood from moribund mice or mice at the end of study in each of the seven treatment groups. Cells were co-stained with antibodies including anti-CD3, anti-CD19CAR, anti-HER1t, and anti-IL-15, followed by flow cytometric analysis. Flow plots were gated on singlets, viable hCD45+, and CD3⁺ events to analyze respective transgene frequencies. Percent cells are displayed in each gate. FIGS. 29B-29D are bar graphs showing CD19CAR⁺CD3⁺ frequency as a percentage of viable CD45⁺CD3⁺ cells in peripheral blood (PB) (FIG. 29B), bone marrow (BM), (FIG. 29C), and spleen (FIG. 29D) for each of the Mock PBMC (Group B), Mock CD3 (Group C), dTp Control (Group D), Plasmid A (P, 5e6) (Group E), Plasmid A (T, 1e6) (Group F), and Plasmid A (T, 0.5e6) (Group G) treatment groups. Due to the absence of CD3 engraftment in Tumor Only (Group A) mice, this group was excluded from presentation. Circles represent individual mice, and bars depict mean and range. Error bars represent the SD and may be obscured by the symbols.

FIG. 30 is a set of representative 2-parameter flow plots showing expression of CD19CAR vs. HER1t in cells from peripheral blood from moribund mice or mice at the end of study in each of the seven treatment groups. Cells were co-stained with antibodies including anti-CD3, anti-CD19CAR, anti-HER1t, and anti-IL-15, followed by flow cytometric analysis. Displayed flow plots were gated on singlets, viable hCD45⁺, and CD3⁺ events. Percent cells are shown in each quadrant.

FIG. 31 is a set of representative 2-parameter flow plots showing expression of HER1t vs. mbIL15 in cells from peripheral blood from moribund mice or mice at the end of study in each of the seven treatment groups. Cells were co-stained with antibodies including anti-CD3, anti-CD19CAR, anti-HER1t, and anti-IL-15, followed by flow cytometric analysis. Displayed flow plots were gated on singlets, viable hCD45⁺, and CD3⁺ events. Percent cells are shown in each quadrant.

FIGS. 32A and 32B are sets of representative 2-parameter flow plots showing expression of CD45RO vs. CCR7 (FIG. 32A) or CD45RO vs. CD27 (FIG. 32B) in cells from peripheral blood from moribund mice or mice at the end of study in each of the dTp Control (P, 5e6) (Group D), Plasmid A (P, 5e6) (Group E), Plasmid A (T, 1e6) (Group F), and Plasmid A (T, 0.5e6) (Group G) treatment groups. Cells were co-stained with antibodies including anti-CD3, anti-CD19CAR, anti-CD45RO, anti-CCR7, and anti-CD27, followed by flow cytometric analysis. Displayed flow plots were gated on Singlets, viable hCD45⁺, and CD3⁺CD19CAR⁺ events.

FIGS. 33A and 33B are bar graphs representing the data shown in FIGS. 32A and 32B, respectively. Circles represent individual mice, and floating bars depict minimum and maximum values, with the line representing the mean.

### 5. DETAILED DESCRIPTION

The instant disclosure provides recombinant polycistronic nucleic acid vectors comprising at least three cistrons, wherein from 5′ to 3′ the first cistron encodes an anti-CD19 chimeric antigen receptor (CAR) (e.g., CD19CAR), the second cistron encodes a fusion protein that comprises IL-15 and IL-15Rα (e.g., mbIL15), or a functional fragment or functional variant thereof, and the third cistron encodes a marker protein (e.g., HER1t); and wherein the first and second cistrons are separated by a polynucleotide sequence that comprises an F2A element and the second cistron and third cistrons are separated by a polynucleotide sequence that comprises a T2A element. Also provided are immune effector cells comprising these vectors, immune effector cells engineered ex vivo utilizing the vectors to express the three proteins encoded by the vectors, pharmaceutical compositions comprising these vectors or engineered immune effector cells made utilizing these vectors, and methods of treating a subject using these vectors or engineered immune effector cells made utilizing these vectors.

The polycistronic vectors described herein are particularly useful in methods of manufacturing populations of engineered cells (e.g., immune effector cells) that are substantially homogeneous compared to the prior art systems that utilized at least two vectors for the expression of three proteins. It has been further shown, that, surprisingly, the 5′ to 3′ order of the cistrons, i.e., 5′-anti-CD19 CAR-F2A element-IL-15/IL-15Rα fusion-T2A element-marker protein-3′, provides superior expression of the three protein coding polynucleotide sequences, i.e., anti-CD19 CAR, IL-15/IL-15Rα fusion, and marker protein, on the surface of T cells, compared to alternative orientations.

### 5.1 Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an,” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

As used herein, the terms “about” and “approximately,” when used to modify a numeric value or numeric range, indicate that deviations of 5% to 10% above (e.g., up to 5% to 10% above) and 5% to 10% below (e.g., up to 5% to 10% below) the value or range remain within the intended meaning of the recited value or range.

As used herein, the term “cistron” refers to a polynucleotide sequence from which a transgene product can be produced.

As used herein, the term “polycistronic vector” refers to a polynucleotide vector that comprises a polycistronic expression cassette.

As used herein, the term “polycistronic expression cassette” refers to a polynucleotide sequence wherein the expression of three or more transgenes is regulated by common transcriptional regulatory elements (e.g., a common promoter) and can simultaneously express three or more separate proteins from the same mRNA. Exemplary polycistronic vectors, without limitation, include tricistronic vectors (containing three cistrons) and tetracistronic vectors (containing four cistrons).

As used herein, the term “transcriptional regulatory element” refers to a polynucleotide sequence that mediates regulation of transcription of another polynucleotide sequence. Exemplary transcriptional regulatory elements include, but are not limited to, promoters and enhancers.

As used herein, the term “F2A element” refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 141 or 142; (ii) encodes the amino acid sequence of SEQ ID NO: 137 or 138; or (iii) encodes the amino acid sequence of SEQ ID NO: 137 or 138, comprising 1, 2, or 3 amino acid modifications. In some embodiments, when positioned in a vector between a first polynucleotide sequence encoding a first protein and a second polynucleotide sequence encoding a second protein, the F2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the F2A element, e.g., such that the penultimate residue (e.g., glycine) becomes the C-terminal residue of the first protein and the ultimate residue (e.g., proline) becomes the N-terminal residue of the second protein. In some embodiments, the F2A element additionally comprises, at its 5′ end, a polynucleotide sequence that encodes a furin cleavage site, e.g., RAKR (SEQ ID NO: 187).

As used herein, the term “T2A element” refers to a refers to a polynucleotide that (i) comprises a polynucleotide sequence at least 75%, 80%, 85%, 90%, 95%, 96%, 97%, 98%, 99%, or 100% identical to the polynucleotide sequence of SEQ ID NO: 143, 144, 145, or 165; (ii) encodes the amino acid sequence of SEQ ID NO: 139, 140, or 182; or (iii) encodes the amino acid sequence of SEQ ID NO: 139, 140, or 182, comprising 1, 2, or 3 amino acid modifications. In some embodiments, when positioned in a vector between a first polynucleotide sequence encoding a first protein and a second polynucleotide sequence encoding a second protein, the T2A element is capable of mediating the translation of the first polynucleotide sequence and the second polynucleotide sequence as two distinct polypeptides from the same mRNA molecule by preventing the synthesis of a peptide bond, e.g., between the penultimate residue (e.g., glycine) and the ultimate residue (e.g., proline) at the C terminus of the translation product of the T2A element, e.g., such that the penultimate residue (e.g., glycine) becomes the C-terminal residue of the first protein and the ultimate residue (e.g., proline) becomes the N-terminal residue of the second protein. In some embodiments, the T2A element additionally comprises, at its 5′ end, a polynucleotide sequence that encodes a furin cleavage site, e.g., RAKR (SEQ ID NO: 187).

As used herein, the terms “inverted terminal repeat,” “ITR,” “inverted repeat/direct repeat,” and “IR/DR” are used interchangeably and refer to a polynucleotide sequence, e.g., of about 230 nucleotides (e.g., 220, 221, 222, 223, 224, 225, 226, 227, 228, 229, 230, 231, 232, 233, 234, 235, 236, 237, 238, 239, or 240 nucleotides), flanking (e.g., with or without an intervening polynucleotide sequence) one end of an expression cassette (e.g., a polycistronic expression cassette) that can be cleaved by a transposase polypeptide when used in combination with a corresponding, e.g., reverse-complementary (e.g., perfectly or imperfectly reverse-complementary) polynucleotide sequence, e.g., of about 230 nucleotides (e.g., 220, 221, 222, 223, 224, 225, 226, 227, 228, 229, 230, 231, 232, 233, 234, 235, 236, 237, 238, 239, or 240 nucleotides), flanking (e.g., with or without an intervening polynucleotide sequence) the opposite end of the expression cassette (e.g., a polycistronic expression cassette) (e.g., as described in Cui et al., J. Mol. Biol. 2002; 318(5):1221-35, the contents of which are incorporated by reference in their entirety herein). In some embodiments, an ITR, e.g., an ITR of a DNA transposon (e.g., a Sleeping Beauty transposon, a piggyBac transposon, a TcBuster transposon, and a Tol2 transposon) contains two direct repeats (“DRs”), e.g., imperfect direct repeats, e.g., of about 30 nucleotides (e.g., 25, 26, 27, 28, 29, 30, 31, 32, 33, 34 or 35 nucleotides), located at each end of the ITR. The terms “ITR” and “DR,” when used in reference to a single- or double-stranded DNA vector, refer to the DNA sequence of the sense strand. A transposase polypeptide may recognize the sense strand and/or the antisense strand of DNA.

As used herein, the term “Left ITR,” when used in reference to a linear single- or double-stranded DNA vector, refers to the ITR positioned 5′ of the polycistronic expression cassette. As used herein, the term “Right ITR,” when used in reference to a linear single- or double-stranded DNA vector, refers to the ITR positioned 3′ of the polycistronic expression cassette. When a circular vector is used, the Left ITR is closer to the 5′ end of the polycistronic expression cassette than the Right ITR, and the Right ITR is closer to the 3′ end of the polycistronic expression cassette than the Left ITR.

As used herein, the term “operably linked” refers to a linkage of polynucleotide sequence elements or amino acid sequence elements in a functional relationship. For example, a polynucleotide sequence is operably linked when it is placed into a functional relationship with another polynucleotide sequence. In some embodiments, a transcription regulatory polynucleotide sequence e.g., a promoter, enhancer, or other expression control element is operably-linked to a polynucleotide sequence that encodes a protein if it affects the transcription of the polynucleotide sequence that encodes the protein.

The term “polynucleotide” as used herein refers to a polymer of DNA or RNA. The polynucleotide sequence can be single-stranded or double-stranded; contain natural, non-natural, or altered nucleotides; and contain a natural, non-natural, or altered internucleotide linkage, such as a phosphoroamidate linkage or a phosphorothioate linkage, instead of the phosphodiester found between the nucleotides of an unmodified polynucleotide sequence. Polynucleotide sequences include, but are not limited to, all polynucleotide sequences which are obtained by any means available in the art, including, without limitation, recombinant means, e.g., the cloning of polynucleotide sequences from a recombinant library or a cell genome, using ordinary cloning technology and polymerase chain reaction, and the like, and by synthetic means.

The terms “amino acid sequence” and “polypeptide” as used interchangeably herein and refer to a polymer of amino acids connected by one or more peptide bonds.

The term “functional variant” as used herein in reference to a protein or polypeptide refers to a protein that comprises at least one amino acid modification (e.g., a substitution, deletion, addition) compared to the amino acid sequence of a reference protein, that retains at least one particular function. In some embodiments, the reference protein is a wild type protein. For example, a functional variant of an IL-2 protein can refer to an IL-2 protein comprising an amino acid substitution compared to a wild type IL-2 protein that retains the ability to bind the intermediate affinity IL-2 receptor but abrogates the ability of the protein to bind the high affinity IL-2 receptor. Not all functions of the reference wild type protein need be retained by the functional variant of the protein. In some instances, one or more functions are selectively reduced or eliminated.

The term “functional fragment” as used herein in reference to a protein or polypeptide refers to a fragment of a reference protein that retains at least one particular function. For example, a functional fragment of an anti-HER2 antibody can refer to a fragment of the anti-HER2 antibody that retains the ability to specifically bind the HER2 antigen. Not all functions of the reference protein need be retained by a functional fragment of the protein. In some instances, one or more functions are selectively reduced or eliminated.

As used herein, the term “modification,” with reference to a polynucleotide sequence, refers to a polynucleotide sequence that comprises at least one substitution, alteration, inversion, addition, or deletion of nucleotide compared to a reference polynucleotide sequence. As used herein, the term “modification,” with reference to an amino acid sequence refers to an amino acid sequence that comprises at least one substitution, alteration, inversion, addition, or deletion of an amino acid residue compared to a reference amino acid sequence.

As used herein, the term “derived from,” with reference to a polynucleotide sequence refers to a polynucleotide sequence that has at least 85% sequence identity to a reference naturally occurring nucleic acid sequence from which it is derived. The term “derived from,” with reference to an amino acid sequence refers to an amino acid sequence that has at least 85% sequence identity to a reference naturally occurring amino acid sequence from which it is derived. The term “derived from” as used herein does not denote any specific process or method for obtaining the polynucleotide or amino acid sequence. For example, the polynucleotide or amino acid sequence can be chemically synthesized.

As used herein, the terms “antibody” and “antibodies” include full-length antibodies, antigen-binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), and antigen-binding fragments of any of the above, and conjugates or fusion proteins comprising any of the above. In certain embodiments, antibodies described herein refer to polyclonal antibody populations. Antibodies can be of any type (e.g., IgG, IgE, IgM, IgD, IgA or IgY), any class (e.g., IgG₁, IgG₂, IgG₃, IgG₄, IgA₁ or IgA₂), or any subclass (e.g., IgG₂ₐ or IgG_{2b}) of immunoglobulin molecule. In certain embodiments, antibodies described herein are IgG antibodies, or a class (e.g., human IgG₁ or IgG₄) or subclass thereof. In a specific embodiment, the antibody is a humanized monoclonal antibody. In another specific embodiment, the antibody is a human monoclonal antibody.

As used herein, the terms “VH region” and “VL region” refer, respectively, to single antibody heavy and light chain variable regions, comprising FR (Framework Regions) 1, 2, 3 and 4 and CDR (Complementarity Determining Regions) 1, 2 and 3 (see Kabat et al., (1991) Sequences of Proteins of Immunological Interest (NIH Publication No. 91-3242, Bethesda), which is herein incorporated by reference in its entirety).

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), all of which are herein incorporated by reference in their entireties. Unless otherwise specified, the term “CDR” is a CDR as defined by Kabat et al., J. Biol. Chem. 252, 6609-6616 (1977) and Kabat et al., Sequences of protein of immunological interest. (1991).

As used herein, the term “framework (FR) amino acid residues” refers to those amino acids in the framework region of an antibody variable region. The term “framework region” or “FR region” as used herein, includes the amino acid residues that are part of the variable region, but are not part of the CDRs (e.g., using the Kabat definition of CDRs).

As used herein, the terms “variable region” refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

As used herein, the terms “constant region” and “constant domain” are interchangeable and are common in the art. The constant region is an antibody portion, e.g., a carboxyl terminal portion of a light and/or heavy chain which is not directly involved in binding of an antibody to antigen but which can exhibit various effector functions, such as interaction with an Fc receptor (e.g., Fc gamma receptor). The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

As used herein, the term “EU numbering system” refers to the EU numbering convention for the constant regions of an antibody, as described in Edelman, G. M. et al., Proc. Natl. Acad. USA, 63, 78-85 (1969) and Kabat et al, Sequences of Proteins of Immunological Interest, U.S. Dept. Health and Human Services, 5th edition, 1991, each of which is herein incorporated by reference in its entirety.

As used herein, the term “specifically binds” refers to molecules that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, ID), or other assays known in the art. In a specific embodiment, molecules that specifically bind to an antigen bind to the antigen with a KA that is at least 2 logs (e.g., factors of 10), 2.5 logs, 3 logs, 4 logs or greater than the KA when the molecules bind non-specifically to another antigen. The skilled worker will appreciate that an antibody, as described herein, can specifically bind to more than one antigen (e.g., via different regions of the antibody molecule).

As used herein, the term “linked to” refers to covalent or noncovalent binding between two molecules or moieties. The skilled worker will appreciate that when a first molecule or moiety is linked to a second molecule or moiety, the linkage need not be direct, but instead, can be via an intervening molecule or moiety. For example, when a heavy chain variable region of a full-length antibody is linked to a ligand-binding moiety, the ligand-binding moiety can bind a constant region (e.g., a heavy chain constant region) of the full-length antibody (e.g., via a peptide bond), rather than bind directly to the heavy chain variable region.

As used herein, the term “chimeric antigen receptor” or “CAR” refers to transmembrane proteins that comprise an antigen-binding domain, operably linked to a transmembrane domain, operably linked to a cytoplasmic domain that comprises at least one intracellular signaling domain. CARs can be expressed on the surface of a host cell (e.g., an immune effector cell) in order to mediate activation upon binding to the target antigen in vivo. In some embodiments, the CAR specifically binds CD19. In some embodiments, the CAR specifically binds human CD19 (hCD19).

As used herein, the term “CD19” (also known as B lymphocyte antigen CD19, cluster of differentiation 19, and B lymphocyte surface antigen B4) refers to a protein that in humans is encoded by the CD19 gene. As used herein, the term “human CD19” or hCD19 refers to a CD19 protein encoded by a human CD19 gene (e.g., a wild-type human CD19 gene). Exemplary wild-type human CD19 proteins are provided by GenBank™ accession numbers AAB60697.1, AAA69966.1, and BAB60954.1.

As used herein, the term “extracellular” refers to the portion or portions of a transmembrane protein that are located outside of a cell. In some embodiments, the transmembrane protein is a recombinant transmembrane protein. In some embodiments, the recombinant transmembrane protein is a CAR.

As used herein, the term “antigen-binding domain” with respect to a CAR refers to a domain of the CAR that comprises any suitable antibody- or non-antibody-based molecule that specifically binds an antigen. In some embodiments, the antigen is expressed on the surface of a cell. In some embodiments, the antigen is CD19. In some embodiments, the antigen is hCD19. In some embodiments, the antibody-based molecule comprises a single chain variable fragment (scFv).

As used herein, the term “extracellular antigen-binding domain” with respect to a CAR refers to an antigen-binding domain located outside of a cell. In some embodiments, the antigen-binding domain is operably linked to a transmembrane domain that is operably linked to a cytoplasmic domain that comprises at least one intracellular signaling domain and the antigen-binding domain is oriented so that it is located outside a cell with the CAR is expressed in a cell.

As used herein, the term “transmembrane domain” with respect to a CAR refers to the portion or portions of the CAR that are embedded in the plasma membrane of a cell when the CAR is expressed in the cell.

As used herein, the term “cytoplasmic domain” with respect to a CAR refers to the portion or portions of a CAR that are located in the cytoplasm of a cell when the CAR is expressed in the cell.

As used herein, the term “intracellular signaling domain” refers to a portion of the cytoplasmic domain of the CAR that comprises the primary signaling domain and/or the co-stimulatory domain.

As used herein, the term “primary signaling domain” refers to the intracellular portion of a signaling molecule that is responsible for mediating intracellular signaling events.

As used herein, the term “co-stimulatory domain” refers to the intracellular portion of a co-stimulatory molecule that is responsible for mediating intracellular signaling events.

As used herein, the term “cytokine” refers to a molecule that mediates and/or regulates a biological or cellular function or process (e.g., immunity, inflammation, and hematopoiesis). As used herein, cytokines include, but are not limited to, lymphokines, chemokines, monokines, and interleukins. The term cytokine as used herein also encompasses functional variants and functional variants of wild-type cytokines.

As used herein, the term “marker” protein or polypeptide refers to a protein or polypeptide that can be expressed on the surface of a cell, which can be utilized to mark or deplete cells expressing the marker protein or polypeptide. In some embodiments, depletion of cells expressing the marker protein or polypeptide is performed through the administration of a molecule that specifically binds the marker protein or polypeptide (e.g., an antibody that mediates antibody mediated cellular cytotoxicity).

As used herein, the term “immune effector cell” refers to a cell that is involved in the promotion of an immune effector function. Examples of immune effector cells include, but are not limited to, T cells (e.g., alpha/beta T cells and gamma/delta T cells, CD4⁺ T cells, CD8⁺ T cells, natural killer T (NK-T) cells), natural killer (NK) cells, B cells, mast cells, and myeloid-derived phagocytes.

As used herein, the term “immune effector function” refers to a specialized function of an immune effector cell. The effector function of any given immune effector cell can be different. For example, an effector function of a CD8+ T cell is cytolytic activity, and an effector function of a CD4+ T cell is secretion of a cytokine.

As used herein, the term “treat,” “treating,” and “treatment” refer to therapeutic or preventative measures described herein. The methods of “treatment” employ administration of a recombinant vector comprising a polycistronic expression cassette to a cell, and in some embodiments, administering the engineered cell to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “subject” includes any human or non-human animal. In one embodiment, the subject is a human or non-human mammal. In one embodiment, the subject is a human.

The determination of “percent identity” between two sequences (e.g., amino acid sequences or nucleic acid sequences) can be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877, each of which is herein incorporated by reference in its entirety. Such an algorithm is incorporated into the NBLAST and XBLAST programs of Altschul S F et al., (1990) J Mol Biol 215: 403, which is herein incorporated by reference in its entirety. BLAST nucleotide searches can be performed with the NBLAST nucleotide program parameters set, e.g., for score=100, wordlength=12 to obtain nucleotide sequences homologous to a nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389-3402, which is herein incorporated by reference in its entirety. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11-17, which is herein incorporated by reference in its entirety. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

### 5.2 Chimeric Antigen Receptors (CARs)

CARs are transmembrane proteins that comprise an antigen-binding domain, operably linked to a transmembrane domain, operably linked to a cytoplasmic domain that comprises at least one intracellular signaling domain. CARs can be expressed on the surface of a host cell (e.g., an immune effector cell) in order to mediate activation upon binding to the target antigen in vivo. In some embodiments, the CAR specifically binds CD19. In some embodiments, the CAR specifically binds human CD19 (hCD19).

### 5.2.1 hCD19 Binding Domains

hCD19 binding domains include any suitable antibody or non-antibody-based molecule that specifically binds hCD19 expressed on the surface of a cell. Exemplary hCD19 binding domains include, but are not limited to, antibodies and functional fragments and functional variants thereof. In some embodiments, the hCD19 binding domain comprises a single chain variable fragment (scFv), Fab, F(ab′)2, Fv, full-length antibody, a diabody, or an adnectin. In some embodiments, the hCD19 binding domain comprises a scFv.

In some embodiments, the hCD19 binding domain comprises a heavy chain variable region (VH) and a light chain variable region (VL). In some embodiments, the hCD19 binding domain comprises a VH and a VL that are operably linked via a peptide linker. In some embodiments, the peptide linker comprises glycine (G) and serine (S).

In some embodiments, the peptide linker comprises the amino acid sequence of SEQ ID NO: 9, or an amino acid sequence comprising 1, 2, 3, 4 or 5 amino acid modifications to the amino acid sequence of SEQ ID NO: 9. In some embodiments, the amino acid sequence of the peptide linker consists of the amino acid sequence of SEQ ID NO: 9, or an amino acid sequence comprising 1, 2, 3, 4 or 5 amino acid modifications to the amino acid sequence of SEQ ID NO: 9.

In some embodiments, the peptide linker comprises the amino acid sequence of SEQ ID NO: 17, or an amino acid sequence comprising 1, 2, 3, 4 or 5 amino acid modifications to the amino acid sequence of SEQ ID NO: 17. In some embodiments, the amino acid sequence of the peptide linker consists of the amino acid sequence of SEQ ID NO: 17, or an amino acid sequence comprising 1, 2, 3, 4 or 5 amino acid modifications to the amino acid sequence of SEQ ID NO: 17.

In some embodiments, the linker is encoded by a polynucleotide sequence at least 75%, 80%, 85%, 90% 95%, 96%, 97%, 98%, 99%, or 100% identical to the polynucleotide of SEQ ID NO: 27. In some embodiments, the linker is encoded by the polynucleotide of SEQ ID NO: 27.

In some embodiments, the linker is encoded by a polynucleotide sequence at least 75%, 80%, 85%, 90% 95%, 96%, 97%, 98%, 99%, or 100% identical to the polynucleotide of SEQ ID NO: 35. In some embodiments, the linker is encoded by the polynucleotide of SEQ ID NO: 35.

In some embodiments, the VH comprises three complementarity determining regions (CDRs): VH CDR1, VH CDR2, and VH CDR3. In some embodiments, the VH comprises the VH CDR1, VH CDR2, and VH CDR3 set forth in SEQ ID NO: 2. In some embodiments, the amino acid sequence of VH CDR1 comprises the amino acid sequence of SEQ ID NO: 6, or an amino acid sequence comprising 1, 2, or 3 amino acid modifications to the amino acid sequence of SEQ ID NO: 6; the amino acid sequence of VH CDR2 comprises the amino acid sequence of SEQ ID NO: 7, or an amino acid sequence comprising 1, 2, or 3 amino acid modifications to the amino acid sequence of SEQ ID NO: 7; the amino acid sequence of VH CDR3 comprises the amino acid sequence of SEQ ID NO: 8, or an amino acid sequence comprising 1, 2, or 3 amino acid modifications to the amino acid sequence of SEQ ID NO: 8. In some embodiments, the amino acid sequence of VH CDR1 comprises the amino acid sequence of SEQ ID NO: 6; the amino acid sequence of VH CDR2 comprises the amino acid sequence of SEQ ID NO: 7; and the amino acid sequence of VH CDR3 comprises the amino acid sequence of SEQ ID NO: 8. In some embodiments, the amino acid sequence of VH CDR1 consists of the amino acid sequence of SEQ ID NO: 6; the amino acid sequence of VH CDR2 consists of the amino acid sequence of SEQ ID NO: 7; and the amino acid sequence of VH CDR3 consists of the amino acid sequence of SEQ ID NO: 8.

In some embodiments, the VL comprises three CDRs: VL CDR1, VL CDR2, and VL CDR3. In some embodiments, the VL comprises the VL CDR1, VL CDR2, and VL CDR3 of SEQ ID NO: 1. In some embodiments, the amino acid sequence of VL CDR1 comprises the amino acid sequence of SEQ ID NO: 3, or an amino acid sequence comprising 1, 2, or 3 amino acid modifications to the amino acid sequence of SEQ ID NO: 3; the amino acid sequence of VL CDR2 comprises the amino acid sequence of SEQ ID NO: 4, or an amino acid sequence comprising 1, 2, or 3 amino acid modifications to the amino acid sequence of SEQ ID NO: 4; the amino acid sequence of VL CDR3 comprises the amino acid sequence of SEQ ID NO: 5, or an amino acid sequence comprising 1, 2, or 3 amino acid modifications to the amino acid sequence of SEQ ID NO: 5. In some embodiments, the amino acid sequence of VL CDR1 comprises the amino acid sequence of SEQ ID NO: 3; the amino acid sequence of VL CDR2 comprises the amino acid sequence of SEQ ID NO: 4; and the amino acid sequence of VL CDR3 comprises the amino acid sequence of SEQ ID NO: 5. In some embodiments, the amino acid sequence of VL CDR1 consists of the amino acid sequence of SEQ ID NO: 3; the amino acid sequence of VL CDR2 consists of the amino acid sequence of SEQ ID NO: 4; and the amino acid sequence of VL CDR3 consists of the amino acid sequence of SEQ ID NO: 5.

In some embodiments, the VH comprises the VH CDR1, VH CDR2, and VH CDR3 of SEQ ID NO: 2; and the VL comprises the VL CDR1, VL CDR2, and VL CDR3 of SEQ ID NO: 1. In some embodiments, the amino acid sequence of VH CDR1 comprises the amino acid sequence of SEQ ID NO: 6, or an amino acid sequence comprising 1, 2, or 3 amino acid modifications to the amino acid sequence of SEQ ID NO: 6; the amino acid sequence of VH CDR2 comprises the amino acid sequence of SEQ ID NO: 7, or an amino acid sequence comprising 1, 2, or 3 amino acid modifications to the amino acid sequence of SEQ ID NO: 7; the amino acid sequence of VH CDR3 comprises the amino acid sequence of SEQ ID NO: 8, or an amino acid sequence comprising 1, 2, or 3 amino acid modifications to the amino acid sequence of SEQ ID NO: 8; and the amino acid sequence of VL CDR1 comprises the amino acid sequence of SEQ ID NO: 3, or an amino acid sequence comprising 1, 2, or 3 amino acid modifications to the amino acid sequence of SEQ ID NO: 3; the amino acid sequence of VL CDR2 comprises the amino acid sequence of SEQ ID NO: 4, or an amino acid sequence comprising 1, 2, or 3 amino acid modifications to the amino acid sequence of SEQ ID NO: 4; the amino acid sequence of VL CDR3 comprises the amino acid sequence of SEQ ID NO: 5, or an amino acid sequence comprising 1, 2, or 3 amino acid modifications to the amino acid sequence of SEQ ID NO: 5.

In some embodiments, the amino acid sequence of VH CDR1 comprises the amino acid sequence of SEQ ID NO: 6; the amino acid sequence of VH CDR2 comprises the amino acid sequence of SEQ ID NO: 7; and the amino acid sequence of VH CDR3 comprises the amino acid sequence of SEQ ID NO: 8; and the amino acid sequence of VL CDR1 comprises the amino acid sequence of SEQ ID NO: 3; the amino acid sequence of VL CDR2 comprises the amino acid sequence of SEQ ID NO: 4; and the amino acid sequence of VL CDR3 comprises the amino acid sequence of SEQ ID NO: 5.

In some embodiments, the amino acid sequence of VH CDR1 consists of the amino acid sequence of SEQ ID NO: 6; the amino acid sequence of VH CDR2 consists of the amino acid sequence of SEQ ID NO: 7; and the amino acid sequence of VH CDR3 consists of the amino acid sequence of SEQ ID NO: 8; and the amino acid sequence of VL CDR1 consists of the amino acid sequence of SEQ ID NO: 3; the amino acid sequence of VL CDR2 consists of the amino acid sequence of SEQ ID NO: 4; and the amino acid sequence of VL CDR3 consists of the amino acid sequence of SEQ ID NO: 5.

In some embodiments, the VH comprises an amino acid sequence at least 95%, 96%, 97%, 98%, 99% or 100% identical to the amino acid sequence of SEQ ID NO: 2. In some embodiments, the VH comprises the amino acid sequence of SEQ ID NO: 2. In some embodiments, the amino acid sequence of the VH consists of a sequence at least 95%, 96%, 97%, 98%, 99% or 100% identical to the amino acid sequence of SEQ ID NO: 2. In some embodiments, the amino acid sequence of the VH consists of the amino acid sequence of SEQ ID NO: 2.

In some embodiments, the VL comprises an amino acid sequence at least 95%, 96%, 97%, 98%, 99% or 100% identical to the amino acid sequence of SEQ ID NO: 1. In some embodiments, the VL comprises the amino acid sequence of SEQ ID NO: 1. In some embodiments, the amino acid sequence of the VL consists of a sequence at least 95%, 96%, 97%, 98%, 99% or 100% identical to the amino acid sequence of SEQ ID NO: 1. In some embodiments, the amino acid sequence of the VL consists of the amino acid sequence of SEQ ID NO: 1.

In some embodiments, the VH comprises an amino acid sequence at least 95%, 96%, 97%, 98%, 99% or 100% identical to the amino acid sequence of SEQ ID NO: 2; and the VL comprises an amino acid sequence at least 95%, 96%, 97%, 98%, 99% or 100% identical to the amino acid sequence of SEQ ID NO: 1. In some embodiments, the VH comprises the amino acid sequence of SEQ ID NO: 2; and the VL comprises the amino acid sequence of SEQ ID NO: 1. In some embodiments, the amino acid sequence of the VH consists of a sequence at least 95%, 96%, 97%, 98%, 99% or 100% identical to the amino acid sequence of SEQ ID NO: 2; and the amino acid sequence of the VL consists of a sequence at least 95%, 96%, 97%, 98%, 99% or 100% identical to the amino acid sequence of SEQ ID NO: 1. In some embodiments, the amino acid sequence of the VH consists of the amino acid sequence of SEQ ID NO: 2; and the amino acid sequence of the VL consists of the amino acid sequence of SEQ ID NO: 1.

In some embodiments, the hCD19 binding domain comprises an amino acid sequence at least 95%, 96%, 97%, 98%, 99% or 100% identical to the amino acid sequence of SEQ ID NO: 11. In some embodiments, the hCD19 binding domain comprises the amino acid sequence of SEQ ID NO: 11. In some embodiments, the amino acid sequence of the hCD19 binding domain consists of a sequence at least 95%, 96%, 97%, 98%, 99% or 100% identical to the amino acid sequence of SEQ ID NO: 11. In some embodiments, the amino acid sequence of the hCD19 binding domain consists the amino acid sequence of SEQ ID NO: 11. In some embodiments, the hCD19 binding domain comprises an amino acid sequence at least 95%, 96%, 97%, 98%, 99% or 100% identical to the amino acid sequence of SEQ ID NO: 12. In some embodiments, the hCD19 binding domain comprises the amino acid sequence of SEQ ID NO: 12. In some embodiments, the amino acid sequence of the hCD19 binding domain consists of a sequence at least 95%, 96%, 97%, 98%, 99% or 100% identical to the amino acid sequence of SEQ ID NO: 12. In some embodiments, the amino acid sequence of the hCD19 binding domain consists the amino acid sequence of SEQ ID NO: 12. In some embodiments, the hCD19 binding domain comprises an amino acid sequence at least 95%, 96%, 97%, 98%, 99% or 100% identical to the amino acid sequence of SEQ ID NO: 13. In some embodiments, the hCD19 binding domain comprises the amino acid sequence of SEQ ID NO: 13. In some embodiments, the amino acid sequence of the hCD19 binding domain consists of a sequence at least 95%, 96%, 97%, 98%, 99% or 100% identical to the amino acid sequence of SEQ ID NO: 13. In some embodiments, the amino acid sequence of the hCD19 binding domain consists the amino acid sequence of SEQ ID NO: 13. In some embodiments, the hCD19 binding domain comprises an amino acid sequence at least 95%, 96%, 97%, 98%, 99% or 100% identical to the amino acid sequence of SEQ ID NO: 14. In some embodiments, the hCD19 binding domain comprises the amino acid sequence of SEQ ID NO: 14. In some embodiments, the amino acid sequence of the hCD19 binding domain consists of a sequence at least 95%, 96%, 97%, 98%, 99% or 100% identical to the amino acid sequence of SEQ ID NO: 14. In some embodiments, the amino acid sequence of the hCD19 binding domain consists the amino acid sequence of SEQ ID NO: 14. In some embodiments, the hCD19 binding domain comprises an amino acid sequence at least 95%, 96%, 97%, 98%, 99% or 100% identical to the amino acid sequence of SEQ ID NO: 15. In some embodiments, the hCD19 binding domain comprises the amino acid sequence of SEQ ID NO: 15. In some embodiments, the amino acid sequence of the hCD19 binding domain consists of a sequence at least 95%, 96%, 97%, 98%, 99% or 100% identical to the amino acid sequence of SEQ ID NO: 15. In some embodiments, the amino acid sequence of the hCD19 binding domain consists the amino acid sequence of SEQ ID NO: 15. In some embodiments, the hCD19 binding domain comprises an amino acid sequence at least 95%, 96%, 97%, 98%, 99% or 100% identical to the amino acid sequence of SEQ ID NO: 16. In some embodiments, the hCD19 binding domain comprises the amino acid sequence of SEQ ID NO: 16. In some embodiments, the amino acid sequence of the hCD19 binding domain consists of a sequence at least 95%, 96%, 97%, 98%, 99% or 100% identical to the amino acid sequence of SEQ ID NO: 16. In some embodiments, the amino acid sequence of the hCD19 binding domain consists the amino acid sequence of SEQ ID NO: 16.

In some embodiments, the VH comprises: a VH CDR1 encoded by the polynucleotide sequence of SEQ ID NO: 24, or a polynucleotide sequence comprising 1, 2, 3, 4, 5, 6, 7, 8 or 9 nucleotide modifications to the polynucleotide acid sequence of SEQ ID NO: 24; a VH CDR2 encoded by the polynucleotide sequence of SEQ ID NO: 25, or a polynucleotide sequence comprising 1, 2, 3, 4, 5, 6, 7, 8 or 9 nucleotide modifications to the polynucleotide acid sequence of SEQ ID NO: 25; a VH CDR3 encoded by the polynucleotide sequence of SEQ ID NO: 26, or a polynucleotide sequence comprising 1, 2, 3, 4, 5, 6, 7, 8 or 9 nucleotide modifications to the polynucleotide acid sequence of SEQ ID NO: 26. In some embodiments, the VH comprises a VH CDR1 encoded by the polynucleotide sequence of SEQ ID NO: 24; a VH CDR2 encoded by the polynucleotide sequence of SEQ ID NO: 25; and a VH CDR3 encoded by the polynucleotide sequence of SEQ ID NO: 26.

In some embodiments, the VL comprises: a VL CDR1 encoded by the polynucleotide sequence of SEQ ID NO: 21, or a polynucleotide sequence comprising 1, 2, 3, 4, 5, 6, 7, 8 or 9 nucleotide modifications to the polynucleotide acid sequence of SEQ ID NO: 21; a VL CDR2 encoded by the polynucleotide sequence of SEQ ID NO: 22, or a polynucleotide sequence comprising 1, 2, 3, 4, 5, 6, 7, 8 or 9 nucleotide modifications to the polynucleotide acid sequence of SEQ ID NO: 22; a VL CDR3 encoded by the polynucleotide sequence of SEQ ID NO: 23, or a polynucleotide sequence comprising 1, 2, 3, 4, 5, 6, 7, 8 or 9 nucleotide modifications to the polynucleotide acid sequence of SEQ ID NO: 23. In some embodiments, the VL comprises: a VL CDR1 encoded by the polynucleotide sequence of SEQ ID NO: 21; a VL CDR2 encoded by the polynucleotide sequence of SEQ ID NO: 22; and a VL CDR3 encoded by the polynucleotide sequence of SEQ ID NO: 23.

In some embodiments, the VH comprises: a VH CDR1 encoded by the polynucleotide sequence of SEQ ID NO: 24, or a polynucleotide sequence comprising 1, 2, 3, 4, 5, 6, 7, 8 or 9 nucleotide modifications to the polynucleotide acid sequence of SEQ ID NO: 24; a VH CDR2 encoded by the polynucleotide sequence of SEQ ID NO: 25, or a polynucleotide sequence comprising 1, 2, 3, 4, 5, 6, 7, 8 or 9 nucleotide modifications to the polynucleotide acid sequence of SEQ ID NO: 25; a VH CDR3 encoded by the polynucleotide sequence of SEQ ID NO: 26, or a polynucleotide sequence comprising 1, 2, 3, 4, 5, 6, 7, 8 or 9 nucleotide modifications to the polynucleotide acid sequence of SEQ ID NO: 26; and VL CDR1 encoded by the polynucleotide sequence of SEQ ID NO: 21, or a polynucleotide sequence comprising 1, 2, 3, 4, 5, 6, 7, 8 or 9 nucleotide modifications to the polynucleotide acid sequence of SEQ ID NO: 21; a VL CDR2 encoded by the polynucleotide sequence of SEQ ID NO: 22, or a polynucleotide sequence comprising 1, 2, 3, 4, 5, 6, 7, 8 or 9 nucleotide modifications to the polynucleotide acid sequence of SEQ ID NO: 22; a VL CDR3 encoded by the polynucleotide sequence of SEQ ID NO: 23, or a polynucleotide sequence comprising 1, 2, 3, 4, 5, 6, 7, 8 or 9 nucleotide modifications to the polynucleotide acid sequence of SEQ ID NO: 23.

In some embodiments, the VH comprises a VH CDR1 encoded by the polynucleotide sequence of SEQ ID NO: 24; a VH CDR2 encoded by the polynucleotide sequence of SEQ ID NO: 25; and a VH CDR3 encoded by the polynucleotide sequence of SEQ ID NO: 26; and a VL CDR1 encoded by the polynucleotide sequence of SEQ ID NO: 21; a VL CDR2 encoded by the polynucleotide sequence of SEQ ID NO: 22; and a VL CDR3 encoded by the polynucleotide sequence of SEQ ID NO: 23.

In some embodiments, the VH is encoded by a polynucleotide sequence at least 75%, 80%, 85%, 90%, 95%, 96%, 97%, 98%, 99% or 100% identical to the polynucleotide sequence of SEQ ID NO: 20. In some embodiments, the VH is encoded by the polynucleotide sequence of SEQ ID NO: 20.

In some embodiments, the VL is encoded by a polynucleotide sequence at least 75%, 80%, 85%, 90%, 95%, 96%, 97%, 98%, 99% or 100% identical to the polynucleotide sequence of SEQ ID NO: 19. In some embodiments, the VL is encoded by the polynucleotide sequence of SEQ ID NO: 19.

In some embodiments, the VH is encoded by a polynucleotide sequence at least 75%, 80%, 85%, 90%, 95%, 96%, 97%, 98%, 99% or 100% identical to the polynucleotide sequence of SEQ ID NO: 20; and the VL is encoded by a polynucleotide sequence at least 75%, 80%, 85%, 90%, 95%, 96%, 97%, 98%, 99% or 100% identical to the polynucleotide sequence of SEQ ID NO: 19. In some embodiments, the VH is encoded by the polynucleotide sequence of SEQ ID NO: 20; and the VL that is encoded by the polynucleotide sequence of SEQ ID NO: 19.

In some embodiments, the hCD19 binding domain is encoded by a polynucleotide sequence at least 75%, 80%, 85%, 90%, 95%, 96%, 97%, 98%, 99% or 100% identical to the polynucleotide sequence of SEQ ID NO: 29. In some embodiments, the hCD19 binding domain is encoded by a polynucleotide sequence at least 75%, 80%, 85%, 90%, 95%, 96%, 97%, 98%, 99% or 100% identical to the polynucleotide sequence of SEQ ID NO: 30. In some embodiments, the hCD19 binding domain is encoded by a polynucleotide sequence at least 75%, 80%, 85%, 90%, 95%, 96%, 97%, 98%, 99% or 100% identical to the polynucleotide sequence of SEQ ID NO: 31. In some embodiments, the hCD19 binding domain is encoded by a polynucleotide sequence at least 75%, 80%, 85%, 90%, 95%, 96%, 97%, 98%, 99% or 100% identical to the polynucleotide sequence of SEQ ID NO: 32. In some embodiments, the hCD19 binding domain is encoded by a polynucleotide sequence at least 75%, 80%, 85%, 90%, 95%, 96%, 97%, 98%, 99% or 100% identical to the polynucleotide sequence of SEQ ID NO: 33. In some embodiments, the hCD19 binding domain is encoded by a polynucleotide sequence at least 75%, 80%, 85%, 90%, 95%, 96%, 97%, 98%, 99% or 100% identical to the polynucleotide sequence of SEQ ID NO: 34.

The amino acid sequence and polynucleotide sequence of exemplary hCD19 binding domains are set forth in Table 1, herein.

TABLE 1
Amino acid and polynucleotide sequences of exemplary hCD19 binding domains.

| | | SEQ ID | | SEQ ID |
|---|---|---|---|---|
| Description | Amino Acid Sequence | NO | Polynucleotide Sequence | NO |
| FMC63 VL | DIQMTQTTSSLSASLG | 1 | GACATCCAGATGACCCAGACCACCTCCA | 19 |
| (without N- | DRVTISCRASQDISKY | | GCCTGAGCGCCAGCCTGGGCGACCGGGT | |
| terminal signal | LNWYQQKPDGTVKLLI | | GACCATCAGCTGCCGGGCCAGCCAGGAC | |
| sequence) | YHTSRLHSGVPSRFSG | | ATCAGCAAGTACCTGAACTGGTATCAGC | |
| | SGSGTDYSLTISNLEQ | | AGAAGCCCGACGGCACCGTCAAGCTGCT | |
| | EDIATYFCQQGNTLPY | | GATCTACCACACCAGCCGGCTGCACAGC | |
| | TFGGGTKLEIT | | GGCGTGCCCAGCCGGTTTAGCGGCAGCG | |
| | | | GCTCCGGCACCGACTACAGCCTGACCAT | |
| | | | CTCCAACCTGGAGCAGGAGGACATCGCC | |
| | | | ACCTACTTTTGCCAGCAGGGCAACACAC | |
| | | | TGCCCTACACCTTTGGCGGCGGAACAAA | |
| | | | GCTGGAGATCACC | |
| FMC63 VH | EVKLQESGPGLVAPSQ | 2 | GAGGTGAAGCTGCAGGAGAGCGGCCCTG | 20 |
| (without N- | SLSVTCTVSGVSLPDY | | GCCTGGTGGCCCCCAGCCAGAGCCTGAG | |
| terminal signal | GVSWIRQPPRKGLEWL | | CGTGACCTGTACCGTGTCCGGCGTGTCC | |
| sequence) | GVIWGSETTYYNSALK | | CTGCCCGACTACGGCGTGTCCTGGATCC | |
| | SRLTIIKDNSKSQVFL | | GGCAGCCCCCTAGGAAGGGCCTGGAGTG | |
| | KMNSLQTDDTAIYYCA | | GCTGGGCGTGATCTGGGGCAGCGAGACC | |
| | KHYYYGGSYAMDYWGQ | | ACCTACTACAACAGCGCCCTGAAGAGCC | |
| | GTSVTVSS | | GGCTGACCATCATCAAGGACAACAGCAA | |
| | | | GAGCCAGGTGTTCCTGAAGATGAACAGC | |
| | | | CTGCAGACCGACGACACCGCCATCTACT | |
| | | | ACTGTGCCAAGCACTACTACTACGGCGG | |
| | | | CAGCTACGCCATGGACTACTGGGGCCAG | |
| | | | GGCACCAGCGTGACCGTGTCCAGC | |
| FMC63 VL | RASQDISKYLN | 3 | CGGGCCAGCCAGGACATCAGCAAGTACC | 21 |
| CDR1 | | | TGAAC | |
| FMC63 VL | HTSRLHS | 4 | CACACCAGCCGGCTGCACAGC | 22 |
| CDR2 | | | | |
| FMC63 VL | QQGNTLPYT | 5 | CAGCAGGGCAACACACTGCCCTACACC | 23 |
| CDR3 | | | | |
| FMC63 VH | DYGVS | 6 | GACTACGGCGTGTCC | 24 |
| CDR1 | | | | |
| FMC63 VH | VIWGSETTYYNSALKS | 7 | GTGATCTGGGGCAGCGAGACCACCTACT | 25 |
| CDR2 | | | ACAACAGCGCCCTGAAGAGC | |
| FMC63 VH | HYYYGGSYAMDY | 8 | CACTACTACTACGGCGGCAGCTACGCCA | 26 |
| CDR3 | | | TGGACTAC | |
| Whitlow linker | GSTSGSGKPGSGEGST | 9 | GGCAGCACCTCCGGCAGCGGCAAGCCTG | 27 |
| | KG | | GCAGCGGCGAGGGCAGCACCAAGGGC | |
| Human GM-CSF | MLLLVTSLLLCELPHP | 10 | ATGCTGCTGCTGGTGACCAGCCTGCTGC | 28 |
| receptor a chain | AFLLIP | | TGTGTGAGCTGCCCCACCCCGCCTTTCT | |
| N-terminal | | | GCTGATCCCC | |
| signal sequence | | | | |
| FMC63 VL-VH | MLLLVTSLLLCELPHP | 11 | ATGCTGCTGCTGGTGACCAGCCTGCTGC | 29 |
| scFv (with N- | AFLLIPDIQMTQTTSS | | TGTGTGAGCTGCCCCACCCCGCCTTTCT | |
| terminal signal | LSASLGDRVTISCRAS | | GCTGATCCCCGACATCCAGATGACCCAG | |
| sequence) | QDISKYLNWYQQKPDG | | ACCACCTCCAGCCTGAGCGCCAGCCTGG | |
| | TVKLLIYHTSRLHSGV | | GCGACCGGGTGACCATCAGCTGCCGGGC | |
| | PSRFSGSGSGTDYSLT | | CAGCCAGGACATCAGCAAGTACCTGAAC | |
| | ISNLEQEDIATYFCQQ | | TGGTATCAGCAGAAGCCCGACGGCACCG | |
| | GNTLPYTFGGGTKLEI | | TCAAGCTGCTGATCTACCACACCAGCCG | |
| | TGSTSGSGKPGSGEGS | | GCTGCACAGCGGCGTGCCCAGCCGGTTT | |
| | TKGEVKLQESGPGLVA | | AGCGGCAGCGGCTCCGGCACCGACTACA | |
| | PSQSLSVTCTVSGVSL | | GCCTGACCATCTCCAACCTGGAGCAGGA | |
| | PDYGVSWIRQPPRKGL | | GGACATCGCCACCTACTTTTGCCAGCAG | |
| | EWLGVIWGSETTYYNS | | GGCAACACACTGCCCTACACCTTTGGCG | |
| | ALKSRLTIIKDNSKSQ | | GCGGAACAAAGCTGGAGATCACCGGCAG | |
| | VFLKMNSLQTDDTAIY | | CACCTCCGGCAGCGGCAAGCCTGGCAGC | |
| | YCAKHYYYGGSYAMDY | | GGCGAGGGCAGCACCAAGGGCGAGGTGA | |
| | WGQGTSVTVSS | | AGCTGCAGGAGAGCGGCCCTGGCCTGGT | |
| | | | GGCCCCCAGCCAGAGCCTGAGCGTGACC | |
| | | | TGTACCGTGTCCGGCGTGTCCCTGCCCG | |
| | | | ACTACGGCGTGTCCTGGATCCGGCAGCC | |
| | | | CCCTAGGAAGGGCCTGGAGTGGCTGGGC | |
| | | | GTGATCTGGGGCAGCGAGACCACCTACT | |
| | | | ACAACAGCGCCCTGAAGAGCCGGCTGAC | |
| | | | CATCATCAAGGACAACAGCAAGAGCCAG | |
| | | | GTGTTCCTGAAGATGAACAGCCTGCAGA | |
| | | | CCGACGACACCGCCATCTACTACTGTGC | |
| | | | CAAGCACTACTACTACGGCGGCAGCTAC | |
| | | | GCCATGGACTACTGGGGCCAGGGCACCA | |
| | | | GCGTGACCGTGTCCAGC | |
| FMC63 VL-VH | DIQMTQTTSSLSASLG | 12 | GACATCCAGATGACCCAGACCACCTCCA | 30 |
| scFv (without N- | DRVTISCRASQDISKY | | GCCTGAGCGCCAGCCTGGGCGACCGGGT | |
| terminal signal | LNWYQQKPDGTVKLLI | | GACCATCAGCTGCCGGGCCAGCCAGGAC | |
| sequence) | YHTSRLHSGVPSRFSG | | ATCAGCAAGTACCTGAACTGGTATCAGC | |
| | SGSGTDYSLTISNLEQ | | AGAAGCCCGACGGCACCGTCAAGCTGCT | |
| | EDIATYFCQQGNTLPY | | GATCTACCACACCAGCCGGCTGCACAGC | |
| | TFGGGTKLEITGSTSG | | GGCGTGCCCAGCCGGTTTAGCGGCAGCG | |
| | SGKPGSGEGSTKGEVK | | GCTCCGGCACCGACTACAGCCTGACCAT | |
| | LQESGPGLVAPSQSLS | | CTCCAACCTGGAGCAGGAGGACATCGCC | |
| | VTCTVSGVSLPDYGVS | | ACCTACTTTTGCCAGCAGGGCAACACAC | |
| | WIRQPPRKGLEWLGVI | | TGCCCTACACCTTTGGCGGCGGAACAAA | |
| | WGSETTYYNSALKSRL | | GCTGGAGATCACCGGCAGCACCTCCGGC | |
| | TIIKDNSKSQVFLKMN | | AGCGGCAAGCCTGGCAGCGGCGAGGGCA | |
| | SLQTDDTAIYYCAKHY | | GCACCAAGGGCGAGGTGAAGCTGCAGGA | |
| | YYGGSYAMDYWGQGTS | | GAGCGGCCCTGGCCTGGTGGCCCCCAGC | |
| | VTVSS | | CAGAGCCTGAGCGTGACCTGTACCGTGT | |
| | | | CCGGCGTGTCCCTGCCCGACTACGGCGT | |
| | | | GTCCTGGATCCGGCAGCCCCCTAGGAAG | |
| | | | GGCCTGGAGTGGCTGGGCGTGATCTGGG | |
| | | | GCAGCGAGACCACCTACTACAACAGCGC | |
| | | | CCTGAAGAGCCGGCTGACCATCATCAAG | |
| | | | GACAACAGCAAGAGCCAGGTGTTCCTGA | |
| | | | AGATGAACAGCCTGCAGACCGACGACAC | |
| | | | CGCCATCTACTACTGTGCCAAGCACTAC | |
| | | | TACTACGGCGGCAGCTACGCCATGGACT | |
| | | | ACTGGGGCCAGGGCACCAGCGTGACCGT | |
| | | | GTCCAGC | |
| FMC63 VH-VL | MLLLVTSLLLCELPHP | 13 | ATGCTGCTGCTGGTGACCAGCCTGCTGC | 31 |
| scFv (with N- | AFLLIPEVKLQESGPG | | TGTGTGAGCTGCCCCACCCCGCCTTTCT | |
| terminal signal | LVAPSQSLSVTCTVSG | | GCTGATCCCCGAGGTGAAGCTGCAGGAG | |
| sequence) | VSLPDYGVSWIRQPPR | | AGCGGCCCTGGCCTGGTGGCCCCCAGCC | |
| | KGLEWLGVIWGSETTY | | AGAGCCTGAGCGTGACCTGTACCGTGTC | |
| | YNSALKSRLTIIKDNS | | CGGCGTGTCCCTGCCCGACTACGGCGTG | |
| | KSQVFLKMNSLQTDDT | | TCCTGGATCCGGCAGCCCCCTAGGAAGG | |
| | AIYYCAKHYYYGGSYA | | GCCTGGAGTGGCTGGGCGTGATCTGGGG | |
| | MDYWGQGTSVTVSSGS | | CAGCGAGACCACCTACTACAACAGCGCC | |
| | TSGSGKPGSGEGSTKG | | CTGAAGAGCCGGCTGACCATCATCAAGG | |
| | DIQMTQTTSSLSASLG | | ACAACAGCAAGAGCCAGGTGTTCCTGAA | |
| | DRVTISCRASQDISKY | | GATGAACAGCCTGCAGACCGACGACACC | |
| | LNWYQQKPDGTVKLLI | | GCCATCTACTACTGTGCCAAGCACTACT | |
| | YHTSRLHSGVPSRFSG | | ACTACGGCGGCAGCTACGCCATGGACTA | |
| | SGSGTDYSLTISNLEQ | | CTGGGGCCAGGGCACCAGCGTGACCGTG | |
| | EDIATYFCQQGNTLPY | | TCCAGCGGCAGCACCTCCGGCAGCGGCA | |
| | TFGGGTKLEIT | | AGCCTGGCAGCGGCGAGGGCAGCACCAA | |
| | | | GGGCGACATCCAGATGACCCAGACCACC | |
| | | | TCCAGCCTGAGCGCCAGCCTGGGCGACC | |
| | | | GGGTGACCATCAGCTGCCGGGCCAGCCA | |
| | | | GGACATCAGCAAGTACCTGAACTGGTAT | |
| | | | CAGCAGAAGCCCGACGGCACCGTCAAGC | |
| | | | TGCTGATCTACCACACCAGCCGGCTGCA | |
| | | | CAGCGGCGTGCCCAGCCGGTTTAGCGGC | |
| | | | AGCGGCTCCGGCACCGACTACAGCCTGA | |
| | | | CCATCTCCAACCTGGAGCAGGAGGACAT | |
| | | | CGCCACCTACTTTTGCCAGCAGGGCAAC | |
| | | | ACACTGCCCTACACCTTTGGCGGCGGAA | |
| | | | CAAAGCTGGAGATCACC | |
| FMC63 VH-VL | EVKLQESGPGLVAPSQ | 14 | GAGGTGAAGCTGCAGGAGAGCGGCCCTG | 32 |
| scFv (without N- | SLSVTCTVSGVSLPDY | | GCCTGGTGGCCCCCAGCCAGAGCCTGAG | |
| terminal signal | GVSWIRQPPRKGLEWL | | CGTGACCTGTACCGTGTCCGGCGTGTCC | |
| sequence) | GVIWGSETTYYNSALK | | CTGCCCGACTACGGCGTGTCCTGGATCC | |
| | SRLTIIKDNSKSQVEL | | GGCAGCCCCCTAGGAAGGGCCTGGAGTG | |
| | KMNSLQTDDTAIYYCA | | GCTGGGCGTGATCTGGGGCAGCGAGACC | |
| | KHYYYGGSYAMDYWGQ | | ACCTACTACAACAGCGCCCTGAAGAGCC | |
| | GTSVTVSSGSTSGSGK | | GGCTGACCATCATCAAGGACAACAGCAA | |
| | PGSGEGSTKGDIQMTQ | | GAGCCAGGTGTTCCTGAAGATGAACAGC | |
| | TTSSLSASLGDRVTIS | | CTGCAGACCGACGACACCGCCATCTACT | |
| | CRASQDISKYLNWYQQ | | ACTGTGCCAAGCACTACTACTACGGCGG | |
| | KPDGTVKLLIYHTSRL | | CAGCTACGCCATGGACTACTGGGGCCAG | |
| | HSGVPSRFSGSGSGTD | | GGCACCAGCGTGACCGTGTCCAGCGGCA | |
| | YSLTISNLEQEDIATY | | GCACCTCCGGCAGCGGCAAGCCTGGCAG | |
| | FCQQGNTLPYTFGGGT | | CGGCGAGGGCAGCACCAAGGGCGACATC | |
| | KLEIT | | CAGATGACCCAGACCACCTCCAGCCTGA | |
| | | | GCGCCAGCCTGGGCGACCGGGTGACCAT | |
| | | | CAGCTGCCGGGCCAGCCAGGACATCAGC | |
| | | | AAGTACCTGAACTGGTATCAGCAGAAGC | |
| | | | CCGACGGCACCGTCAAGCTGCTGATCTA | |
| | | | CCACACCAGCCGGCTGCACAGCGGCGTG | |
| | | | CCCAGCCGGTTTAGCGGCAGCGGCTCCG | |
| | | | GCACCGACTACAGCCTGACCATCTCCAA | |
| | | | CCTGGAGCAGGAGGACATCGCCACCTAC | |
| | | | TTTTGCCAGCAGGGCAACACACTGCCCT | |
| | | | ACACCTTTGGCGGCGGAACAAAGCTGGA | |
| | | | GATCACC | |
| FMC63 VL-VH | MALPVTALLLPLALLL | 15 | ATGGCCTTACCAGTGACCGCCTTGCTCC | 33 |
| scFv Variant A | HAARPDIQMTQTTSSL | | TGCCGCTGGCCTTGCTGCTCCACGCCGC | |
| (with N-terminal | SASLGDRVTISCRASQ | | CAGGCCGGACATCCAGATGACACAGACT | |
| signal sequence) | DISKYLNWYQQKPDGT | | ACATCCTCCCTGTCTGCCTCTCTGGGAG | |
| | VKLLIYHTSRLHSGVP | | ACAGAGTCACCATCAGTTGCAGGGCAAG | |
| | SRFSGSGSGTDYSLTI | | TCAGGACATTAGTAAATATTTAAATTGG | |
| | SNLEQEDIATYFCQQG | | TATCAGCAGAAACCAGATGGAACTGTTA | |
| | NTLPYTFGGGTKLEIT | | AACTCCTGATCTACCATACATCAAGATT | |
| | GGGGSGGGGSGGGGSE | | ACACTCAGGAGTCCCATCAAGGTTCAGT | |
| | VKLQESGPGLVAPSQS | | GGCAGTGGGTCTGGAACAGATTATTCTC | |
| | LSVTCTVSGVSLPDYG | | TCACCATTAGCAACCTGGAGCAAGAAGA | |
| | VSWIRQPPRKGLEWLG | | TATTGCCACTTACTTTTGCCAACAGGGT | |
| | VIWGSETTYYNSALKS | | AATACGCTTCCGTACACGTTCGGAGGGG | |
| | RLTIIKDNSKSQVFLK | | GGACCAAGCTGGAGATCACAGGTGGCGG | |
| | MNSLQTDDTAIYYCAK | | TGGCTCGGGCGGTGGTGGGTCGGGTGGC | |
| | HYYYGGSYAMDYWGQG | | GGCGGATCTGAGGTGAAACTGCAGGAGT | |
| | TSVTVSS | | CAGGACCTGGCCTGGTGGCGCCCTCACA | |
| | | | GAGCCTGTCCGTCACATGCACTGTCTCA | |
| | | | GGGGTCTCATTACCCGACTATGGTGTAA | |
| | | | GCTGGATTCGCCAGCCTCCACGAAAGGG | |
| | | | TCTGGAGTGGCTGGGAGTAATATGGGGT | |
| | | | AGTGAAACCACATACTATAATTCAGCTC | |
| | | | TCAAATCCAGACTGACCATCATCAAGGA | |
| | | | CAACTCCAAGAGCCAAGTTTTCTTAAAA | |
| | | | ATGAACAGTCTGCAAACTGATGACACAG | |
| | | | CCATTTACTACTGTGCCAAACATTATTA | |
| | | | CTACGGTGGTAGCTATGCTATGGACTAC | |
| | | | TGGGGCCAAGGAACCTCAGTCACCGTCT | |
| | | | CCTCA | |
| FMC63 VL-VH | DIQMTQTTSSLSASLG | 16 | GACATCCAGATGACACAGACTACATCCT | 34 |
| scFv Variant A | DRVTISCRASQDISKY | | CCCTGTCTGCCTCTCTGGGAGACAGAGT | |
| (without N- | LNWYQQKPDGTVKLLI | | CACCATCAGTTGCAGGGCAAGTCAGGAC | |
| terminal signal | YHTSRLHSGVPSRFSG | | ATTAGTAAATATTTAAATTGGTATCAGC | |
| sequence) | SGSGTDYSLTISNLEQ | | AGAAACCAGATGGAACTGTTAAACTCCT | |
| | EDIATYFCQQGNTLPY | | GATCTACCATACATCAAGATTACACTCA | |
| | TFGGGTKLEITGGGGS | | GGAGTCCCATCAAGGTTCAGTGGCAGTG | |
| | GGGGSGGGGSEVKLQE | | GGTCTGGAACAGATTATTCTCTCACCAT | |
| | SGPGLVAPSQSLSVTC | | TAGCAACCTGGAGCAAGAAGATATTGCC | |
| | TVSGVSLPDYGVSWIR | | ACTTACTTTTGCCAACAGGGTAATACGC | |
| | QPPRKGLEWLGVIWGS | | TTCCGTACACGTTCGGAGGGGGGACCAA | |
| | ETTYYNSALKSRLTII | | GCTGGAGATCACAGGTGGCGGTGGCTCG | |
| | KDNSKSQVFLKMNSLQ | | GGCGGTGGTGGGTCGGGTGGCGGCGGAT | |
| | TDDTAIYYCAKHYYYG | | CTGAGGTGAAACTGCAGGAGTCAGGACC | |
| | GSYAMDYWGQGTSVTV | | TGGCCTGGTGGCGCCCTCACAGAGCCTG | |
| | SS | | TCCGTCACATGCACTGTCTCAGGGGTCT | |
| | | | CATTACCCGACTATGGTGTAAGCTGGAT | |
| | | | TCGCCAGCCTCCACGAAAGGGTCTGGAG | |
| | | | TGGCTGGGAGTAATATGGGGTAGTGAAA | |
| | | | CCACATACTATAATTCAGCTCTCAAATC | |
| | | | CAGACTGACCATCATCAAGGACAACTCC | |
| | | | AAGAGCCAAGTTTTCTTAAAAATGAACA | |
| | | | GTCTGCAAACTGATGACACAGCCATTTA | |
| | | | CTACTGTGCCAAACATTATTACTACGGT | |
| | | | GGTAGCTATGCTATGGACTACTGGGGCC | |
| | | | AAGGAACCTCAGTCACCGTCTCCTCA | |
| Variant A linker | GGGGSGGGGSGGGGS | 17 | GGTGGCGGTGGCTCGGGCGGTGGTGGGT | 35 |
| | | | CGGGTGGCGGCGGATCT | |
| Variant A N- | MALPVTALLLPLALLL | 18 | ATGGCCTTACCAGTGACCGCCTTGCTCC | 36 |
| terminal signal | HAARP | | TGCCGCTGGCCTTGCTGCTCCACGCCGC | |
| sequence | | | CAGGCCG | |

### 5.2.2 Hinge Domains

In some embodiments, the CAR comprises an amino acid sequence positioned between the antigen-binding domain and the transmembrane domain referred to herein as a hinge domain. The hinge domain can provide optimal distance of the antigen-binding domain from the membrane of the cell when the CAR is expressed on the cell surface. The hinge domain can also provide optimal flexibility for the antigen-binding domain to bind to its target antigen. In some embodiments, the hinge domain is derived from the extracellular region of a naturally occurring protein expressed on the surface of an immune effector cell. In some embodiments, the hinge domain is derived from the hinge domain of a naturally occurring protein expressed on the surface of an immune effector cell. In some embodiments, the immune effector cell is a T cell. In some embodiments, the T cell is a CD4+ T cell. In some embodiments, the T cell is a CD8+ T cell.

In some embodiments, the hinge domain is directly operably linked to the C terminus of the antigen-binding domain. In some embodiments, the hinge domain is indirectly operably linked to the C terminus of the antigen-binding domain. In some embodiments, the hinge domain is indirectly operably linked to the C terminus of the antigen-binding domain via a peptide linker. In some embodiments, the hinge domain is directly operably linked to the N terminus of the transmembrane domain. In some embodiments, the hinge domain is indirectly operably linked to the N terminus of the transmembrane domain. In some embodiments, the hinge domain is indirectly operably linked to the N terminus of the transmembrane domain via a peptide linker.

In some embodiments, the hinge domain is derived from human CD8α (hCD8α). In some embodiments, the hinge domain comprises the hinge domain of hCD8α. In some embodiments, the hinge domain comprises an amino acid sequence at least 95%, 96%, 97%, 98%, 99%, or 100% identical to the amino acid sequence of SEQ ID NO: 37. In some embodiments, the hinge domain comprises the amino acid sequence of SEQ ID NO: 37. In some embodiments, the amino acid sequence of the hinge domain consists of a sequence at least 95%, 96%, 97%, 98%, 99%, or 100% identical to the amino acid sequence of SEQ ID NO: 37. In some embodiments, the amino acid sequence of the hinge domain consists of the amino acid sequence of SEQ ID NO: 37.

In some embodiments, the hinge domain comprises an amino acid sequence at least 95%, 96%, 97%, 98%, 99%, or 100% identical to the amino acid sequence of SEQ ID NO: 38. In some embodiments, the hinge domain comprises the amino acid sequence of SEQ ID NO: 38. In some embodiments, the amino acid sequence of the hinge domain consists of a sequence at least 95%, 96%, 97%, 98%, 99%, or 100% identical to the amino acid sequence of SEQ ID NO: 38. In some embodiments, the amino acid sequence of the hinge domain consists of the amino acid sequence of SEQ ID NO: 38.

In some embodiments, the hinge domain is encoded by a polynucleotide sequence at least 75%, 80%, 85%, 90%, 95%, 96%, 97%, 98%, 99% or 100% identical to the polynucleotide sequence of SEQ ID NO: 40. In some embodiments, the hinge domain is encoded by the polynucleotide sequence of SEQ ID NO: 40.

In some embodiments, the hinge domain is encoded by a polynucleotide sequence at least 75%, 80%, 85%, 90%, 95%, 96%, 97%, 98%, 99% or 100% identical to the polynucleotide sequence of SEQ ID NO: 41. In some embodiments, the hinge domain is encoded by the polynucleotide sequence of SEQ ID NO: 41.

In some embodiments, the hinge domain is derived from human CD28 (hCD28). In some embodiments, the hinge domain comprises the hinge domain of hCD28. In some embodiments, the hinge domain comprises an amino acid sequence at least 95%, 96%, 97%, 98%, 99%, or 100% identical to the amino acid sequence of SEQ ID NO: 39. In some embodiments, the hinge domain comprises the amino acid sequence of SEQ ID NO: 39. In some embodiments, the amino acid sequence of the hinge domain consists of a sequence at least 95%, 96%, 97%, 98%, 99%, or 100% identical to the amino acid sequence of SEQ ID NO: 39. In some embodiments, the amino acid sequence of the hinge domain consists of the amino acid sequence of SEQ ID NO: 39. In some embodiments, the hinge domain is encoded by a polynucleotide sequence at least 75%, 80%, 85%, 90%, 95%, 96%, 97%, 98%, 99% or 100% identical to the polynucleotide sequence of SEQ ID NO: 42. In some embodiments, the hinge domain is encoded the polynucleotide sequence of SEQ ID NO: 42.

The amino acid sequence and polynucleotide sequence of exemplary hinge domains are set forth in Table 2, herein.

TABLE 2
Amino acid and polynucleotide sequences of exemplary hinge domains.

| | | SEQ ID | | SEQ ID |
|---|---|---|---|---|
| Description | Amino Acid Sequence | NO | Polynucleotide Sequence | NO |
| hCD8α hinge | KPTTTPAPRPPTPAPTIAS | 37 | AAGCCCACCACCACCCCTGCC | 40 |
| domain | QPLSLRPEACRPAAGGAVH | | CCTAGACCTCCAACCCCAGCC | |
| | TRGLDFACD | | CCTACAATCGCCAGCCAGCCC | |
| | | | CTGAGCCTGAGGCCCGAAGCC | |
| | | | TGTAGACCTGCCGCTGGCGGA | |
| | | | GCCGTGCACACCAGAGGCCTG | |
| | | | GATTTCGCCTGCGAC | |
| hCD8α hinge | TTTPAPRPPTPAPTIASQP | 38 | ACCACGACGCCAGCGCCGCGA | 41 |
| domain | LSLRPEACRPAAGGAVHTR | | CCACCAACACCGGCGCCCACC | |
| (modified) | GLDFACD | | ATCGCGTCGCAGCCCCTGTCC | |
| | | | CTGCGCCCAGAGGCGTGCCGG | |
| | | | CCAGCGGCGGGGGGCGCAGTG | |
| | | | CACACGAGGGGGCTGGACTTC | |
| | | | GCCTGTGAT | |
| hCD28 hinge | AAAIEVMYPPPYLDNEKSN | 39 | GCGGCCGCAATTGAAGTTATG | 42 |
| domain | GTIIHVKGKHLCPSPLFPG | | TATCCTCCTCCTTACCTAGAC | |
| | PSKP | | AATGAGAAGAGCAATGGAACC | |
| | | | ATTATCCATGTGAAAGGGAAA | |
| | | | CACCTTTGTCCAAGTCCCCTA | |
| | | | TTTCCCGGACCTTCTAAGCCC | |

### 5.2.3 Transmembrane Domains

The transmembrane domain of the CAR functions to embed the CAR in the plasma membrane of a cell. In some embodiments, the transmembrane domain is operably linked to the C terminus of the antigen-binding domain. In some embodiments, the transmembrane domain is directly operably linked to the C terminus of the antigen-binding domain. In some embodiments, the transmembrane domain is indirectly operably linked to the C terminus of the antigen-binding domain. In some embodiments, the transmembrane domain is indirectly operably linked to the C terminus of the antigen-binding domain via a peptide linker. In some embodiments, the transmembrane domain is indirectly operably linked to the C terminus of the antigen-binding domain via a hinge domain.

In some embodiments, the transmembrane domain is operably linked to the C terminus of the hinge domain. In some embodiments, the transmembrane domain is directly operably linked to the C terminus of the hinge domain. In some embodiments, the transmembrane domain is indirectly operably linked to the C terminus of the hinge domain. In some embodiments, the transmembrane domain is indirectly operably linked to the C terminus of the hinge domain via a peptide linker.

In some embodiments, the transmembrane domain is operably linked to the N terminus of the cytoplasmic domain. In some embodiments, the transmembrane domain is directly operably linked to the N terminus of the cytoplasmic domain. In some embodiments, the transmembrane domain is indirectly operably linked to the N terminus of the cytoplasmic domain. In some embodiments, the transmembrane domain is indirectly operably linked to the N terminus of the cytoplasmic domain via a peptide linker.

In some embodiments, the transmembrane domain is derived from the transmembrane domain of a naturally occurring transmembrane protein expressed on the surface of an immune effector cell. In some embodiments, the immune effector cell is a T cell. In some embodiments, the T cell is a CD8⁺ T cell. In some embodiments, the T cell is a CD4⁺ T cell. In some embodiments, the transmembrane domain and the hinge domain are derived from the same naturally occurring transmembrane protein expressed on the surface of an immune effector cell.

In some embodiments, the transmembrane is derived from the transmembrane domain of a protein selected from the group consisting of CD8α, CD28, TCRα, TCRβ, TCRζ, CD3ε, CD45, CD4, CDS, CD9, CD16, CD22, CD33, CD37, CD64, CD80, CD86, CD134, CD137, and CD154.

Alternatively, the transmembrane domain can be synthetic (i.e., not derived from a naturally occurring transmembrane protein). In some embodiments, the synthetic transmembrane domain comprises predominantly hydrophobic amino acid residues (e.g., leucine and valine). In some embodiments, a triplet of phenylalanine, tryptophan and valine will be found at each end of the synthetic transmembrane domain.

In some embodiments, the transmembrane domain comprises the transmembrane domain of hCD8α, or functional fragment or functional variant thereof. In some embodiments, the transmembrane domain comprises an amino acid sequence at least 95%, 96%, 97%, 98%, 99%, or 100% identical to the amino acid sequence of SEQ ID NO: 43. In some embodiments, the transmembrane domain comprises the amino acid sequence of SEQ ID NO: 43. In some embodiments, the amino acid sequence of the transmembrane domain consists of a sequence at least 95%, 96%, 97%, 98%, 99%, or 100% identical to the amino acid sequence of SEQ ID NO: 43. In some embodiments, the amino acid sequence of the transmembrane domain consists of the amino acid sequence of SEQ ID NO: 43.

In some embodiments, the transmembrane domain comprises an amino acid sequence at least 95%, 96%, 97%, 98%, 99%, or 100% identical to the amino acid sequence of SEQ ID NO: 44. In some embodiments, the transmembrane domain comprises the amino acid sequence of SEQ ID NO: 44. In some embodiments, the amino acid sequence of the transmembrane domain consists of a sequence at least 95%, 96%, 97%, 98%, 99%, or 100% identical to the amino acid sequence of SEQ ID NO: 44. In some embodiments, the amino acid sequence of the transmembrane domain consists of the amino acid sequence of SEQ ID NO: 44.

In some embodiments, the transmembrane domain comprises the transmembrane domain of hCD28, or functional fragment or functional variant thereof. In some embodiments, the transmembrane domain comprises an amino acid sequence at least 95%, 96%, 97%, 98%, 99%, or 100% identical to the amino acid sequence of SEQ ID NO: 45. In some embodiments, the transmembrane domain comprises the amino acid sequence of SEQ ID NO: 45. In some embodiments, the amino acid sequence of the transmembrane domain consists of a sequence at least 95%, 96%, 97%, 98%, 99%, or 100% identical to the amino acid sequence of SEQ ID NO: 45. In some embodiments, the amino acid sequence of the transmembrane domain consists of the amino acid sequence of SEQ ID NO: 45.

In some embodiments, the transmembrane domain is encoded by a polynucleotide sequence at least 75%, 80%, 85%, 90%, 95%, 96%, 97%, 98%, 99% or 100% identical to the polynucleotide sequence of SEQ ID NO: 49. In some embodiments, the transmembrane domain is encoded by the polynucleotide sequence of SEQ ID NO: 49. In some embodiments, the transmembrane domain is encoded by a polynucleotide sequence at least 75%, 80%, 85%, 90%, 95%, 96%, 97%, 98%, 99% or 100% identical to the polynucleotide sequence of SEQ ID NO: 50. In some embodiments, the transmembrane domain is encoded by the polynucleotide sequence of SEQ ID NO: 50. In some embodiments, the transmembrane domain is encoded by a polynucleotide sequence at least 75%, 80%, 85%, 90%, 95%, 96%, 97%, 98%, 99% or 100% identical to the polynucleotide sequence of SEQ ID NO: 51. In some embodiments, the transmembrane domain is encoded by the polynucleotide sequence of SEQ ID NO: 51. In some embodiments, the transmembrane domain is encoded by a polynucleotide sequence at least 75%, 80%, 85%, 90%, 95%, 96%, 97%, 98%, 99% or 100% identical to the polynucleotide sequence of SEQ ID NO: 52. In some embodiments, the transmembrane domain is encoded by the polynucleotide sequence of SEQ ID NO: 52.

In some embodiments, the CAR comprises a hinge region and transmembrane domain that together comprise an amino acid sequence at least 95%, 96%, 97%, 98%, 99%, or 100% identical to the amino acid sequence of SEQ ID NO: 46. In some embodiments, the CAR comprises a hinge region and transmembrane domain that together comprise the amino acid sequence of SEQ ID NO: 46. In some embodiments, the amino acid sequence of the hinge region and transmembrane domain together consist of a sequence at least 95%, 96%, 97%, 98%, 99%, or 100% identical to the amino acid sequence of SEQ ID NO: 46. In some embodiments, the amino acid sequence of the hinge region and transmembrane domain together consist of the amino acid sequence of SEQ ID NO: 46.

In some embodiments, the CAR comprises a hinge region and transmembrane domain that together comprise an amino acid sequence at least 95%, 96%, 97%, 98%, 99%, or 100% identical to the amino acid sequence of SEQ ID NO: 47. In some embodiments, the CAR comprises a hinge region and transmembrane domain that together comprise the amino acid sequence of SEQ ID NO: 47. In some embodiments, the amino acid sequence of the hinge region and transmembrane domain together consist of a sequence at least 95%, 96%, 97%, 98%, 99%, or 100% identical to the amino acid sequence of SEQ ID NO: 47. In some embodiments, the amino acid sequence of the hinge region and transmembrane domain together consist of the amino acid sequence of SEQ ID NO: 47.

In some embodiments, the CAR comprises a hinge region and transmembrane domain that together comprise an amino acid sequence at least 95%, 96%, 97%, 98%, 99%, or 100% identical to the amino acid sequence of SEQ ID NO: 48. In some embodiments, the CAR comprises a hinge region and transmembrane domain that together comprise the amino acid sequence of SEQ ID NO: 48. In some embodiments, the amino acid sequence of the hinge region and transmembrane domain together consist of a sequence at least 95%, 96%, 97%, 98%, 99%, or 100% identical to the amino acid sequence of SEQ ID NO: 48. In some embodiments, the amino acid sequence of the hinge region and transmembrane domain together consist of the amino acid sequence of SEQ ID NO: 48.

In some embodiments, the hinge region and transmembrane domain together are encoded by a polynucleotide sequence at least 75%, 80%, 85%, 90%, 95%, 96%, 97%, 98%, 99% or 100% identical to the polynucleotide sequence of SEQ ID NO: 53. In some embodiments, the hinge region and transmembrane domain together are encoded by the polynucleotide sequence of SEQ ID NO: 53. In some embodiments, the hinge region and transmembrane domain together are encoded by a polynucleotide sequence at least 75%, 80%, 85%, 90%, 95%, 96%, 97%, 98%, 99% or 100% identical to the polynucleotide sequence of SEQ ID NO: 54. In some embodiments, the hinge region and transmembrane domain together are encoded by the polynucleotide sequence of SEQ ID NO: 54. In some embodiments, the hinge region and transmembrane domain together are encoded by a polynucleotide sequence at least 75%, 80%, 85%, 90%, 95%, 96%, 97%, 98%, 99% or 100% identical to the polynucleotide sequence of SEQ ID NO: 55. In some embodiments, the hinge region and transmembrane domain together are encoded by the polynucleotide sequence of SEQ ID NO: 55. In some embodiments, the hinge region and transmembrane domain that together are encoded by a polynucleotide sequence at least 75%, 80%, 85%, 90%, 95%, 96%, 97%, 98%, 99% or 100% identical to the polynucleotide sequence of SEQ ID NO: 56. In some embodiments, the hinge region and transmembrane domain together are encoded by the polynucleotide sequence of SEQ ID NO: 56.

The amino acid sequence and polynucleotide sequence of exemplary transmembrane domains and hinge plus transmembrane domains are set forth in Table 3, herein.

TABLE 3
Amino acid and polynucleotide sequences of exemplary transmembrane domains,
and hinge region and transmembrane domain fusions.

| | | SEQ ID | | SEQ ID |
|---|---|---|---|---|
| Description | Amino Acid Sequence | NO | Polynucleotide Sequence | NO |
| Human CD8α | IYIWAPLAGTCGVLLLSLV | 43 | ATCTACATCTGGGCCCCTCTG | 49 |
| transmembrane | ITLYCNHRN | | GCCGGCACCTGTGGCGTGCTG | |
| domain | | | CTGCTGAGCCTGGTCATCACC | |
| | | | CTGTACTGCAACCACCGGAAT | |
| | | | ATCTACATCTGGGCACCTCTG | 50 |
| | | | GCCGGCACCTGTGGCGTGCTG | |
| | | | CTGCTGAGCCTGGTCATCACC | |
| | | | CTGTACTGCAACCACCGGAAT | |
| Human CD8α | IYIWAPLAGTCGVLLLSLV | 44 | ATCTACATCTGGGCGCCCTTG | 51 |
| transmembrane | ITLYC | | GCCGGGACTTGTGGGGTCCTT | |
| domain | | | CTCCTGTCACTGGTTATCACC | |
| (modified) | | | CTTTACTGC | |
| Human CD28 | FWVLVVVGGVLACYSLLVT | 45 | TTTTGGGTGCTGGTGGTGGTT | 52 |
| transmembrane | VAFIIFWV | | GGGGGAGTCCTGGCTTGCTAT | |
| domain | | | AGCTTGCTAGTAACAGTGGCC | |
| | | | TTTATTATTTTCTGGGTG | |
| Human CD8α | KPTTTPAPRPPTPAPTIAS | 46 | AAGCCCACCACCACCCCTGCC | 53 |
| hinge and human | QPLSLRPEACRPAAGGAVH | | CCTAGACCTCCAACCCCAGCC | |
| CD8α | TRGLDFACDIYIWAPLAGT | | CCTACAATCGCCAGCCAGCCC | |
| transmembrane | CGVLLLSLVITLYCNHRN | | CTGAGCCTGAGGCCCGAAGCC | |
| domain | | | TGTAGACCTGCCGCTGGCGGA | |
| | | | GCCGTGCACACCAGAGGCCTG | |
| | | | GATTTCGCCTGCGACATCTAC | |
| | | | ATCTGGGCCCCTCTGGCCGGC | |
| | | | ACCTGTGGCGTGCTGCTGCTG | |
| | | | AGCCTGGTCATCACCCTGTAC | |
| | | | TGCAACCACCGGAAT | |
| | | | AAGCCCACCACCACCCCTGCC | 54 |
| | | | CCTAGACCTCCAACCCCAGCC | |
| | | | CCTACAATCGCCAGCCAGCCC | |
| | | | CTGAGCCTGAGGCCCGAAGCC | |
| | | | TGTAGACCTGCCGCTGGCGGA | |
| | | | GCCGTGCACACCAGAGGCCTG | |
| | | | GATTTCGCCTGCGACATCTAC | |
| | | | ATCTGGGCACCTCTGGCCGGC | |
| | | | ACCTGTGGCGTGCTGCTGCTG | |
| | | | AGCCTGGTCATCACCCTGTAC | |
| | | | TGCAACCACCGGAAT | |
| Human CD8α | TTTPAPRPPTPAPTIASQP | 47 | ACCACGACGCCAGCGCCGCGA | 55 |
| hinge (modified) | LSLRPEACRPAAGGAVHTR | | CCACCAACACCGGCGCCCACC | |
| and human CD8α | GLDFACDIYIWAPLAGTCG | | ATCGCGTCGCAGCCCCTGTCC | |
| transmembrane | VLLLSLVITLYC | | CTGCGCCCAGAGGCGTGCCGG | |
| domain | | | CCAGCGGCGGGGGGCGCAGTG | |
| (modified) | | | CACACGAGGGGGCTGGACTTC | |
| | | | GCCTGTGATATCTACATCTGG | |
| | | | GCGCCCTTGGCCGGGACTTGT | |
| | | | GGGGTCCTTCTCCTGTCACTG | |
| | | | GTTATCACCCTTTACTGC | |
| Human CD28 | AAAIEVMYPPPYLDNEKSN | 48 | GCGGCCGCAATTGAAGTTATG | 56 |
| hinge and human | GTIIHVKGKHLCPSPLFPG | | TATCCTCCTCCTTACCTAGAC | |
| CD28 | PSKPFWVLVVVGGVLACYS | | AATGAGAAGAGCAATGGAACC | |
| transmembrane | LLVTVAFIIFWV | | ATTATCCATGTGAAAGGGAAA | |
| domain | | | CACCTTTGTCCAAGTCCCCTA | |
| | | | TTTCCCGGACCTTCTAAGCCC | |
| | | | TTTTGGGTGCTGGTGGTGGTT | |
| | | | GGGGGAGTCCTGGCTTGCTAT | |
| | | | AGCTTGCTAGTAACAGTGGCC | |
| | | | TTTATTATTTTCTGGGTG | |

### 5.2.4 Cytoplasmic Domains

The cytoplasmic domain of a CAR described herein comprises at least a primary signaling domain that initiates antigen-dependent primary activation and optionally one or more co-stimulatory domains to provide a costimulatory signal.

In some embodiments, the cytoplasmic domain is operably linked to the C terminus of the transmembrane domain. In some embodiments, the cytoplasmic domain is directly operably linked to the C terminus of the transmembrane domain. In some embodiments, the cytoplasmic domain is indirectly operably linked to the C terminus of the transmembrane domain. In some embodiments, the cytoplasmic domain is indirectly operably linked to the C terminus of the transmembrane domain via a peptide linker.

In some embodiments, the primary signaling domain comprises at least one immunoreceptor tyrosine-based activation motif (ITAM). Exemplary primary signaling domains include, but are not limited to, the signaling domains of CD3ζ, CD3γ, CD3δ, CD3ε, FcRγ, FcRβ, CDS, CD22, CD79a, CD79b, and CD66d, and functional fragments and functional variants thereof. In some embodiments, the primary signaling domain is derived from CD3ζ, CD3γ, CD3δ, CD3ε, FcRγ, FcRβ, CDS, CD22, CD79a, CD79b, or CD66d. In some embodiments, the primary signaling domain comprises the CD3ζ intracellular signaling domain or a functional fragment or functional variant thereof. In some embodiments, the primary signaling domain is derived from human CD3ζ.

In some embodiments, the cytoplasmic domain comprising a primary signaling domain comprises an amino acid sequence at least at least 95%, 96%, 97%, 98%, 99%, or 100% identical to the amino acid sequence of SEQ ID NO: 60. In some embodiments, the cytoplasmic domain comprising a primary signaling domain comprises the amino acid sequence of SEQ ID NO: 60. In some embodiments, the amino acid sequence of the cytoplasmic domain comprising a primary signaling domain consists of a sequence at least 95%, 96%, 97%, 98%, 99%, or 100% identical to the amino acid sequence of SEQ ID NO: 60. In some embodiments, the amino acid sequence of the cytoplasmic domain comprising a primary signaling domain consists of the amino acid sequence of SEQ ID NO: 60.

In some embodiments, the cytoplasmic domain comprising a primary signaling domain is encoded by a polynucleotide sequence at least 75%, 80%, 85%, 90%, 95%, 96%, 97%, 98%, 99% or 100% identical to the polynucleotide sequence of SEQ ID NO: 67. In some embodiments, the cytoplasmic domain comprising a primary signaling domain is encoded by polynucleotide sequence of SEQ ID NO: 67. In some embodiments, the cytoplasmic domain comprising a primary signaling domain is encoded by a polynucleotide sequence at least 75%, 80%, 85%, 90%, 95%, 96%, 97%, 98%, 99% or 100% identical to the polynucleotide sequence of SEQ ID NO: 68. In some embodiments, the cytoplasmic domain comprising a primary signaling domain is encoded by the polynucleotide sequence of SEQ ID NO: 68.

In some embodiments, the cytoplasmic domain comprises at least one co-stimulatory domain. In some embodiments, the cytoplasmic domain comprises a plurality of costimulatory domains. In some embodiments, the cytoplasmic domain comprises a primary signaling domain and one co-stimulatory domain. In some embodiments, the cytoplasmic domain comprises a primary signaling domain and two co-stimulatory domains, wherein the two co-stimulatory domains can be the same or different. In some embodiments, the cytoplasmic domain comprises a primary signaling domain and three co-stimulatory domains, wherein the three co-stimulatory domains can each individually be the same or different from another one of the three co-stimulatory domains.

In some embodiments, the cytoplasmic domain comprises a co-stimulatory domain, or functional fragment or variant thereof, of a protein selected from the group consisting of CD28, 4-IBB, OX40, CD27, CD30, CD40, PD-I, ICOS, LFA1, CD2, CD7, LIGHT, NKG2C, B7-H3, DAP10, and DAPI2. In some embodiments, the protein is CD28. In some embodiments, the protein is 4-1BB.

In some embodiments the cytoplasmic domain comprises the co-stimulatory domain of CD28, or a functional fragment or functional variant thereof. In some embodiments, the cytoplasmic domain comprises an amino acid sequence at least 95%, 96%, 97%, 98%, 99%, or 100% identical to the amino acid sequence of SEQ ID NO: 57. In some embodiments, the cytoplasmic domain comprises the amino acid sequence of SEQ ID NO: 57. In some embodiments, the cytoplasmic domain comprises an amino acid sequence at least 95%, 96%, 97%, 98%, 99%, or 100% identical to the amino acid sequence of SEQ ID NO: 58. In some embodiments, the cytoplasmic domain comprises the amino acid sequence of SEQ ID NO: 58. In some embodiments, the amino acid sequence of the cytoplasmic domain consists of a sequence at least 95%, 96%, 97%, 98%, 99%, or 100% identical to the amino acid sequence of SEQ ID NO: 57. In some embodiments, the amino acid sequence of the cytoplasmic domain consists of the amino acid sequence of SEQ ID NO: 57. In some embodiments, the amino acid sequence of the cytoplasmic domain consists of a sequence at least 95%, 96%, 97%, 98%, 99%, or 100% identical to the amino acid sequence of SEQ ID NO: 58. In some embodiments, the amino acid sequence of the cytoplasmic domain consists of the amino acid sequence of SEQ ID NO: 58.

In some embodiments, the cytoplasmic domain is encoded by a polynucleotide sequence at least 75%, 80%, 85%, 90%, 95%, 96%, 97%, 98%, 99% or 100% identical to the polynucleotide sequence of SEQ ID NO: 64. In some embodiments, the cytoplasmic domain is encoded by the polynucleotide sequence of SEQ ID NO: 64. In some embodiments, the cytoplasmic domain is encoded by a polynucleotide sequence at least 75%, 80%, 85%, 90%, 95%, 96%, 97%, 98%, 99% or 100% identical to the polynucleotide sequence of SEQ ID NO: 65. In some embodiments, the cytoplasmic domain is encoded by the polynucleotide sequence of SEQ ID NO: 65.

In some embodiments the cytoplasmic domain comprises the co-stimulatory domain of 4-1BB, or a functional fragment or functional variant thereof. In some embodiments, the cytoplasmic domain comprises an amino acid sequence at least 95%, 96%, 97%, 98%, 99%, or 100% identical to the amino acid sequence of SEQ ID NO: 59. In some embodiments, the cytoplasmic domain comprises the amino acid sequence of SEQ ID NO: 59. In some embodiments, the amino acid sequence of the cytoplasmic domain consists of a sequence at least 95%, 96%, 97%, 98%, 99%, or 100% identical to the amino acid sequence of SEQ ID NO: 59. In some embodiments, the amino acid sequence of the cytoplasmic domain consists of the amino acid sequence of SEQ ID NO: 59.

In some embodiments, the cytoplasmic domain is encoded by a polynucleotide sequence at least 75%, 80%, 85%, 90%, 95%, 96%, 97%, 98%, 99% or 100% identical to the polynucleotide sequence of SEQ ID NO: 66. In some embodiments, the cytoplasmic domain is encoded by the polynucleotide sequence of SEQ ID NO: 66.

The primary signaling domain can be operably linked directly or indirectly to one or more co-stimulatory domains. In some embodiments, the primary signaling is directly operably linked to a co-stimulatory domain. In some embodiments, the primary signaling domain is indirectly operably linked to a co-stimulatory domain. In some embodiments, the primary signaling domain is indirectly operably linked to a co-stimulatory domain via a peptide linker. In some embodiments, the co-stimulatory domain is operably linked to the N terminus of the primary signaling domain. In some embodiments, the co-stimulatory domain is directly operably linked to the N terminus of the primary signaling domain. In some embodiments, the co-stimulatory domain is indirectly operably linked to the N terminus of the primary signaling domain. In some embodiments, the co-stimulatory domain is indirectly operably linked to the N terminus of the primary signaling domain via a peptide linker.

The primary signaling domain can be operably linked directly or indirectly to the transmembrane domain. In some embodiments, the primary signaling domain is operably directly linked to the transmembrane domain. In some embodiments, the primary signaling domain is operably indirectly linked to the transmembrane domain. In some embodiments, the primary signaling domain is operably indirectly linked to the transmembrane domain through a peptide linker.

The co-stimulatory domain can be operably linked directly or indirectly to the transmembrane domain. In some embodiments, the co-stimulatory domain is operably directly linked to the transmembrane domain. In some embodiments, the co-stimulatory domain is operably indirectly linked to the transmembrane domain. In some embodiments, the co-stimulatory domain is operably indirectly linked to the transmembrane domain through a peptide linker.

In some embodiments, the intracellular signaling domain comprises the co-stimulatory domain of CD28, or a functional variant or functional fragment thereof, and the signaling domain of CD3ζ, or a functional fragment or functional variant thereof. In some embodiments, the cytoplasmic domain comprises an amino acid sequence at least 95%, 96%, 97%, 98%, 99%, or 100% identical to the amino acid sequence of SEQ ID NO: 61. In some embodiments, the cytoplasmic domain comprises the amino acid sequence of SEQ ID NO: 61. In some embodiments, the cytoplasmic domain comprises an amino acid sequence at least 95%, 96%, 97%, 98%, 99%, or 100% identical to the amino acid sequence of SEQ ID NO: 63. In some embodiments, the cytoplasmic domain comprises the amino acid sequence of SEQ ID NO: 63.

In some embodiments, the amino acid sequence of the cytoplasmic domain consists of a sequence at least 95%, 96%, 97%, 98%, 99%, or 100% identical to the amino acid sequence of SEQ ID NO: 61. In some embodiments, the amino acid sequence of the cytoplasmic domain consists of the amino acid sequence of SEQ ID NO: 61. In some embodiments, the amino acid sequence of the cytoplasmic domain consists of a sequence at least 95%, 96%, 97%, 98%, 99%, or 100% identical to the amino acid sequence of SEQ ID NO: 63. In some embodiments, the amino acid sequence of the cytoplasmic domain consists of the amino acid sequence of SEQ ID NO: 63.

In some embodiments, the cytoplasmic domain is encoded by a polynucleotide sequence at least 75%, 80%, 85%, 90%, 95%, 96%, 97%, 98%, 99% or 100% identical to the polynucleotide sequence of SEQ ID NO: 69. In some embodiments, the cytoplasmic domain is encoded by the polynucleotide sequence of SEQ ID NO: 69. In some embodiments, the cytoplasmic domain is encoded by a polynucleotide sequence at least 75%, 80%, 85%, 90%, 95%, 96%, 97%, 98%, 99% or 100% identical to the polynucleotide sequence of SEQ ID NO: 71. In some embodiments, the cytoplasmic domain is encoded by the polynucleotide sequence of SEQ ID NO: 71.

In some embodiments, the intracellular signaling domain comprises the co-stimulatory domain of 4-1BB, or a functional variant or functional fragment thereof, and the primary signaling domain of CD3ζ, or a functional fragment or functional variant thereof. In some embodiments, the cytoplasmic domain comprises an amino acid sequence at least 95%, 96%, 97%, 98%, 99%, or 100% identical to the amino acid sequence of SEQ ID NO: 62. In some embodiments, the cytoplasmic domain comprises the amino acid sequence of SEQ ID NO: 62. In some embodiments, the amino acid sequence of the cytoplasmic domain consists of a sequence at least 95%, 96%, 97%, 98%, 99%, or 100% identical to the amino acid sequence of SEQ ID NO: 62. In some embodiments, the amino acid sequence of the cytoplasmic domain consists of the amino acid sequence of SEQ ID NO: 62.

In some embodiments, the cytoplasmic domain is encoded by a polynucleotide sequence at least 75%, 80%, 85%, 90%, 95%, 96%, 97%, 98%, 99% or 100% identical to the polynucleotide sequence of SEQ ID NO: 70. In some embodiments, the cytoplasmic domain is encoded by the polynucleotide sequence of SEQ ID NO: 70.

The amino acid sequence and polynucleotide sequence of exemplary cytoplasmic domain comprising primary signaling domains, co-stimulatory domains, and intracellular signaling domains are set forth in Table 4, herein.

TABLE 4
Amino acid and polynucleotide sequences of exemplary cytoplasmic domains.

| | | SEQ ID | | SEQ ID |
|---|---|---|---|---|
| Description | Amino Acid Sequence | NO | Polynucleotide Sequence | NO |
| Human CD28 | RSKRSRGGHSDYMNMTPRR | 57 | AGGAGCAAGCGGAGCAGAGGC | 64 |
| cytoplasmic | PGPTRKHYQPYAPPRDFAA | | GGCCACAGCGACTACATGAAC | |
| domain | YRS | | ATGACCCCCCGGAGGCCTGGC | |
| (modified) | | | CCCACCCGGAAGCACTACCAG | |
| | | | CCCTACGCCCCTCCCAGGGAC | |
| | | | TTCGCCGCCTACCGGAGC | |
| Human CD28 | RSKRSRLLHSDYMNMTPRR | 58 | AGGAGTAAGAGGAGCAGGCTC | 65 |
| cytoplasmic | PGPTRKHYQPYAPPRDFAA | | CTGCACAGTGACTACATGAAC | |
| domain | YRS | | ATGACTCCCCGCCGCCCCGGG | |
| | | | CCCACCCGCAAGCATTACCAG | |
| | | | CCCTATGCCCCACCACGCGAC | |
| | | | TTCGCAGCCTATCGCTCC | |
| Human 4-1BB | KRGRKKLLYIFKQPFMRPV | 59 | AAACGGGGCAGAAAGAAACTC | 66 |
| cytoplasmic | QTTQEEDGCSCRFPEEEEG | | CTGTATATATTCAAACAACCA | |
| domain | GCEL | | TTTATGAGACCAGTACAAACT | |
| | | | ACTCAAGAGGAAGATGGCTGT | |
| | | | AGCTGCCGATTTCCAGAAGAA | |
| | | | GAAGAAGGAGGATGTGAACTG | |
| Human CD3ζ | RVKFSRSADAPAYQQGQNQ | 60 | CGGGTGAAGTTCAGCCGGAGC | 67 |
| cytoplasmic | LYNELNLGRREEYDVLDKR | | GCCGACGCCCCTGCCTACCAG | |
| domain | RGRDPEMGGKPRRKNPQEG | | CAGGGCCAGAACCAGCTGTAC | |
| | LYNELQKDKMAEAYSEIGM | | AACGAGCTGAACCTGGGCCGG | |
| | KGERRRGKGHDGLYQGLST | | AGGGAGGAGTACGACGTGCTG | |
| | ATKDTYDALHMQALPPR | | GACAAGCGGAGAGGCCGGGAC | |
| | | | CCTGAGATGGGCGGCAAGCCC | |
| | | | CGGAGAAAGAACCCTCAGGAG | |
| | | | GGCCTGTATAACGAACTGCAG | |
| | | | AAAGACAAGATGGCCGAGGCC | |
| | | | TACAGCGAGATCGGCATGAAG | |
| | | | GGCGAGCGGCGGAGGGGCAAG | |
| | | | GGCCACGACGGCCTGTACCAG | |
| | | | GGCCTGAGCACCGCCACCAAG | |
| | | | GATACCTACGACGCCCTGCAC | |
| | | | ATGCAGGCCCTGCCCCCCAGA | |
| | | | AGAGTGAAGTTCAGCAGGAGC | 68 |
| | | | GCAGACGCCCCCGCGTACCAG | |
| | | | CAGGGCCAGAACCAGCTCTAT | |
| | | | AACGAGCTCAATCTAGGACGA | |
| | | | AGAGAGGAGTACGATGTTTTG | |
| | | | GACAAGAGACGTGGCCGGGAC | |
| | | | CCTGAGATGGGGGGAAAGCCG | |
| | | | AGAAGGAAGAACCCTCAGGAA | |
| | | | GGCCTGTACAATGAACTGCAG | |
| | | | AAAGATAAGATGGCGGAGGCC | |
| | | | TACAGTGAGATTGGGATGAAA | |
| | | | GGCGAGCGCCGGAGGGGCAAG | |
| | | | GGGCACGATGGCCTTTACCAG | |
| | | | GGTCTCAGTACAGCCACCAAG | |
| | | | GACACCTACGACGCCCTTCAC | |
| | | | ATGCAGGCCCTGCCCCCTCGC | |
| Human CD28 | RSKRSRGGHSDYMNMTPRR | 61 | AGGAGCAAGCGGAGCAGAGGC | 69 |
| cytoplasmic | PGPTRKHYQPYAPPRDFAA | | GGCCACAGCGACTACATGAAC | |
| domain | YRSRVKFSRSADAPAYQQG | | ATGACCCCCCGGAGGCCTGGC | |
| (modified) | QNQLYNELNLGRREEYDVL | | CCCACCCGGAAGCACTACCAG | |
| and | DKRRGRDPEMGGKPRRKNP | | CCCTACGCCCCTCCCAGGGAC | |
| human CD3ζ | QEGLYNELQKDKMAEAYSE | | TTCGCCGCCTACCGGAGCCGG | |
| cytoplasmic | IGMKGERRRGKGHDGLYQG | | GTGAAGTTCAGCCGGAGCGCC | |
| domain | LSTATKDTYDALHMQALPP | | GACGCCCCTGCCTACCAGCAG | |
| | R | | GGCCAGAACCAGCTGTACAAC | |
| | | | GAGCTGAACCTGGGCCGGAGG | |
| | | | GAGGAGTACGACGTGCTGGAC | |
| | | | AAGCGGAGAGGCCGGGACCCT | |
| | | | GAGATGGGCGGCAAGCCCCGG | |
| | | | AGAAAGAACCCTCAGGAGGGC | |
| | | | CTGTATAACGAACTGCAGAAA | |
| | | | GACAAGATGGCCGAGGCCTAC | |
| | | | AGCGAGATCGGCATGAAGGGC | |
| | | | GAGCGGCGGAGGGGCAAGGGC | |
| | | | CACGACGGCCTGTACCAGGGC | |
| | | | CTGAGCACCGCCACCAAGGAT | |
| | | | ACCTACGACGCCCTGCACATG | |
| | | | CAGGCCCTGCCCCCCAGA | |
| Human 4-1BB | KRGRKKLLYIFKQPFMRPV | 62 | AAACGGGGCAGAAAGAAACTC | 70 |
| cytoplasmic | QTTQEEDGCSCRFPEEEEG | | CTGTATATATTCAAACAACCA | |
| domain and | GCELRVKFSRSADAPAYQQ | | TTTATGAGACCAGTACAAACT | |
| human CD3ζ | GQNQLYNELNLGRREEYDV | | ACTCAAGAGGAAGATGGCTGT | |
| cytoplasmic | LDKRRGRDPEMGGKPRRKN | | AGCTGCCGATTTCCAGAAGAA | |
| domain | PQEGLYNELQKDKMAEAYS | | GAAGAAGGAGGATGTGAACTG | |
| | EIGMKGERRRGKGHDGLYQ | | AGAGTGAAGTTCAGCAGGAGC | |
| | GLSTATKDTYDALHMQALP | | GCAGACGCCCCCGCGTACCAG | |
| | PR | | CAGGGCCAGAACCAGCTCTAT | |
| | | | AACGAGCTCAATCTAGGACGA | |
| | | | AGAGAGGAGTACGATGTTTTG | |
| | | | GACAAGAGACGTGGCCGGGAC | |
| | | | CCTGAGATGGGGGGAAAGCCG | |
| | | | AGAAGGAAGAACCCTCAGGAA | |
| | | | GGCCTGTACAATGAACTGCAG | |
| | | | AAAGATAAGATGGCGGAGGCC | |
| | | | TACAGTGAGATTGGGATGAAA | |
| | | | GGCGAGCGCCGGAGGGGCAAG | |
| | | | GGGCACGATGGCCTTTACCAG | |
| | | | GGTCTCAGTACAGCCACCAAG | |
| | | | GACACCTACGACGCCCTTCAC | |
| | | | ATGCAGGCCCTGCCCCCTCGC | |
| Human CD28 | RSKRSRLLHSDYMNMTPRR | 63 | AGGAGTAAGAGGAGCAGGCTC | 71 |
| cytoplasmic | PGPTRKHYQPYAPPRDFAA | | CTGCACAGTGACTACATGAAC | |
| domain and | YRSRVKFSRSADAPAYQQG | | ATGACTCCCCGCCGCCCCGGG | |
| human CD3ζ | QNQLYNELNLGRREEYDVL | | CCCACCCGCAAGCATTACCAG | |
| cytoplasmic | DKRRGRDPEMGGKPRRKNP | | CCCTATGCCCCACCACGCGAC | |
| domain | QEGLYNELQKDKMAEAYSE | | TTCGCAGCCTATCGCTCCAGA | |
| | IGMKGERRRGKGHDGLYQG | | GTGAAGTTCAGCAGGAGCGCA | |
| | LSTATKDTYDALHMQALPP | | GACGCCCCCGCGTACCAGCAG | |
| | R | | GGCCAGAACCAGCTCTATAAC | |
| | | | GAGCTCAATCTAGGACGAAGA | |
| | | | GAGGAGTACGATGTTTTGGAC | |
| | | | AAGAGACGTGGCCGGGACCCT | |
| | | | GAGATGGGGGGAAAGCCGAGA | |
| | | | AGGAAGAACCCTCAGGAAGGC | |
| | | | CTGTACAATGAACTGCAGAAA | |
| | | | GATAAGATGGCGGAGGCCTAC | |
| | | | AGTGAGATTGGGATGAAAGGC | |
| | | | GAGCGCCGGAGGGGCAAGGGG | |
| | | | CACGATGGCCTTTACCAGGGT | |
| | | | CTCAGTACAGCCACCAAGGAC | |
| | | | ACCTACGACGCCCTTCACATG | |
| | | | CAGGCCCTGCCCCCTCGC | |

### 5.2.5 Exemplary CD19-Specific CARs

The amino acid and polynucleotide sequences of exemplary CD19 specific CARs are provided in Table 5, herein. In some embodiments, the CAR comprises an amino acid sequence at least 95%, 96%, 97%, 98%, 99%, or 100% identical to the amino acid sequence of SEQ ID NO: 72, 73, 74, 75, 76, 77, 78, 79, 80 or 81. In some embodiments, the CAR comprises an amino acid sequence at least 95%, 96%, 97%, 98%, 99%, or 100% identical to the amino acid sequence of SEQ ID NO: 72. In some embodiments, the CAR comprises an amino acid sequence at least 95%, 96%, 97%, 98%, 99%, or 100% identical to the amino acid sequence of SEQ ID NO: 73. In some embodiments, the CAR comprises an amino acid sequence at least 80%, 85%, 90%, 91%, 92%, 93%, 94%, 95%, 96%, 97%, 98%, 99%, or 100% identical to the amino acid sequence of SEQ ID NO: 74. In some embodiments, the CAR comprises an amino acid sequence at least 95%, 96%, 97%, 98%, 99%, or 100% identical to the amino acid sequence of SEQ ID NO: 75. In some embodiments, the CAR comprises an amino acid sequence at least 95%, 96%, 97%, 98%, 99%, or 100% identical to the amino acid sequence of SEQ ID NO: 76. In some embodiments, the CAR comprises an amino acid sequence at 95%, 96%, 97%, 98%, 99%, or 100% identical to the amino acid sequence of SEQ ID NO: 77. In some embodiments, the CAR comprises an amino acid sequence at 95%, 96%, 97%, 98%, 99%, or 100% identical to the amino acid sequence of SEQ ID NO: 78. In some embodiments, the CAR comprises an amino acid sequence at least 95%, 96%, 97%, 98%, 99%, or 100% identical to the amino acid sequence of SEQ ID NO: 79. In some embodiments, the CAR comprises an amino acid sequence at least 95%, 96%, 97%, 98%, 99%, or 100% identical to the amino acid sequence of SEQ ID NO: 80. In some embodiments, the CAR comprises an amino acid sequence at least 95%, 96%, 97%, 98%, 99%, or 100% identical to the amino acid sequence of SEQ ID NO: 81.

In some embodiments, the CAR comprises the amino acid sequence of SEQ ID NO: 72, 73, 74, 75, 76, 77, 78, 79, 80, or 81. In some embodiments, the CAR comprises the amino acid sequence of SEQ ID NO: 72. In some embodiments, the CAR comprises the amino acid sequence of SEQ ID NO: 73. In some embodiments, the CAR comprises the amino acid sequence of SEQ ID NO: 74. In some embodiments, the CAR comprises the amino acid sequence of SEQ ID NO: 75. In some embodiments, the CAR comprises the amino acid sequence of SEQ ID NO: 76. In some embodiments, the CAR comprises the amino acid sequence of SEQ ID NO: 77. In some embodiments, the CAR comprises the amino acid sequence of SEQ ID NO: 78. In some embodiments, the CAR comprises the amino acid sequence of SEQ ID NO: 79. In some embodiments, the CAR comprises the amino acid sequence of SEQ ID NO: 80. In some embodiments, the CAR comprises the amino acid sequence of SEQ ID NO: 81.

In some embodiments, the amino acid sequence of the CAR consists of a sequence at least 95%, 96%, 97%, 98%, 99%, or 100% identical to the amino acid sequence of SEQ ID NO: 72, 73, 74, 75, 76, 77, 78, 79, 80, or 81. In some embodiments, the amino acid sequence of the CAR consists of a sequence at least 95%, 96%, 97%, 98%, 99%, or 100% identical to the amino acid sequence of SEQ ID NO: 72. In some embodiments, the amino acid sequence of the CAR consists of a sequence at least 95%, 96%, 97%, 98%, 99%, or 100% identical to the amino acid sequence of SEQ ID NO: 73. In some embodiments, the amino acid sequence of the CAR consists of a sequence at least 95%, 96%, 97%, 98%, 99%, or 100% identical to the amino acid sequence of SEQ ID NO: 74. In some embodiments, the amino acid sequence of the CAR consists of a sequence at least 95%, 96%, 97%, 98%, 99%, or 100% identical to the amino acid sequence of SEQ ID NO: 75. In some embodiments, the amino acid sequence of the CAR consists of a sequence at least 95%, 96%, 97%, 98%, 99%, or 100% identical to the amino acid sequence of SEQ ID NO: 76. In some embodiments, the amino acid sequence of the CAR consists of a sequence at least 95%, 96%, 97%, 98%, 99%, or 100% identical to the amino acid sequence of SEQ ID NO: 77. In some embodiments, the amino acid sequence of the CAR consists of a sequence at least 95%, 96%, 97%, 98%, 99%, or 100% identical to the amino acid sequence of SEQ ID NO: 78. In some embodiments, the amino acid sequence of the CAR consists of a sequence at least 95%, 96%, 97%, 98%, 99%, or 100% identical to the amino acid sequence of SEQ ID NO: 79. In some embodiments, the amino acid sequence of the CAR consists of a sequence at least 95%, 96%, 97%, 98%, 99%, or 100% identical to the amino acid sequence of SEQ ID NO: 80. In some embodiments, the amino acid sequence of the CAR consists of a sequence at least 95%, 96%, 97%, 98%, 99%, or 100% identical to the amino acid sequence of SEQ ID NO: 81.

In some embodiments, the amino acid sequence of the CAR consists of the amino acid sequence of SEQ ID NO: 72, 73, 74, 75, 76, 77, 78, 79, 80, or 81. In some embodiments, the amino acid sequence of the CAR consists of the amino acid sequence of SEQ ID NO: 72. In some embodiments, the amino acid sequence of the CAR consists of the amino acid sequence of SEQ ID NO: 73. In some embodiments, the amino acid sequence of the CAR consists of the amino acid sequence of SEQ ID NO: 74. In some embodiments, the amino acid sequence of the CAR consists of the amino acid sequence of SEQ ID NO: 75. In some embodiments, the amino acid sequence of the CAR consists of the amino acid sequence of SEQ ID NO: 76. In some embodiments, the amino acid sequence of the CAR consists of the amino acid sequence of SEQ ID NO: 77. In some embodiments, the amino acid sequence of the CAR consists of the amino acid sequence of SEQ ID NO: 78. In some embodiments, the amino acid sequence of the CAR consists of the amino acid sequence of SEQ ID NO: 79. In some embodiments, the amino acid sequence of the CAR consists of the amino acid sequence of SEQ ID NO: 80. In some embodiments, the amino acid sequence of the CAR consists of the amino acid sequence of SEQ ID NO: 81.

In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 82, 83, 84, 86, 87, 88, 90, 91, 92, 93, 94, or 95. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 82. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 83. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 84. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 86. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 87. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 88. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 89. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 90. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 91. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 92. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 93. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 94. In some embodiments, the CAR is encoded by a polynucleotide sequence at least 75%, 80%, 85%, 90%, 91%, 92%, 93%, 94%, 95%, 96%, 97%, 98%, 99%, or 100% identical to the polynucleotide sequence of SEQ ID NO: 95.

In some embodiments, the CAR is encoded by the polynucleotide sequence of SEQ ID NO: 82, 83, 84, 86, 87, 88, 90, 91, 92, 93, 94, or 95. In some embodiments, the CAR is encoded by the polynucleotide sequence of SEQ ID NO: 82. In some embodiments, the CAR is encoded by a polynucleotide sequence comprising the polynucleotide sequence of SEQ ID NO: 83. In some embodiments, the CAR is encoded by the polynucleotide sequence of SEQ ID NO: 84. In some embodiments, the CAR is encoded by the polynucleotide sequence of SEQ ID NO: 86. In some embodiments, the CAR is encoded by a polynucleotide sequence comprising the polynucleotide sequence of SEQ ID NO: 87. In some embodiments, the CAR is encoded by the polynucleotide sequence of SEQ ID NO: 88. In some embodiments, the CAR is encoded by the polynucleotide sequence of SEQ ID NO: 90. In some embodiments, the CAR is encoded by a polynucleotide sequence comprising the polynucleotide sequence of SEQ ID NO: 91. In some embodiments, the CAR is encoded by the polynucleotide sequence of SEQ ID NO: 92. In some embodiments, the CAR is encoded by a polynucleotide sequence comprising the polynucleotide sequence of SEQ ID NO: 93. In some embodiments, the CAR is encoded by the polynucleotide sequence of SEQ ID NO: 94. In some embodiments, the CAR is encoded by the polynucleotide sequence of SEQ ID NO: 95.

In some embodiments, the CAR comprises the amino acid sequence of CAR CTL019. In some embodiments, the CAR is CAR CTL019. In some embodiments, the CAR comprises the amino acid sequence of the CAR expressed by the CAR T-cell tisagenlecleucel. In some embodiments, the CAR is the CAR expressed by the CAR T-cell tisagenlecleucel. In some embodiments, the CAR comprises the amino acid sequence of the CAR expressed by the CAR T-cell KYMRIAH®. In some embodiments, the CAR is the CAR expressed by the CAR T-cell KYMRIAH®. In some embodiments, the CAR comprises the amino acid sequence of CAR KTE-C19. In some embodiments, the CAR is CAR KTE-C19. In some embodiments, the CAR comprises the amino acid sequence of the CAR expressed by the CAR T-cell axicabtagene ciloleucel. In some embodiments, the CAR is the CAR expressed by the CAR T-cell axicabtagene ciloleucel. In some embodiments, the CAR comprises the amino acid sequence of the CAR expressed by the CAR T-cell YESCARTA®. In some embodiments, the CAR is the CAR expressed by the CAR T-cell YESCARTA®.

Additional exemplary CD19 specific CARs are disclosed in e.g., U.S. Pat. No. 89,006,682, WO2019213282, US20200268860, WO2020227177, U.S. Ser. No. 10/457,730, WO2019159193, U.S. Ser. No. 10/287,350, U.S. Ser. No. 10/221,245, US20190125799, WO2018201794, US20170368098, US20160145337, U.S. Pat. No. 9,701,758, WO2014153270, WO2012079000, WO2019160956, WO2019161796, WO2020222176, WO2020219848, US20190135894, U.S. Ser. No. 10/774,388, WO2020180882, U.S. Ser. No. 10/765,701, WO2020172641, WO2020172440, WO2016149578, WO2020124021, WO2020108646, WO2020108643, WO2020113188, WO2020108644, WO2020108645, WO2020108642, U.S. Ser. No. 10/669,549, WO2020102770, U.S. Ser. No. 10/501,539, WO2020069409, U.S. Ser. No. 10/603,380, U.S. Ser. No. 10/533,055, WO2020010235, WO2019246546, the full contents of each of which is incorporated by reference herein.

TABLE 5
Amino acid and polynucleotide sequences of exemplary hCD19 specific CARs.

| | | SEQ | | SEQ |
|---|---|---|---|---|
| | | ID | | ID |
| Description | Amino Acid Sequence | NO | Polynucleotide Sequence | NO |
| CD19CAR | MLLLVTSLLLCELPHP | 72 | ATGCTGCTGCTGGTGACCAGCCTGCTGCTG | 82 |
| (with N- | AFLLIPDIQMTQTTSS | | TGTGAGCTGCCCCACCCCGCCTTTCTGCTG | |
| terminal signal | LSASLGDRVTISCRAS | | ATCCCCGACATCCAGATGACCCAGACCACC | |
| sequence and | QDISKYLNWYQQKPDG | | TCCAGCCTGAGCGCCAGCCTGGGCGACCGG | |
| without C- | TVKLLIYHTSRLHSGV | | GTGACCATCAGCTGCCGGGCCAGCCAGGAC | |
| terminal tail) | PSRFSGSGSGTDYSLT | | ATCAGCAAGTACCTGAACTGGTATCAGCAG | |
| | ISNLEQEDIATYFCQQ | | AAGCCCGACGGCACCGTCAAGCTGCTGATC | |
| | GNTLPYTFGGGTKLEI | | TACCACACCAGCCGGCTGCACAGCGGCGTG | |
| | TGSTSGSGKPGSGEGS | | CCCAGCCGGTTTAGCGGCAGCGGCTCCGGC | |
| | TKGEVKLQESGPGLVA | | ACCGACTACAGCCTGACCATCTCCAACCTG | |
| | PSQSLSVTCTVSGVSL | | GAGCAGGAGGACATCGCCACCTACTTTTGC | |
| | PDYGVSWIRQPPRKGL | | CAGCAGGGCAACACACTGCCCTACACCTTT | |
| | EWLGVIWGSETTYYNS | | GGCGGCGGAACAAAGCTGGAGATCACCGGC | |
| | ALKSRLTIIKDNSKSQ | | AGCACCTCCGGCAGCGGCAAGCCTGGCAGC | |
| | VFLKMNSLQTDDTAIY | | GGCGAGGGCAGCACCAAGGGCGAGGTGAAG | |
| | YCAKHYYYGGSYAMDY | | CTGCAGGAGAGCGGCCCTGGCCTGGTGGCC | |
| | WGQGTSVTVSSKPTTT | | CCCAGCCAGAGCCTGAGCGTGACCTGTACC | |
| | PAPRPPTPAPTIASQP | | GTGTCCGGCGTGTCCCTGCCCGACTACGGC | |
| | LSLRPEACRPAAGGAV | | GTGTCCTGGATCCGGCAGCCCCCTAGGAAG | |
| | HTRGLDFACDIYIWAP | | GGCCTGGAGTGGCTGGGCGTGATCTGGGGC | |
| | LAGTCGVLLLSLVITL | | AGCGAGACCACCTACTACAACAGCGCCCTG | |
| | YCNHRNRSKRSRGGHS | | AAGAGCCGGCTGACCATCATCAAGGACAAC | |
| | DYMNMTPRRPGPTRKH | | AGCAAGAGCCAGGTGTTCCTGAAGATGAAC | |
| | YQPYAPPRDFAAYRSR | | AGCCTGCAGACCGACGACACCGCCATCTAC | |
| | VKFSRSADAPAYQQGQ | | TACTGTGCCAAGCACTACTACTACGGCGGC | |
| | NQLYNELNLGRREEYD | | AGCTACGCCATGGACTACTGGGGCCAGGGC | |
| | VLDKRRGRDPEMGGKP | | ACCAGCGTGACCGTGTCCAGCAAGCCCACC | |
| | RRKNPQEGLYNELQKD | | ACCACCCCTGCCCCTAGACCTCCAACCCCA | |
| | KMAEAYSEIGMKGERR | | GCCCCTACAATCGCCAGCCAGCCCCTGAGC | |
| | RGKGHDGLYQGLSTAT | | CTGAGGCCCGAAGCCTGTAGACCTGCCGCT | |
| | KDTYDALHMQALPPR | | GGCGGAGCCGTGCACACCAGAGGCCTGGAT | |
| | | | TTCGCCTGCGACATCTACATCTGGGCCCCT | |
| | | | CTGGCCGGCACCTGTGGCGTGCTGCTGCTG | |
| | | | AGCCTGGTCATCACCCTGTACTGCAACCAC | |
| | | | CGGAATAGGAGCAAGCGGAGCAGAGGCGGC | |
| | | | CACAGCGACTACATGAACATGACCCCCCGG | |
| | | | AGGCCTGGCCCCACCCGGAAGCACTACCAG | |
| | | | CCCTACGCCCCTCCCAGGGACTTCGCCGCC | |
| | | | TACCGGAGCCGGGTGAAGTTCAGCCGGAGC | |
| | | | GCCGACGCCCCTGCCTACCAGCAGGGCCAG | |
| | | | AACCAGCTGTACAACGAGCTGAACCTGGGC | |
| | | | CGGAGGGAGGAGTACGACGTGCTGGACAAG | |
| | | | CGGAGAGGCCGGGACCCTGAGATGGGCGGC | |
| | | | AAGCCCCGGAGAAAGAACCCTCAGGAGGGC | |
| | | | CTGTATAACGAACTGCAGAAAGACAAGATG | |
| | | | GCCGAGGCCTACAGCGAGATCGGCATGAAG | |
| | | | GGCGAGCGGCGGAGGGGCAAGGGCCACGAC | |
| | | | GGCCTGTACCAGGGCCTGAGCACCGCCACC | |
| | | | AAGGATACCTACGACGCCCTGCACATGCAG | |
| | | | GCCCTGCCCCCCAGA | |
| | | | ATGCTGCTGCTGGTGACCAGCCTGCTGCTG | 83 |
| | | | TGTGAGCTGCCCCACCCCGCCTTTCTGCTG | |
| | | | ATCCCCGACATCCAGATGACCCAGACCACC | |
| | | | TCCAGCCTGAGCGCCAGCCTGGGCGACCGG | |
| | | | GTGACCATCAGCTGCCGGGCCAGCCAGGAC | |
| | | | ATCAGCAAGTACCTGAACTGGTATCAGCAG | |
| | | | AAGCCCGACGGCACCGTCAAGCTGCTGATC | |
| | | | TACCACACCAGCCGGCTGCACAGCGGCGTG | |
| | | | CCCAGCCGGTTTAGCGGCAGCGGCTCCGGC | |
| | | | ACCGACTACAGCCTGACCATCTCCAACCTG | |
| | | | GAGCAGGAGGACATCGCCACCTACTTTTGC | |
| | | | CAGCAGGGCAACACACTGCCCTACACCTTT | |
| | | | GGCGGCGGAACAAAGCTGGAGATCACCGGC | |
| | | | AGCACCTCCGGCAGCGGCAAGCCTGGCAGC | |
| | | | GGCGAGGGCAGCACCAAGGGCGAGGTGAAG | |
| | | | CTGCAGGAGAGCGGCCCTGGCCTGGTGGCC | |
| | | | CCCAGCCAGAGCCTGAGCGTGACCTGTACC | |
| | | | GTGTCCGGCGTGTCCCTGCCCGACTACGGC | |
| | | | GTGTCCTGGATCCGGCAGCCCCCTAGGAAG | |
| | | | GGCCTGGAGTGGCTGGGCGTGATCTGGGGC | |
| | | | AGCGAGACCACCTACTACAACAGCGCCCTG | |
| | | | AAGAGCCGGCTGACCATCATCAAGGACAAC | |
| | | | AGCAAGAGCCAGGTGTTCCTGAAGATGAAC | |
| | | | AGCCTGCAGACCGACGACACCGCCATCTAC | |
| | | | TACTGTGCCAAGCACTACTACTACGGCGGC | |
| | | | AGCTACGCCATGGACTACTGGGGCCAGGGC | |
| | | | ACCAGCGTGACCGTGTCCAGCAAGCCCACC | |
| | | | ACCACCCCTGCCCCTAGACCTCCAACCCCA | |
| | | | GCCCCTACAATCGCCAGCCAGCCCCTGAGC | |
| | | | CTGAGGCCCGAAGCCTGTAGACCTGCCGCT | |
| | | | GGCGGAGCCGTGCACACCAGAGGCCTGGAT | |
| | | | TTCGCCTGCGACATCTACATCTGGGCACCT | |
| | | | CTGGCCGGCACCTGTGGCGTGCTGCTGCTG | |
| | | | AGCCTGGTCATCACCCTGTACTGCAACCAC | |
| | | | CGGAATAGGAGCAAGCGGAGCAGAGGCGGC | |
| | | | CACAGCGACTACATGAACATGACCCCCCGG | |
| | | | AGGCCTGGCCCCACCCGGAAGCACTACCAG | |
| | | | CCCTACGCCCCTCCCAGGGACTTCGCCGCC | |
| | | | TACCGGAGCCGGGTGAAGTTCAGCCGGAGC | |
| | | | GCCGACGCCCCTGCCTACCAGCAGGGCCAG | |
| | | | AACCAGCTGTACAACGAGCTGAACCTGGGC | |
| | | | CGGAGGGAGGAGTACGACGTGCTGGACAAG | |
| | | | CGGAGAGGCCGGGACCCTGAGATGGGCGGC | |
| | | | AAGCCCCGGAGAAAGAACCCTCAGGAGGGC | |
| | | | CTGTATAACGAACTGCAGAAAGACAAGATG | |
| | | | GCCGAGGCCTACAGCGAGATCGGCATGAAG | |
| | | | GGCGAGCGGCGGAGGGGCAAGGGCCACGAC | |
| | | | GGCCTGTACCAGGGCCTGAGCACCGCCACC | |
| | | | AAGGATACCTACGACGCCCTGCACATGCAG | |
| | | | GCCCTGCCCCCCAGA | |
| CD19CAR | MLLLVTSLLLCELPHP | 73 | ATGCTGCTGCTGGTGACCAGCCTGCTGCTG | 84 |
| (with N- | AFLLIPDIQMTQTTSS | | TGTGAGCTGCCCCACCCCGCCTTTCTGCTG | |
| terminal signal | LSASLGDRVTISCRAS | | ATCCCCGACATCCAGATGACCCAGACCACC | |
| sequence and | QDISKYLNWYQQKPDG | | TCCAGCCTGAGCGCCAGCCTGGGCGACCGG | |
| with C- | TVKLLIYHTSRLHSGV | | GTGACCATCAGCTGCCGGGCCAGCCAGGAC | |
| terminal tail) | PSRFSGSGSGTDYSLT | | ATCAGCAAGTACCTGAACTGGTATCAGCAG | |
| | ISNLEQEDIATYFCQQ | | AAGCCCGACGGCACCGTCAAGCTGCTGATC | |
| | GNTLPYTFGGGTKLEI | | TACCACACCAGCCGGCTGCACAGCGGCGTG | |
| | TGSTSGSGKPGSGEGS | | CCCAGCCGGTTTAGCGGCAGCGGCTCCGGC | |
| | TKGEVKLQESGPGLVA | | ACCGACTACAGCCTGACCATCTCCAACCTG | |
| | PSQSLSVTCTVSGVSL | | GAGCAGGAGGACATCGCCACCTACTTTTGC | |
| | PDYGVSWIRQPPRKGL | | CAGCAGGGCAACACACTGCCCTACACCTTT | |
| | EWLGVIWGSETTYYNS | | GGCGGCGGAACAAAGCTGGAGATCACCGGC | |
| | ALKSRLTIIKDNSKSQ | | AGCACCTCCGGCAGCGGCAAGCCTGGCAGC | |
| | VFLKMNSLQTDDTAIY | | GGCGAGGGCAGCACCAAGGGCGAGGTGAAG | |
| | YCAKHYYYGGSYAMDY | | CTGCAGGAGAGCGGCCCTGGCCTGGTGGCC | |
| | WGQGTSVTVSSKPTTT | | CCCAGCCAGAGCCTGAGCGTGACCTGTACC | |
| | PAPRPPTPAPTIASQP | | GTGTCCGGCGTGTCCCTGCCCGACTACGGC | |
| | LSLRPEACRPAAGGAV | | GTGTCCTGGATCCGGCAGCCCCCTAGGAAG | |
| | HTRGLDFACDIYIWAP | | GGCCTGGAGTGGCTGGGCGTGATCTGGGGC | |
| | LAGTCGVLLLSLVITL | | AGCGAGACCACCTACTACAACAGCGCCCTG | |
| | YCNHRNRSKRSRGGHS | | AAGAGCCGGCTGACCATCATCAAGGACAAC | |
| | DYMNMTPRRPGPTRKH | | AGCAAGAGCCAGGTGTTCCTGAAGATGAAC | |
| | YQPYAPPRDFAAYRSR | | AGCCTGCAGACCGACGACACCGCCATCTAC | |
| | VKFSRSADAPAYQQGQ | | TACTGTGCCAAGCACTACTACTACGGCGGC | |
| | NQLYNELNLGRREEYD | | AGCTACGCCATGGACTACTGGGGCCAGGGC | |
| | VLDKRRGRDPEMGGKP | | ACCAGCGTGACCGTGTCCAGCAAGCCCACC | |
| | RRKNPQEGLYNELQKD | | ACCACCCCTGCCCCTAGACCTCCAACCCCA | |
| | KMAEAYSEIGMKGERR | | GCCCCTACAATCGCCAGCCAGCCCCTGAGC | |
| | RGKGHDGLYQGLSTAT | | CTGAGGCCCGAAGCCTGTAGACCTGCCGCT | |
| | KDTYDALHMQALPPRG | | GGCGGAGCCGTGCACACCAGAGGCCTGGAT | |
| | SGVKQTLNFDLLKLAG | | TTCGCCTGCGACATCTACATCTGGGCCCCT | |
| | DVESNPG | | CTGGCCGGCACCTGTGGCGTGCTGCTGCTG | |
| | | | AGCCTGGTCATCACCCTGTACTGCAACCAC | |
| | | | CGGAATAGGAGCAAGCGGAGCAGAGGCGGC | |
| | | | CACAGCGACTACATGAACATGACCCCCCGG | |
| | | | AGGCCTGGCCCCACCCGGAAGCACTACCAG | |
| | | | CCCTACGCCCCTCCCAGGGACTTCGCCGCC | |
| | | | TACCGGAGCCGGGTGAAGTTCAGCCGGAGC | |
| | | | GCCGACGCCCCTGCCTACCAGCAGGGCCAG | |
| | | | AACCAGCTGTACAACGAGCTGAACCTGGGC | |
| | | | CGGAGGGAGGAGTACGACGTGCTGGACAAG | |
| | | | CGGAGAGGCCGGGACCCTGAGATGGGCGGC | |
| | | | AAGCCCCGGAGAAAGAACCCTCAGGAGGGC | |
| | | | CTGTATAACGAACTGCAGAAAGACAAGATG | |
| | | | GCCGAGGCCTACAGCGAGATCGGCATGAAG | |
| | | | GGCGAGCGGCGGAGGGGCAAGGGCCACGAC | |
| | | | GGCCTGTACCAGGGCCTGAGCACCGCCACC | |
| | | | AAGGATACCTACGACGCCCTGCACATGCAG | |
| | | | GCCCTGCCCCCCAGAGGCTCCGGAGTGAAG | |
| | | | CAGACCCTGAATTTCGACCTGCTGAAGCTG | |
| | | | GCCGGGGACGTGGAGAGCAACCCTGGC | |
| CD19CAR | DIQMTQTTSSLSASLG | 74 | GACATCCAGATGACCCAGACCACCTCCAGC | 86 |
| (without N- | DRVTISCRASQDISKY | | CTGAGCGCCAGCCTGGGCGACCGGGTGACC | |
| terminal signal | LNWYQQKPDGTVKLLI | | ATCAGCTGCCGGGCCAGCCAGGACATCAGC | |
| sequence and | YHTSRLHSGVPSRFSG | | AAGTACCTGAACTGGTATCAGCAGAAGCCC | |
| without C- | SGSGTDYSLTISNLEQ | | GACGGCACCGTCAAGCTGCTGATCTACCAC | |
| terminal tail) | EDIATYFCQQGNTLPY | | ACCAGCCGGCTGCACAGCGGCGTGCCCAGC | |
| | TFGGGTKLEITGSTSG | | CGGTTTAGCGGCAGCGGCTCCGGCACCGAC | |
| | SGKPGSGEGSTKGEVK | | TACAGCCTGACCATCTCCAACCTGGAGCAG | |
| | LQESGPGLVAPSQSLS | | GAGGACATCGCCACCTACTTTTGCCAGCAG | |
| | VTCTVSGVSLPDYGVS | | GGCAACACACTGCCCTACACCTTTGGCGGC | |
| | WIRQPPRKGLEWLGVI | | GGAACAAAGCTGGAGATCACCGGCAGCACC | |
| | WGSETTYYNSALKSRL | | TCCGGCAGCGGCAAGCCTGGCAGCGGCGAG | |
| | TIIKDNSKSQVFLKMN | | GGCAGCACCAAGGGCGAGGTGAAGCTGCAG | |
| | SLQTDDTAIYYCAKHY | | GAGAGCGGCCCTGGCCTGGTGGCCCCCAGC | |
| | YYGGSYAMDYWGQGTS | | CAGAGCCTGAGCGTGACCTGTACCGTGTCC | |
| | VTVSSKPTTTPAPRPP | | GGCGTGTCCCTGCCCGACTACGGCGTGTCC | |
| | TPAPTIASQPLSLRPE | | TGGATCCGGCAGCCCCCTAGGAAGGGCCTG | |
| | ACRPAAGGAVHTRGLD | | GAGTGGCTGGGCGTGATCTGGGGCAGCGAG | |
| | FACDIYIWAPLAGTCG | | ACCACCTACTACAACAGCGCCCTGAAGAGC | |
| | VLLLSLVITLYCNHRN | | CGGCTGACCATCATCAAGGACAACAGCAAG | |
| | RSKRSRGGHSDYMNMT | | AGCCAGGTGTTCCTGAAGATGAACAGCCTG | |
| | PRRPGPTRKHYQPYAP | | CAGACCGACGACACCGCCATCTACTACTGT | |
| | PRDFAAYRSRVKESRS | | GCCAAGCACTACTACTACGGCGGCAGCTAC | |
| | ADAPAYQQGQNQLYNE | | GCCATGGACTACTGGGGCCAGGGCACCAGC | |
| | LNLGRREEYDVLDKRR | | GTGACCGTGTCCAGCAAGCCCACCACCACC | |
| | GRDPEMGGKPRRKNPQ | | CCTGCCCCTAGACCTCCAACCCCAGCCCCT | |
| | EGLYNELQKDKMAEAY | | ACAATCGCCAGCCAGCCCCTGAGCCTGAGG | |
| | SEIGMKGERRRGKGHD | | CCCGAAGCCTGTAGACCTGCCGCTGGCGGA | |
| | GLYQGLSTATKDTYDA | | GCCGTGCACACCAGAGGCCTGGATTTCGCC | |
| | LHMQALPPR | | TGCGACATCTACATCTGGGCCCCTCTGGCC | |
| | | | GGCACCTGTGGCGTGCTGCTGCTGAGCCTG | |
| | | | GTCATCACCCTGTACTGCAACCACCGGAAT | |
| | | | AGGAGCAAGCGGAGCAGAGGCGGCCACAGC | |
| | | | GACTACATGAACATGACCCCCCGGAGGCCT | |
| | | | GGCCCCACCCGGAAGCACTACCAGCCCTAC | |
| | | | GCCCCTCCCAGGGACTTCGCCGCCTACCGG | |
| | | | AGCCGGGTGAAGTTCAGCCGGAGCGCCGAC | |
| | | | GCCCCTGCCTACCAGCAGGGCCAGAACCAG | |
| | | | CTGTACAACGAGCTGAACCTGGGCCGGAGG | |
| | | | GAGGAGTACGACGTGCTGGACAAGCGGAGA | |
| | | | GGCCGGGACCCTGAGATGGGCGGCAAGCCC | |
| | | | CGGAGAAAGAACCCTCAGGAGGGCCTGTAT | |
| | | | AACGAACTGCAGAAAGACAAGATGGCCGAG | |
| | | | GCCTACAGCGAGATCGGCATGAAGGGCGAG | |
| | | | CGGCGGAGGGGCAAGGGCCACGACGGCCTG | |
| | | | TACCAGGGCCTGAGCACCGCCACCAAGGAT | |
| | | | ACCTACGACGCCCTGCACATGCAGGCCCTG | |
| | | | CCCCCCAGA | |
| | | | GACATCCAGATGACCCAGACCACCTCCAGC | 87 |
| | | | CTGAGCGCCAGCCTGGGCGACCGGGTGACC | |
| | | | ATCAGCTGCCGGGCCAGCCAGGACATCAGC | |
| | | | AAGTACCTGAACTGGTATCAGCAGAAGCCC | |
| | | | GACGGCACCGTCAAGCTGCTGATCTACCAC | |
| | | | ACCAGCCGGCTGCACAGCGGCGTGCCCAGC | |
| | | | CGGTTTAGCGGCAGCGGCTCCGGCACCGAC | |
| | | | TACAGCCTGACCATCTCCAACCTGGAGCAG | |
| | | | GAGGACATCGCCACCTACTTTTGCCAGCAG | |
| | | | GGCAACACACTGCCCTACACCTTTGGCGGC | |
| | | | GGAACAAAGCTGGAGATCACCGGCAGCACC | |
| | | | TCCGGCAGCGGCAAGCCTGGCAGCGGCGAG | |
| | | | GGCAGCACCAAGGGCGAGGTGAAGCTGCAG | |
| | | | GAGAGCGGCCCTGGCCTGGTGGCCCCCAGC | |
| | | | CAGAGCCTGAGCGTGACCTGTACCGTGTCC | |
| | | | GGCGTGTCCCTGCCCGACTACGGCGTGTCC | |
| | | | TGGATCCGGCAGCCCCCTAGGAAGGGCCTG | |
| | | | GAGTGGCTGGGCGTGATCTGGGGCAGCGAG | |
| | | | ACCACCTACTACAACAGCGCCCTGAAGAGC | |
| | | | CGGCTGACCATCATCAAGGACAACAGCAAG | |
| | | | AGCCAGGTGTTCCTGAAGATGAACAGCCTG | |
| | | | CAGACCGACGACACCGCCATCTACTACTGT | |
| | | | GCCAAGCACTACTACTACGGCGGCAGCTAC | |
| | | | GCCATGGACTACTGGGGCCAGGGCACCAGC | |
| | | | GTGACCGTGTCCAGCAAGCCCACCACCACC | |
| | | | CCTGCCCCTAGACCTCCAACCCCAGCCCCT | |
| | | | ACAATCGCCAGCCAGCCCCTGAGCCTGAGG | |
| | | | CCCGAAGCCTGTAGACCTGCCGCTGGCGGA | |
| | | | GCCGTGCACACCAGAGGCCTGGATTTCGCC | |
| | | | TGCGACATCTACATCTGGGCACCTCTGGCC | |
| | | | GGCACCTGTGGCGTGCTGCTGCTGAGCCTG | |
| | | | GTCATCACCCTGTACTGCAACCACCGGAAT | |
| | | | AGGAGCAAGCGGAGCAGAGGCGGCCACAGC | |
| | | | GACTACATGAACATGACCCCCCGGAGGCCT | |
| | | | GGCCCCACCCGGAAGCACTACCAGCCCTAC | |
| | | | GCCCCTCCCAGGGACTTCGCCGCCTACCGG | |
| | | | AGCCGGGTGAAGTTCAGCCGGAGCGCCGAC | |
| | | | GCCCCTGCCTACCAGCAGGGCCAGAACCAG | |
| | | | CTGTACAACGAGCTGAACCTGGGCCGGAGG | |
| | | | GAGGAGTACGACGTGCTGGACAAGCGGAGA | |
| | | | GGCCGGGACCCTGAGATGGGCGGCAAGCCC | |
| | | | CGGAGAAAGAACCCTCAGGAGGGCCTGTAT | |
| | | | AACGAACTGCAGAAAGACAAGATGGCCGAG | |
| | | | GCCTACAGCGAGATCGGCATGAAGGGCGAG | |
| | | | CGGCGGAGGGGCAAGGGCCACGACGGCCTG | |
| | | | TACCAGGGCCTGAGCACCGCCACCAAGGAT | |
| | | | ACCTACGACGCCCTGCACATGCAGGCCCTG | |
| | | | CCCCCCAGA | |
| CD19CAR | DIQMTQTTSSLSASLG | 75 | GACATCCAGATGACCCAGACCACCTCCAGC | 88 |
| (without N- | DRVTISCRASQDISKY | | CTGAGCGCCAGCCTGGGCGACCGGGTGACC | |
| terminal signal | LNWYQQKPDGTVKLLI | | ATCAGCTGCCGGGCCAGCCAGGACATCAGC | |
| sequence and | YHTSRLHSGVPSRFSG | | AAGTACCTGAACTGGTATCAGCAGAAGCCC | |
| with C- | SGSGTDYSLTISNLEQ | | GACGGCACCGTCAAGCTGCTGATCTACCAC | |
| terminal tail) | EDIATYFCQQGNTLPY | | ACCAGCCGGCTGCACAGCGGCGTGCCCAGC | |
| | TFGGGTKLEITGSTSG | | CGGTTTAGCGGCAGCGGCTCCGGCACCGAC | |
| | SGKPGSGEGSTKGEVK | | TACAGCCTGACCATCTCCAACCTGGAGCAG | |
| | LQESGPGLVAPSQSLS | | GAGGACATCGCCACCTACTTTTGCCAGCAG | |
| | VTCTVSGVSLPDYGVS | | GGCAACACACTGCCCTACACCTTTGGCGGC | |
| | WIRQPPRKGLEWLGVI | | GGAACAAAGCTGGAGATCACCGGCAGCACC | |
| | WGSETTYYNSALKSRL | | TCCGGCAGCGGCAAGCCTGGCAGCGGCGAG | |
| | TIIKDNSKSQVFLKMN | | GGCAGCACCAAGGGCGAGGTGAAGCTGCAG | |
| | SLQTDDTAIYYCAKHY | | GAGAGCGGCCCTGGCCTGGTGGCCCCCAGC | |
| | YYGGSYAMDYWGQGTS | | CAGAGCCTGAGCGTGACCTGTACCGTGTCC | |
| | VTVSSKPTTTPAPRPP | | GGCGTGTCCCTGCCCGACTACGGCGTGTCC | |
| | TPAPTIASQPLSLRPE | | TGGATCCGGCAGCCCCCTAGGAAGGGCCTG | |
| | ACRPAAGGAVHTRGLD | | GAGTGGCTGGGCGTGATCTGGGGCAGCGAG | |
| | FACDIYIWAPLAGTCG | | ACCACCTACTACAACAGCGCCCTGAAGAGC | |
| | VLLLSLVITLYCNHRN | | CGGCTGACCATCATCAAGGACAACAGCAAG | |
| | RSKRSRGGHSDYMNMT | | AGCCAGGTGTTCCTGAAGATGAACAGCCTG | |
| | PRRPGPTRKHYQPYAP | | CAGACCGACGACACCGCCATCTACTACTGT | |
| | PRDFAAYRSRVKFSRS | | GCCAAGCACTACTACTACGGCGGCAGCTAC | |
| | ADAPAYQQGQNQLYNE | | GCCATGGACTACTGGGGCCAGGGCACCAGC | |
| | LNLGRREEYDVLDKRR | | GTGACCGTGTCCAGCAAGCCCACCACCACC | |
| | GRDPEMGGKPRRKNPQ | | CCTGCCCCTAGACCTCCAACCCCAGCCCCT | |
| | EGLYNELQKDKMAEAY | | ACAATCGCCAGCCAGCCCCTGAGCCTGAGG | |
| | SEIGMKGERRRGKGHD | | CCCGAAGCCTGTAGACCTGCCGCTGGCGGA | |
| | GLYQGLSTATKDTYDA | | GCCGTGCACACCAGAGGCCTGGATTTCGCC | |
| | LHMQALPPRGSGVKQT | | TGCGACATCTACATCTGGGCCCCTCTGGCC | |
| | LNFDLLKLAGDVESNP | | GGCACCTGTGGCGTGCTGCTGCTGAGCCTG | |
| | G | | GTCATCACCCTGTACTGCAACCACCGGAAT | |
| | | | AGGAGCAAGCGGAGCAGAGGCGGCCACAGC | |
| | | | GACTACATGAACATGACCCCCCGGAGGCCT | |
| | | | GGCCCCACCCGGAAGCACTACCAGCCCTAC | |
| | | | GCCCCTCCCAGGGACTTCGCCGCCTACCGG | |
| | | | AGCCGGGTGAAGTTCAGCCGGAGCGCCGAC | |
| | | | GCCCCTGCCTACCAGCAGGGCCAGAACCAG | |
| | | | CTGTACAACGAGCTGAACCTGGGCCGGAGG | |
| | | | GAGGAGTACGACGTGCTGGACAAGCGGAGA | |
| | | | GGCCGGGACCCTGAGATGGGCGGCAAGCCC | |
| | | | CGGAGAAAGAACCCTCAGGAGGGCCTGTAT | |
| | | | AACGAACTGCAGAAAGACAAGATGGCCGAG | |
| | | | GCCTACAGCGAGATCGGCATGAAGGGCGAG | |
| | | | CGGCGGAGGGGCAAGGGCCACGACGGCCTG | |
| | | | TACCAGGGCCTGAGCACCGCCACCAAGGAT | |
| | | | ACCTACGACGCCCTGCACATGCAGGCCCTG | |
| | | | CCCCCCAGAGGCTCCGGAGTGAAGCAGACC | |
| | | | CTGAATTTCGACCTGCTGAAGCTGGCCGGG | |
| | | | GACGTGGAGAGCAACCCTGGC | |
| CAR Variant 1 | MLLLVTSLLLCELPHP | 76 | ATGCTGCTGCTGGTGACCAGCCTGCTGCTG | 90 |
| (with N- | AFLLIPEVKLQESGPG | | TGTGAGCTGCCCCACCCCGCCTTTCTGCTG | |
| terminal signal | LVAPSQSLSVTCTVSG | | ATCCCCGAGGTGAAGCTGCAGGAGAGCGGC | |
| sequence) | VSLPDYGVSWIRQPPR | | CCTGGCCTGGTGGCCCCCAGCCAGAGCCTG | |
| | KGLEWLGVIWGSETTY | | AGCGTGACCTGTACCGTGTCCGGCGTGTCC | |
| | YNSALKSRLTIIKDNS | | CTGCCCGACTACGGCGTGTCCTGGATCCGG | |
| | KSQVFLKMNSLQTDDT | | CAGCCCCCTAGGAAGGGCCTGGAGTGGCTG | |
| | AIYYCAKHYYYGGSYA | | GGCGTGATCTGGGGCAGCGAGACCACCTAC | |
| | MDYWGQGTSVTVSSGS | | TACAACAGCGCCCTGAAGAGCCGGCTGACC | |
| | TSGSGKPGSGEGSTKG | | ATCATCAAGGACAACAGCAAGAGCCAGGTG | |
| | DIQMTQTTSSLSASLG | | TTCCTGAAGATGAACAGCCTGCAGACCGAC | |
| | DRVTISCRASQDISKY | | GACACCGCCATCTACTACTGTGCCAAGCAC | |
| | LNWYQQKPDGTVKLLI | | TACTACTACGGCGGCAGCTACGCCATGGAC | |
| | YHTSRLHSGVPSRFSG | | TACTGGGGCCAGGGCACCAGCGTGACCGTG | |
| | SGSGTDYSLTISNLEQ | | TCCAGCGGCAGCACCTCCGGCAGCGGCAAG | |
| | EDIATYFCQQGNTLPY | | CCTGGCAGCGGCGAGGGCAGCACCAAGGGC | |
| | TFGGGTKLEITKPTTT | | GACATCCAGATGACCCAGACCACCTCCAGC | |
| | PAPRPPTPAPTIASQP | | CTGAGCGCCAGCCTGGGCGACCGGGTGACC | |
| | LSLRPEACRPAAGGAV | | ATCAGCTGCCGGGCCAGCCAGGACATCAGC | |
| | HTRGLDFACDIYIWAP | | AAGTACCTGAACTGGTATCAGCAGAAGCCC | |
| | LAGTCGVLLLSLVITL | | GACGGCACCGTCAAGCTGCTGATCTACCAC | |
| | YCNHRNRSKRSRGGHS | | ACCAGCCGGCTGCACAGCGGCGTGCCCAGC | |
| | DYMNMTPRRPGPTRKH | | CGGTTTAGCGGCAGCGGCTCCGGCACCGAC | |
| | YQPYAPPRDFAAYRSR | | TACAGCCTGACCATCTCCAACCTGGAGCAG | |
| | VKFSRSADAPAYQQGQ | | GAGGACATCGCCACCTACTTTTGCCAGCAG | |
| | NQLYNELNLGRREEYD | | GGCAACACACTGCCCTACACCTTTGGCGGC | |
| | VLDKRRGRDPEMGGKP | | GGAACAAAGCTGGAGATCACCAAGCCCACC | |
| | RRKNPQEGLYNELQKD | | ACCACCCCTGCCCCTAGACCTCCAACCCCA | |
| | KMAEAYSEIGMKGERR | | GCCCCTACAATCGCCAGCCAGCCCCTGAGC | |
| | RGKGHDGLYQGLSTAT | | CTGAGGCCCGAAGCCTGTAGACCTGCCGCT | |
| | KDTYDALHMQALPPR | | GGCGGAGCCGTGCACACCAGAGGCCTGGAT | |
| | | | TTCGCCTGCGACATCTACATCTGGGCACCT | |
| | | | CTGGCCGGCACCTGTGGCGTGCTGCTGCTG | |
| | | | AGCCTGGTCATCACCCTGTACTGCAACCAC | |
| | | | CGGAATAGGAGCAAGCGGAGCAGAGGCGGC | |
| | | | CACAGCGACTACATGAACATGACCCCCCGG | |
| | | | AGGCCTGGCCCCACCCGGAAGCACTACCAG | |
| | | | CCCTACGCCCCTCCCAGGGACTTCGCCGCC | |
| | | | TACCGGAGCCGGGTGAAGTTCAGCCGGAGC | |
| | | | GCCGACGCCCCTGCCTACCAGCAGGGCCAG | |
| | | | AACCAGCTGTACAACGAGCTGAACCTGGGC | |
| | | | CGGAGGGAGGAGTACGACGTGCTGGACAAG | |
| | | | CGGAGAGGCCGGGACCCTGAGATGGGCGGC | |
| | | | AAGCCCCGGAGAAAGAACCCTCAGGAGGGC | |
| | | | CTGTATAACGAACTGCAGAAAGACAAGATG | |
| | | | GCCGAGGCCTACAGCGAGATCGGCATGAAG | |
| | | | GGCGAGCGGCGGAGGGGCAAGGGCCACGAC | |
| | | | GGCCTGTACCAGGGCCTGAGCACCGCCACC | |
| | | | AAGGATACCTACGACGCCCTGCACATGCAG | |
| | | | GCCCTGCCCCCCAGA | |
| CAR Variant 1 | EVKLQESGPGLVAPSQ | 77 | GAGGTGAAGCTGCAGGAGAGCGGCCCTGGC | 91 |
| (without N- | SLSVTCTVSGVSLPDY | | CTGGTGGCCCCCAGCCAGAGCCTGAGCGTG | |
| terminal signal | GVSWIRQPPRKGLEWL | | ACCTGTACCGTGTCCGGCGTGTCCCTGCCC | |
| sequence) | GVIWGSETTYYNSALK | | GACTACGGCGTGTCCTGGATCCGGCAGCCC | |
| | SRLTIIKDNSKSQVFL | | CCTAGGAAGGGCCTGGAGTGGCTGGGCGTG | |
| | KMNSLQTDDTAIYYCA | | ATCTGGGGCAGCGAGACCACCTACTACAAC | |
| | KHYYYGGSYAMDYWGQ | | AGCGCCCTGAAGAGCCGGCTGACCATCATC | |
| | GTSVTVSSGSTSGSGK | | AAGGACAACAGCAAGAGCCAGGTGTTCCTG | |
| | PGSGEGSTKGDIQMTQ | | AAGATGAACAGCCTGCAGACCGACGACACC | |
| | TTSSLSASLGDRVTIS | | GCCATCTACTACTGTGCCAAGCACTACTAC | |
| | CRASQDISKYLNWYQQ | | TACGGCGGCAGCTACGCCATGGACTACTGG | |
| | KPDGTVKLLIYHTSRL | | GGCCAGGGCACCAGCGTGACCGTGTCCAGC | |
| | HSGVPSRFSGSGSGTD | | GGCAGCACCTCCGGCAGCGGCAAGCCTGGC | |
| | YSLTISNLEQEDIATY | | AGCGGCGAGGGCAGCACCAAGGGCGACATC | |
| | FCQQGNTLPYTFGGGT | | CAGATGACCCAGACCACCTCCAGCCTGAGC | |
| | KLEITKPTTTPAPRPP | | GCCAGCCTGGGCGACCGGGTGACCATCAGC | |
| | TPAPTIASQPLSLRPE | | TGCCGGGCCAGCCAGGACATCAGCAAGTAC | |
| | ACRPAAGGAVHTRGLD | | CTGAACTGGTATCAGCAGAAGCCCGACGGC | |
| | FACDIYIWAPLAGTCG | | ACCGTCAAGCTGCTGATCTACCACACCAGC | |
| | VLLLSLVITLYCNHRN | | CGGCTGCACAGCGGCGTGCCCAGCCGGTTT | |
| | RSKRSRGGHSDYMNMT | | AGCGGCAGCGGCTCCGGCACCGACTACAGC | |
| | PRRPGPTRKHYQPYAP | | CTGACCATCTCCAACCTGGAGCAGGAGGAC | |
| | PRDFAAYRSRVKESRS | | ATCGCCACCTACTTTTGCCAGCAGGGCAAC | |
| | ADAPAYQQGQNQLYNE | | ACACTGCCCTACACCTTTGGCGGCGGAACA | |
| | LNLGRREEYDVLDKRR | | AAGCTGGAGATCACCAAGCCCACCACCACC | |
| | GRDPEMGGKPRRKNPQ | | CCTGCCCCTAGACCTCCAACCCCAGCCCCT | |
| | EGLYNELQKDKMAEAY | | ACAATCGCCAGCCAGCCCCTGAGCCTGAGG | |
| | SEIGMKGERRRGKGHD | | CCCGAAGCCTGTAGACCTGCCGCTGGCGGA | |
| | GLYQGLSTATKDTYDA | | GCCGTGCACACCAGAGGCCTGGATTTCGCC | |
| | LHMQALPPR | | TGCGACATCTACATCTGGGCACCTCTGGCC | |
| | | | GGCACCTGTGGCGTGCTGCTGCTGAGCCTG | |
| | | | GTCATCACCCTGTACTGCAACCACCGGAAT | |
| | | | AGGAGCAAGCGGAGCAGAGGCGGCCACAGC | |
| | | | GACTACATGAACATGACCCCCCGGAGGCCT | |
| | | | GGCCCCACCCGGAAGCACTACCAGCCCTAC | |
| | | | GCCCCTCCCAGGGACTTCGCCGCCTACCGG | |
| | | | AGCCGGGTGAAGTTCAGCCGGAGCGCCGAC | |
| | | | GCCCCTGCCTACCAGCAGGGCCAGAACCAG | |
| | | | CTGTACAACGAGCTGAACCTGGGCCGGAGG | |
| | | | GAGGAGTACGACGTGCTGGACAAGCGGAGA | |
| | | | GGCCGGGACCCTGAGATGGGCGGCAAGCCC | |
| | | | CGGAGAAAGAACCCTCAGGAGGGCCTGTAT | |
| | | | AACGAACTGCAGAAAGACAAGATGGCCGAG | |
| | | | GCCTACAGCGAGATCGGCATGAAGGGCGAG | |
| | | | CGGCGGAGGGGCAAGGGCCACGACGGCCTG | |
| | | | TACCAGGGCCTGAGCACCGCCACCAAGGAT | |
| | | | ACCTACGACGCCCTGCACATGCAGGCCCTG | |
| | | | CCCCCCAGA | |
| CAR Variant 2 | MALPVTALLLPLALLL | 78 | ATGGCCTTACCAGTGACCGCCTTGCTCCTG | 92 |
| (with N- | HAARPDIQMTQTTSSL | | CCGCTGGCCTTGCTGCTCCACGCCGCCAGG | |
| terminal signal | SASLGDRVTISCRASQ | | CCGGACATCCAGATGACACAGACTACATCC | |
| sequence) | DISKYLNWYQQKPDGT | | TCCCTGTCTGCCTCTCTGGGAGACAGAGTC | |
| | VKLLIYHTSRLHSGVP | | ACCATCAGTTGCAGGGCAAGTCAGGACATT | |
| | SRFSGSGSGTDYSLTI | | AGTAAATATTTAAATTGGTATCAGCAGAAA | |
| | SNLEQEDIATYFCQQG | | CCAGATGGAACTGTTAAACTCCTGATCTAC | |
| | NTLPYTFGGGTKLEIT | | CATACATCAAGATTACACTCAGGAGTCCCA | |
| | GGGGSGGGGSGGGGSE | | TCAAGGTTCAGTGGCAGTGGGTCTGGAACA | |
| | VKLQESGPGLVAPSQS | | GATTATTCTCTCACCATTAGCAACCTGGAG | |
| | LSVTCTVSGVSLPDYG | | CAAGAAGATATTGCCACTTACTTTTGCCAA | |
| | VSWIRQPPRKGLEWLG | | CAGGGTAATACGCTTCCGTACACGTTCGGA | |
| | VIWGSETTYYNSALKS | | GGGGGGACCAAGCTGGAGATCACAGGTGGC | |
| | RLTIIKDNSKSQVFLK | | GGTGGCTCGGGCGGTGGTGGGTCGGGTGGC | |
| | MNSLQTDDTAIYYCAK | | GGCGGATCTGAGGTGAAACTGCAGGAGTCA | |
| | HYYYGGSYAMDYWGQG | | GGACCTGGCCTGGTGGCGCCCTCACAGAGC | |
| | TSVTVSSTTTPAPRPP | | CTGTCCGTCACATGCACTGTCTCAGGGGTC | |
| | TPAPTIASQPLSLRPE | | TCATTACCCGACTATGGTGTAAGCTGGATT | |
| | ACRPAAGGAVHTRGLD | | CGCCAGCCTCCACGAAAGGGTCTGGAGTGG | |
| | FACDIYIWAPLAGTCG | | CTGGGAGTAATATGGGGTAGTGAAACCACA | |
| | VLLLSLVITLYCKRGR | | TACTATAATTCAGCTCTCAAATCCAGACTG | |
| | KKLLYIFKQPFMRPVQ | | ACCATCATCAAGGACAACTCCAAGAGCCAA | |
| | TTQEEDGCSCRFPEEE | | GTTTTCTTAAAAATGAACAGTCTGCAAACT | |
| | EGGCELRVKFSRSADA | | GATGACACAGCCATTTACTACTGTGCCAAA | |
| | PAYKQGQNQLYNELNL | | CATTATTACTACGGTGGTAGCTATGCTATG | |
| | GRREEYDVLDKRRGRD | | GACTACTGGGGCCAAGGAACCTCAGTCACC | |
| | PEMGGKPRRKNPQEGL | | GTCTCCTCAACCACGACGCCAGCGCCGCGA | |
| | YNELQKDKMAEAYSEI | | CCACCAACACCGGCGCCCACCATCGCGTCG | |
| | GMKGERRRGKGHDGLY | | CAGCCCCTGTCCCTGCGCCCAGAGGCGTGC | |
| | QGLSTATKDTYDALHM | | CGGCCAGCGGCGGGGGGCGCAGTGCACACG | |
| | QALPPR | | AGGGGGCTGGACTTCGCCTGTGATATCTAC | |
| | | | ATCTGGGCGCCCTTGGCCGGGACTTGTGGG | |
| | | | GTCCTTCTCCTGTCACTGGTTATCACCCTT | |
| | | | TACTGCAAACGGGGCAGAAAGAAACTCCTG | |
| | | | TATATATTCAAACAACCATTTATGAGACCA | |
| | | | GTACAAACTACTCAAGAGGAAGATGGCTGT | |
| | | | AGCTGCCGATTTCCAGAAGAAGAAGAAGGA | |
| | | | GGATGTGAACTGAGAGTGAAGTTCAGCAGG | |
| | | | AGCGCAGACGCCCCCGCGTACAAGCAGGGC | |
| | | | CAGAACCAGCTCTATAACGAGCTCAATCTA | |
| | | | GGACGAAGAGAGGAGTACGATGTTTTGGAC | |
| | | | AAGAGACGTGGCCGGGACCCTGAGATGGGG | |
| | | | GGAAAGCCGAGAAGGAAGAACCCTCAGGAA | |
| | | | GGCCTGTACAATGAACTGCAGAAAGATAAG | |
| | | | ATGGCGGAGGCCTACAGTGAGATTGGGATG | |
| | | | AAAGGCGAGCGCCGGAGGGGCAAGGGGCAC | |
| | | | GATGGCCTTTACCAGGGTCTCAGTACAGCC | |
| | | | ACCAAGGACACCTACGACGCCCTTCACATG | |
| | | | CAGGCCCTGCCCCCTCGC | |
| CAR Variant 2 | DIQMTQTTSSLSASLG | 79 | GACATCCAGATGACACAGACTACATCCTCC | 93 |
| (without N- | DRVTISCRASQDISKY | | CTGTCTGCCTCTCTGGGAGACAGAGTCACC | |
| terminal signal | LNWYQQKPDGTVKLLI | | ATCAGTTGCAGGGCAAGTCAGGACATTAGT | |
| sequence) | YHTSRLHSGVPSRESG | | AAATATTTAAATTGGTATCAGCAGAAACCA | |
| | SGSGTDYSLTISNLEQ | | GATGGAACTGTTAAACTCCTGATCTACCAT | |
| | EDIATYFCQQGNTLPY | | ACATCAAGATTACACTCAGGAGTCCCATCA | |
| | TFGGGTKLEITGGGGS | | AGGTTCAGTGGCAGTGGGTCTGGAACAGAT | |
| | GGGGSGGGGSEVKLQE | | TATTCTCTCACCATTAGCAACCTGGAGCAA | |
| | SGPGLVAPSQSLSVTC | | GAAGATATTGCCACTTACTTTTGCCAACAG | |
| | TVSGVSLPDYGVSWIR | | GGTAATACGCTTCCGTACACGTTCGGAGGG | |
| | QPPRKGLEWLGVIWGS | | GGGACCAAGCTGGAGATCACAGGTGGCGGT | |
| | ETTYYNSALKSRLTII | | GGCTCGGGCGGTGGTGGGTCGGGTGGCGGC | |
| | KDNSKSQVFLKMNSLQ | | GGATCTGAGGTGAAACTGCAGGAGTCAGGA | |
| | TDDTAIYYCAKHYYYG | | CCTGGCCTGGTGGCGCCCTCACAGAGCCTG | |
| | GSYAMDYWGQGTSVTV | | TCCGTCACATGCACTGTCTCAGGGGTCTCA | |
| | SSTTTPAPRPPTPAPT | | TTACCCGACTATGGTGTAAGCTGGATTCGC | |
| | IASQPLSLRPEACRPA | | CAGCCTCCACGAAAGGGTCTGGAGTGGCTG | |
| | AGGAVHTRGLDFACDI | | GGAGTAATATGGGGTAGTGAAACCACATAC | |
| | YIWAPLAGTCGVLLLS | | TATAATTCAGCTCTCAAATCCAGACTGACC | |
| | LVITLYCKRGRKKLLY | | ATCATCAAGGACAACTCCAAGAGCCAAGTT | |
| | IFKQPFMRPVQTTQEE | | TTCTTAAAAATGAACAGTCTGCAAACTGAT | |
| | DGCCRFPEEEEGGCE | | GACACAGCCATTTACTACTGTGCCAAACAT | |
| | LRVKFSRSADAPAYKQ | | TATTACTACGGTGGTAGCTATGCTATGGAC | |
| | GQNQLYNELNLGRREE | | TACTGGGGCCAAGGAACCTCAGTCACCGTC | |
| | YDVLDKRRGRDPEMGG | | TCCTCAACCACGACGCCAGCGCCGCGACCA | |
| | KPRRKNPQEGLYNELQ | | CCAACACCGGCGCCCACCATCGCGTCGCAG | |
| | KDKMAEAYSEIGMKGE | | CCCCTGTCCCTGCGCCCAGAGGCGTGCCGG | |
| | RRRGKGHDGLYQGLST | | CCAGCGGCGGGGGGCGCAGTGCACACGAGG | |
| | ATKDTYDALHMQALPP | | GGGCTGGACTTCGCCTGTGATATCTACATC | |
| | R | | TGGGCGCCCTTGGCCGGGACTTGTGGGGTC | |
| | | | CTTCTCCTGTCACTGGTTATCACCCTTTAC | |
| | | | TGCAAACGGGGCAGAAAGAAACTCCTGTAT | |
| | | | ATATTCAAACAACCATTTATGAGACCAGTA | |
| | | | CAAACTACTCAAGAGGAAGATGGCTGTAGC | |
| | | | TGCCGATTTCCAGAAGAAGAAGAAGGAGGA | |
| | | | TGTGAACTGAGAGTGAAGTTCAGCAGGAGC | |
| | | | GCAGACGCCCCCGCGTACAAGCAGGGCCAG | |
| | | | AACCAGCTCTATAACGAGCTCAATCTAGGA | |
| | | | CGAAGAGAGGAGTACGATGTTTTGGACAAG | |
| | | | AGACGTGGCCGGGACCCTGAGATGGGGGGA | |
| | | | AAGCCGAGAAGGAAGAACCCTCAGGAAGGC | |
| | | | CTGTACAATGAACTGCAGAAAGATAAGATG | |
| | | | GCGGAGGCCTACAGTGAGATTGGGATGAAA | |
| | | | GGCGAGCGCCGGAGGGGCAAGGGGCACGAT | |
| | | | GGCCTTTACCAGGGTCTCAGTACAGCCACC | |
| | | | AAGGACACCTACGACGCCCTTCACATGCAG | |
| | | | GCCCTGCCCCCTCGC | |
| CAR Variant 3 | MLLLVTSLLLCELPHP | 80 | ATGCTTCTCCTGGTGACAAGCCTTCTGCTC | 94 |
| (with N- | AFLLIPDIQMTQTTSS | | TGTGAGTTACCACACCCAGCATTCCTCCTG | |
| terminal signal | LSASLGDRVTISCRAS | | ATCCCAGACATCCAGATGACACAGACTACA | |
| sequence) | QDISKYLNWYQQKPDG | | TCCTCCCTGTCTGCCTCTCTGGGAGACAGA | |
| | TVKLLIYHTSRLHSGV | | GTCACCATCAGTTGCAGGGCAAGTCAGGAC | |
| | PSRFSGSGSGTDYSLT | | ATTAGTAAATATTTAAATTGGTATCAGCAG | |
| | ISNLEQEDIATYFCQQ | | AAACCAGATGGAACTGTTAAACTCCTGATC | |
| | GNTLPYTFGGGTKLEI | | TACCATACATCAAGATTACACTCAGGAGTC | |
| | TGSTSGSGKPGSGEGS | | CCATCAAGGTTCAGTGGCAGTGGGTCTGGA | |
| | TKGEVKLQESGPGLVA | | ACAGATTATTCTCTCACCATTAGCAACCTG | |
| | PSQSLSVTCTVSGVSL | | GAGCAAGAAGATATTGCCACTTACTTTTGC | |
| | PDYGVSWIRQPPRKGL | | CAACAGGGTAATACGCTTCCGTACACGTTC | |
| | EWLGVIWGSETTYYNS | | GGAGGGGGGACTAAGTTGGAAATAACAGGC | |
| | ALKSRLTIIKDNSKSQ | | TCCACCTCTGGATCCGGCAAGCCCGGATCT | |
| | VFLKMNSLQTDDTAIY | | GGCGAGGGATCCACCAAGGGCGAGGTGAAA | |
| | YCAKHYYYGGSYAMDY | | CTGCAGGAGTCAGGACCTGGCCTGGTGGCG | |
| | WGQGTSVTVSSAAAIE | | CCCTCACAGAGCCTGTCCGTCACATGCACT | |
| | VMYPPPYLDNEKSNGT | | GTCTCAGGGGTCTCATTACCCGACTATGGT | |
| | IIHVKGKHLCPSPLFP | | GTAAGCTGGATTCGCCAGCCTCCACGAAAG | |
| | GPSKPFWVLVVVGGVL | | GGTCTGGAGTGGCTGGGAGTAATATGGGGT | |
| | ACYSLLVTVAFIIFWV | | AGTGAAACCACATACTATAATTCAGCTCTC | |
| | RSKRSRLLHSDYMNMT | | AAATCCAGACTGACCATCATCAAGGACAAC | |
| | PRRPGPTRKHYQPYAP | | TCCAAGAGCCAAGTTTTCTTAAAAATGAAC | |
| | PRDFAAYRSRVKESRS | | AGTCTGCAAACTGATGACACAGCCATTTAC | |
| | ADAPAYQQGQNQLYNE | | TACTGTGCCAAACATTATTACTACGGTGGT | |
| | LNLGRREEYDVLDKRR | | AGCTATGCTATGGACTACTGGGGTCAAGGA | |
| | GRDPEMGGKPRRKNPQ | | ACCTCAGTCACCGTCTCCTCAGCGGCCGCA | |
| | EGLYNELQKDKMAEAY | | ATTGAAGTTATGTATCCTCCTCCTTACCTA | |
| | SEIGMKGERRRGKGHD | | GACAATGAGAAGAGCAATGGAACCATTATC | |
| | GLYQGLSTATKDTYDA | | CATGTGAAAGGGAAACACCTTTGTCCAAGT | |
| | LHMQALPPR | | CCCCTATTTCCCGGACCTTCTAAGCCCTTT | |
| | | | TGGGTGCTGGTGGTGGTTGGGGGAGTCCTG | |
| | | | GCTTGCTATAGCTTGCTAGTAACAGTGGCC | |
| | | | TTTATTATTTTCTGGGTGAGGAGTAAGAGG | |
| | | | AGCAGGCTCCTGCACAGTGACTACATGAAC | |
| | | | ATGACTCCCCGCCGCCCCGGGCCCACCCGC | |
| | | | AAGCATTACCAGCCCTATGCCCCACCACGC | |
| | | | GACTTCGCAGCCTATCGCTCCAGAGTGAAG | |
| | | | TTCAGCAGGAGCGCAGACGCCCCCGCGTAC | |
| | | | CAGCAGGGCCAGAACCAGCTCTATAACGAG | |
| | | | CTCAATCTAGGACGAAGAGAGGAGTACGAT | |
| | | | GTTTTGGACAAGAGACGTGGCCGGGACCCT | |
| | | | GAGATGGGGGGAAAGCCGAGAAGGAAGAAC | |
| | | | CCTCAGGAAGGCCTGTACAATGAACTGCAG | |
| | | | AAAGATAAGATGGCGGAGGCCTACAGTGAG | |
| | | | ATTGGGATGAAAGGCGAGCGCCGGAGGGGC | |
| | | | AAGGGGCACGATGGCCTTTACCAGGGTCTC | |
| | | | AGTACAGCCACCAAGGACACCTACGACGCC | |
| | | | CTTCACATGCAGGCCCTGCCCCCTCGC | |
| CAR Variant 3 | DIQMTQTTSSLSASLG | 81 | GACATCCAGATGACACAGACTACATCCTCC | 95 |
| (without N- | DRVTISCRASQDISKY | | CTGTCTGCCTCTCTGGGAGACAGAGTCACC | |
| terminal signal | LNWYQQKPDGTVKLLI | | ATCAGTTGCAGGGCAAGTCAGGACATTAGT | |
| sequence) | YHTSRLHSGVPSRFSG | | AAATATTTAAATTGGTATCAGCAGAAACCA | |
| | SGSGTDYSLTISNLEQ | | GATGGAACTGTTAAACTCCTGATCTACCAT | |
| | EDIATYFCQQGNTLPY | | ACATCAAGATTACACTCAGGAGTCCCATCA | |
| | TFGGGTKLEITGSTSG | | AGGTTCAGTGGCAGTGGGTCTGGAACAGAT | |
| | SGKPGSGEGSTKGEVK | | TATTCTCTCACCATTAGCAACCTGGAGCAA | |
| | LQESGPGLVAPSQSLS | | GAAGATATTGCCACTTACTTTTGCCAACAG | |
| | VTCTVSGVSLPDYGVS | | GGTAATACGCTTCCGTACACGTTCGGAGGG | |
| | WIRQPPRKGLEWLGVI | | GGGACTAAGTTGGAAATAACAGGCTCCACC | |
| | WGSETTYYNSALKSRL | | TCTGGATCCGGCAAGCCCGGATCTGGCGAG | |
| | TIIKDNSKSQVFLKMN | | GGATCCACCAAGGGCGAGGTGAAACTGCAG | |
| | SLQTDDTAIYYCAKHY | | GAGTCAGGACCTGGCCTGGTGGCGCCCTCA | |
| | YYGGSYAMDYWGQGTS | | CAGAGCCTGTCCGTCACATGCACTGTCTCA | |
| | VTVSSAAAIEVMYPPP | | GGGGTCTCATTACCCGACTATGGTGTAAGC | |
| | YLDNEKSNGTIIHVKG | | TGGATTCGCCAGCCTCCACGAAAGGGTCTG | |
| | KHLCPSPLFPGPSKPF | | GAGTGGCTGGGAGTAATATGGGGTAGTGAA | |
| | WVLVVVGGVLACYSLL | | ACCACATACTATAATTCAGCTCTCAAATCC | |
| | VTVAFIIFWVRSKRSR | | AGACTGACCATCATCAAGGACAACTCCAAG | |
| | LLHSDYMNMTPRRPGP | | AGCCAAGTTTTCTTAAAAATGAACAGTCTG | |
| | TRKHYQPYAPPRDFAA | | CAAACTGATGACACAGCCATTTACTACTGT | |
| | YRSRVKFSRSADAPAY | | GCCAAACATTATTACTACGGTGGTAGCTAT | |
| | QQGQNQLYNELNLGRR | | GCTATGGACTACTGGGGTCAAGGAACCTCA | |
| | EEYDVLDKRRGRDPEM | | GTCACCGTCTCCTCAGCGGCCGCAATTGAA | |
| | GGKPRRKNPQEGLYNE | | GTTATGTATCCTCCTCCTTACCTAGACAAT | |
| | LQKDKMAEAYSEIGMK | | GAGAAGAGCAATGGAACCATTATCCATGTG | |
| | GERRRGKGHDGLYQGL | | AAAGGGAAACACCTTTGTCCAAGTCCCCTA | |
| | STATKDTYDALHMQAL | | TTTCCCGGACCTTCTAAGCCCTTTTGGGTG | |
| | PPR | | CTGGTGGTGGTTGGGGGAGTCCTGGCTTGC | |
| | | | TATAGCTTGCTAGTAACAGTGGCCTTTATT | |
| | | | ATTTTCTGGGTGAGGAGTAAGAGGAGCAGG | |
| | | | CTCCTGCACAGTGACTACATGAACATGACT | |
| | | | CCCCGCCGCCCCGGGCCCACCCGCAAGCAT | |
| | | | TACCAGCCCTATGCCCCACCACGCGACTTC | |
| | | | GCAGCCTATCGCTCCAGAGTGAAGTTCAGC | |
| | | | AGGAGCGCAGACGCCCCCGCGTACCAGCAG | |
| | | | GGCCAGAACCAGCTCTATAACGAGCTCAAT | |
| | | | CTAGGACGAAGAGAGGAGTACGATGTTTTG | |
| | | | GACAAGAGACGTGGCCGGGACCCTGAGATG | |
| | | | GGGGGAAAGCCGAGAAGGAAGAACCCTCAG | |
| | | | GAAGGCCTGTACAATGAACTGCAGAAAGAT | |
| | | | AAGATGGCGGAGGCCTACAGTGAGATTGGG | |
| | | | ATGAAAGGCGAGCGCCGGAGGGGCAAGGGG | |
| | | | CACGATGGCCTTTACCAGGGTCTCAGTACA | |
| | | | GCCACCAAGGACACCTACGACGCCCTTCAC | |
| | | | ATGCAGGCCCTGCCCCCTCGC | |

### 5.3 Cytokines

The disclosure also provides recombinant vectors that include cytokines. In some embodiments, the cytokine is an interleukin. Exemplary interleukins include, but are not limited to, IL-15, IL-1, IL-2, IL-3, IL-4, IL-5, IL-6, IL-7, IL-8, IL-9, IL-10, IL-11, IL-12, IL-13, IL-14, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, and functional variants and functional fragments thereof. In some embodiments, the cytokine is soluble. In some embodiments, the cytokine is membrane bound.

In some embodiments, the cytokine is a fusion protein comprising a soluble cytokine, or a functional fragment or functional variant thereof, operably linked to a soluble form of a cognate receptor of the cytokine, or a functional fragment or functional variant thereof. In some embodiments, fusion protein comprises human IL-15 (hIL-15) operably linked to a soluble form of the human IL-15Rα receptor (hIL-15Rα). This fusion protein is also referred to herein as IL-15 superagonist (IL-15 SA). In some embodiments, hIL-15 is directly operably linked to hIL-15Rα. In some embodiments, hIL-15 is indirectly operably linked to the soluble form of hIL-15Rα. In some embodiments, hIL-15 is indirectly operably linked to the soluble form of hIL-15Rα via a peptide linker. In some embodiments, the fusion protein is ALT-803, an IL-15/IL-15Ra Fc fusion protein. ALT-803 is disclosed in WO 2008/143794, the full contents of which is incorporated by reference herein.

In some embodiments, the cytokine is a fusion protein comprising a soluble cytokine, or a functional fragment or functional variant thereof, operably linked to a membrane bound form of a cognate receptor of the cytokine, or a functional fragment or functional variant thereof. In some embodiments, fusion protein comprises human IL-15 (hIL-15) operably linked to human IL-15Rα receptor (hIL-15Rα). This fusion protein is also referred to herein as membrane bound IL-15 (mbIL15). In some embodiments, hIL-15 is directly operably linked to hIL-15Rα. In some embodiments, hIL-15 is indirectly operably linked to hIL-15Rα. In some embodiments, hIL-15 is indirectly operably linked to hIL-15Rα via a peptide linker.

In some embodiments, the peptide linker comprises the amino acid sequence of SEQ ID NO: 125, or an amino acid sequence comprising 1, 2, 3, 4 or 5 amino acid modifications to the amino acid sequence of SEQ ID NO: 125. In some embodiments, the linker comprises the amino acid sequence of SEQ ID NO: 125. In some embodiments, the amino acid of the linker consists of the amino acid sequence of SEQ ID NO: 125, or an amino acid sequence comprising 1, 2, 3, 4 or 5 amino acid modifications to the amino acid sequence of SEQ ID NO: 125. In some embodiments, the amino acid of the linker consists of the amino acid sequence of SEQ ID NO: 125.

In some embodiments, the linker is encoded by a polynucleotide sequence at least 95%, 96%, 97%, 98%, 99%, or 100% identical to the polynucleotide sequence of SEQ ID NO: 136. In some embodiments, the linker is encoded by the polynucleotide sequence of SEQ ID NO: 136.

In some embodiments, hIL-15 comprises an amino acid sequence at least 95%, 96%, 97%, 98%, 99% or 100% identical to the amino acid sequence of SEQ ID NO: 123. In some embodiments, hIL-15 comprises the amino acid sequence of SEQ ID NO: 123. In some embodiments, the amino acid sequence of hIL-15 consists of a sequence at least 95%, 96%, 97%, 98%, 99% or 100% identical to the amino acid sequence of SEQ ID NO: 123. In some embodiments, the amino acid sequence of hIL-15 consists of the amino acid sequence of SEQ ID NO: 123.

In some embodiments, IL-15 is encoded by a polynucleotide sequence at least 75%, 80%, 85%, 90%, 95%, 96%, 97%, 98%, 99%, or 100% identical to the polynucleotide sequence of SEQ ID NO: 134. In some embodiments, IL-15 is encoded by the polynucleotide sequence of SEQ ID NO: 134.

In some embodiments, hIL-15Rα comprises an amino acid sequence at least 95%, 96%, 97%, 98%, 99% or 100% identical to the amino acid sequence of SEQ ID NO: 124. In some embodiments, hIL-15Rα comprises the amino acid sequence of SEQ ID NO: 124. In some embodiments, the amino acid sequence of hIL-15Rα consists of a sequence at least 95%, 96%, 97%, 98%, 99% or 100% identical to the amino acid sequence of SEQ ID NO: 124. In some embodiments, the amino acid sequence of hIL-15Rα consists of the amino acid sequence of SEQ ID NO: 124.

In some embodiments, hIL-15Rα is encoded by a polynucleotide sequence at least 75%, 80%, 85%, 90%, 95%, 96%, 97%, 98%, 99%, or 100% identical to the polynucleotide sequence of SEQ ID NO: 135. In some embodiments, hIL-15Rα is encoded by the polynucleotide sequence of SEQ ID NO: 135. In some embodiments, hIL-15Rα is encoded by a polynucleotide sequence at least 75%, 80%, 85%, 90%, 95%, 96%, 97%, 98%, 99%, or 100% identical to the polynucleotide sequence of SEQ ID NO: 163. In some embodiments, hIL-15Rα is encoded by the polynucleotide sequence of SEQ ID NO: 163.

In some embodiments, the fusion protein comprises an amino acid sequence at least 95%, 96%, 97%, 98%, 99% or 100% identical to SEQ ID NO: 119, 120, 121, 122, 180, or 183. In some embodiments, the fusion protein comprises an amino acid sequence at least 95%, 96%, 97%, 98%, 99% or 100% identical to SEQ ID NO: 119. In some embodiments, the fusion protein comprises an amino acid sequence at least 95%, 96%, 97%, 98%, 99% or 100% identical to SEQ ID NO: 120. In some embodiments, the fusion protein comprises an amino acid sequence at least 95%, 96%, 97%, 98%, 99% or 100% identical to SEQ ID NO: 121. In some embodiments, the fusion protein comprises an amino acid sequence at least 95%, 96%, 97%, 98%, 99% or 100% identical to SEQ ID NO: 122. In some embodiments, the fusion protein comprises an amino acid sequence at least 95%, 96%, 97%, 98%, 99% or 100% identical to SEQ ID NO: 180. In some embodiments, the fusion protein comprises an amino acid sequence at least 95%, 96%, 97%, 98%, 99% or 100% identical to SEQ ID NO: 183. In some embodiments, the fusion protein comprises the amino acid sequence of SEQ ID NO: 119, 120, 121, 122, 180, or 183. In some embodiments, the fusion protein comprises the amino acid sequence of SEQ ID NO: 119. In some embodiments, the fusion protein comprises the amino acid sequence of SEQ ID NO: 120. In some embodiments, the fusion protein comprises the amino acid sequence of SEQ ID NO: 121. In some embodiments, the fusion protein comprises the amino acid sequence of SEQ ID NO: 122. In some embodiments, the fusion protein comprises the amino acid sequence of SEQ ID NO: 180. In some embodiments, the fusion protein comprises the amino acid sequence of SEQ ID NO: 183.

In some embodiments, the amino acid sequence of the fusion protein consists of a sequence at least 95%, 96%, 97%, 98%, 99% or 100% identical to the amino acid sequence of SEQ ID NO: 119, 120, 121, 122, 180, or 183. In some embodiments, the amino acid sequence of the fusion protein consists of a sequence at least 95%, 96%, 97%, 98%, 99% or 100% identical to the amino acid sequence of SEQ ID NO: 119. In some embodiments, the amino acid sequence of the fusion protein consists of a sequence at least 95%, 96%, 97%, 98%, 99% or 100% identical to the amino acid sequence of SEQ ID NO: 120. In some embodiments, the amino acid sequence of the fusion protein consists of a sequence at least 95%, 96%, 97%, 98%, 99% or 100% identical to the amino acid sequence of SEQ ID NO: 121. In some embodiments, the amino acid sequence of the fusion protein consists of a sequence at least 95%, 96%, 97%, 98%, 99% or 100% identical to the amino acid sequence of SEQ ID NO: 122. In some embodiments, the amino acid sequence of the fusion protein consists of a sequence at least 95%, 96%, 97%, 98%, 99% or 100% identical to the amino acid sequence of SEQ ID NO: 180. In some embodiments, the amino acid sequence of the fusion protein consists of a sequence at least 95%, 96%, 97%, 98%, 99% or 100% identical to the amino acid sequence of SEQ ID NO: 183. In some embodiments, the amino acid sequence of the fusion protein consists of the amino acid sequence of SEQ ID NO: 119, 120, 121, 122, 180, or 183. In some embodiments, the amino acid sequence of the fusion protein consists of the amino acid sequence of SEQ ID NO: 119. In some embodiments, the amino acid sequence of the fusion protein consists of the amino acid sequence of SEQ ID NO: 120. In some embodiments, the amino acid sequence of the fusion protein consists of the amino acid sequence of SEQ ID NO: 121. In some embodiments, the amino acid sequence of the fusion protein consists of the amino acid sequence of SEQ ID NO: 122. In some embodiments, the amino acid sequence of the fusion protein consists of the amino acid sequence of SEQ ID NO: 180. In some embodiments, the amino acid sequence of the fusion protein consists of the amino acid sequence of SEQ ID NO: 183.

In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 126, 127, 128, 129, 130, 131, 132, or 181. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 126. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 127. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 128. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 129. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 130. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 131. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 132. In some embodiments, the fusion protein is encoded by a polynucleotide sequence at least 75%, 80%, 85%, 90%, 95%, 96%, 97%, 98%, 99% or 100% identical to the polynucleotide sequence of SEQ ID NO: 181.

In some embodiments, the fusion protein is encoded by the polynucleotide sequence of SEQ ID NO: 126, 127, 128, 129, 130, 131, 132, or 181. In some embodiments, the fusion protein is encoded by the polynucleotide sequence of SEQ ID NO: 126. In some embodiments, the fusion protein is encoded by the polynucleotide sequence of SEQ ID NO: 127. In some embodiments, the fusion protein is encoded by the polynucleotide sequence of SEQ ID NO: 128. In some embodiments, the fusion protein is encoded by the polynucleotide sequence of SEQ ID NO: 129. In some embodiments, the fusion protein is encoded by the polynucleotide sequence of SEQ ID NO: 130. In some embodiments, the fusion protein is encoded by the polynucleotide sequence of SEQ ID NO: 131. In some embodiments, the fusion protein is encoded by a polynucleotide sequence comprising the polynucleotide sequence of SEQ ID NO: 132. In some embodiments, the fusion protein is encoded by the polynucleotide sequence of SEQ ID NO: 132. In some embodiments, the fusion protein is encoded by the polynucleotide sequence of SEQ ID NO: 181.

Exemplary cytokine fusion proteins and components thereof are disclosed in Table 6. Additional exemplary mbIL15 fusions are disclosed in Hurton et al., “Tethered IL-15 augments antitumor activity and promotes a stem-cell memory subset in tumor-specific T cells,” PNAS, 113(48) E7788-E7797 (2016), the entire contents of which are incorporated by reference herein.

The amino acid sequence and polynucleotide sequence of exemplary cytokine fusion proteins and component polypeptides are provided in Table 6, herein.

TABLE 6
Amino acid and polynucleotide sequences of exemplary cytokine fusion proteins and
component polypeptides.

| | | SEQ ID | | SEQ ID |
|---|---|---|---|---|
| Description | Amino Acid Sequence | NO | Polynucleotide Sequence | NO |
| mbIL15 (with N- | MDWTWILFLVAAATRVHSN | 119 | ATGGATTGGACCTGGATTC | 126 |
| terminal signal | WVNVISDLKKIEDLIQSMH | | TGTTTCTGGTGGCCGCTGC | |
| sequence and | IDATLYTESDVHPSCKVTA | | CACAAGAGTGCACAGCAAC | |
| without C- | MKCFLLELQVISLESGDAS | | TGGGTGAATGTGATCAGCG | |
| terminal tail) | IHDTVENLIILANNSLSSN | | ACCTGAAGAAGATCGAGGA | |
| | GNVTESGCKECEELEEKNI | | TCTGATCCAGAGCATGCAC | |
| | KEFLQSFVHIVQMFINTSS | | ATTGATGCCACCCTGTACA | |
| | GGGSGGGGSGGGGSGGGGS | | CAGAATCTGATGTGCACCC | |
| | GGGSLQITCPPPMSVEHAD | | TAGCTGTAAAGTGACCGCC | |
| | IWVKSYSLYSRERYICNSG | | ATGAAGTGTTTTCTGCTGG | |
| | FKRKAGTSSLTECVLNKAT | | AGCTGCAGGTGATTTCTCT | |
| | NVAHWTTPSLKCIRDPALV | | GGAAAGCGGAGATGCCTCT | |
| | HQRPAPPSTVTTAGVTPQP | | ATCCACGACACAGTGGAGA | |
| | ESLSPSGKEPAASSPSSNN | | ATCTGATCATCCTGGCCAA | |
| | TAATTAAIVPGSQLMPSKS | | CAATAGCCTGAGCAGCAAT | |
| | PSTGTTEISSHESSHGTPS | | GGCAATGTGACAGAGTCTG | |
| | QTTAKNWELTASASHQPPG | | GCTGTAAGGAGTGTGAGGA | |
| | VYPQGHSDTTVAISTSTVL | | GCTGGAGGAGAAGAACATC | |
| | LCGLSAVSLLACYLKSRQT | | AAGGAGTTTCTGCAGAGCT | |
| | PPLASVEMEAMEALPVTWG | | TTGTGCACATCGTGCAGAT | |
| | TSSRDEDLENCSHHL | | GTTCATCAATACAAGCTCT | |
| | | | GGCGGAGGATCTGGAGGAG | |
| | | | GCGGATCTGGAGGAGGAGG | |
| | | | CAGTGGAGGCGGAGGATCT | |
| | | | GGCGGAGGATCTCTGCAGA | |
| | | | TTACATGCCCTCCTCCAAT | |
| | | | GTCTGTGGAGCACGCCGAT | |
| | | | ATTTGGGTGAAGTCCTACA | |
| | | | GCCTGTACAGCAGAGAGAG | |
| | | | ATACATCTGCAACAGCGGC | |
| | | | TTTAAGAGAAAGGCCGGCA | |
| | | | CCTCTTCTCTGACAGAGTG | |
| | | | CGTGCTGAATAAGGCCACA | |
| | | | AATGTGGCCCACTGGACAA | |
| | | | CACCTAGCCTGAAGTGCAT | |
| | | | TAGAGATCCTGCCCTGGTC | |
| | | | CACCAGAGGCCTGCCCCTC | |
| | | | CATCTACAGTGACAACAGC | |
| | | | CGGAGTGACACCTCAGCCT | |
| | | | GAATCTCTGAGCCCTTCTG | |
| | | | GAAAAGAACCTGCCGCCAG | |
| | | | CTCTCCTAGCTCTAATAAT | |
| | | | ACCGCCGCCACAACAGCCG | |
| | | | CCATTGTGCCTGGATCTCA | |
| | | | GCTGATGCCTAGCAAGTCT | |
| | | | CCTAGCACAGGCACAACAG | |
| | | | AGATCAGCAGCCACGAATC | |
| | | | TTCTCACGGAACACCTTCT | |
| | | | CAGACCACCGCCAAGAATT | |
| | | | GGGAGCTGACAGCCTCTGC | |
| | | | CTCTCACCAGCCTCCAGGA | |
| | | | GTGTATCCTCAGGGCCACT | |
| | | | CTGATACAACAGTGGCCAT | |
| | | | CAGCACATCTACAGTGCTG | |
| | | | CTGTGTGGACTGTCTGCCG | |
| | | | TGTCTCTGCTGGCCTGTTA | |
| | | | CCTGAAGTCTAGACAGACA | |
| | | | CCTCCTCTGGCCTCTGTGG | |
| | | | AGATGGAGGCCATGGAAGC | |
| | | | CCTGCCTGTGACATGGGGA | |
| | | | ACAAGCAGCAGAGATGAAG | |
| | | | ACCTGGAGAATTGTTCTCA | |
| | | | CCACCTG | |
| | | | ATGGATTGGACCTGGATTC | 127 |
| | | | TGTTTCTGGTGGCCGCTGC | |
| | | | CACAAGAGTGCACAGCAAC | |
| | | | TGGGTGAATGTGATCAGCG | |
| | | | ACCTGAAGAAGATCGAGGA | |
| | | | TCTGATCCAGAGCATGCAC | |
| | | | ATTGATGCCACCCTGTACA | |
| | | | CAGAATCTGATGTGCACCC | |
| | | | TAGCTGTAAAGTGACCGCC | |
| | | | ATGAAGTGTTTTCTGCTGG | |
| | | | AGCTGCAGGTGATTTCTCT | |
| | | | GGAAAGCGGAGATGCCTCT | |
| | | | ATCCACGACACAGTGGAGA | |
| | | | ATCTGATCATCCTGGCCAA | |
| | | | CAATAGCCTGAGCAGCAAT | |
| | | | GGCAATGTGACAGAGTCTG | |
| | | | GCTGTAAGGAGTGTGAGGA | |
| | | | GCTGGAGGAGAAGAACATC | |
| | | | AAGGAGTTTCTGCAGAGCT | |
| | | | TTGTGCACATCGTGCAGAT | |
| | | | GTTCATCAATACAAGCTCT | |
| | | | GGCGGAGGATCTGGAGGAG | |
| | | | GCGGATCTGGAGGAGGAGG | |
| | | | CAGTGGAGGCGGAGGATCT | |
| | | | GGCGGAGGATCTCTGCAGA | |
| | | | TTACATGCCCTCCTCCAAT | |
| | | | GTCTGTGGAGCACGCCGAT | |
| | | | ATTTGGGTGAAGTCCTACA | |
| | | | GCCTGTACAGCAGAGAGAG | |
| | | | ATACATCTGCAACAGCGGC | |
| | | | TTTAAGAGAAAGGCCGGCA | |
| | | | CCTCTTCTCTGACAGAGTG | |
| | | | CGTGCTGAATAAGGCCACA | |
| | | | AATGTGGCCCACTGGACAA | |
| | | | CACCTAGCCTGAAGTGCAT | |
| | | | TAGAGATCCTGCCCTGGTC | |
| | | | CACCAGAGGCCTGCCCCTC | |
| | | | CATCTACAGTGACAACAGC | |
| | | | CGGAGTGACACCTCAGCCT | |
| | | | GAATCTCTGAGCCCTTCTG | |
| | | | GAAAAGAACCTGCCGCCAG | |
| | | | CTCTCCTAGCTCTAATAAT | |
| | | | ACCGCCGCCACAACAGCCG | |
| | | | CCATTGTGCCTGGATCTCA | |
| | | | GCTGATGCCTAGCAAGTCT | |
| | | | CCTAGCACAGGCACAACAG | |
| | | | AGATCAGCAGCCACGAATC | |
| | | | TTCTCACGGAACACCTTCT | |
| | | | CAGACCACCGCCAAGAATT | |
| | | | GGGAGCTGACAGCCTCTGC | |
| | | | CTCTCACCAGCCTCCAGGA | |
| | | | GTGTATCCTCAGGGCCACT | |
| | | | CTGATACAACAGTGGCCAT | |
| | | | CAGCACATCTACAGTGCTG | |
| | | | CTGTGTGGACTGTCTGCCG | |
| | | | TGTCTCTGCTGGCCTGTTA | |
| | | | CCTGAAGTCTAGACAGACA | |
| | | | CCTCCTCTGGCCTCTGTGG | |
| | | | AGATGGAGGCCATGGAAGC | |
| | | | CCTGCCTGTGACATGGGGA | |
| | | | ACAAGCAGCAGAGATGAGG | |
| | | | ACCTGGAGAATTGTTCTCA | |
| | | | CCACCTG | |
| mbIL15 (with | PMDWTWILFLVAAATRVHS | 180 | CCCATGGATTGGACCTGGA | 181 |
| variant N-terminal | NWVNVISDLKKIEDLIQSM | | TTCTGTTTCTGGTGGCCGC | |
| signal sequence | HIDATLYTESDVHPSCKVT | | TGCCACAAGAGTGCACAGC | |
| and without C- | AMKCFLLELQVISLESGDA | | AACTGGGTGAATGTGATCA | |
| terminal tail) | SIHDTVENLIILANNSLSS | | GCGACCTGAAGAAGATCGA | |
| | NGNVTESGCKECEELEEKN | | GGATCTGATCCAGAGCATG | |
| | IKEFLQSFVHIVQMFINTS | | CACATTGATGCCACCCTGT | |
| | SGGGSGGGGSGGGGSGGGG | | ACACAGAATCTGATGTGCA | |
| | SGGGSLQITCPPPMSVEHA | | CCCTAGCTGTAAAGTGACC | |
| | DIWVKSYSLYSRERYICNS | | GCCATGAAGTGTTTTCTGC | |
| | GFKRKAGTSSLTECVLNKA | | TGGAGCTGCAGGTGATTTC | |
| | TNVAHWTTPSLKCIRDPAL | | TCTGGAAAGCGGAGATGCC | |
| | VHQRPAPPSTVTTAGVTPQ | | TCTATCCACGACACAGTGG | |
| | PESLSPSGKEPAASSPSSN | | AGAATCTGATCATCCTGGC | |
| | NTAATTAAIVPGSQLMPSK | | CAACAATAGCCTGAGCAGC | |
| | SPSTGTTEISSHESSHGTP | | AATGGCAATGTGACAGAGT | |
| | SQTTAKNWELTASASHQPP | | CTGGCTGTAAGGAGTGTGA | |
| | GVYPQGHSDTTVAISTSTV | | GGAGCTGGAGGAGAAGAAC | |
| | LLCGLSAVSLLACYLKSRQ | | ATCAAGGAGTTTCTGCAGA | |
| | TPPLASVEMEAMEALPVTW | | GCTTTGTGCACATCGTGCA | |
| | GTSSRDEDLENCSHHL | | GATGTTCATCAATACAAGC | |
| | | | TCTGGCGGAGGATCTGGAG | |
| | | | GAGGCGGATCTGGAGGAGG | |
| | | | AGGCAGTGGAGGCGGAGGA | |
| | | | TCTGGCGGAGGATCTCTGC | |
| | | | AGATTACATGCCCTCCTCC | |
| | | | AATGTCTGTGGAGCACGCC | |
| | | | GATATTTGGGTGAAGTCCT | |
| | | | ACAGCCTGTACAGCAGAGA | |
| | | | GAGATACATCTGCAACAGC | |
| | | | GGCTTTAAGAGAAAGGCCG | |
| | | | GCACCTCTTCTCTGACAGA | |
| | | | GTGCGTGCTGAATAAGGCC | |
| | | | ACAAATGTGGCCCACTGGA | |
| | | | CAACACCTAGCCTGAAGTG | |
| | | | CATTAGAGATCCTGCCCTG | |
| | | | GTCCACCAGAGGCCTGCCC | |
| | | | CTCCATCTACAGTGACAAC | |
| | | | AGCCGGAGTGACACCTCAG | |
| | | | CCTGAATCTCTGAGCCCTT | |
| | | | CTGGAAAAGAACCTGCCGC | |
| | | | CAGCTCTCCTAGCTCTAAT | |
| | | | AATACCGCCGCCACAACAG | |
| | | | CCGCCATTGTGCCTGGATC | |
| | | | TCAGCTGATGCCTAGCAAG | |
| | | | TCTCCTAGCACAGGCACAA | |
| | | | CAGAGATCAGCAGCCACGA | |
| | | | ATCTTCTCACGGAACACCT | |
| | | | TCTCAGACCACCGCCAAGA | |
| | | | ATTGGGAGCTGACAGCCTC | |
| | | | TGCCTCTCACCAGCCTCCA | |
| | | | GGAGTGTATCCTCAGGGCC | |
| | | | ACTCTGATACAACAGTGGC | |
| | | | CATCAGCACATCTACAGTG | |
| | | | CTGCTGTGTGGACTGTCTG | |
| | | | CCGTGTCTCTGCTGGCCTG | |
| | | | TTACCTGAAGTCTAGACAG | |
| | | | ACACCTCCTCTGGCCTCTG | |
| | | | TGGAGATGGAGGCCATGGA | |
| | | | AGCCCTGCCTGTGACATGG | |
| | | | GGAACAAGCAGCAGAGATG | |
| | | | AAGACCTGGAGAATTGTTC | |
| | | | TCACCACCTG | |
| mbIL15 (with N- | MDWTWILFLVAAATRVHSN | 120 | ATGGATTGGACCTGGATTC | 128 |
| terminal signal | WVNVISDLKKIEDLIQSMH | | TGTTTCTGGTGGCCGCTGC | |
| sequence and with | IDATLYTESDVHPSCKVTA | | CACAAGAGTGCACAGCAAC | |
| C-terminal tail) | MKCFLLELQVISLESGDAS | | TGGGTGAATGTGATCAGCG | |
| | IHDTVENLIILANNSLSSN | | ACCTGAAGAAGATCGAGGA | |
| | GNVTESGCKECEELEEKNI | | TCTGATCCAGAGCATGCAC | |
| | KEFLQSFVHIVQMFINTSS | | ATTGATGCCACCCTGTACA | |
| | GGGSGGGGSGGGGSGGGGS | | CAGAATCTGATGTGCACCC | |
| | GGGSLQITCPPPMSVEHAD | | TAGCTGTAAAGTGACCGCC | |
| | IWVKSYSLYSRERYICNSG | | ATGAAGTGTTTTCTGCTGG | |
| | FKRKAGTSSLTECVLNKAT | | AGCTGCAGGTGATTTCTCT | |
| | NVAHWTTPSLKCIRDPALV | | GGAAAGCGGAGATGCCTCT | |
| | HQRPAPPSTVTTAGVTPQP | | ATCCACGACACAGTGGAGA | |
| | ESLSPSGKEPAASSPSSNN | | ATCTGATCATCCTGGCCAA | |
| | TAATTAAIVPGSQLMPSKS | | CAATAGCCTGAGCAGCAAT | |
| | PSTGTTEISSHESSHGTPS | | GGCAATGTGACAGAGTCTG | |
| | QTTAKNWELTASASHQPPG | | GCTGTAAGGAGTGTGAGGA | |
| | VYPQGHSDTTVAISTSTVL | | GCTGGAGGAGAAGAACATC | |
| | LCGLSAVSLLACYLKSRQT | | AAGGAGTTTCTGCAGAGCT | |
| | PPLASVEMEAMEALPVTWG | | TTGTGCACATCGTGCAGAT | |
| | TSSRDEDLENCSHHLLEGG | | GTTCATCAATACAAGCTCT | |
| | GEGRGSLLTCGDVEENPG | | GGCGGAGGATCTGGAGGAG | |
| | | | GCGGATCTGGAGGAGGAGG | |
| | | | CAGTGGAGGCGGAGGATCT | |
| | | | GGCGGAGGATCTCTGCAGA | |
| | | | TTACATGCCCTCCTCCAAT | |
| | | | GTCTGTGGAGCACGCCGAT | |
| | | | ATTTGGGTGAAGTCCTACA | |
| | | | GCCTGTACAGCAGAGAGAG | |
| | | | ATACATCTGCAACAGCGGC | |
| | | | TTTAAGAGAAAGGCCGGCA | |
| | | | CCTCTTCTCTGACAGAGTG | |
| | | | CGTGCTGAATAAGGCCACA | |
| | | | AATGTGGCCCACTGGACAA | |
| | | | CACCTAGCCTGAAGTGCAT | |
| | | | TAGAGATCCTGCCCTGGTC | |
| | | | CACCAGAGGCCTGCCCCTC | |
| | | | CATCTACAGTGACAACAGC | |
| | | | CGGAGTGACACCTCAGCCT | |
| | | | GAATCTCTGAGCCCTTCTG | |
| | | | GAAAAGAACCTGCCGCCAG | |
| | | | CTCTCCTAGCTCTAATAAT | |
| | | | ACCGCCGCCACAACAGCCG | |
| | | | CCATTGTGCCTGGATCTCA | |
| | | | GCTGATGCCTAGCAAGTCT | |
| | | | CCTAGCACAGGCACAACAG | |
| | | | AGATCAGCAGCCACGAATC | |
| | | | TTCTCACGGAACACCTTCT | |
| | | | CAGACCACCGCCAAGAATT | |
| | | | GGGAGCTGACAGCCTCTGC | |
| | | | CTCTCACCAGCCTCCAGGA | |
| | | | GTGTATCCTCAGGGCCACT | |
| | | | CTGATACAACAGTGGCCAT | |
| | | | CAGCACATCTACAGTGCTG | |
| | | | CTGTGTGGACTGTCTGCCG | |
| | | | TGTCTCTGCTGGCCTGTTA | |
| | | | CCTGAAGTCTAGACAGACA | |
| | | | CCTCCTCTGGCCTCTGTGG | |
| | | | AGATGGAGGCCATGGAAGC | |
| | | | CCTGCCTGTGACATGGGGA | |
| | | | ACAAGCAGCAGAGATGAAG | |
| | | | ACCTGGAGAATTGTTCTCA | |
| | | | CCACCTGCTGGAGGGCGGC | |
| | | | GGAGAGGGCAGAGGAAGTC | |
| | | | TTCTAACATGCGGTGACGT | |
| | | | GGAGGAGAATCCCGGC | |
| mbIL15 (with | PMDWTWILFLVAAATRVHS | 183 | CCCATGGATTGGACCTGGA | 129 |
| variant N-terminal | NWVNVISDLKKIEDLIQSM | | TTCTGTTTCTGGTGGCCGC | |
| signal sequence | HIDATLYTESDVHPSCKVT | | TGCCACAAGAGTGCACAGC | |
| and with C- | AMKCFLLELQVISLESGDA | | AACTGGGTGAATGTGATCA | |
| terminal tail) | SIHDTVENLIILANNSLSS | | GCGACCTGAAGAAGATCGA | |
| | NGNVTESGCKECEELEEKN | | GGATCTGATCCAGAGCATG | |
| | IKEFLQSFVHIVQMFINTS | | CACATTGATGCCACCCTGT | |
| | SGGGSGGGGSGGGGSGGGG | | ACACAGAATCTGATGTGCA | |
| | SGGGSLQITCPPPMSVEHA | | CCCTAGCTGTAAAGTGACC | |
| | DIWVKSYSLYSRERYICNS | | GCCATGAAGTGTTTTCTGC | |
| | GFKRKAGTSSLTECVLNKA | | TGGAGCTGCAGGTGATTTC | |
| | TNVAHWTTPSLKCIRDPAL | | TCTGGAAAGCGGAGATGCC | |
| | VHQRPAPPSTVTTAGVTPQ | | TCTATCCACGACACAGTGG | |
| | PESLSPSGKEPAASSPSSN | | AGAATCTGATCATCCTGGC | |
| | NTAATTAAIVPGSQLMPSK | | CAACAATAGCCTGAGCAGC | |
| | SPSTGTTEISSHESSHGTP | | AATGGCAATGTGACAGAGT | |
| | SQTTAKNWELTASASHQPP | | CTGGCTGTAAGGAGTGTGA | |
| | GVYPQGHSDTTVAISTSTV | | GGAGCTGGAGGAGAAGAAC | |
| | LLCGLSAVSLLACYLKSRQ | | ATCAAGGAGTTTCTGCAGA | |
| | TPPLASVEMEAMEALPVTW | | GCTTTGTGCACATCGTGCA | |
| | GTSSRDEDLENCSHHLLEG | | GATGTTCATCAATACAAGC | |
| | GGEGRGSLLTCGDVEENPG | | TCTGGCGGAGGATCTGGAG | |
| | | | GAGGCGGATCTGGAGGAGG | |
| | | | AGGCAGTGGAGGCGGAGGA | |
| | | | TCTGGCGGAGGATCTCTGC | |
| | | | AGATTACATGCCCTCCTCC | |
| | | | AATGTCTGTGGAGCACGCC | |
| | | | GATATTTGGGTGAAGTCCT | |
| | | | ACAGCCTGTACAGCAGAGA | |
| | | | GAGATACATCTGCAACAGC | |
| | | | GGCTTTAAGAGAAAGGCCG | |
| | | | GCACCTCTTCTCTGACAGA | |
| | | | GTGCGTGCTGAATAAGGCC | |
| | | | ACAAATGTGGCCCACTGGA | |
| | | | CAACACCTAGCCTGAAGTG | |
| | | | CATTAGAGATCCTGCCCTG | |
| | | | GTCCACCAGAGGCCTGCCC | |
| | | | CTCCATCTACAGTGACAAC | |
| | | | AGCCGGAGTGACACCTCAG | |
| | | | CCTGAATCTCTGAGCCCTT | |
| | | | CTGGAAAAGAACCTGCCGC | |
| | | | CAGCTCTCCTAGCTCTAAT | |
| | | | AATACCGCCGCCACAACAG | |
| | | | CCGCCATTGTGCCTGGATC | |
| | | | TCAGCTGATGCCTAGCAAG | |
| | | | TCTCCTAGCACAGGCACAA | |
| | | | CAGAGATCAGCAGCCACGA | |
| | | | ATCTTCTCACGGAACACCT | |
| | | | TCTCAGACCACCGCCAAGA | |
| | | | ATTGGGAGCTGACAGCCTC | |
| | | | TGCCTCTCACCAGCCTCCA | |
| | | | GGAGTGTATCCTCAGGGCC | |
| | | | ACTCTGATACAACAGTGGC | |
| | | | CATCAGCACATCTACAGTG | |
| | | | CTGCTGTGTGGACTGTCTG | |
| | | | CCGTGTCTCTGCTGGCCTG | |
| | | | TTACCTGAAGTCTAGACAG | |
| | | | ACACCTCCTCTGGCCTCTG | |
| | | | TGGAGATGGAGGCCATGGA | |
| | | | AGCCCTGCCTGTGACATGG | |
| | | | GGAACAAGCAGCAGAGATG | |
| | | | AAGACCTGGAGAATTGTTC | |
| | | | TCACCACCTGCTGGAGGGC | |
| | | | GGCGGAGAGGGCAGAGGAA | |
| | | | GTCTTCTAACATGCGGTGA | |
| | | | CGTGGAGGAGAATCCCGGC | |
| mbIL15 (without | NWVNVISDLKKIEDLIQSM | 121 | AACTGGGTGAATGTGATCA | 130 |
| N-terminal signal | HIDATLYTESDVHPSCKVT | | GCGACCTGAAGAAGATCGA | |
| sequence and | AMKCFLLELQVISLESGDA | | GGATCTGATCCAGAGCATG | |
| without C- | SIHDTVENLIILANNSLSS | | CACATTGATGCCACCCTGT | |
| terminal tail) | NGNVTESGCKECEELEEKN | | ACACAGAATCTGATGTGCA | |
| | IKEFLQSFVHIVQMFINTS | | CCCTAGCTGTAAAGTGACC | |
| | SGGGSGGGGSGGGGSGGGG | | GCCATGAAGTGTTTTCTGC | |
| | SGGGSLQITCPPPMSVEHA | | TGGAGCTGCAGGTGATTTC | |
| | DIWVKSYSLYSRERYICNS | | TCTGGAAAGCGGAGATGCC | |
| | GFKRKAGTSSLTECVLNKA | | TCTATCCACGACACAGTGG | |
| | TNVAHWTTPSLKCIRDPAL | | AGAATCTGATCATCCTGGC | |
| | VHQRPAPPSTVTTAGVTPQ | | CAACAATAGCCTGAGCAGC | |
| | PESLSPSGKEPAASSPSSN | | AATGGCAATGTGACAGAGT | |
| | NTAATTAAIVPGSQLMPSK | | CTGGCTGTAAGGAGTGTGA | |
| | SPSTGTTEISSHESSHGTP | | GGAGCTGGAGGAGAAGAAC | |
| | SQTTAKNWELTASASHQPP | | ATCAAGGAGTTTCTGCAGA | |
| | GVYPQGHSDTTVAISTSTV | | GCTTTGTGCACATCGTGCA | |
| | LLCGLSAVSLLACYLKSRQ | | GATGTTCATCAATACAAGC | |
| | TPPLASVEMEAMEALPVTW | | TCTGGCGGAGGATCTGGAG | |
| | GTSSRDEDLENCSHHL | | GAGGCGGATCTGGAGGAGG | |
| | | | AGGCAGTGGAGGCGGAGGA | |
| | | | TCTGGCGGAGGATCTCTGC | |
| | | | AGATTACATGCCCTCCTCC | |
| | | | AATGTCTGTGGAGCACGCC | |
| | | | GATATTTGGGTGAAGTCCT | |
| | | | ACAGCCTGTACAGCAGAGA | |
| | | | GAGATACATCTGCAACAGC | |
| | | | GGCTTTAAGAGAAAGGCCG | |
| | | | GCACCTCTTCTCTGACAGA | |
| | | | GTGCGTGCTGAATAAGGCC | |
| | | | ACAAATGTGGCCCACTGGA | |
| | | | CAACACCTAGCCTGAAGTG | |
| | | | CATTAGAGATCCTGCCCTG | |
| | | | GTCCACCAGAGGCCTGCCC | |
| | | | CTCCATCTACAGTGACAAC | |
| | | | AGCCGGAGTGACACCTCAG | |
| | | | CCTGAATCTCTGAGCCCTT | |
| | | | CTGGAAAAGAACCTGCCGC | |
| | | | CAGCTCTCCTAGCTCTAAT | |
| | | | AATACCGCCGCCACAACAG | |
| | | | CCGCCATTGTGCCTGGATC | |
| | | | TCAGCTGATGCCTAGCAAG | |
| | | | TCTCCTAGCACAGGCACAA | |
| | | | CAGAGATCAGCAGCCACGA | |
| | | | ATCTTCTCACGGAACACCT | |
| | | | TCTCAGACCACCGCCAAGA | |
| | | | ATTGGGAGCTGACAGCCTC | |
| | | | TGCCTCTCACCAGCCTCCA | |
| | | | GGAGTGTATCCTCAGGGCC | |
| | | | ACTCTGATACAACAGTGGC | |
| | | | CATCAGCACATCTACAGTG | |
| | | | CTGCTGTGTGGACTGTCTG | |
| | | | CCGTGTCTCTGCTGGCCTG | |
| | | | TTACCTGAAGTCTAGACAG | |
| | | | ACACCTCCTCTGGCCTCTG | |
| | | | TGGAGATGGAGGCCATGGA | |
| | | | AGCCCTGCCTGTGACATGG | |
| | | | GGAACAAGCAGCAGAGATG | |
| | | | AAGACCTGGAGAATTGTTC | |
| | | | TCACCACCTG | |
| | | | AACTGGGTGAATGTGATCA | 131 |
| | | | GCGACCTGAAGAAGATCGA | |
| | | | GGATCTGATCCAGAGCATG | |
| | | | CACATTGATGCCACCCTGT | |
| | | | ACACAGAATCTGATGTGCA | |
| | | | CCCTAGCTGTAAAGTGACC | |
| | | | GCCATGAAGTGTTTTCTGC | |
| | | | TGGAGCTGCAGGTGATTTC | |
| | | | TCTGGAAAGCGGAGATGCC | |
| | | | TCTATCCACGACACAGTGG | |
| | | | AGAATCTGATCATCCTGGC | |
| | | | CAACAATAGCCTGAGCAGC | |
| | | | AATGGCAATGTGACAGAGT | |
| | | | CTGGCTGTAAGGAGTGTGA | |
| | | | GGAGCTGGAGGAGAAGAAC | |
| | | | ATCAAGGAGTTTCTGCAGA | |
| | | | GCTTTGTGCACATCGTGCA | |
| | | | GATGTTCATCAATACAAGC | |
| | | | TCTGGCGGAGGATCTGGAG | |
| | | | GAGGCGGATCTGGAGGAGG | |
| | | | AGGCAGTGGAGGCGGAGGA | |
| | | | TCTGGCGGAGGATCTCTGC | |
| | | | AGATTACATGCCCTCCTCC | |
| | | | AATGTCTGTGGAGCACGCC | |
| | | | GATATTTGGGTGAAGTCCT | |
| | | | ACAGCCTGTACAGCAGAGA | |
| | | | GAGATACATCTGCAACAGC | |
| | | | GGCTTTAAGAGAAAGGCCG | |
| | | | GCACCTCTTCTCTGACAGA | |
| | | | GTGCGTGCTGAATAAGGCC | |
| | | | ACAAATGTGGCCCACTGGA | |
| | | | CAACACCTAGCCTGAAGTG | |
| | | | CATTAGAGATCCTGCCCTG | |
| | | | GTCCACCAGAGGCCTGCCC | |
| | | | CTCCATCTACAGTGACAAC | |
| | | | AGCCGGAGTGACACCTCAG | |
| | | | CCTGAATCTCTGAGCCCTT | |
| | | | CTGGAAAAGAACCTGCCGC | |
| | | | CAGCTCTCCTAGCTCTAAT | |
| | | | AATACCGCCGCCACAACAG | |
| | | | CCGCCATTGTGCCTGGATC | |
| | | | TCAGCTGATGCCTAGCAAG | |
| | | | TCTCCTAGCACAGGCACAA | |
| | | | CAGAGATCAGCAGCCACGA | |
| | | | ATCTTCTCACGGAACACCT | |
| | | | TCTCAGACCACCGCCAAGA | |
| | | | ATTGGGAGCTGACAGCCTC | |
| | | | TGCCTCTCACCAGCCTCCA | |
| | | | GGAGTGTATCCTCAGGGCC | |
| | | | ACTCTGATACAACAGTGGC | |
| | | | CATCAGCACATCTACAGTG | |
| | | | CTGCTGTGTGGACTGTCTG | |
| | | | CCGTGTCTCTGCTGGCCTG | |
| | | | TTACCTGAAGTCTAGACAG | |
| | | | ACACCTCCTCTGGCCTCTG | |
| | | | TGGAGATGGAGGCCATGGA | |
| | | | AGCCCTGCCTGTGACATGG | |
| | | | GGAACAAGCAGCAGAGATG | |
| | | | AGGACCTGGAGAATTGTTC | |
| | | | TCACCACCTG | |
| mbIL15 (without | NWVNVISDLKKIEDLIQSM | 122 | AACTGGGTGAATGTGATCA | 132 |
| N-terminal signal | HIDATLYTESDVHPSCKVT | | GCGACCTGAAGAAGATCGA | |
| sequence and with | AMKCFLLELQVISLESGDA | | GGATCTGATCCAGAGCATG | |
| C-terminal tail) | SIHDTVENLIILANNSLSS | | CACATTGATGCCACCCTGT | |
| | NGNVTESGCKECEELEEKN | | ACACAGAATCTGATGTGCA | |
| | IKEFLQSFVHIVQMFINTS | | CCCTAGCTGTAAAGTGACC | |
| | SGGGSGGGGSGGGGSGGGG | | GCCATGAAGTGTTTTCTGC | |
| | SGGGSLQITCPPPMSVEHA | | TGGAGCTGCAGGTGATTTC | |
| | DIWVKSYSLYSRERYICNS | | TCTGGAAAGCGGAGATGCC | |
| | GFKRKAGTSSLTECVLNKA | | TCTATCCACGACACAGTGG | |
| | TNVAHWTTPSLKCIRDPAL | | AGAATCTGATCATCCTGGC | |
| | VHQRPAPPSTVTTAGVTPQ | | CAACAATAGCCTGAGCAGC | |
| | PESLSPSGKEPAASSPSSN | | AATGGCAATGTGACAGAGT | |
| | NTAATTAAIVPGSQLMPSK | | CTGGCTGTAAGGAGTGTGA | |
| | SPSTGTTEISSHESSHGTP | | GGAGCTGGAGGAGAAGAAC | |
| | SQTTAKNWELTASASHQPP | | ATCAAGGAGTTTCTGCAGA | |
| | GVYPQGHSDTTVAISTSTV | | GCTTTGTGCACATCGTGCA | |
| | LLCGLSAVSLLACYLKSRQ | | GATGTTCATCAATACAAGC | |
| | TPPLASVEMEAMEALPVTW | | TCTGGCGGAGGATCTGGAG | |
| | GTSSRDEDLENCSHHLLEG | | GAGGCGGATCTGGAGGAGG | |
| | GGEGRGSLLTCGDVEENPG | | AGGCAGTGGAGGCGGAGGA | |
| | | | TCTGGCGGAGGATCTCTGC | |
| | | | AGATTACATGCCCTCCTCC | |
| | | | AATGTCTGTGGAGCACGCC | |
| | | | GATATTTGGGTGAAGTCCT | |
| | | | ACAGCCTGTACAGCAGAGA | |
| | | | GAGATACATCTGCAACAGC | |
| | | | GGCTTTAAGAGAAAGGCCG | |
| | | | GCACCTCTTCTCTGACAGA | |
| | | | GTGCGTGCTGAATAAGGCC | |
| | | | ACAAATGTGGCCCACTGGA | |
| | | | CAACACCTAGCCTGAAGTG | |
| | | | CATTAGAGATCCTGCCCTG | |
| | | | GTCCACCAGAGGCCTGCCC | |
| | | | CTCCATCTACAGTGACAAC | |
| | | | AGCCGGAGTGACACCTCAG | |
| | | | CCTGAATCTCTGAGCCCTT | |
| | | | CTGGAAAAGAACCTGCCGC | |
| | | | CAGCTCTCCTAGCTCTAAT | |
| | | | AATACCGCCGCCACAACAG | |
| | | | CCGCCATTGTGCCTGGATC | |
| | | | TCAGCTGATGCCTAGCAAG | |
| | | | TCTCCTAGCACAGGCACAA | |
| | | | CAGAGATCAGCAGCCACGA | |
| | | | ATCTTCTCACGGAACACCT | |
| | | | TCTCAGACCACCGCCAAGA | |
| | | | ATTGGGAGCTGACAGCCTC | |
| | | | TGCCTCTCACCAGCCTCCA | |
| | | | GGAGTGTATCCTCAGGGCC | |
| | | | ACTCTGATACAACAGTGGC | |
| | | | CATCAGCACATCTACAGTG | |
| | | | CTGCTGTGTGGACTGTCTG | |
| | | | CCGTGTCTCTGCTGGCCTG | |
| | | | TTACCTGAAGTCTAGACAG | |
| | | | ACACCTCCTCTGGCCTCTG | |
| | | | TGGAGATGGAGGCCATGGA | |
| | | | AGCCCTGCCTGTGACATGG | |
| | | | GGAACAAGCAGCAGAGATG | |
| | | | AAGACCTGGAGAATTGTTC | |
| | | | TCACCACCTGCTGGAGGGC | |
| | | | GGCGGAGAGGGCAGAGGAA | |
| | | | GTCTTCTAACATGCGGTGA | |
| | | | CGTGGAGGAGAATCCCGGC | |
| Soluble hIL-15 | NWVNVISDLKKIEDLIQSM | 123 | AACTGGGTGAATGTGATCA | 134 |
| | HIDATLYTESDVHPSCKVT | | GCGACCTGAAGAAGATCGA | |
| | AMKCFLLELQVISLESGDA | | GGATCTGATCCAGAGCATG | |
| | SIHDTVENLIILANNSLSS | | CACATTGATGCCACCCTGT | |
| | NGNVTESGCKECEELEEKN | | ACACAGAATCTGATGTGCA | |
| | IKEFLQSFVHIVQMFINTS | | CCCTAGCTGTAAAGTGACC | |
| | | | GCCATGAAGTGTTTTCTGC | |
| | | | TGGAGCTGCAGGTGATTTC | |
| | | | TCTGGAAAGCGGAGATGCC | |
| | | | TCTATCCACGACACAGTGG | |
| | | | AGAATCTGATCATCCTGGC | |
| | | | CAACAATAGCCTGAGCAGC | |
| | | | AATGGCAATGTGACAGAGT | |
| | | | CTGGCTGTAAGGAGTGTGA | |
| | | | GGAGCTGGAGGAGAAGAAC | |
| | | | ATCAAGGAGTTTCTGCAGA | |
| | | | GCTTTGTGCACATCGTGCA | |
| | | | GATGTTCATCAATACAAGC | |
| hIL-15Rα | ITCPPPMSVEHADIWVKSY | 124 | ATTACATGCCCTCCTCCAA | 135 |
| | SLYSRERYICNSGFKRKAG | | TGTCTGTGGAGCACGCCGA | |
| | TSSLTECVLNKATNVAHWT | | TATTTGGGTGAAGTCCTAC | |
| | TPSLKCIRDPALVHQRPAP | | AGCCTGTACAGCAGAGAGA | |
| | PSTVTTAGVTPQPESLSPS | | GATACATCTGCAACAGCGG | |
| | GKEPAASSPSSNNTAATTA | | CTTTAAGAGAAAGGCCGGC | |
| | AIVPGSQLMPSKSPSTGTT | | ACCTCTTCTCTGACAGAGT | |
| | EISSHESSHGTPSQTTAKN | | GCGTGCTGAATAAGGCCAC | |
| | WELTASASHQPPGVYPQGH | | AAATGTGGCCCACTGGACA | |
| | SDTTVAISTSTVLLCGLSA | | ACACCTAGCCTGAAGTGCA | |
| | VSLLACYLKSRQTPPLASV | | TTAGAGATCCTGCCCTGGT | |
| | EMEAMEALPVTWGTSSRDE | | CCACCAGAGGCCTGCCCCT | |
| | DLENCSHHL | | CCATCTACAGTGACAACAG | |
| | | | CCGGAGTGACACCTCAGCC | |
| | | | TGAATCTCTGAGCCCTTCT | |
| | | | GGAAAAGAACCTGCCGCCA | |
| | | | GCTCTCCTAGCTCTAATAA | |
| | | | TACCGCCGCCACAACAGCC | |
| | | | GCCATTGTGCCTGGATCTC | |
| | | | AGCTGATGCCTAGCAAGTC | |
| | | | TCCTAGCACAGGCACAACA | |
| | | | GAGATCAGCAGCCACGAAT | |
| | | | CTTCTCACGGAACACCTTC | |
| | | | TCAGACCACCGCCAAGAAT | |
| | | | TGGGAGCTGACAGCCTCTG | |
| | | | CCTCTCACCAGCCTCCAGG | |
| | | | AGTGTATCCTCAGGGCCAC | |
| | | | TCTGATACAACAGTGGCCA | |
| | | | TCAGCACATCTACAGTGCT | |
| | | | GCTGTGTGGACTGTCTGCC | |
| | | | GTGTCTCTGCTGGCCTGTT | |
| | | | ACCTGAAGTCTAGACAGAC | |
| | | | ACCTCCTCTGGCCTCTGTG | |
| | | | GAGATGGAGGCCATGGAAG | |
| | | | CCCTGCCTGTGACATGGGG | |
| | | | AACAAGCAGCAGAGATGAA | |
| | | | GACCTGGAGAATTGTTCTC | |
| | | | ACCACCTG | |
| | | | ATTACATGCCCTCCTCCAA | 163 |
| | | | TGTCTGTGGAGCACGCCGA | |
| | | | TATTTGGGTGAAGTCCTAC | |
| | | | AGCCTGTACAGCAGAGAGA | |
| | | | GATACATCTGCAACAGCGG | |
| | | | CTTTAAGAGAAAGGCCGGC | |
| | | | ACCTCTTCTCTGACAGAGT | |
| | | | GCGTGCTGAATAAGGCCAC | |
| | | | AAATGTGGCCCACTGGACA | |
| | | | ACACCTAGCCTGAAGTGCA | |
| | | | TTAGAGATCCTGCCCTGGT | |
| | | | CCACCAGAGGCCTGCCCCT | |
| | | | CCATCTACAGTGACAACAG | |
| | | | CCGGAGTGACACCTCAGCC | |
| | | | TGAATCTCTGAGCCCTTCT | |
| | | | GGAAAAGAACCTGCCGCCA | |
| | | | GCTCTCCTAGCTCTAATAA | |
| | | | TACCGCCGCCACAACAGCC | |
| | | | GCCATTGTGCCTGGATCTC | |
| | | | AGCTGATGCCTAGCAAGTC | |
| | | | TCCTAGCACAGGCACAACA | |
| | | | GAGATCAGCAGCCACGAAT | |
| | | | CTTCTCACGGAACACCTTC | |
| | | | TCAGACCACCGCCAAGAAT | |
| | | | TGGGAGCTGACAGCCTCTG | |
| | | | CCTCTCACCAGCCTCCAGG | |
| | | | AGTGTATCCTCAGGGCCAC | |
| | | | TCTGATACAACAGTGGCCA | |
| | | | TCAGCACATCTACAGTGCT | |
| | | | GCTGTGTGGACTGTCTGCC | |
| | | | GTGTCTCTGCTGGCCTGTT | |
| | | | ACCTGAAGTCTAGACAGAC | |
| | | | ACCTCCTCTGGCCTCTGTG | |
| | | | GAGATGGAGGCCATGGAAG | |
| | | | CCCTGCCTGTGACATGGGG | |
| | | | AACAAGCAGCAGAGATGAG | |
| | | | GACCTGGAGAATTGTTCTC | |
| | | | ACCACCTG | |
| Linker | SGGGSGGGGSGGGGSGGGG | 125 | TCTGGCGGAGGATCTGGAG | 136 |
| | SGGGSLQ | | GAGGCGGATCTGGAGGAGG | |
| | | | AGGCAGTGGAGGCGGAGGA | |
| | | | TCTGGCGGAGGATCTCTGC | |
| | | | AG | |
| IgE N-terminal | MDWTWILFLVAAATRVHS | 176 | ATGGATTGGACCTGGATTC | 177 |
| signal sequence | | | TGTTTCTGGTGGCCGCTGC | |
| | | | CACAAGAGTGCACAGC | |

### 5.4 Marker Proteins

The marker proteins described herein function to allows for the selective depletion of anti-CD19 CAR expressing cells in vivo, through the administration of an agent, e.g., an antibody, that specifically binds to the marker protein and mediates or catalyzes killing of the anti-CD19 CAR expressing cell. In some embodiments, marker proteins are expressed on the surface of the cell expressing the anti-CD19 CAR.

In some embodiments, the marker protein comprises the extracellular domain of a cell surface protein, or a functional fragment or functional variant thereof. In some embodiments, the cell surface protein is human epidermal growth factor receptor 1 (hHER1). In some embodiments, the marker protein comprises a truncated HER1 protein that is able to be bound by an anti-hHER1 antibody. In some embodiments, the marker protein comprises a variant of a truncated hHER1 protein that is able to be bound by an anti-hHER1 antibody. In some embodiments, the hHER1 marker protein provides a safety mechanism by allowing for depletion of infused CAR-T cells through administering an antibody that recognizes the hHER1 marker protein expressed on the surface of anti-CD19 CAR expressing cells. An exemplary antibody that binds the hHER1 marker protein is cetuximab.

In some embodiments, the hHER1 marker protein comprises from N terminus to C terminus: domain III of hHER1, or a functional fragment or functional variant thereof; an N-terminal portion of domain IV of hHER1; and the transmembrane region of human CD28.

In some embodiments, domain III of hHER1 comprises the amino acid sequence of SEQ ID NO: 98; or the amino acid sequence of SEQ ID NO: 98, comprising 1, 2, or 3 amino acid modifications. In some embodiments, the amino acid sequence of domain III of hHER1 consists of the amino acid sequence of SEQ ID NO: 98; or the amino acid sequence of SEQ ID NO: 98, comprising 1, 2, or 3 amino acid modifications.

In some embodiments, domain III of hHER1 is encoded by a polynucleotide sequence at least 75%, 80%, 85%, 90%, 95%, 96%, 97%, 98%, 99% or 100% identical to the polynucleotide sequence of SEQ ID NO: 110. In some embodiments, domain III of hHER1 is encoded by the polynucleotide sequence of SEQ ID NO: 110. In some embodiments, domain III of hHER1 is encoded by a polynucleotide sequence at least 75%, 80%, 85%, 90%, 95%, 96%, 97%, 98%, 99% or 100% identical to the polynucleotide sequence of SEQ ID NO: 164. In some embodiments, domain III of hHER1 is encoded by the polynucleotide sequence of SEQ ID NO: 164.

In some embodiments, the N-terminal portion of domain IV of hHER1 comprises amino acids 1-40, 1-39, 1-38, 1-37, 1-36, 1-35, 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, or 1-10 of SEQ ID NO: 99. In some embodiments, the C terminus of domain III of hHER1 is directly fused to the N terminus of the N-terminal portion of domain IV of hHER1.

In some embodiments, the C terminus of the N-terminal portion of domain IV of hHER1 is indirectly fused to the N terminus of the CD28 transmembrane domain via a peptide linker. In some embodiments, the peptide linker comprises glycine and serine amino acid residues. In some embodiments, the peptide linker is from about 5-25, 5-20, 5-15, 5-10, 10-20, or 10-15 amino acids in length.

In some embodiments, the peptide linker comprises the amino acid sequence of SEQ ID NO: 102, or an amino acid sequence comprising 1, 2, 3, 4 or 5 amino acid modifications to the amino acid sequence of SEQ ID NO: 102. In some embodiments, the peptide linker comprises the amino acid sequence of SEQ ID NO: 102. In some embodiments, the amino acid sequence of the peptide linker consists of the amino acid sequence of SEQ ID NO: 102, or an amino acid sequence comprising 1, 2, 3, 4 or 5 amino acid modifications to the amino acid sequence of SEQ ID NO: 102. In some embodiments, the amino acid sequence of the peptide linker consists of the amino acid sequence of SEQ ID NO: 102. In some embodiments, the peptide linker is encoded by a polynucleotide sequence at least 75%, 80%, 85%, 90%, 95%, 96%, 97%, 98%, 99% or 100% identical to the polynucleotide sequence of SEQ ID NO: 114. In some embodiments, the peptide linker is encoded by the polynucleotide sequence of SEQ ID NO: 114.

In some embodiments, the marker protein comprises an amino acid sequence at least 95%, 96%, 97%, 98%, 99% or 100% identical to the amino acid sequence of SEQ ID NO: 96, 97, 103, 104, 166, or 167. In some embodiments, the marker protein comprises an amino acid sequence at least 95%, 96%, 97%, 98%, 99% or 100% identical to the amino acid sequence of SEQ ID NO: 96. In some embodiments, the marker protein comprises an amino acid sequence at least 95%, 96%, 97%, 98%, 99% or 100% identical to the amino acid sequence of SEQ ID NO: 97. In some embodiments, the marker protein comprises an amino acid sequence at least 95%, 96%, 97%, 98%, 99% or 100% identical to the amino acid sequence of SEQ ID NO: 103. In some embodiments, the marker protein comprises an amino acid sequence at least 95%, 96%, 97%, 98%, 99% or 100% identical to the amino acid sequence of SEQ ID NO: 104. In some embodiments, the marker protein comprises an amino acid sequence at least 95%, 96%, 97%, 98%, 99% or 100% identical to the amino acid sequence of SEQ ID NO: 166. In some embodiments, the marker protein comprises an amino acid sequence at least 95%, 96%, 97%, 98%, 99% or 100% identical to the amino acid sequence of SEQ ID NO: 167.

In some embodiments, the marker protein comprises the amino acid sequence of SEQ ID NO: 96. In some embodiments, the marker protein comprises the amino acid sequence of SEQ ID NO: 97. In some embodiments, the marker protein comprises the amino acid sequence of SEQ ID NO: 96, 97, 103, or 104. In some embodiments, the marker protein comprises the amino acid sequence of SEQ ID NO: 103. In some embodiments, the marker protein comprises the amino acid sequence of SEQ ID NO: 104. In some embodiments, the marker protein comprises the amino acid sequence of SEQ ID NO: 166. In some embodiments, the marker protein comprises the amino acid sequence of SEQ ID NO: 167.

In some embodiments, the marker protein consists of an amino acid sequence at least 95%, 96%, 97%, 98%, 99% or 100% identical to the amino acid sequence of SEQ ID NO: 96, 97, 103, 104, 166, or 167. In some embodiments, the marker protein consists of an amino acid sequence at least 95%, 96%, 97%, 98%, 99% or 100% identical to the amino acid sequence of SEQ ID NO: 96. In some embodiments, the marker protein consists of an amino acid sequence at least 95%, 96%, 97%, 98%, 99% or 100% identical to the amino acid sequence of SEQ ID NO: 97. In some embodiments, the marker protein consists of an amino acid sequence at least 95%, 96%, 97%, 98%, 99% or 100% identical to the amino acid sequence of SEQ ID NO: 103. In some embodiments, the marker protein consists of an amino acid sequence at least 95%, 96%, 97%, 98%, 99% or 100% identical to the amino acid sequence of SEQ ID NO: 104. In some embodiments, the marker protein consists of an amino acid sequence at least 95%, 96%, 97%, 98%, 99% or 100% identical to the amino acid sequence of SEQ ID NO: 166. In some embodiments, the marker protein consists of an amino acid sequence at least 95%, 96%, 97%, 98%, 99% or 100% identical to the amino acid sequence of SEQ ID NO: 167.

In some embodiments, the marker protein consists of the amino acid sequence of SEQ ID NO: 96, 97, 103, 104, 166, or 167. In some embodiments, the marker protein consists of the amino acid sequence of SEQ ID NO: 96. In some embodiments, the marker protein consists of the amino acid sequence of SEQ ID NO: 97. In some embodiments, the marker protein consists of the amino acid sequence of SEQ ID NO: 103. In some embodiments, the marker protein consists of the amino acid sequence of SEQ ID NO: 104. In some embodiments, the marker protein consists of the amino acid sequence of SEQ ID NO: 166. In some embodiments, the marker protein consists of the amino acid sequence of SEQ ID NO: 167.

In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 107, 162, 108, 109, 115, 116, 173, or 174. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 107. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 162. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 108. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 109. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 115. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 116. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 173. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 174.

In some embodiments, the maker protein is encoded by the polynucleotide sequence of SEQ ID NO: 107, 162, 108, 109, 115, 116, 173, or 174. In some embodiments, the maker protein is encoded by the polynucleotide sequence of SEQ ID NO: 107. In some embodiments, the maker protein is encoded by a polynucleotide sequence comprising the polynucleotide sequence of SEQ ID NO: 162. In some embodiments, the maker protein is encoded by the polynucleotide sequence of SEQ ID NO: 108. In some embodiments, the maker protein is encoded by a polynucleotide sequence comprising the polynucleotide sequence of SEQ ID NO: 109. In some embodiments, the maker protein is encoded by the polynucleotide sequence of SEQ ID NO: 115. In some embodiments, the maker protein is encoded by the polynucleotide sequence of SEQ ID NO: 116. In some embodiments, the maker protein is encoded by the polynucleotide sequence of SEQ ID NO: 173. In some embodiments, the maker protein is encoded by the polynucleotide sequence of SEQ ID NO: 174.

In some embodiments, the marker protein is derived from human CD20 (hCD20). In some embodiments, the marker protein comprises a truncated hCD20 protein that comprises the extracellular region (hCD20t), or a functional fragment or functional variant thereof. In some embodiments, the hCD20 marker protein provides a safety mechanism by allowing for depletion of infused CAR-T cells through administering an antibody that recognizes the hCD20 marker protein expressed on the surface of CAR expressing cells. An exemplary antibody that binds the hCD20 marker protein is rituximab.

In some embodiments, the marker protein comprises an amino acid sequence at least 95%, 96%, 97%, 98%, 99% or 100% identical to the amino acid sequence of SEQ ID NO: 105. In some embodiments, the marker protein comprises an amino acid sequence at least 95%, 96%, 97%, 98%, 99% or 100% identical to the amino acid sequence of SEQ ID NO: 106. In some embodiments, the marker protein comprises the amino acid sequence of SEQ ID NO: 105. In some embodiments, the marker protein comprises the amino acid sequence of SEQ ID NO: 106.

In some embodiments, the amino acid sequence of the marker protein consists of a sequence at least 95%, 96%, 97%, 98%, 99% or 100% identical to the amino acid sequence of SEQ ID NO: 105. In some embodiments, the amino acid sequence of the marker protein consists of a sequence at least 95%, 96%, 97%, 98%, 99% or 100% identical to the amino acid sequence of SEQ ID NO: 106. In some embodiments, the amino acid sequence of the marker protein consists of the amino acid sequence of SEQ ID NO: 105. In some embodiments, the amino acid sequence of the marker protein consists of the amino acid sequence of SEQ ID NO: 106.

In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 117 or 118. In some embodiments, the marker protein is encoded by a polynucleotide sequence at least 75%, 80%, 85%, 90%, 95%, 96%, 97%, 98%, 99% or 1000% identical to the polynucleotide sequence of SEQ ID NO: 117. In some embodiments, the marker protein is encoded by a polynucleotide sequence at least 75%, 80%, 85%, 90%, 95%, 96%, 97%, 98%, 99% or 100% identical to the polynucleotide sequence of SEQ ID NO: 118. In some embodiments, the maker protein is encoded by the polynucleotide sequence of SEQ ID NO: 117 or 118. In some embodiments, the maker protein is encoded by the polynucleotide sequence of SEQ ID NO: 117. In some embodiments, the maker protein is encoded by the polynucleotide sequence of SEQ ID NO: 118.

The amino acid sequence and polynucleotide sequence of exemplary marker proteins are provided in Table 7, herein.

TABLE 7
Amino acid and polynucleotide sequences of exemplary marker proteins.

| | | SEQ ID | Polynucleotide | SEQ ID |
|---|---|---|---|---|
| Description | Amino Acid Sequence | NO | Sequence | NO |
| HER1t (with N- | MRLPAQLLGLLMLWVPGSS | 96 | ATGAGGCTCCCTGCTCAGC | 107 |
| terminal signal | GRKVCNGIGIGEFKDSLSI | | TCCTGGGGCTGCTAATGCT | |
| sequence) | NATNIKHFKNCTSISGDLH | | CTGGGTGCCAGGATCCAGT | |
| | ILPVAFRGDSFTHTPPLDP | | GGGCGCAAAGTGTGTAACG | |
| | QELDILKTVKEITGELLIQ | | GAATAGGTATTGGTGAATT | |
| | AWPENRTDLHAFENLEIIR | | TAAAGACTCACTCTCCATA | |
| | GRTKQHGQFSLAVVSLNIT | | AATGCTACGAATATTAAAC | |
| | SLGLRSLKEISDGDVIISG | | ACTTCAAAAACTGCACCTC | |
| | NKNLCYANTINWKKLFGTS | | CATCAGTGGCGATCTCCAC | |
| | GQKTKIISNRGENSCKATG | | ATCCTGCCGGTGGCATTTA | |
| | QVCHALCSPEGCWGPEPRD | | GGGGTGACTCCTTCACACA | |
| | CVSGGGGSGGGGSGGGGSG | | TACTCCTCCTCTGGATCCA | |
| | GGGSFWVLVVVGGVLACYS | | CAGGAACTGGATATTCTGA | |
| | LLVTVAFIIFWVRSKRS | | AAACCGTAAAGGAAATCAC | |
| | | | AGGGTTTTTGCTGATTCAG | |
| | | | GCTTGGCCTGAAAACAGGA | |
| | | | CGGACCTCCATGCCTTTGA | |
| | | | GAACCTAGAAATCATACGC | |
| | | | GGCAGGACCAAGCAACATG | |
| | | | GTCAGTTTTCTCTTGCAGT | |
| | | | CGTCAGCCTGAACATAACA | |
| | | | TCCTTGGGATTACGCTCCC | |
| | | | TCAAGGAGATAAGTGATGG | |
| | | | AGATGTGATAATTTCAGGA | |
| | | | AACAAAAATTTGTGCTATG | |
| | | | CAAATACAATAAACTGGAA | |
| | | | AAAACTGTTTGGTACCTCC | |
| | | | GGTCAGAAAACCAAAATTA | |
| | | | TAAGCAACAGAGGTGAAAA | |
| | | | CAGCTGCAAGGCCACAGGC | |
| | | | CAGGTCTGCCATGCCTTGT | |
| | | | GCTCCCCCGAGGGCTGCTG | |
| | | | GGGCCCGGAGCCCAGGGAC | |
| | | | TGCGTCTCTGGTGGCGGTG | |
| | | | GCTCGGGCGGTGGTGGGTC | |
| | | | GGGTGGCGGCGGATCTGGT | |
| | | | GGCGGTGGCTCGTTTTGGG | |
| | | | TGCTGGTGGTGGTTGGTGG | |
| | | | AGTCCTGGCTTGCTATAGC | |
| | | | TTGCTAGTAACAGTGGCCT | |
| | | | TTATTATTTTCTGGGTGAG | |
| | | | GAGTAAGAGGAGC | |
| | | | ATGAGGCTCCCTGCTCAGC | 162 |
| | | | TCCTGGGGCTGCTAATGCT | |
| | | | CTGGGTCCCAGGATCCAGT | |
| | | | GGGCGCAAAGTGTGTAACG | |
| | | | GAATAGGTATTGGTGAATT | |
| | | | TAAAGACTCACTCTCCATA | |
| | | | AATGCTACGAATATTAAAC | |
| | | | ACTTCAAAAACTGCACCTC | |
| | | | CATCAGTGGCGATCTCCAC | |
| | | | ATCCTGCCGGTGGCATTTA | |
| | | | GGGGTGACTCCTTCACACA | |
| | | | TACTCCTCCTCTGGATCCA | |
| | | | CAGGAACTGGATATTCTGA | |
| | | | AAACCGTAAAGGAAATCAC | |
| | | | AGGGTTTTTGCTGATTCAG | |
| | | | GCTTGGCCTGAAAACAGGA | |
| | | | CGGACCTCCATGCCTTTGA | |
| | | | GAACCTAGAAATCATACGC | |
| | | | GGCAGGACCAAGCAACATG | |
| | | | GTCAGTTTTCTCTTGCAGT | |
| | | | CGTCAGCCTGAACATAACA | |
| | | | TCCTTGGGATTACGCTCCC | |
| | | | TCAAGGAGATAAGTGATGG | |
| | | | AGATGTGATAATTTCAGGA | |
| | | | AACAAAAATTTGTGCTATG | |
| | | | CAAATACAATAAACTGGAA | |
| | | | AAAACTGTTTGGGACCTCC | |
| | | | GGTCAGAAAACCAAAATTA | |
| | | | TAAGCAACAGAGGTGAAAA | |
| | | | CAGCTGCAAGGCCACAGGC | |
| | | | CAGGTCTGCCATGCCTTGT | |
| | | | GCTCCCCCGAGGGCTGCTG | |
| | | | GGGCCCGGAGCCCAGGGAC | |
| | | | TGCGTCTCTGGTGGCGGTG | |
| | | | GCTCGGGCGGTGGTGGGTC | |
| | | | GGGTGGCGGCGGATCTGGT | |
| | | | GGCGGTGGCTCGTTTTGGG | |
| | | | TGCTGGTGGTGGTTGGTGG | |
| | | | AGTCCTGGCTTGCTATAGC | |
| | | | TTGCTAGTAACAGTGGCCT | |
| | | | TTATTATTTTCTGGGTGAG | |
| | | | GAGTAAGAGGAGC | |
| HER1t (with | MRMRLPAQLLGLLMLWVPG | 166 | ATGAGGATGAGGCTCCCTG | 173 |
| Variant 1 N- | SSGRKVCNGIGIGEFKDSL | | CTCAGCTCCTGGGGCTGCT | |
| terminal signal | SINATNIKHFKNCTSISGD | | AATGCTCTGGGTCCCAGGA | |
| sequence) | LHILPVAFRGDSFTHTPPL | | TCCAGTGGGCGCAAAGTGT | |
| | DPQELDILKTVKEITGELL | | GTAACGGAATAGGTATTGG | |
| | IQAWPENRTDLHAFENLEI | | TGAATTTAAAGACTCACTC | |
| | IRGRTKQHGQFSLAVVSLN | | TCCATAAATGCTACGAATA | |
| | ITSLGLRSLKEISDGDVII | | TTAAACACTTCAAAAACTG | |
| | SGNKNLCYANTINWKKLFG | | CACCTCCATCAGTGGCGAT | |
| | TSGQKTKIISNRGENSCKA | | CTCCACATCCTGCCGGTGG | |
| | TGQVCHALCSPEGCWGPEP | | CATTTAGGGGTGACTCCTT | |
| | RDCVSGGGGSGGGGSGGGG | | CACACATACTCCTCCTCTG | |
| | SGGGGSFWVLVVVGGVLAC | | GATCCACAGGAACTGGATA | |
| | YSLLVTVAFIIFWVRSKRS | | TTCTGAAAACCGTAAAGGA | |
| | | | AATCACAGGGTTTTTGCTG | |
| | | | ATTCAGGCTTGGCCTGAAA | |
| | | | ACAGGACGGACCTCCATGC | |
| | | | CTTTGAGAACCTAGAAATC | |
| | | | ATACGCGGCAGGACCAAGC | |
| | | | AACATGGTCAGTTTTCTCT | |
| | | | TGCAGTCGTCAGCCTGAAC | |
| | | | ATAACATCCTTGGGATTAC | |
| | | | GCTCCCTCAAGGAGATAAG | |
| | | | TGATGGAGATGTGATAATT | |
| | | | TCAGGAAACAAAAATTTGT | |
| | | | GCTATGCAAATACAATAAA | |
| | | | CTGGAAAAAACTGTTTGGG | |
| | | | ACCTCCGGTCAGAAAACCA | |
| | | | AAATTATAAGCAACAGAGG | |
| | | | TGAAAACAGCTGCAAGGCC | |
| | | | ACAGGCCAGGTCTGCCATG | |
| | | | CCTTGTGCTCCCCCGAGGG | |
| | | | CTGCTGGGGCCCGGAGCCC | |
| | | | AGGGACTGCGTCTCTGGTG | |
| | | | GCGGTGGCTCGGGCGGTGG | |
| | | | TGGGTCGGGTGGCGGCGGA | |
| | | | TCTGGTGGCGGTGGCTCGT | |
| | | | TTTGGGTGCTGGTGGTGGT | |
| | | | TGGTGGAGTCCTGGCTTGC | |
| | | | TATAGCTTGCTAGTAACAG | |
| | | | TGGCCTTTATTATTTTCTG | |
| | | | GGTGAGGAGTAAGAGGAGC | |
| HER1t (with | PRMRLPAQLLGLLMLWVPG | 167 | CCTAGGATGAGGCTCCCTG | 174 |
| Variant 2 N- | SSGRKVCNGIGIGEFKDSL | | CTCAGCTCCTGGGGCTGCT | |
| terminal signal | SINATNIKHFKNCTSISGD | | AATGCTCTGGGTGCCAGGA | |
| sequence) | LHILPVAFRGDSFTHTPPL | | TCCAGTGGGCGCAAAGTGT | |
| | DPQELDILKTVKEITGELL | | GTAACGGAATAGGTATTGG | |
| | IQAWPENRTDLHAFENLEI | | TGAATTTAAAGACTCACTC | |
| | IRGRTKQHGQFSLAVVSLN | | TCCATAAATGCTACGAATA | |
| | ITSLGLRSLKEISDGDVII | | TTAAACACTTCAAAAACTG | |
| | SGNKNLCYANTINWKKLFG | | CACCTCCATCAGTGGCGAT | |
| | TSGQKTKIISNRGENSCKA | | CTCCACATCCTGCCGGTGG | |
| | TGQVCHALCSPEGCWGPEP | | CATTTAGGGGTGACTCCTT | |
| | RDCVSGGGGSGGGGSGGGG | | CACACATACTCCTCCTCTG | |
| | SGGGGSFWVLVVVGGVLAC | | GATCCACAGGAACTGGATA | |
| | YSLLVTVAFIIFWVRSKRS | | TTCTGAAAACCGTAAAGGA | |
| | | | AATCACAGGGTTTTTGCTG | |
| | | | ATTCAGGCTTGGCCTGAAA | |
| | | | ACAGGACGGACCTCCATGC | |
| | | | CTTTGAGAACCTAGAAATC | |
| | | | ATACGCGGCAGGACCAAGC | |
| | | | AACATGGTCAGTTTTCTCT | |
| | | | TGCAGTCGTCAGCCTGAAC | |
| | | | ATAACATCCTTGGGATTAC | |
| | | | GCTCCCTCAAGGAGATAAG | |
| | | | TGATGGAGATGTGATAATT | |
| | | | TCAGGAAACAAAAATTTGT | |
| | | | GCTATGCAAATACAATAAA | |
| | | | CTGGAAAAAACTGTTTGGT | |
| | | | ACCTCCGGTCAGAAAACCA | |
| | | | AAATTATAAGCAACAGAGG | |
| | | | TGAAAACAGCTGCAAGGCC | |
| | | | ACAGGCCAGGTCTGCCATG | |
| | | | CCTTGTGCTCCCCCGAGGG | |
| | | | CTGCTGGGGCCCGGAGCCC | |
| | | | AGGGACTGCGTCTCTGGTG | |
| | | | GCGGTGGCTCGGGCGGTGG | |
| | | | TGGGTCGGGTGGCGGCGGA | |
| | | | TCTGGTGGCGGTGGCTCGT | |
| | | | TTTGGGTGCTGGTGGTGGT | |
| | | | TGGTGGAGTCCTGGCTTGC | |
| | | | TATAGCTTGCTAGTAACAG | |
| | | | TGGCCTTTATTATTTTCTG | |
| | | | GGTGAGGAGTAAGAGGAGC | |
| HER1t (without | RKVCNGIGIGEFKDSLSIN | 97 | CGCAAAGTGTGTAACGGAA | 108 |
| N-terminal signal | ATNIKHFKNCTSISGDLHI | | TAGGTATTGGTGAATTTAA | |
| sequence) | LPVAFRGDSFTHTPPLDPQ | | AGACTCACTCTCCATAAAT | |
| | ELDILKTVKEITGFLLIQA | | GCTACGAATATTAAACACT | |
| | WPENRTDLHAFENLEIIRG | | TCAAAAACTGCACCTCCAT | |
| | RTKQHGQFSLAVVSLNITS | | CAGTGGCGATCTCCACATC | |
| | LGLRSLKEISDGDVIISGN | | CTGCCGGTGGCATTTAGGG | |
| | KNLCYANTINWKKLFGTSG | | GTGACTCCTTCACACATAC | |
| | QKTKIISNRGENSCKATGQ | | TCCTCCTCTGGATCCACAG | |
| | VCHALCSPEGCWGPEPRDC | | GAACTGGATATTCTGAAAA | |
| | VSGGGGSGGGGSGGGGSGG | | CCGTAAAGGAAATCACAGG | |
| | GGSFWVLVVVGGVLACYSL | | GTTTTTGCTGATTCAGGCT | |
| | LVTVAFIIFWVRSKRS | | TGGCCTGAAAACAGGACGG | |
| | | | ACCTCCATGCCTTTGAGAA | |
| | | | CCTAGAAATCATACGCGGC | |
| | | | AGGACCAAGCAACATGGTC | |
| | | | AGTTTTCTCTTGCAGTCGT | |
| | | | CAGCCTGAACATAACATCC | |
| | | | TTGGGATTACGCTCCCTCA | |
| | | | AGGAGATAAGTGATGGAGA | |
| | | | TGTGATAATTTCAGGAAAC | |
| | | | AAAAATTTGTGCTATGCAA | |
| | | | ATACAATAAACTGGAAAAA | |
| | | | ACTGTTTGGTACCTCCGGT | |
| | | | CAGAAAACCAAAATTATAA | |
| | | | GCAACAGAGGTGAAAACAG | |
| | | | CTGCAAGGCCACAGGCCAG | |
| | | | GTCTGCCATGCCTTGTGCT | |
| | | | CCCCCGAGGGCTGCTGGGG | |
| | | | CCCGGAGCCCAGGGACTGC | |
| | | | GTCTCTGGTGGCGGTGGCT | |
| | | | CGGGCGGTGGTGGGTCGGG | |
| | | | TGGCGGCGGATCTGGTGGC | |
| | | | GGTGGCTCGTTTTGGGTGC | |
| | | | TGGTGGTGGTTGGTGGAGT | |
| | | | CCTGGCTTGCTATAGCTTG | |
| | | | CTAGTAACAGTGGCCTTTA | |
| | | | TTATTTTCTGGGTGAGGAG | |
| | | | TAAGAGGAGC | |
| | | | CGCAAAGTGTGTAACGGAA | 109 |
| | | | TAGGTATTGGTGAATTTAA | |
| | | | AGACTCACTCTCCATAAAT | |
| | | | GCTACGAATATTAAACACT | |
| | | | TCAAAAACTGCACCTCCAT | |
| | | | CAGTGGCGATCTCCACATC | |
| | | | CTGCCGGTGGCATTTAGGG | |
| | | | GTGACTCCTTCACACATAC | |
| | | | TCCTCCTCTGGATCCACAG | |
| | | | GAACTGGATATTCTGAAAA | |
| | | | CCGTAAAGGAAATCACAGG | |
| | | | GTTTTTGCTGATTCAGGCT | |
| | | | TGGCCTGAAAACAGGACGG | |
| | | | ACCTCCATGCCTTTGAGAA | |
| | | | CCTAGAAATCATACGCGGC | |
| | | | AGGACCAAGCAACATGGTC | |
| | | | AGTTTTCTCTTGCAGTCGT | |
| | | | CAGCCTGAACATAACATCC | |
| | | | TTGGGATTACGCTCCCTCA | |
| | | | AGGAGATAAGTGATGGAGA | |
| | | | TGTGATAATTTCAGGAAAC | |
| | | | AAAAATTTGTGCTATGCAA | |
| | | | ATACAATAAACTGGAAAAA | |
| | | | ACTGTTTGGGACCTCCGGT | |
| | | | CAGAAAACCAAAATTATAA | |
| | | | GCAACAGAGGTGAAAACAG | |
| | | | CTGCAAGGCCACAGGCCAG | |
| | | | GTCTGCCATGCCTTGTGCT | |
| | | | CCCCCGAGGGCTGCTGGGG | |
| | | | CCCGGAGCCCAGGGACTGC | |
| | | | GTCTCTGGTGGCGGTGGCT | |
| | | | CGGGCGGTGGTGGGTCGGG | |
| | | | TGGCGGCGGATCTGGTGGC | |
| | | | GGTGGCTCGTTTTGGGTGC | |
| | | | TGGTGGTGGTTGGTGGAGT | |
| | | | CCTGGCTTGCTATAGCTTG | |
| | | | CTAGTAACAGTGGCCTTTA | |
| | | | TTATTTTCTGGGTGAGGAG | |
| | | | TAAGAGGAGC | |
| Domain III of | RKVCNGIGIGEFKDSLSIN | 98 | CGCAAAGTGTGTAACGGAA | 110 |
| hHER1 | ATNIKHFKNCTSISGDLHI | | TAGGTATTGGTGAATTTAA | |
| | LPVAFRGDSFTHTPPLDPQ | | AGACTCACTCTCCATAAAT | |
| | ELDILKTVKEITGFLLIQA | | GCTACGAATATTAAACACT | |
| | WPENRTDLHAFENLEIIRG | | TCAAAAACTGCACCTCCAT | |
| | RTKQHGOFSLAVVSLNITS | | CAGTGGCGATCTCCACATC | |
| | LGLRSLKEISDGDVIISGN | | CTGCCGGTGGCATTTAGGG | |
| | KNLCYANTINWKKLFGTSG | | GTGACTCCTTCACACATAC | |
| | QKTKIISNRGENSCKATGQ | | TCCTCCTCTGGATCCACAG | |
| | | | GAACTGGATATTCTGAAAA | |
| | | | CCGTAAAGGAAATCACAGG | |
| | | | GTTTTTGCTGATTCAGGCT | |
| | | | TGGCCTGAAAACAGGACGG | |
| | | | ACCTCCATGCCTTTGAGAA | |
| | | | CCTAGAAATCATACGCGGC | |
| | | | AGGACCAAGCAACATGGTC | |
| | | | AGTTTTCTCTTGCAGTCGT | |
| | | | CAGCCTGAACATAACATCC | |
| | | | TTGGGATTACGCTCCCTCA | |
| | | | AGGAGATAAGTGATGGAGA | |
| | | | TGTGATAATTTCAGGAAAC | |
| | | | AAAAATTTGTGCTATGCAA | |
| | | | ATACAATAAACTGGAAAAA | |
| | | | ACTGTTTGGTACCTCCGGT | |
| | | | CAGAAAACCAAAATTATAA | |
| | | | GCAACAGAGGTGAAAACAG | |
| | | | CTGCAAGGCCACAGGCCAG | |
| | | | CGCAAAGTGTGTAACGGAA | 164 |
| | | | TAGGTATTGGTGAATTTAA | |
| | | | AGACTCACTCTCCATAAAT | |
| | | | GCTACGAATATTAAACACT | |
| | | | TCAAAAACTGCACCTCCAT | |
| | | | CAGTGGCGATCTCCACATC | |
| | | | CTGCCGGTGGCATTTAGGG | |
| | | | GTGACTCCTTCACACATAC | |
| | | | TCCTCCTCTGGATCCACAG | |
| | | | GAACTGGATATTCTGAAAA | |
| | | | CCGTAAAGGAAATCACAGG | |
| | | | GTTTTTGCTGATTCAGGCT | |
| | | | TGGCCTGAAAACAGGACGG | |
| | | | ACCTCCATGCCTTTGAGAA | |
| | | | CCTAGAAATCATACGCGGC | |
| | | | AGGACCAAGCAACATGGTC | |
| | | | AGTTTTCTCTTGCAGTCGT | |
| | | | CAGCCTGAACATAACATCC | |
| | | | TTGGGATTACGCTCCCTCA | |
| | | | AGGAGATAAGTGATGGAGA | |
| | | | TGTGATAATTTCAGGAAAC | |
| | | | AAAAATTTGTGCTATGCAA | |
| | | | ATACAATAAACTGGAAAAA | |
| | | | ACTGTTTGGGACCTCCGGT | |
| | | | CAGAAAACCAAAATTATAA | |
| | | | GCAACAGAGGTGAAAACAG | |
| | | | CTGCAAGGCCACAGGCCAG | |
| Domain IV of | VCHALCSPEGCWGPEPRDC | 99 | GTCTGCCATGCCTTGTGCT | 111 |
| hHER1 | VSCRNVSRGRECVDKCNLL | | CCCCCGAGGGCTGCTGGGG | |
| | EGEPREFVENSECIOCHPE | | CCCGGAGCCCAGGGACTGC | |
| | CLPQAMNITCTGRGPDNCI | | GTCTCTTGCCGGAATGTCA | |
| | QCAHYIDGPHCVKTCPAGV | | GCCGAGGCAGGGAATGCGT | |
| | MGENNTLVWKYADAGHVCH | | GGACAAGTGCAACCTTCTG | |
| | LCHPNCTYGCTGPGLEGCP | | GAGGGTGAGCCAAGGGAGT | |
| | TNGPKIPS | | TTGTGGAGAACTCTGAGTG | |
| | | | CATACAGTGCCACCCAGAG | |
| | | | TGCCTGCCTCAGGCCATGA | |
| | | | ACATCACCTGCACAGGACG | |
| | | | GGGACCAGACAACTGTATC | |
| | | | CAGTGTGCCCACTACATTG | |
| | | | ACGGCCCCCACTGCGTCAA | |
| | | | GACCTGCCCGGCAGGAGTC | |
| | | | ATGGGAGAAAACAACACCC | |
| | | | TGGTCTGGAAGTACGCAGA | |
| | | | CGCCGGCCATGTGTGCCAC | |
| | | | CTGTGCCATCCAAACTGCA | |
| | | | CCTACGGATGCACTGGGCC | |
| | | | AGGTCTTGAAGGCTGTCCA | |
| | | | ACGAATGGGCCTAAGATCC | |
| | | | CGTCC | |
| Truncated domain | VCHALCSPEGCWGPEPRDC | 100 | GTCTGCCATGCCTTGTGCT | 112 |
| IV of hHER1 | VS | | CCCCCGAGGGCTGCTGGGG | |
| | | | CCCGGAGCCCAGGGACTGC | |
| | | | GTCTCT | |
| CD28 | FWVLVVVGGVLACYSLLVT | 101 | TTTTGGGTGCTGGTGGTGG | 113 |
| transmembrane | VAFIIFWVRSKRS | | TTGGTGGAGTCCTGGCTTG | |
| domain | | | CTATAGCTTGCTAGTAACA | |
| | | | GTGGCCTTTATTATTTTCT | |
| | | | GGGTGAGGAGTAAGAGGAG | |
| | | | C | |
| Linker | GGGGSGGGGSGGGGSGGGG | 102 | GGTGGCGGTGGCTCGGGCG | 114 |
| | S | | GTGGTGGGTCGGGTGGCGG | |
| | | | CGGATCTGGTGGCGGTGGC | |
| | | | TCG | |
| Igk N-terminal | MRLPAQLLGLLMLWVPGSS | 169 | ATGAGGCTCCCTGCTCAGC | 185 |
| | | | TCCTGGGGCTGCTAATGCT | |
| | | | CTGGGTGCCAGGATCCAGT | |
| | | | GGG | |
| signal sequence | G | | ATGAGGCTCCCTGCTCAGC | 186 |
| | | | TCCTGGGGCTGCTAATGCT | |
| | | | CTGGGTCCCAGGATCCAGT | |
| | | | GGG | |
| Igk Variant 1 N- | MRMRLPAQLLGLLMLWVPG | 170 | ATGAGGATGAGGCTCCCTG | 178 |
| terminal signal | SSG | | CTCAGCTCCTGGGGCTGCT | |
| sequence | | | AATGCTCTGGGTCCCAGGA | |
| | | | TCCAGTGGG | |
| Igk Variant 2 N- | PRMRLPAQLLGLLMLWVPG | 171 | CCTAGGATGAGGCTCCCTG | 179 |
| terminal signal | SSG | | CTCAGCTCCTGGGGCTGCT | |
| sequence | | | AATGCTCTGGGTGCCAGGA | |
| | | | TCCAGTGGG | |
| HER1t-2 (with N- | MRLPAQLLGLLMLWVPGSS | 103 | ATGAGGCTCCCTGCTCAGC | 115 |
| terminal signal | GRKVCNGIGIGEFKDSLSI | | TCCTGGGGCTGCTAATGCT | |
| sequence) | NATNIKHFKNCTSISGDLH | | CTGGGTGCCAGGATCCAGT | |
| | ILPVAFRGDSFTHTPPLDP | | GGGCGCAAAGTGTGTAACG | |
| | QELDILKTVKEITGFLLIQ | | GAATAGGTATTGGTGAATT | |
| | AWPENRTDLHAFENLEIIR | | TAAAGACTCACTCTCCATA | |
| | GRTKQHGQFSLAVVSLNIT | | AATGCTACGAATATTAAAC | |
| | SLGLRSLKEISDGDVIISG | | ACTTCAAAAACTGCACCTC | |
| | NKNLCYANTINWKKLFGTS | | CATCAGTGGCGATCTCCAC | |
| | GQKTKIISNRGENSCKATG | | ATCCTGCCGGTGGCATTTA | |
| | QVCHALCSPEGCWGPEPRD | | GGGGTGACTCCTTCACACA | |
| | CVSCRNVSRGRECVDKCNL | | TACTCCTCCTCTGGATCCA | |
| | LEGEPREFVENSECIQCHP | | CAGGAACTGGATATTCTGA | |
| | ECLPQAMNITCTGRGPDNC | | AAACCGTAAAGGAAATCAC | |
| | IQCAHYIDGPHCVKTCPAG | | AGGGTTTTTGCTGATTCAG | |
| | VMGENNTLVWKYADAGHVC | | GCTTGGCCTGAAAACAGGA | |
| | HLCHPNCTYGCTGPGLEGC | | CGGACCTCCATGCCTTTGA | |
| | PTNGPKIPSIATGMVGALL | | GAACCTAGAAATCATACGC | |
| | LLLVVALGIGLFM | | GGCAGGACCAAGCAACATG | |
| | | | GTCAGTTTTCTCTTGCAGT | |
| | | | CGTCAGCCTGAACATAACA | |
| | | | TCCTTGGGATTACGCTCCC | |
| | | | TCAAGGAGATAAGTGATGG | |
| | | | AGATGTGATAATTTCAGGA | |
| | | | AACAAAAATTTGTGCTATG | |
| | | | CAAATACAATAAACTGGAA | |
| | | | AAAACTGTTTGGGACCTCC | |
| | | | GGTCAGAAAACCAAAATTA | |
| | | | TAAGCAACAGAGGTGAAAA | |
| | | | CAGCTGCAAGGCCACAGGC | |
| | | | CAGGTCTGCCATGCCTTGT | |
| | | | GCTCCCCCGAGGGCTGCTG | |
| | | | GGGCCCGGAGCCCAGGGAC | |
| | | | TGCGTCTCTTGCCGGAATG | |
| | | | TCAGCCGAGGCAGGGAATG | |
| | | | CGTGGACAAGTGCAACCTT | |
| | | | CTGGAGGGTGAGCCAAGGG | |
| | | | AGTTTGTGGAGAACTCTGA | |
| | | | GTGCATACAGTGCCACCCA | |
| | | | GAGTGCCTGCCTCAGGCCA | |
| | | | TGAACATCACCTGCACAGG | |
| | | | ACGGGGACCAGACAACTGT | |
| | | | ATCCAGTGTGCCCACTACA | |
| | | | TTGACGGCCCCCACTGCGT | |
| | | | CAAGACCTGCCCGGCAGGA | |
| | | | GTCATGGGAGAAAACAACA | |
| | | | CCCTGGTCTGGAAGTACGC | |
| | | | AGACGCCGGCCATGTGTGC | |
| | | | CACCTGTGCCATCCAAACT | |
| | | | GCACCTACGGATGCACTGG | |
| | | | GCCAGGTCTTGAAGGCTGT | |
| | | | CCAACGAATGGGCCTAAGA | |
| | | | TCCCGTCCATCGCCACTGG | |
| | | | GATGGTGGGGGCCCTCCTC | |
| | | | TTGCTGCTGGTGGTGGCCC | |
| | | | TGGGGATCGGCCTCTTCAT | |
| | | | G | |
| HER1t-2 (without | RKVCNGIGIGEFKDSLSIN | 104 | CGCAAAGTGTGTAACGGAA | 116 |
| N-terminal signal | ATNIKHFKNCTSISGDLHI | | TAGGTATTGGTGAATTTAA | |
| sequence) | LPVAFRGDSFTHTPPLDPQ | | AGACTCACTCTCCATAAAT | |
| | ELDILKTVKEITGFLLIQA | | GCTACGAATATTAAACACT | |
| | WPENRTDLHAFENLEIIRG | | TCAAAAACTGCACCTCCAT | |
| | RTKQHGQFSLAVVSLNITS | | CAGTGGCGATCTCCACATC | |
| | LGLRSLKEISDGDVIISGN | | CTGCCGGTGGCATTTAGGG | |
| | KNLCYANTINWKKLFGTSG | | GTGACTCCTTCACACATAC | |
| | QKTKIISNRGENSCKATGQ | | TCCTCCTCTGGATCCACAG | |
| | VCHALCSPEGCWGPEPRDC | | GAACTGGATATTCTGAAAA | |
| | VSCRNVSRGRECVDKCNLL | | CCGTAAAGGAAATCACAGG | |
| | EGEPREFVENSECIQCHPE | | GTTTTTGCTGATTCAGGCT | |
| | CLPQAMNITCTGRGPDNCI | | TGGCCTGAAAACAGGACGG | |
| | QCAHYIDGPHCVKTCPAGV | | ACCTCCATGCCTTTGAGAA | |
| | MGENNTLVWKYADAGHVCH | | CCTAGAAATCATACGCGGC | |
| | LCHPNCTYGCTGPGLEGCP | | AGGACCAAGCAACATGGTC | |
| | TNGPKIPSIATGMVGALLL | | AGTTTTCTCTTGCAGTCGT | |
| | LLVVALGIGLFM | | CAGCCTGAACATAACATCC | |
| | | | TTGGGATTACGCTCCCTCA | |
| | | | AGGAGATAAGTGATGGAGA | |
| | | | TGTGATAATTTCAGGAAAC | |
| | | | AAAAATTTGTGCTATGCAA | |
| | | | ATACAATAAACTGGAAAAA | |
| | | | ACTGTTTGGGACCTCCGGT | |
| | | | CAGAAAACCAAAATTATAA | |
| | | | GCAACAGAGGTGAAAACAG | |
| | | | CTGCAAGGCCACAGGCCAG | |
| | | | GTCTGCCATGCCTTGTGCT | |
| | | | CCCCCGAGGGCTGCTGGGG | |
| | | | CCCGGAGCCCAGGGACTGC | |
| | | | GTCTCTTGCCGGAATGTCA | |
| | | | GCCGAGGCAGGGAATGCGT | |
| | | | GGACAAGTGCAACCTTCTG | |
| | | | GAGGGTGAGCCAAGGGAGT | |
| | | | TTGTGGAGAACTCTGAGTG | |
| | | | CATACAGTGCCACCCAGAG | |
| | | | TGCCTGCCTCAGGCCATGA | |
| | | | ACATCACCTGCACAGGACG | |
| | | | GGGACCAGACAACTGTATC | |
| | | | CAGTGTGCCCACTACATTG | |
| | | | ACGGCCCCCACTGCGTCAA | |
| | | | GACCTGCCCGGCAGGAGTC | |
| | | | ATGGGAGAAAACAACACCC | |
| | | | TGGTCTGGAAGTACGCAGA | |
| | | | CGCCGGCCATGTGTGCCAC | |
| | | | CTGTGCCATCCAAACTGCA | |
| | | | CCTACGGATGCACTGGGCC | |
| | | | AGGTCTTGAAGGCTGTCCA | |
| | | | ACGAATGGGCCTAAGATCC | |
| | | | CGTCCATCGCCACTGGGAT | |
| | | | GGTGGGGGCCCTCCTCTTG | |
| | | | CTGCTGGTGGTGGCCCTGG | |
| | | | GGATCGGCCTCTTCATG | |
| hCD20 (full | MTTPRNSVNGTFPAEPMKG | 105 | ATGACAACACCCAGAAATT | 117 |
| length) | PIAMQSGPKPLFRRMSSLV | | CAGTAAATGGGACTTTCCC | |
| | GPTQSFFMRESKTLGAVQI | | GGCAGAGCCAATGAAAGGC | |
| | MNGLFHIALGGLLMIPAGI | | CCTATTGCTATGCAATCTG | |
| | YAPICVTVWYPLWGGIMYI | | GTCCAAAACCACTCTTCAG | |
| | ISGSLLAATEKNSRKCLVK | | GAGGATGTCTTCACTGGTG | |
| | GKMIMNSLSLFAAISGMIL | | GGCCCCACGCAAAGCTTCT | |
| | SIMDILNIKISHFLKMESL | | TCATGAGGGAATCTAAGAC | |
| | NFIRAHTPYINIYNCEPAN | | TTTGGGGGCTGTCCAGATT | |
| | PSEKNSPSTQYCYSIQSLF | | ATGAATGGGCTCTTCCACA | |
| | LGILSVMLIFAFFQELVIA | | TTGCCCTGGGGGGTCTTCT | |
| | GIVENEWKRTCSRPKSNIV | | GATGATCCCAGCAGGGATC | |
| | LLSAEEKKEQTIEIKEEVV | | TATGCACCCATCTGTGTGA | |
| | GLTETSSQPKNEEDIEIIP | | CTGTGTGGTACCCTCTCTG | |
| | IQEEEEEETETNFPEPPQD | | GGGAGGCATTATGTATATT | |
| | QESSPIENDSSP | | ATTTCCGGATCACTCCTGG | |
| | | | CAGCAACGGAGAAAAACTC | |
| | | | CAGGAAGTGTTTGGTCAAA | |
| | | | GGAAAAATGATAATGAATT | |
| | | | CATTGAGCCTCTTTGCTGC | |
| | | | CATTTCTGGAATGATTCTT | |
| | | | TCAATCATGGACATACTTA | |
| | | | ATATTAAAATTTCCCATTT | |
| | | | TTTAAAAATGGAGAGTCTG | |
| | | | AATTTTATTAGAGCTCACA | |
| | | | CACCATATATTAACATATA | |
| | | | CAACTGTGAACCAGCTAAT | |
| | | | CCCTCTGAGAAAAACTCCC | |
| | | | CATCTACCCAATACTGTTA | |
| | | | CAGCATACAATCTCTGTTC | |
| | | | TTGGGCATTTTGTCAGTGA | |
| | | | TGCTGATCTTTGCCTTCTT | |
| | | | CCAGGAACTTGTAATAGCT | |
| | | | GGCATCGTTGAGAATGAAT | |
| | | | GGAAAAGAACGTGCTCCAG | |
| | | | ACCCAAATCTAACATAGTT | |
| | | | CTCCTGTCAGCAGAAGAAA | |
| | | | AAAAAGAACAGACTATTGA | |
| | | | AATAAAAGAAGAAGTGGTT | |
| | | | GGGCTAACTGAAACATCTT | |
| | | | CCCAACCAAAGAATGAAGA | |
| | | | AGACATTGAAATTATTCCA | |
| | | | ATCCAAGAAGAGGAAGAAG | |
| | | | AAGAAACAGAGACGAACTT | |
| | | | TCCAGAACCTCCCCAAGAT | |
| | | | CAGGAATCCTCACCAATAG | |
| | | | AAAATGACAGCTCTCCT | |
| hCD20t-1 | MTTPRNSVNGTFPAEPMKG | 106 | ATGACCACACCACGGAACT | 118 |
| | PIAMQSGPKPLFRRMSSLV | | CTGTGAATGGCACCTTCCC | |
| | GPTQSFFMRESKTLGAVQI | | AGCAGAGCCAATGAAGGGA | |
| | MNGLFHIALGGLLMIPAGI | | CCAATCGCAATGCAGAGCG | |
| | YAPICVTVWYPLWGGIMYI | | GACCCAAGCCTCTGTTTCG | |
| | ISGSLLAATEKNSRKCLVK | | GAGAATGAGCTCCCTGGTG | |
| | GKMIMNSLSLFAAISGMIL | | GGCCCAACCCAGTCCTTCT | |
| | SIMDILNIKISHFLKMESL | | TTATGAGAGAGTCTAAGAC | |
| | NFIRAHTPYINIYNCEPAN | | ACTGGGCGCCGTGCAGATC | |
| | PSEKNSPSTQYCYSIQSLF | | ATGAACGGACTGTTCCACA | |
| | LGILSVMLIFAFFQELVIA | | TCGCCCTGGGAGGACTGCT | |
| | GIVENEWKRTCSRPKSNIV | | GATGATCCCAGCCGGCATC | |
| | LLSAEEKKEQTIEIKEEVV | | TACGCCCCTATCTGCGTGA | |
| | GLTETSSOPKNEEDIE | | CCGTGTGGTACCCTCTGTG | |
| | | | GGGCGGCATCATGTATATC | |
| | | | ATCTCCGGCTCTCTGCTGG | |
| | | | CCGCCACAGAGAAGAACAG | |
| | | | CAGGAAGTGTCTGGTGAAG | |
| | | | GGCAAGATGATCATGAATA | |
| | | | GCCTGTCCCTGTTTGCCGC | |
| | | | CATCTCTGGCATGATCCTG | |
| | | | AGCATCATGGACATCCTGA | |
| | | | ACATCAAGATCAGCCACTT | |
| | | | CCTGAAGATGGAGAGCCTG | |
| | | | AACTTCATCAGAGCCCACA | |
| | | | CCCCTTACATCAACATCTA | |
| | | | TAATTGCGAGCCTGCCAAC | |
| | | | CCATCCGAGAAGAATTCTC | |
| | | | CAAGCACACAGTACTGTTA | |
| | | | TTCCATCCAGTCTCTGTTC | |
| | | | CTGGGCATCCTGTCTGTGA | |
| | | | TGCTGATCTTTGCCTTCTT | |
| | | | TCAGGAGCTGGTCATCGCC | |
| | | | GGCATCGTGGAGAACGAGT | |
| | | | GGAAGAGGACCTGCAGCCG | |
| | | | CCCCAAGTCCAATATCGTG | |
| | | | CTGCTGTCCGCCGAGGAGA | |
| | | | AGAAGGAGCAGACAATCGA | |
| | | | GATCAAGGAGGAGGTGGTG | |
| | | | GGCCTGACCGAGACATCTA | |
| | | | GCCAGCCTAAGAATGAGGA | |
| | | | GGATATCGAG | |

### 5.5 Vectors

In one aspect, provided herein are recombinant vectors comprising a polycistronic expression cassette that comprises at least three cistrons. In some embodiments, the polycistronic expression cassette comprises at least 4, 5, or 6 cistrons. In some embodiments, the polycistronic expression cassette comprises 3 cistrons. In some embodiments, the polycistronic expression cassette comprises 4 cistrons. In some embodiments, the polycistronic expression cassette comprises 5 cistrons.

In some embodiments, the vector is a non-viral vector. Exemplary non-viral vectors include, but are not limited to, plasmid DNA, episomal plasmid, minicircle, ministring, oligonucleotides (e.g., mRNA, naked DNA). In some embodiments, the polycistronic vector is a DNA plasmid vector.

In some embodiments, the vector is a viral vector. Viral vectors can be replication competent or replication incompetent. Viral vectors can be integrating or non-integrating. A number of viral based systems have been developed for gene transfer into mammalian cells, and a suitable viral vector can be selected by a person of ordinary skill in the art. Exemplary viral vectors include, but are not limited to, adenovirus vectors (e.g., adenovirus 5), adeno-associated virus (AAV) vectors (e.g., AAV2, 3, 5, 6, 8, 9), retrovirus vectors (MMSV, MSCV), lentivirus vectors (e.g., HIV-1, HIV-2), gammaretrovirus vectors, herpes virus vectors (e.g., HSV1, HSV2), alphavirus vectors (e.g., SFV, SIN, VEE, M1), flavivirus (e.g., Kunjin, West Nile, Dengue virus), rhabdovirus vectors (e.g., rabies virus, VSV), measles virus vector (e.g., MV-Edm), Newcastle disease virus vectors, poxvirus vectors (e.g., VV), measles virus, and picornavirus vectors (e.g., Coxsackievirus).

In one aspect, the vector comprises a polycistronic expression cassette that comprises from 5′ to 3′: a first polynucleotide sequence that encodes a chimeric antigen receptor (CAR); a second polynucleotide sequence that comprises an F2A element; a third polynucleotide sequence that encodes a cytokine; a fourth polynucleotide sequence that comprises a T2A element; and a fifth polynucleotide sequence that encodes a marker protein.

In some embodiments, the F2A element comprises a polynucleotide sequence that encodes an amino acid sequence at least 95%, 96%, 97%, 98%, 99%, or 100% identical to the amino acid sequence of SEQ ID NO: 137. In some embodiments, the F2A element comprises a polynucleotide sequence that encodes the amino acid sequence of SEQ ID NO: 137. In some embodiments, the F2A element comprises a polynucleotide sequence at least 75%, 80%, 85%, 90%, 95%, 96%, 97%, 98%, 99%, or 100% identical to the polynucleotide sequence of SEQ ID NO: 141. In some embodiments, the F2A element comprises the polynucleotide sequence of SEQ ID NO: 141.

In some embodiments, the F2A element comprises a polynucleotide sequence that encodes an amino acid sequence at least 95%, 96%, 97%, 98%, 99%, or 100% identical to the amino acid sequence of SEQ ID NO: 138. In some embodiments, the F2A element comprises a polynucleotide sequence that encodes the amino acid sequence of SEQ ID NO: 138. In some embodiments, the F2A element comprises a polynucleotide sequence at least 75%, 80%, 85%, 90%, 95%, 96%, 97%, 98%, 99%, or 100% identical to the polynucleotide sequence of SEQ ID NO: 142. In some embodiments, the F2A element comprises the polynucleotide sequence of SEQ ID NO: 142.

In some embodiments, the T2A element comprises a polynucleotide sequence that encodes an amino acid sequence at least 95%, 96%, 97%, 98%, 99%, or 100% identical to the amino acid sequence of SEQ ID NO: 139. In some embodiments, the T2A element comprises a polynucleotide sequence that encodes the amino acid sequence of SEQ ID NO: 139. In some embodiments, the T2A element comprises a polynucleotide sequence at least 75%, 80%, 85%, 90%, 95%, 96%, 97%, 98%, 99%, or 100% identical to the polynucleotide sequence of SEQ ID NO: 143. In some embodiments, the T2A element comprises the polynucleotide sequence of SEQ ID NO: 143.

In some embodiments, the T2A element comprises a polynucleotide sequence that encodes an amino acid sequence at least 95%, 96%, 97%, 98%, 99%, or 100% identical to the amino acid sequence of SEQ ID NO: 140 or 182. In some embodiments, the T2A element comprises a polynucleotide sequence that encodes an amino acid sequence at least 95%, 96%, 97%, 98%, 99%, or 100% identical to the amino acid sequence of SEQ ID NO: 140. In some embodiments, the T2A element comprises a polynucleotide sequence that encodes an amino acid sequence at least 95%, 96%, 97%, 98%, 99%, or 100% identical to the amino acid sequence of SEQ ID NO: 182. In some embodiments, the T2A element comprises a polynucleotide sequence that the amino acid sequence of SEQ ID NO: 140 or 182. In some embodiments, the T2A element comprises a polynucleotide sequence that encodes the amino acid sequence of SEQ ID NO: 140. In some embodiments, the T2A element comprises a polynucleotide sequence that encodes the amino acid sequence of SEQ ID NO: 182.

In some embodiments, the T2A element comprises a polynucleotide sequence at least 75%, 80%, 85%, 90%, 95%, 96%, 97%, 98%, 99%, or 100% identical to the polynucleotide sequence of SEQ ID NO: 144, 145, or 165. In some embodiments, the T2A element comprises a polynucleotide sequence at least 75%, 80%, 85%, 90%, 95%, 96%, 97%, 98%, 99%, or 100% identical to the polynucleotide sequence of SEQ ID NO: 144. In some embodiments, the T2A element comprises a polynucleotide sequence at least 75%, 80%, 85%, 90%, 95%, 96%, 97%, 98%, 99%, or 100% identical to the polynucleotide sequence of SEQ ID NO: 145. In some embodiments, the T2A element comprises a polynucleotide sequence at least 75%, 80%, 85%, 90%, 95%, 96%, 97%, 98%, 99%, or 100% identical to the polynucleotide sequence of SEQ ID NO: 165. In some embodiments, the T2A element comprises the polynucleotide sequence of SEQ ID NO: 144, 145, or 165. In some embodiments, the T2A element comprises the polynucleotide sequence of SEQ ID NO: 144. In some embodiments, the T2A element comprises the polynucleotide sequence of SEQ ID NO: 145. In some embodiments, the T2A element comprises the polynucleotide sequence of SEQ ID NO: 165.

Exemplary polynucleotide sequences encoding F2A and P2A elements are provided in Table 8, herein.

TABLE 8
Amino acid and polynucleotide sequences of exemplary 2A elements.

| | | SEQ ID | | SEQ ID |
|---|---|---|---|---|
| Description | Amino Acid Sequence | NO | Polynucleotide Sequence | NO |
| F2A | VKQTLNFDLLKLAGDVESN | 137 | GTGAAGCAGACCCTGAATTT | 141 |
| | PGP | | CGACCTGCTGAAGCTGGCCG | |
| | | | GGGACGTGGAGAGCAACCCT | |
| | | | GGCCCC | |
| F2A (with flanking | GSGVKQTLNFDLLKLAGDV | 138 | GGCTCCGGAGTGAAGCAGAC | 142 |
| residues) | ESNPGP | | CCTGAATTTCGACCTGCTGA | |
| | | | AGCTGGCCGGGGACGTGGAG | |
| | | | AGCAACCCTGGCCCC | |
| T2A | EGRGSLLTCGDVEENPGP | 139 | GAGGGCAGAGGAAGTCTTCT | 143 |
| | | | AACATGCGGTGACGTGGAGG | |
| | | | AGAATCCCGGCCCT | |
| T2A (with flanking | LEGGGEGRGSLLTCGDVEE | 140 | CTGGAGGGCGGCGGAGAGGG | 144 |
| residues) | NPGPR | | CAGAGGAAGTCTTCTAACAT | |
| | | | GCGGTGACGTGGAGGAGAAT | |
| | | | CCCGGCCCTAGG | |
| | | | CTCGAGGGCGGCGGAGAGGG | 145 |
| | | | CAGAGGAAGTCTTCTAACAT | |
| | | | GCGGTGACGTGGAGGAGAAT | |
| | | | CCCGGCCCTAGG | |
| T2A (with variant | LEGGGEGRGSLLTCGDVEE | 182 | CTCGAGGGCGGCGGAGAGGG | 165 |
| flanking residues) | NPGP | | CAGAGGAAGTCTTCTAACAT | |
| | | | GCGGTGACGTGGAGGAGAAT | |
| | | | CCCGGCCCT | |

In some embodiments, the vector or polycistronic expression cassette comprises one or more additional elements. Additional elements include, but are not limited to, promoters, enhancers, polyadenylation (polyA) sequences, and selection genes.

In some embodiments, the vector comprises a polynucleotide sequence that encodes for a selectable marker that confers a specific trait on cells in which the selectable marker is expressed enabling artificial selection of those cells. Exemplary selectable markers include, but are not limited to, antibiotic resistance genes, e.g., resistance to kanamycin, ampicillin, or triclosan.

In some embodiments, the polycistronic expression cassette comprises a transcriptional regulatory element. Exemplary transcriptional regulatory elements include, but are not limited to promoters and enhancers. In some embodiments, the polycistronic expression cassette comprises a promoter sequence 5′ of the first 5′ cistron. In some embodiments, the promoter comprises a polynucleotide sequence at least 75%, 80%, 85%, 90%, 95%, 96%, 97%, 98%, 99%, or 100% identical to the polynucleotide sequence of SEQ ID NO: 146. In some embodiments, the promoter comprises the polynucleotide sequence of SEQ ID NO: 146. In some embodiments, the polynucleotide sequence of the promoter consists of a polynucleotide sequence at least 75%, 80%, 85%, 90%, 95%, 96%, 97%, 98%, 99% or 100% identical to the polynucleotide sequence of SEQ ID NO: 146. In some embodiments, the polynucleotide sequence of the promoter consists the polynucleotide sequence of SEQ ID NO: 146.

In some embodiments, the polycistronic expression cassette comprises a polyA sequence 3′ of the 3′ terminal cistron. In some embodiments, the polyA sequence comprises a polynucleotide sequence at least 75%, 80%, 85%, 90%, 95%, 96%, 97%, 98%, 99%, or 100% identical to the polynucleotide sequence of SEQ ID NO: 148. In some embodiments, the polyA sequence comprises the nucleic acid sequence of SEQ ID NO: 148. In some embodiments, the polyA sequence consists of a sequence at least 75%, 80%, 85%, 90%, 95%, 96%, 97%, 98%, 99%, or 100% identical to the polynucleotide sequence of SEQ ID NO: 148. In some embodiments, the polyA sequence consists of the nucleic acid sequence of SEQ ID NO: 148.

The polynucleotide sequence of exemplary promoters and polyA sequences are provided in Table 9, herein.

TABLE 9
Polynucleotide sequence of exemplary promoters and polyA sequences.

| Description | Nucleic Acid Sequence | SEQ ID NO |
|---|---|---|
| hEF-1a Hybrid | GGATCTGCGATCGCTCCGGTGCCCGTCAGTGGGCAGAGCGCACATC | 146 |
| Promoter | GCCCACAGTCCCCGAGAAGTTGGGGGGAGGGGTCGGCAATTGAACC | |
| | GGTGCCTAGAGAAGGTGGCGCGGGGTAAACTGGGAAAGTGATGTCG | |
| | TGTACTGGCTCCGCCTTTTTCCCGAGGGTGGGGGAGAACCGTATAT | |
| | AAGTGCAGTAGTCGCCGTGAACGTTCTTTTTCGCAACGGGTTTGCC | |
| | GCCAGAACACAGCTGAAGCTTCGAGGGGCTCGCATCTCTCCTTCAC | |
| | GCGCCCGCCGCCCTACCTGAGGCCGCCATCCACGCCGGTTGAGTCG | |
| | CGTTCTGCCGCCTCCCGCCTGTGGTGCCTCCTGAACTGCGTCCGCC | |
| | GTCTAGGTAAGTTTAAAGCTCAGGTCGAGACCGGGCCTTTGTCCGG | |
| | CGCTCCCTTGGAGCCTACCTAGACTCAGCCGGCTCTCCACGCTTTG | |
| | CCTGACCCTGCTTGCTCAACTCTACGTCTTTGTTTCGTTTTCTGTT | |
| | CTGCGCCGTTACAGATCCAAGCTGTGACCGGCGCCTACCTGAGAT | |
| BGH poly A | GATCTGCTGTGCCTTCTAGTTGCCAGCCATCTGTTGTTTGCCCCTC | 148 |
| sequence | CCCCGTGCCTTCCTTGACCCTGGAAGGTGCCACTCCCACTGTCCTT | |
| | TCCTAATAAAATGAGGAAATTGCATCGCATTGTCTGAGTAGGTGTC | |
| | ATTCTATTCTGGGGGGTGGGGTGGGGCAGGACAGCAAGGGGGAGGA | |
| | TTGGGAAGACAATAGCAGGCATGCTGGGGATGCGGTGGGCTCTATG | |
| | G | |

The polynucleotide sequence of exemplary polycistronic expression cassettes are provided in Table 10, herein. In some embodiments, the polycistronic expression cassette comprises a polynucleotide sequence at least 75%, 80%, 85%, 90%, 95%, 96%, 97%, 98%, 99%, or 100% identical to the polynucleotide sequence of SEQ ID NO: 149, 150, or 151. In some embodiments, the polycistronic expression cassette comprises a polynucleotide sequence at least 75%, 80%, 85%, 90%, 95%, 96%, 97%, 98%, 99%, or 100% identical to the polynucleotide sequence of SEQ ID NO: 149. In some embodiments, the polycistronic expression cassette comprises a polynucleotide sequence at least 75%, 80%, 85%, 90%, 95%, 96%, 97%, 98%, 99% or 100% identical to the polynucleotide sequence of SEQ ID NO: 150. In some embodiments, the polycistronic expression cassette comprises a polynucleotide sequence at least 75%, 80%, 85%, 90%, 95%, 96%, 97%, 98%, 99% or 100% identical to the polynucleotide sequence of SEQ ID NO: 151.

In some embodiments, the polycistronic expression cassette comprises the polynucleotide sequence of SEQ ID NO: 149, 150, or 151. In some embodiments, the polycistronic expression cassette comprises the polynucleotide sequence of SEQ ID NO: 149. In some embodiments, the polycistronic expression cassette comprises the polynucleotide sequence of SEQ ID NO: 150. In some embodiments, the polycistronic expression cassette comprises the polynucleotide sequence of SEQ ID NO: 151.

TABLE 10
Polynucleotide sequence of exemplary polycistronic expression cassettes.

| Name | Polynucleotide Sequence | SEQ ID NO |
|---|---|---|
| Cassette 1 | ATGCTGCTGCTGGTGACCAGCCTGCTGCTGTGTGAGCTGCCCCACC | 149 |
| (from Plasmid A and | CCGCCTTTCTGCTGATCCCCGACATCCAGATGACCCAGACCACCTC | |
| Plasmid D; CD19CAR- | CAGCCTGAGCGCCAGCCTGGGCGACCGGGTGACCATCAGCTGCCGG | |
| F2A-mbIL15-T2A- | GCCAGCCAGGACATCAGCAAGTACCTGAACTGGTATCAGCAGAAGC | |
| HER1t) | CCGACGGCACCGTCAAGCTGCTGATCTACCACACCAGCCGGCTGCA | |
| | CAGCGGCGTGCCCAGCCGGTTTAGCGGCAGCGGCTCCGGCACCGAC | |
| | TACAGCCTGACCATCTCCAACCTGGAGCAGGAGGACATCGCCACCT | |
| | ACTTTTGCCAGCAGGGCAACACACTGCCCTACACCTTTGGCGGCGG | |
| | AACAAAGCTGGAGATCACCGGCAGCACCTCCGGCAGCGGCAAGCCT | |
| | GGCAGCGGCGAGGGCAGCACCAAGGGCGAGGTGAAGCTGCAGGAGA | |
| | GCGGCCCTGGCCTGGTGGCCCCCAGCCAGAGCCTGAGCGTGACCTG | |
| | TACCGTGTCCGGCGTGTCCCTGCCCGACTACGGCGTGTCCTGGATC | |
| | CGGCAGCCCCCTAGGAAGGGCCTGGAGTGGCTGGGCGTGATCTGGG | |
| | GCAGCGAGACCACCTACTACAACAGCGCCCTGAAGAGCCGGCTGAC | |
| | CATCATCAAGGACAACAGCAAGAGCCAGGTGTTCCTGAAGATGAAC | |
| | AGCCTGCAGACCGACGACACCGCCATCTACTACTGTGCCAAGCACT | |
| | ACTACTACGGCGGCAGCTACGCCATGGACTACTGGGGCCAGGGCAC | |
| | CAGCGTGACCGTGTCCAGCAAGCCCACCACCACCCCTGCCCCTAGA | |
| | CCTCCAACCCCAGCCCCTACAATCGCCAGCCAGCCCCTGAGCCTGA | |
| | GGCCCGAAGCCTGTAGACCTGCCGCTGGCGGAGCCGTGCACACCAG | |
| | AGGCCTGGATTTCGCCTGCGACATCTACATCTGGGCCCCTCTGGCC | |
| | GGCACCTGTGGCGTGCTGCTGCTGAGCCTGGTCATCACCCTGTACT | |
| | GCAACCACCGGAATAGGAGCAAGCGGAGCAGAGGCGGCCACAGCGA | |
| | CTACATGAACATGACCCCCCGGAGGCCTGGCCCCACCCGGAAGCAC | |
| | TACCAGCCCTACGCCCCTCCCAGGGACTTCGCCGCCTACCGGAGCC | |
| | GGGTGAAGTTCAGCCGGAGCGCCGACGCCCCTGCCTACCAGCAGGG | |
| | CCAGAACCAGCTGTACAACGAGCTGAACCTGGGCCGGAGGGAGGAG | |
| | TACGACGTGCTGGACAAGCGGAGAGGCCGGGACCCTGAGATGGGCG | |
| | GCAAGCCCCGGAGAAAGAACCCTCAGGAGGGCCTGTATAACGAACT | |
| | GCAGAAAGACAAGATGGCCGAGGCCTACAGCGAGATCGGCATGAAG | |
| | GGCGAGCGGCGGAGGGGCAAGGGCCACGACGGCCTGTACCAGGGCC | |
| | TGAGCACCGCCACCAAGGATACCTACGACGCCCTGCACATGCAGGC | |
| | CCTGCCCCCCAGAGGCTCCGGAGTGAAGCAGACCCTGAATTTCGAC | |
| | CTGCTGAAGCTGGCCGGGGACGTGGAGAGCAACCCTGGCCCCATGG | |
| | ATTGGACCTGGATTCTGTTTCTGGTGGCCGCTGCCACAAGAGTGCA | |
| | CAGCAACTGGGTGAATGTGATCAGCGACCTGAAGAAGATCGAGGAT | |
| | CTGATCCAGAGCATGCACATTGATGCCACCCTGTACACAGAATCTG | |
| | ATGTGCACCCTAGCTGTAAAGTGACCGCCATGAAGTGTTTTCTGCT | |
| | GGAGCTGCAGGTGATTTCTCTGGAAAGCGGAGATGCCTCTATCCAC | |
| | GACACAGTGGAGAATCTGATCATCCTGGCCAACAATAGCCTGAGCA | |
| | GCAATGGCAATGTGACAGAGTCTGGCTGTAAGGAGTGTGAGGAGCT | |
| | GGAGGAGAAGAACATCAAGGAGTTTCTGCAGAGCTTTGTGCACATC | |
| | GTGCAGATGTTCATCAATACAAGCTCTGGCGGAGGATCTGGAGGAG | |
| | GCGGATCTGGAGGAGGAGGCAGTGGAGGCGGAGGATCTGGCGGAGG | |
| | ATCTCTGCAGATTACATGCCCTCCTCCAATGTCTGTGGAGCACGCC | |
| | GATATTTGGGTGAAGTCCTACAGCCTGTACAGCAGAGAGAGATACA | |
| | TCTGCAACAGCGGCTTTAAGAGAAAGGCCGGCACCTCTTCTCTGAC | |
| | AGAGTGCGTGCTGAATAAGGCCACAAATGTGGCCCACTGGACAACA | |
| | CCTAGCCTGAAGTGCATTAGAGATCCTGCCCTGGTCCACCAGAGGC | |
| | CTGCCCCTCCATCTACAGTGACAACAGCCGGAGTGACACCTCAGCC | |
| | TGAATCTCTGAGCCCTTCTGGAAAAGAACCTGCCGCCAGCTCTCCT | |
| | AGCTCTAATAATACCGCCGCCACAACAGCCGCCATTGTGCCTGGAT | |
| | CTCAGCTGATGCCTAGCAAGTCTCCTAGCACAGGCACAACAGAGAT | |
| | CAGCAGCCACGAATCTTCTCACGGAACACCTTCTCAGACCACCGCC | |
| | AAGAATTGGGAGCTGACAGCCTCTGCCTCTCACCAGCCTCCAGGAG | |
| | TGTATCCTCAGGGCCACTCTGATACAACAGTGGCCATCAGCACATC | |
| | TACAGTGCTGCTGTGTGGACTGTCTGCCGTGTCTCTGCTGGCCTGT | |
| | TACCTGAAGTCTAGACAGACACCTCCTCTGGCCTCTGTGGAGATGG | |
| | AGGCCATGGAAGCCCTGCCTGTGACATGGGGAACAAGCAGCAGAGA | |
| | TGAAGACCTGGAGAATTGTTCTCACCACCTGCTGGAGGGCGGCGGA | |
| | GAGGGCAGAGGAAGTCTTCTAACATGCGGTGACGTGGAGGAGAATC | |
| | CCGGCCCTAGGATGAGGCTCCCTGCTCAGCTCCTGGGGCTGCTAAT | |
| | GCTCTGGGTGCCAGGATCCAGTGGGCGCAAAGTGTGTAACGGAATA | |
| | GGTATTGGTGAATTTAAAGACTCACTCTCCATAAATGCTACGAATA | |
| | TTAAACACTTCAAAAACTGCACCTCCATCAGTGGCGATCTCCACAT | |
| | CCTGCCGGTGGCATTTAGGGGTGACTCCTTCACACATACTCCTCCT | |
| | CTGGATCCACAGGAACTGGATATTCTGAAAACCGTAAAGGAAATCA | |
| | CAGGGTTTTTGCTGATTCAGGCTTGGCCTGAAAACAGGACGGACCT | |
| | CCATGCCTTTGAGAACCTAGAAATCATACGCGGCAGGACCAAGCAA | |
| | CATGGTCAGTTTTCTCTTGCAGTCGTCAGCCTGAACATAACATCCT | |
| | TGGGATTACGCTCCCTCAAGGAGATAAGTGATGGAGATGTGATAAT | |
| | TTCAGGAAACAAAAATTTGTGCTATGCAAATACAATAAACTGGAAA | |
| | AAACTGTTTGGTACCTCCGGTCAGAAAACCAAAATTATAAGCAACA | |
| | GAGGTGAAAACAGCTGCAAGGCCACAGGCCAGGTCTGCCATGCCTT | |
| | GTGCTCCCCCGAGGGCTGCTGGGGCCCGGAGCCCAGGGACTGCGTC | |
| | TCTGGTGGCGGTGGCTCGGGCGGTGGTGGGTCGGGTGGCGGCGGAT | |
| | CTGGTGGCGGTGGCTCGTTTTGGGTGCTGGTGGTGGTTGGTGGAGT | |
| | CCTGGCTTGCTATAGCTTGCTAGTAACAGTGGCCTTTATTATTTTC | |
| | TGGGTGAGGAGTAAGAGGAGC | |
| Cassette 2 | ATGGATTGGACCTGGATTCTGTTTCTGGTGGCCGCTGCCACAAGAG | 150 |
| (from Plasmid B and | TGCACAGCAACTGGGTGAATGTGATCAGCGACCTGAAGAAGATCGA | |
| Plasmid E; mbIL15- | GGATCTGATCCAGAGCATGCACATTGATGCCACCCTGTACACAGAA | |
| T2A-HER1t-F2A- | TCTGATGTGCACCCTAGCTGTAAAGTGACCGCCATGAAGTGTTTTC | |
| CD19CAR) | TGCTGGAGCTGCAGGTGATTTCTCTGGAAAGCGGAGATGCCTCTAT | |
| | CCACGACACAGTGGAGAATCTGATCATCCTGGCCAACAATAGCCTG | |
| | AGCAGCAATGGCAATGTGACAGAGTCTGGCTGTAAGGAGTGTGAGG | |
| | AGCTGGAGGAGAAGAACATCAAGGAGTTTCTGCAGAGCTTTGTGCA | |
| | CATCGTGCAGATGTTCATCAATACAAGCTCTGGCGGAGGATCTGGA | |
| | GGAGGCGGATCTGGAGGAGGAGGCAGTGGAGGCGGAGGATCTGGCG | |
| | GAGGATCTCTGCAGATTACATGCCCTCCTCCAATGTCTGTGGAGCA | |
| | CGCCGATATTTGGGTGAAGTCCTACAGCCTGTACAGCAGAGAGAGA | |
| | TACATCTGCAACAGCGGCTTTAAGAGAAAGGCCGGCACCTCTTCTC | |
| | TGACAGAGTGCGTGCTGAATAAGGCCACAAATGTGGCCCACTGGAC | |
| | AACACCTAGCCTGAAGTGCATTAGAGATCCTGCCCTGGTCCACCAG | |
| | AGGCCTGCCCCTCCATCTACAGTGACAACAGCCGGAGTGACACCTC | |
| | AGCCTGAATCTCTGAGCCCTTCTGGAAAAGAACCTGCCGCCAGCTC | |
| | TCCTAGCTCTAATAATACCGCCGCCACAACAGCCGCCATTGTGCCT | |
| | GGATCTCAGCTGATGCCTAGCAAGTCTCCTAGCACAGGCACAACAG | |
| | AGATCAGCAGCCACGAATCTTCTCACGGAACACCTTCTCAGACCAC | |
| | CGCCAAGAATTGGGAGCTGACAGCCTCTGCCTCTCACCAGCCTCCA | |
| | GGAGTGTATCCTCAGGGCCACTCTGATACAACAGTGGCCATCAGCA | |
| | CATCTACAGTGCTGCTGTGTGGACTGTCTGCCGTGTCTCTGCTGGC | |
| | CTGTTACCTGAAGTCTAGACAGACACCTCCTCTGGCCTCTGTGGAG | |
| | ATGGAGGCCATGGAAGCCCTGCCTGTGACATGGGGAACAAGCAGCA | |
| | GAGATGAGGACCTGGAGAATTGTTCTCACCACCTGCTCGAGGGCGG | |
| | CGGAGAGGGCAGAGGAAGTCTTCTAACATGCGGTGACGTGGAGGAG | |
| | AATCCCGGCCCTAGGATGAGGCTCCCTGCTCAGCTCCTGGGGCTGC | |
| | TAATGCTCTGGGTCCCAGGATCCAGTGGGCGCAAAGTGTGTAACGG | |
| | AATAGGTATTGGTGAATTTAAAGACTCACTCTCCATAAATGCTACG | |
| | AATATTAAACACTTCAAAAACTGCACCTCCATCAGTGGCGATCTCC | |
| | ACATCCTGCCGGTGGCATTTAGGGGTGACTCCTTCACACATACTCC | |
| | TCCTCTGGATCCACAGGAACTGGATATTCTGAAAACCGTAAAGGAA | |
| | ATCACAGGGTTTTTGCTGATTCAGGCTTGGCCTGAAAACAGGACGG | |
| | ACCTCCATGCCTTTGAGAACCTAGAAATCATACGCGGCAGGACCAA | |
| | GCAACATGGTCAGTTTTCTCTTGCAGTCGTCAGCCTGAACATAACA | |
| | TCCTTGGGATTACGCTCCCTCAAGGAGATAAGTGATGGAGATGTGA | |
| | TAATTTCAGGAAACAAAAATTTGTGCTATGCAAATACAATAAACTG | |
| | GAAAAAACTGTTTGGGACCTCCGGTCAGAAAACCAAAATTATAAGC | |
| | AACAGAGGTGAAAACAGCTGCAAGGCCACAGGCCAGGTCTGCCATG | |
| | CCTTGTGCTCCCCCGAGGGCTGCTGGGGCCCGGAGCCCAGGGACTG | |
| | CGTCTCTGGTGGCGGTGGCTCGGGCGGTGGTGGGTCGGGTGGCGGC | |
| | GGATCTGGTGGCGGTGGCTCGTTTTGGGTGCTGGTGGTGGTTGGTG | |
| | GAGTCCTGGCTTGCTATAGCTTGCTAGTAACAGTGGCCTTTATTAT | |
| | TTTCTGGGTGAGGAGTAAGAGGAGCGGCTCCGGAGTGAAGCAGACC | |
| | CTGAATTTCGACCTGCTGAAGCTGGCCGGGGACGTGGAGAGCAACC | |
| | CTGGCCCCATGCTGCTGCTGGTGACCAGCCTGCTGCTGTGTGAGCT | |
| | GCCCCACCCCGCCTTTCTGCTGATCCCCGACATCCAGATGACCCAG | |
| | ACCACCTCCAGCCTGAGCGCCAGCCTGGGCGACCGGGTGACCATCA | |
| | GCTGCCGGGCCAGCCAGGACATCAGCAAGTACCTGAACTGGTATCA | |
| | GCAGAAGCCCGACGGCACCGTCAAGCTGCTGATCTACCACACCAGC | |
| | CGGCTGCACAGCGGCGTGCCCAGCCGGTTTAGCGGCAGCGGCTCCG | |
| | GCACCGACTACAGCCTGACCATCTCCAACCTGGAGCAGGAGGACAT | |
| | CGCCACCTACTTTTGCCAGCAGGGCAACACACTGCCCTACACCTTT | |
| | GGCGGCGGAACAAAGCTGGAGATCACCGGCAGCACCTCCGGCAGCG | |
| | GCAAGCCTGGCAGCGGCGAGGGCAGCACCAAGGGCGAGGTGAAGCT | |
| | GCAGGAGAGCGGCCCTGGCCTGGTGGCCCCCAGCCAGAGCCTGAGC | |
| | GTGACCTGTACCGTGTCCGGCGTGTCCCTGCCCGACTACGGCGTGT | |
| | CCTGGATCCGGCAGCCCCCTAGGAAGGGCCTGGAGTGGCTGGGCGT | |
| | GATCTGGGGCAGCGAGACCACCTACTACAACAGCGCCCTGAAGAGC | |
| | CGGCTGACCATCATCAAGGACAACAGCAAGAGCCAGGTGTTCCTGA | |
| | AGATGAACAGCCTGCAGACCGACGACACCGCCATCTACTACTGTGC | |
| | CAAGCACTACTACTACGGCGGCAGCTACGCCATGGACTACTGGGGC | |
| | CAGGGCACCAGCGTGACCGTGTCCAGCAAGCCCACCACCACCCCTG | |
| | CCCCTAGACCTCCAACCCCAGCCCCTACAATCGCCAGCCAGCCCCT | |
| | GAGCCTGAGGCCCGAAGCCTGTAGACCTGCCGCTGGCGGAGCCGTG | |
| | CACACCAGAGGCCTGGATTTCGCCTGCGACATCTACATCTGGGCAC | |
| | CTCTGGCCGGCACCTGTGGCGTGCTGCTGCTGAGCCTGGTCATCAC | |
| | CCTGTACTGCAACCACCGGAATAGGAGCAAGCGGAGCAGAGGCGGC | |
| | CACAGCGACTACATGAACATGACCCCCCGGAGGCCTGGCCCCACCC | |
| | GGAAGCACTACCAGCCCTACGCCCCTCCCAGGGACTTCGCCGCCTA | |
| | CCGGAGCCGGGTGAAGTTCAGCCGGAGCGCCGACGCCCCTGCCTAC | |
| | CAGCAGGGCCAGAACCAGCTGTACAACGAGCTGAACCTGGGCCGGA | |
| | GGGAGGAGTACGACGTGCTGGACAAGCGGAGAGGCCGGGACCCTGA | |
| | GATGGGCGGCAAGCCCCGGAGAAAGAACCCTCAGGAGGGCCTGTAT | |
| | AACGAACTGCAGAAAGACAAGATGGCCGAGGCCTACAGCGAGATCG | |
| | GCATGAAGGGCGAGCGGCGGAGGGGCAAGGGCCACGACGGCCTGTA | |
| | CCAGGGCCTGAGCACCGCCACCAAGGATACCTACGACGCCCTGCAC | |
| | ATGCAGGCCCTGCCCCCCAGA | |
| Cassette 3 | ATGAGGATGAGGCTCCCTGCTCAGCTCCTGGGGCTGCTAATGCTCT | 151 |
| (from Plasmid C and | GGGTCCCAGGATCCAGTGGGCGCAAAGTGTGTAACGGAATAGGTAT | |
| Plasmid F; HER1t- | TGGTGAATTTAAAGACTCACTCTCCATAAATGCTACGAATATTAAA | |
| T2A-mIL15-F2A- | CACTTCAAAAACTGCACCTCCATCAGTGGCGATCTCCACATCCTGC | |
| CD19CAR) | CGGTGGCATTTAGGGGTGACTCCTTCACACATACTCCTCCTCTGGA | |
| | TCCACAGGAACTGGATATTCTGAAAACCGTAAAGGAAATCACAGGG | |
| | TTTTTGCTGATTCAGGCTTGGCCTGAAAACAGGACGGACCTCCATG | |
| | CCTTTGAGAACCTAGAAATCATACGCGGCAGGACCAAGCAACATGG | |
| | TCAGTTTTCTCTTGCAGTCGTCAGCCTGAACATAACATCCTTGGGA | |
| | TTACGCTCCCTCAAGGAGATAAGTGATGGAGATGTGATAATTTCAG | |
| | GAAACAAAAATTTGTGCTATGCAAATACAATAAACTGGAAAAAACT | |
| | GTTTGGGACCTCCGGTCAGAAAACCAAAATTATAAGCAACAGAGGT | |
| | GAAAACAGCTGCAAGGCCACAGGCCAGGTCTGCCATGCCTTGTGCT | |
| | CCCCCGAGGGCTGCTGGGGCCCGGAGCCCAGGGACTGCGTCTCTGG | |
| | TGGCGGTGGCTCGGGCGGTGGTGGGTCGGGTGGCGGCGGATCTGGT | |
| | GGCGGTGGCTCGTTTTGGGTGCTGGTGGTGGTTGGTGGAGTCCTGG | |
| | CTTGCTATAGCTTGCTAGTAACAGTGGCCTTTATTATTTTCTGGGT | |
| | GAGGAGTAAGAGGAGCCTCGAGGGCGGCGGAGAGGGCAGAGGAAGT | |
| | CTTCTAACATGCGGTGACGTGGAGGAGAATCCCGGCCCTATGGATT | |
| | GGACCTGGATTCTGTTTCTGGTGGCCGCTGCCACAAGAGTGCACAG | |
| | CAACTGGGTGAATGTGATCAGCGACCTGAAGAAGATCGAGGATCTG | |
| | ATCCAGAGCATGCACATTGATGCCACCCTGTACACAGAATCTGATG | |
| | TGCACCCTAGCTGTAAAGTGACCGCCATGAAGTGTTTTCTGCTGGA | |
| | GCTGCAGGTGATTTCTCTGGAAAGCGGAGATGCCTCTATCCACGAC | |
| | ACAGTGGAGAATCTGATCATCCTGGCCAACAATAGCCTGAGCAGCA | |
| | ATGGCAATGTGACAGAGTCTGGCTGTAAGGAGTGTGAGGAGCTGGA | |
| | GGAGAAGAACATCAAGGAGTTTCTGCAGAGCTTTGTGCACATCGTG | |
| | CAGATGTTCATCAATACAAGCTCTGGCGGAGGATCTGGAGGAGGCG | |
| | GATCTGGAGGAGGAGGCAGTGGAGGCGGAGGATCTGGCGGAGGATC | |
| | TCTGCAGATTACATGCCCTCCTCCAATGTCTGTGGAGCACGCCGAT | |
| | ATTTGGGTGAAGTCCTACAGCCTGTACAGCAGAGAGAGATACATCT | |
| | GCAACAGCGGCTTTAAGAGAAAGGCCGGCACCTCTTCTCTGACAGA | |
| | GTGCGTGCTGAATAAGGCCACAAATGTGGCCCACTGGACAACACCT | |
| | AGCCTGAAGTGCATTAGAGATCCTGCCCTGGTCCACCAGAGGCCTG | |
| | CCCCTCCATCTACAGTGACAACAGCCGGAGTGACACCTCAGCCTGA | |
| | ATCTCTGAGCCCTTCTGGAAAAGAACCTGCCGCCAGCTCTCCTAGC | |
| | TCTAATAATACCGCCGCCACAACAGCCGCCATTGTGCCTGGATCTC | |
| | AGCTGATGCCTAGCAAGTCTCCTAGCACAGGCACAACAGAGATCAG | |
| | CAGCCACGAATCTTCTCACGGAACACCTTCTCAGACCACCGCCAAG | |
| | AATTGGGAGCTGACAGCCTCTGCCTCTCACCAGCCTCCAGGAGTGT | |
| | ATCCTCAGGGCCACTCTGATACAACAGTGGCCATCAGCACATCTAC | |
| | AGTGCTGCTGTGTGGACTGTCTGCCGTGTCTCTGCTGGCCTGTTAC | |
| | CTGAAGTCTAGACAGACACCTCCTCTGGCCTCTGTGGAGATGGAGG | |
| | CCATGGAAGCCCTGCCTGTGACATGGGGAACAAGCAGCAGAGATGA | |
| | GGACCTGGAGAATTGTTCTCACCACCTGGGCTCCGGAGTGAAGCAG | |
| | ACCCTGAATTTCGACCTGCTGAAGCTGGCCGGGGACGTGGAGAGCA | |
| | ACCCTGGCCCCATGCTGCTGCTGGTGACCAGCCTGCTGCTGTGTGA | |
| | GCTGCCCCACCCCGCCTTTCTGCTGATCCCCGACATCCAGATGACC | |
| | CAGACCACCTCCAGCCTGAGCGCCAGCCTGGGCGACCGGGTGACCA | |
| | TCAGCTGCCGGGCCAGCCAGGACATCAGCAAGTACCTGAACTGGTA | |
| | TCAGCAGAAGCCCGACGGCACCGTCAAGCTGCTGATCTACCACACC | |
| | AGCCGGCTGCACAGCGGCGTGCCCAGCCGGTTTAGCGGCAGCGGCT | |
| | CCGGCACCGACTACAGCCTGACCATCTCCAACCTGGAGCAGGAGGA | |
| | CATCGCCACCTACTTTTGCCAGCAGGGCAACACACTGCCCTACACC | |
| | TTTGGCGGCGGAACAAAGCTGGAGATCACCGGCAGCACCTCCGGCA | |
| | GCGGCAAGCCTGGCAGCGGCGAGGGCAGCACCAAGGGCGAGGTGAA | |
| | GCTGCAGGAGAGCGGCCCTGGCCTGGTGGCCCCCAGCCAGAGCCTG | |
| | AGCGTGACCTGTACCGTGTCCGGCGTGTCCCTGCCCGACTACGGCG | |
| | TGTCCTGGATCCGGCAGCCCCCTAGGAAGGGCCTGGAGTGGCTGGG | |
| | CGTGATCTGGGGCAGCGAGACCACCTACTACAACAGCGCCCTGAAG | |
| | AGCCGGCTGACCATCATCAAGGACAACAGCAAGAGCCAGGTGTTCC | |
| | TGAAGATGAACAGCCTGCAGACCGACGACACCGCCATCTACTACTG | |
| | TGCCAAGCACTACTACTACGGCGGCAGCTACGCCATGGACTACTGG | |
| | GGCCAGGGCACCAGCGTGACCGTGTCCAGCAAGCCCACCACCACCC | |
| | CTGCCCCTAGACCTCCAACCCCAGCCCCTACAATCGCCAGCCAGCC | |
| | CCTGAGCCTGAGGCCCGAAGCCTGTAGACCTGCCGCTGGCGGAGCC | |
| | GTGCACACCAGAGGCCTGGATTTCGCCTGCGACATCTACATCTGGG | |
| | CACCTCTGGCCGGCACCTGTGGCGTGCTGCTGCTGAGCCTGGTCAT | |
| | CACCCTGTACTGCAACCACCGGAATAGGAGCAAGCGGAGCAGAGGC | |
| | GGCCACAGCGACTACATGAACATGACCCCCCGGAGGCCTGGCCCCA | |
| | CCCGGAAGCACTACCAGCCCTACGCCCCTCCCAGGGACTTCGCCGC | |
| | CTACCGGAGCCGGGTGAAGTTCAGCCGGAGCGCCGACGCCCCTGCC | |
| | TACCAGCAGGGCCAGAACCAGCTGTACAACGAGCTGAACCTGGGCC | |
| | GGAGGGAGGAGTACGACGTGCTGGACAAGCGGAGAGGCCGGGACCC | |
| | TGAGATGGGCGGCAAGCCCCGGAGAAAGAACCCTCAGGAGGGCCTG | |
| | TATAACGAACTGCAGAAAGACAAGATGGCCGAGGCCTACAGCGAGA | |
| | TCGGCATGAAGGGCGAGCGGCGGAGGGGCAAGGGCCACGACGGCCT | |
| | GTACCAGGGCCTGAGCACCGCCACCAAGGATACCTACGACGCCCTG | |
| | CACATGCAGGCCCTGCCCCCCAGA | |

The amino acid sequence encoded by the polynucleotide sequence of exemplary polycistronic expression cassettes are provided in Table 11, herein. In some embodiments, the polycistronic expression cassette comprises a polynucleotide sequence that encodes an amino acid sequence at least 95%, 96%, 97%, 98%, 99%, or 100% identical to the amino acid sequence of SEQ ID NO: 152, 153, or 154. In some embodiments, the polycistronic expression cassette comprises a polynucleotide sequence that encodes an amino acid sequence at least 9500, 9600, 97%, 98%, 99%, or 100% identical to the amino acid sequence of SEQ ID NO: 152. In some embodiments, the polycistronic expression cassette comprises a polynucleotide sequence that encodes an amino acid sequence at least 95%, 96%, 97%, 98%, 99%, or 100% identical to the amino acid sequence of SEQ ID NO: 153. In some embodiments, the polycistronic expression cassette comprises a polynucleotide sequence that encodes an amino acid sequence at least 950%, 96%, 97%, 98%, 99%, or 100% identical to the amino acid sequence of SEQ ID NO: 154.

In some embodiments, the polycistronic expression cassette comprises a polynucleotide sequence that encodes the amino acid sequence of SEQ ID NO: 152, 153, or 154. In some embodiments, the polycistronic expression cassette comprises a polynucleotide sequence that encodes the amino acid sequence of SEQ ID NO: 152. In some embodiments, the polycistronic expression cassette comprises a polynucleotide sequence that encodes the amino acid sequence of SEQ ID NO: 153. In some embodiments, the polycistronic expression cassette comprises a polynucleotide sequence that encodes the amino acid sequence of SEQ ID NO: 154.

TABLE 11
Amino acid sequence of proteins encoded by exemplary polycistronic expression
cassettes.

| Description | Amino Acid Sequence | SEQ ID NO: |
|---|---|---|
| Translation of expression | MLLLVTSLLLCELPHPAFLLIPDIQMTQTTSSLSASLGDRVTI | 152 |
| cassette from Plasmid A and | SCRASQDISKYLNWYQQKPDGTVKLLIYHTSRLHSGVPSRESG | |
| Plasmid D | SGSGTDYSLTISNLEQEDIATYFCQQGNTLPYTFGGGTKLEIT | |
| (CD19CAR-F2A-mbIL15- | GSTSGSGKPGSGEGSTKGEVKLQESGPGLVAPSQSLSVTCTVS | |
| T2A-HER1t) | GVSLPDYGVSWIRQPPRKGLEWLGVIWGSETTYYNSALKSRLT | |
| | IIKDNSKSQVFLKMNSLQTDDTAIYYCAKHYYYGGSYAMDYWG | |
| | QGTSVTVSSKPTTTPAPRPPTPAPTIASQPLSLRPEACRPAAG | |
| | GAVHTRGLDFACDIYIWAPLAGTCGVLLLSLVITLYCNHRNRS | |
| | KRSRGGHSDYMNMTPRRPGPTRKHYQPYAPPRDFAAYRSRVKF | |
| | SRSADAPAYQQGQNQLYNELNLGRREEYDVLDKRRGRDPEMGG | |
| | KPRRKNPQEGLYNELQKDKMAEAYSEIGMKGERRRGKGHDGLY | |
| | QGLSTATKDTYDALHMQALPPRGSGVKQTLNFDLLKLAGDVES | |
| | NPGPMDWTWILFLVAAATRVHSNWVNVISDLKKIEDLIQSMHI | |
| | DATLYTESDVHPSCKVTAMKCFLLELQVISLESGDASIHDTVE | |
| | NLIILANNSLSSNGNVTESGCKECEELEEKNIKEFLQSFVHIV | |
| | QMFINTSSGGGSGGGGSGGGGSGGGGSGGGSLQITCPPPMSVE | |
| | HADIWVKSYSLYSRERYICNSGFKRKAGTSSLTECVLNKATNV | |
| | AHWTTPSLKCIRDPALVHQRPAPPSTVTTAGVTPQPESLSPSG | |
| | KEPAASSPSSNNTAATTAAIVPGSQLMPSKSPSTGTTEISSHE | |
| | SSHGTPSQTTAKNWELTASASHQPPGVYPQGHSDTTVAISTST | |
| | VLLCGLSAVSLLACYLKSRQTPPLASVEMEAMEALPVTWGTSS | |
| | RDEDLENCSHHLLEGGGEGRGSLLTCGDVEENPGPRMRLPAQL | |
| | LGLLMLWVPGSSGRKVCNGIGIGEFKDSLSINATNIKHFKNCT | |
| | SISGDLHILPVAFRGDSFTHTPPLDPQELDILKTVKEITGELL | |
| | IQAWPENRTDLHAFENLEIIRGRTKQHGQFSLAVVSLNITSLG | |
| | LRSLKEISDGDVIISGNKNLCYANTINWKKLFGTSGQKTKIIS | |
| | NRGENSCKATGQVCHALCSPEGCWGPEPRDCVSGGGGSGGGGS | |
| | GGGGSGGGGSFWVLVVVGGVLACYSLLVTVAFIIFWVRSKRS | |
| Translation of expression | MDWTWILFLVAAATRVHSNWVNVISDLKKIEDLIQSMHIDATL | 153 |
| cassette from Plasmid B and | YTESDVHPSCKVTAMKCFLLELQVISLESGDASIHDTVENLII | |
| Plasmid E | LANNSLSSNGNVTESGCKECEELEEKNIKEFLQSFVHIVQMFI | |
| (mbIL15-T2A-HER1t-F2A- | NTSSGGGSGGGGSGGGGSGGGGSGGGSLQITCPPPMSVEHADI | |
| CD19CAR) | WVKSYSLYSRERYICNSGFKRKAGTSSLTECVLNKATNVAHWT | |
| | TPSLKCIRDPALVHQRPAPPSTVTTAGVTPQPESLSPSGKEPA | |
| | ASSPSSNNTAATTAAIVPGSQLMPSKSPSTGTTEISSHESSHG | |
| | TPSQTTAKNWELTASASHQPPGVYPQGHSDTTVAISTSTVLLC | |
| | GLSAVSLLACYLKSRQTPPLASVEMEAMEALPVTWGTSSRDED | |
| | LENCSHHLLEGGGEGRGSLLTCGDVEENPGPRMRLPAQLLGLL | |
| | MLWVPGSSGRKVCNGIGIGEFKDSLSINATNIKHFKNCTSISG | |
| | DLHILPVAFRGDSFTHTPPLDPQELDILKTVKEITGELLIQAW | |
| | PENRTDLHAFENLEIIRGRTKQHGQFSLAVVSLNITSLGLRSL | |
| | KEISDGDVIISGNKNLCYANTINWKKLFGTSGQKTKIISNRGE | |
| | NSCKATGQVCHALCSPEGCWGPEPRDCVSGGGGSGGGGSGGGG | |
| | SGGGGSFWVLVVVGGVLACYSLLVTVAFIIFWVRSKRSGSGVK | |
| | QTLNFDLLKLAGDVESNPGPMLLLVTSLLLCELPHPAFLLIPD | |
| | IQMTQTTSSLSASLGDRVTISCRASQDISKYLNWYQQKPDGTV | |
| | KLLIYHTSRLHSGVPSRFSGSGSGTDYSLTISNLEQEDIATYF | |
| | CQQGNTLPYTFGGGTKLEITGSTSGSGKPGSGEGSTKGEVKLQ | |
| | ESGPGLVAPSQSLSVTCTVSGVSLPDYGVSWIRQPPRKGLEWL | |
| | GVIWGSETTYYNSALKSRLTIIKDNSKSQVFLKMNSLQTDDTA | |
| | IYYCAKHYYYGGSYAMDYWGQGTSVTVSSKPTTTPAPRPPTPA | |
| | PTIASQPLSLRPEACRPAAGGAVHTRGLDFACDIYIWAPLAGT | |
| | CGVLLLSLVITLYCNHRNRSKRSRGGHSDYMNMTPRRPGPTRK | |
| | HYQPYAPPRDFAAYRSRVKFSRSADAPAYQQGQNQLYNELNLG | |
| | RREEYDVLDKRRGRDPEMGGKPRRKNPQEGLYNELQKDKMAEA | |
| | YSEIGMKGERRRGKGHDGLYQGLSTATKDTYDALHMQALPPR | |
| Translation of expression | MRMRLPAQLLGLLMLWVPGSSGRKVCNGIGIGEFKDSLSINAT | 154 |
| cassette from Plasmid C and | NIKHFKNCTSISGDLHILPVAFRGDSFTHTPPLDPQELDILKT | |
| Plasmid F | VKEITGELLIQAWPENRTDLHAFENLEIIRGRTKQHGQFSLAV | |
| (HER1t-T2A-mbIL15-F2A- | VSLNITSLGLRSLKEISDGDVIISGNKNLCYANTINWKKLFGT | |
| CD19CAR) | SGQKTKIISNRGENSCKATGQVCHALCSPEGCWGPEPRDCVSG | |
| | GGGSGGGGSGGGGSGGGGSFWVLVVVGGVLACYSLLVTVAFII | |
| | FWVRSKRSLEGGGEGRGSLLTCGDVEENPGPMDWTWILFLVAA | |
| | ATRVHSNWVNVISDLKKIEDLIQSMHIDATLYTESDVHPSCKV | |
| | TAMKCFLLELQVISLESGDASIHDTVENLIILANNSLSSNGNV | |
| | TESGCKECEELEEKNIKEFLQSFVHIVQMFINTSSGGGSGGGG | |
| | SGGGGSGGGGSGGGSLQITCPPPMSVEHADIWVKSYSLYSRER | |
| | YICNSGFKRKAGTSSLTECVLNKATNVAHWTTPSLKCIRDPAL | |
| | VHQRPAPPSTVTTAGVTPQPESLSPSGKEPAASSPSSNNTAAT | |
| | TAAIVPGSQLMPSKSPSTGTTEISSHESSHGTPSQTTAKNWEL | |
| | TASASHQPPGVYPQGHSDTTVAISTSTVLLCGLSAVSLLACYL | |
| | KSRQTPPLASVEMEAMEALPVTWGTSSRDEDLENCSHHLGSGV | |
| | KQTLNFDLLKLAGDVESNPGPMLLLVTSLLLCELPHPAFLLIP | |
| | DIQMTQTTSSLSASLGDRVTISCRASQDISKYLNWYQQKPDGT | |
| | VKLLIYHTSRLHSGVPSRFSGSGSGTDYSLTISNLEQEDIATY | |
| | FCQQGNTLPYTFGGGTKLEITGSTSGSGKPGSGEGSTKGEVKL | |
| | QESGPGLVAPSQSLSVTCTVSGVSLPDYGVSWIRQPPRKGLEW | |
| | LGVIWGSETTYYNSALKSRLTIIKDNSKSQVFLKMNSLQTDDT | |
| | AIYYCAKHYYYGGSYAMDYWGQGTSVTVSSKPTTTPAPRPPTP | |
| | APTIASQPLSLRPEACRPAAGGAVHTRGLDFACDIYIWAPLAG | |
| | TCGVLLLSLVITLYCNHRNRSKRSRGGHSDYMNMTPRRPGPTR | |
| | KHYQPYAPPRDFAAYRSRVKFSRSADAPAYQQGQNQLYNELNL | |
| | GRREEYDVLDKRRGRDPEMGGKPRRKNPQEGLYNELQKDKMAE | |
| | AYSEIGMKGERRRGKGHDGLYQGLSTATKDTYDALHMQALPPR | |

### 5.6 Transposon and Transposase Systems

In some embodiments, transgenes of the polycistronic vector are introduced into an immune effector cell via synthetic DNA transposable elements, e.g., a DNA transposon/transposase system, e.g., Sleeping Beauty (SB). SB belongs to the Tc1/mariner superfamily of DNA transposons. DNA transposons translocate from one DNA site to another in a simple, cut-and-paste manner. Transposition is a precise process in which a defined DNA segment is excised from one DNA molecule and moved to another site in the same or different DNA molecule or genome.

Exemplary DNA transposon/transposase systems include, but are not limited to, Sleeping Beauty (see, e.g., U.S. Pat. Nos. 6,489,458, 8,227,432, the contents of each of which are incorporated by reference in their entirety herein), piggy Bac transposon system (see e.g., U.S. Pat. No. 9,228,180, Wilson et al, “PiggyBac Transposon-mediated Gene Transfer in Human Cells,” Molecular Therapy, 15:139-145 (2007), the contents of each of which are incorporated by reference in their entirety herein), piggyBat transposon system (see e.g., Mitra et al., “Functional characterization of piggy Bat from the bat Myotis lucifugus unveils an active mammalian DNA transposon,” Proc. Natl. Acad. Sci USA 110:234-239 (2013), the contents of which are incorporated by reference in their entirety herein), TcBuster (see e.g., Woodard et al. “Comparative Analysis of the Recently Discovered hAT Transposon TcBuster in Human Cells,” PLOS ONE, 7(11): e42666 (November 2012), the contents of which are incorporated by reference in their entirety herein), and the Tol2 transposon system (see e.g., Kawakami, “Tol2: a versatile gene transfer vector in vertebrates,” Genome Biol. 2007; 8(Suppl 1): S7, the contents of each of which are incorporated by reference in their entirety herein). Additional exemplary transposon/transposase systems are provided in U.S. Pat. Nos. 7,148,203; 8,227,432; US20110117072; Mates et al., Nat Genet, 41(6):753-61 (2009); and Ivies et al., Cell, 91(4):501-10, (1997), the contents of each of which are incorporated by reference in their entirety herein).

In some embodiments, the transgenes described herein are introduced into an immune effector cell via the SB transposon/transposase system. The SB transposon system comprises a SB a transposase and SB transposon(s). The SB transposon system can comprise a naturally occurring SB transposase or a derivative, variant, and/or fragment that retains activity, and a naturally occurring SB transposon, or a derivative, variant, and/or fragment that retains activity. An exemplary SB system is described in, Hackett et al., “A Transposon and Transposase System for Human Application,” Mol Ther 18:674-83, (2010)), the entire contents of which are incorporated by reference herein.

In some embodiments, the vector comprises a Left inverted terminal repeat (ITR), i.e., an ITR that is 5′ to an expression cassette, and a Right ITR, i.e., an ITR that is 3′ to an expression cassette. The Left ITR and Right ITR flank the polycistronic expression cassette of the vector. In some embodiments, the Left ITR is in reverse orientation relative to the polycistronic expression cassette, and the Right ITR is in the same orientation relative to the polycistronic expression cassette. In some embodiments, the Right ITR is in reverse orientation relative to the polycistronic expression cassette, and the Left ITR is in the same orientation relative to the polycistronic expression cassette.

In some embodiments, the Left ITR and the Right ITR are ITRs of a DNA transposon selected from the group consisting of a Sleeping Beauty transposon, a piggyBac transposon, TcBuster transposon, and a Tol2 transposon. In some embodiments, the Left ITR and the Right ITR are ITRs of the Sleeping Beauty DNA transposon.

In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 155 or 156. In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 155. In some embodiments, the Left ITR comprises the polynucleotide sequence of SEQ ID NO: 155. In some embodiments, the Left ITR comprises a polynucleotide sequence at least 75%, 80%, 85%, 90%, 95%, 96%, 97%, 98%, 99%, or 100% identical to the polynucleotide sequence of SEQ ID NO: 156. In some embodiments, the Left ITR comprises the polynucleotide sequence of SEQ ID NO: 156. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 157, 159, or 184. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 157. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 159. In some embodiments, the Right ITR comprises a polynucleotide sequence at least 75%, 80%, 85%, 90%, 95%, 96%, 97%, 98%, 99%, or 100% identical to the polynucleotide sequence of SEQ ID NO: 184. In some embodiments, the Right ITR comprises the polynucleotide sequence of SEQ ID NO: 157. In some embodiments, the Right ITR comprises the polynucleotide sequence of SEQ ID NO: 159. In some embodiments, the Right ITR comprises the polynucleotide sequence of SEQ ID NO: 184.

The polynucleotide sequence of exemplary SB ITRs are provided in Table 12, herein.

TABLE 12
Polynucleotide sequence of exemplary SB ITRs.

| Description | Polynucleotide Sequence | SEQ ID NO |
|---|---|---|
| Left ITR-A | AGTTGAAGTCGGAAGTTTACATACACTTAAGTTGGAGTCATTAAAA | 155 |
| | CTCGTTTTTCAACTACTCCACAAATTTCTTGTTAACAAACAATAGT | |
| | TTTGGCAAGTCAGTTAGGACATCTACTTTGTGCATGACACAAGTCA | |
| | TTTTTCCAACAATTGTTTACAGACAGATTATTTCACTTATAATTCA | |
| | CTGTATCACAATTCCAGTGGGTCAGAAGTTTACATACACTAA | |
| Left ITR-B | ATATCAATTGAGTTGAAGTCGGAAGTTTACATACACTTAAGTTGGA | 156 |
| | GTCATTAAAACTCGTTTTTCAACTACACCACAAATTTCTTGTTAAC | |
| | AAACAATAGTTTTGGCAAGTCAGTTAGGACATCTACTTTGTGCATG | |
| | ACACAAGTCATTTTTCCAACAATTGTTTACAGACAGATTATTTCAC | |
| | TTATAATTCACTGTATCACAATTCCAGTGGGTCAGAAGTTTACATA | |
| | CACTAACAATTGATAT | |
| Right ITR-A | TTGAGTGTATGTAAACTTCTGACCCACTGGGAATGTGATGAAAGAA | 157 |
| | ATAAAAGCTGAAATGAATCATTCTCTCTACTATTATTCTGATATTT | |
| | CACATTCTTAAAATAAAGTGGTGATCCTAACTGACCTAAGACAGGG | |
| | AATTTTTACTAGGATTAAATGTCAGGAATTGTGAAAAAGTGAGTTT | |
| | AAATGTATTTGGCTAAGGTGTATGTAAACTTCCGACTTCAACTG | |
| Right ITR-B | TTGAGTGTATGTTAACTTCTGACCCACTGGGAATGTGATGAAAGAA | 184 |
| | ATAAAAGCTGAAATGAATCATTCTCTCTACTATTATTCTGATATTT | |
| | CACATTCTTAAAATAAAGTGGTGATCCTAACTGACCTTAAGACAGG | |
| | GAATCTTTACTCGGATTAAATGTCAGGAATTGTGAAAAAGTGAGTT | |
| | TAAATGTATTTGGCTAAGGTGTATGTAAACTTCCGACTTCAACT | |
| Right ITR-C | ATATCTCGAGTTGAGTGTATGTTAACTTCTGACCCACTGGGAATGT | 159 |
| | GATGAAAGAAATAAAAGCTGAAATGAATCATTCTCTCTACTATTAT | |
| | TCTGATATTTCACATTCTTAAAATAAAGTGGTGATCCTAACTGACC | |
| | TTAAGACAGGGAATCTTTACTCGGATTAAATGTCAGGAATTGTGAA | |
| | AAAGTGAGTTTAAATGTATTTGGCTAAGGTGTATGTAAACTTCCGA | |
| | CTTCAACTCTCGAGATAT | |

In some embodiments, the DNA transposase is a SB transposase. In some embodiments, the SB transposase is selected from the group consisting of SB111, SB100X, hSB110, and hSB81. In some embodiments, the SB transposase is SB11. Exemplary SB transposases are described in U.S. Pat. No. 9,840,696, US20160264949, U.S. Pat. No. 9,228,180, WO2019038197, U.S. Ser. No. 10/174,309, and U.S. Ser. No. 10/570,382, the full contents of each of which is incorporated by reference herein.

In some embodiments, the DNA transposase comprises an amino acid sequence at least 95%, 96%, 97%, 98%, 99% or 100% identical to the amino acid sequence of SEQ ID NO: 160. In some embodiments, the DNA transposase comprises the amino acid sequence of SEQ ID NO: 160. In some embodiments, the amino acid sequence of the DNA transposase consists of a sequence at least 95%, 96%, 97%, 98%, 99% or 100% identical to the amino acid sequence of SEQ ID NO: 160. In some embodiments, the amino acid sequence of the DNA transposase consists of the amino acid sequence of SEQ ID NO: 160.

In some embodiments, the DNA transposase comprises an amino acid sequence that lacks its N-terminal methionine. In some embodiments, the DNA transposase comprises an amino acid sequence at least 95%, 96%, 97%, 98%, 99% or 100% identical to the amino acid sequence of SEQ ID NO: 160 lacking its N-terminal methionine, i.e., amino acids 2-340 of SEQ ID NO: 160. In some embodiments, the DNA transposase comprises the amino acid sequence of SEQ ID NO: 160 lacking its N-terminal methionine, i.e., amino acids 2-340 of SEQ ID NO:160. In some embodiments, the amino acid sequence of the DNA transposase consists of a sequence at least 95%, 96%, 97%, 98%, 99% or 100% identical to the amino acid sequence of SEQ ID NO: 160 lacking its N-terminal methionine, i.e., amino acids 2-340 of SEQ ID NO:160. In some embodiments, the amino acid sequence of the DNA transposase consists of the amino acid sequence of SEQ ID NO: 160 lacking its N-terminal methionine, i.e., amino acids 2-340 of SEQ ID NO:160.

In some embodiments, the DNA transposase is encoded by a polynucleotide sequence at least 75%, 80%, 85%, 90%, 95%, 96%, 97%, 98%, 99%, or 100% identical to the polynucleotide sequence of SEQ ID NO: 161. In some embodiments, the DNA transposase is encoded by the polynucleotide sequence of SEQ ID NO: 161.

In some embodiments, the DNA transposase is encoded by a polynucleotide that is introduced into a cell. In some embodiments, the polynucleotide encoding the DNA transposase is a DNA vector. In some embodiments, the polynucleotide encoding the DNA transposase is a RNA vector. In some embodiments, the DNA transposase is encoded on a first vector and the transgenes are encoded on a second vector. In some embodiments, the DNA transposase is directly introduced to a population of cells as a polypeptide.

The amino acid and polynucleotide sequence of an exemplary SB transposase is provided in Table 13, herein.

TABLE 13
Amino acid and polynucleotide sequence of an exemplary SB transposase.

| | | SEQ ID | | SEQ ID |
|---|---|---|---|---|
| Description | Amino Acid Sequence | NO | Polynucleotide Sequence | NO |
| SB11 | MGKSKEISQDLRKKIVD | 160 | ATGGGAAAATCAAAAGAAATCAGCCAA | 161 |
| | LHKSGSSLGAISKRLKV | | GACCTCAGAAAAAAAATTGTAGACCTC | |
| | PRSSVQTIVRKYKHHGT | | CACAAGTCTGGTTCATCCTTGGGAGCA | |
| | TQPSYRSGRRRVLSPRD | | ATTTCCAAACGCCTGAAAGTACCACGT | |
| | ERTLVRKVQINPRTTAK | | TCATCTGTACAAACAATAGTACGCAAG | |
| | DLVKMLEETGTKVSIST | | TATAAACACCATGGGACCACGCAGCCG | |
| | VKRVLYRHNLKGRSARK | | TCATACCGCTCAGGAAGGAGACGCGTT | |
| | KPLLQNRHKKARLRFAR | | CTGTCTCCTAGAGATGAACGTACTTTG | |
| | AHGDKDRTFWRNVLWSD | | GTGCGAAAAGTGCAAATCAATCCCAGA | |
| | ETKIELFGHNDHRYVWR | | ACAACAGCAAAGGACCTTGTGAAGATG | |
| | KKGEACKPKNTIPTVKH | | CTGGAGGAAACAGGTACAAAAGTATCT | |
| | GGGSIMLWGCFAAGGTG | | ATATCCACAGTAAAACGAGTCCTATAT | |
| | ALHKIDGIMRKENYVDI | | CGACATAACCTGAAAGGCCGCTCAGCA | |
| | LKQHLKTSVRKLKLGRK | | AGGAAGAAGCCACTGCTCCAAAACCGA | |
| | WVFQQDNDPKHTSKHVR | | CATAAGAAAGCCAGACTACGGTTTGCA | |
| | KWLKDNKVKVLEWPSQS | | AGAGCACATGGGGACAAAGATCGTACT | |
| | PDLNPIENLWAELKKRV | | TTTTGGAGAAATGTCCTCTGGTCTGAT | |
| | RARRPTNLTQLHQLCQE | | GAAACAAAAATAGAACTGTTTGGCCAT | |
| | EWAKIHPTYCGKLVEGY | | AATGACCATCGTTATGTTTGGAGGAAG | |
| | PKRLTQVKQFKGNATKY | | AAGGGGGAGGCTTGCAAGCCGAAGAAC | |
| | | | ACCATCCCAACCGTGAAGCACGGGGGT | |
| | | | GGCAGCATCATGTTGTGGGGGTGCTTT | |
| | | | GCTGCAGGAGGGACTGGTGCACTTCAC | |
| | | | AAAATAGATGGCATCATGAGGAAGGAA | |
| | | | AATTATGTGGATATATTGAAGCAACAT | |
| | | | CTCAAGACATCAGTCAGGAAGTTAAAG | |
| | | | CTTGGTCGCAAATGGGTCTTCCAACAA | |
| | | | GACAATGACCCCAAGCATACTTCCAAA | |
| | | | CACGTGAGAAAATGGCTTAAGGACAAC | |
| | | | AAAGTCAAGGTATTGGAGTGGCCATCA | |
| | | | CAAAGCCCTGACCTCAATCCTATAGAA | |
| | | | AATTTGTGGGCAGAACTGAAAAAGCGT | |
| | | | GTGCGAGCAAGGAGGCCTACAAACCTG | |
| | | | ACTCAGTTACACCAGCTCTGTCAGGAG | |
| | | | GAATGGGCCAAAATTCACCCAACTTAT | |
| | | | TGTGGGAAGCTTGTGGAAGGCTACCCG | |
| | | | AAACGTTTGACCCAAGTTAAACAATTT | |
| | | | AAAGGCAATGCTACCAAATAC | |

### 5.7 Immune Effector Cells and Methods of Engineering

In one aspect, provided herein are cells, e.g., immune effector cells, comprising a recombinant vector comprising a polycistronic expression cassette (e.g., a vector described herein). In some embodiments, the immune effector cell is a T cell. In some embodiments, the immune effector cell is a CD4+ T cell. In some embodiments, the immune effector cell is a CD8+ T cell. In one aspect, provided herein is a population immune effector cells comprising a polycistronic vector described herein. In some embodiments, the population of immune effector cells comprises CD4+ T cells and CD8+ T cells. In some embodiments, the population of immune effector cells are an ex vivo culture.

In one aspect, provided herein are methods of introducing a vector described herein into a plurality of cells, e.g., immune effector cells, to produce a plurality of engineered cells, e.g., immune effector cells. Methods of introducing vectors into a cell are well known in the art. In the context of an expression vector, the vector can be readily introduced into a host cell, e.g., mammalian (e.g., human) cell by any method in the art. For example, the expression vector can be transferred into a host cell by transfection or transduction. Exemplary methods for introducing a vector into a host cell, include, but are not limited to, electroporation (also referred to herein as electro-transfer), calcium phosphate precipitation, lipofection, particle bombardment, microinjection, mechanical deformation by passage through a microfluidic device, and the like, see, e.g., Sambrook et al. Molecular Cloning: A Laboratory Manual, Cold Spring Harbor Laboratory, New York (2001), the entire contents of which is incorporated by reference herein. In some embodiments, a polycistronic vector is introduced into an immune effector cell or population of immune effector cells via electroporation. Alternative delivery systems include, e.g., colloidal dispersion systems, such as macromolecule complexes, nanocapsules, microspheres, beads, and lipid-based systems including oil-in-water emulsions, micelles, mixed micelles, and liposomes. In some embodiments, the polycistronic vector is introduced into a population of cells, e.g., immune effector cells, ex vivo, in vitro, or in vivo. In some embodiments, the polycistronic vector is introduced into a population of cells, e.g., immune effector cells, ex vivo.

### 5.7.1 Sources of Immune Effector Cells

Immune effector cells may be obtained from a subject by any suitable method known in the art. For example, T cells (e.g., CD4+ T cells and CD8+ T cells) can be obtained from several sources, including peripheral blood mononuclear cells, bone marrow, lymph node tissue, cord blood, thymus tissue, tissue from a site of infection, ascites, pleural effusion, spleen tissue, and tumors. In some embodiments, immune effector cells (e.g., T cells) are obtained from blood collected from a subject using any number of techniques known to the skilled artisan. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. T cells are isolated from peripheral blood lymphocytes by lysing the red blood cells and depleting the monocytes, for example, by centrifugation through a percoll gradient or by counter flow centrifugal elutriation.

The cells collected by apheresis can be washed to remove the plasma fraction and to place the cells in an appropriate buffer (e.g., phosphate buffered saline (PBS)) or media for subsequent processing steps. The washing step may be accomplished by methods known to those in the art, such as by using a semi-automated “flow-through” centrifuge. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

A specific subpopulation of cells can be further isolated by positive or negative selection techniques (e.g., antibody coated beads, flow cytometry, etc.). In some embodiments, a specific subpopulation of T cells, such as CD3+, CD28+, CD4+, CD8+, CD45RA+, and CD45RO+ T cells, can be further isolated by positive or negative selection techniques (e.g., antibody coated beads, flow cytometry, etc.).

### 5.7.2 Activation and Expansion

In some embodiments, the T cells are activated prior to introduction of a polycistronic vector described herein. In some embodiments, the T cells are activated by contacting the cells with a molecule that specifically binds CD3 optionally in combination with a molecule that specifically binds CD28. Exemplary activation methods include contacting the T cells ex vivo with beads that are covalently coupled with anti-CD3 and optionally anti-CD28 antibodies. In some embodiments, the T cells are expanded post introduction of a polycistronic vector described herein. In some embodiments, the expansion comprises contacting the cells with a molecule that specifically binds CD3 optionally in combination with a molecule that specifically binds CD28. Exemplary activation methods include contacting the T cells ex vivo with beads that are covalently coupled with anti-CD3 and optionally anti-CD28 antibodies.

### 5.7.3 Rapid Personalized Manufacture (RPM)

In one aspect, provided herein are methods of introducing a polycistronic vector described herein into a population of cells to produce a population of engineered cells. In some embodiments, the population of cells comprises immune effector cells. In some embodiments, the immune effector cells are T cells. In some embodiments, the population of cells comprises CD8+ T cells. In some embodiments, the population of cells comprises CD4+ T cells. In some embodiments, the population of cells comprises CD8+ T cells and CD8+ T cells.

In some embodiments, the method comprises introducing into a population of cells a recombinant vector described herein, and a DNA transposase (e.g., a DNA transposase described herein) or a polynucleotide encoding a DNA transposase (e.g., a DNA transposase described herein); and culturing the population of cells under conditions wherein the transposase integrates the polycistronic expression cassette into the genome of the population of cells. In some embodiments, the recombinant vector, and the DNA transposase or polynucleotide encoding said DNA transposase, are introduced to the population of cells using electro-transfer, calcium phosphate precipitation, lipofection, particle bombardment, microinjection, mechanical deformation by passage through a microfluidic device, or a colloidal dispersion system.

In some embodiments, the population of engineered cells is produced in from about 1 to 5 days, 1 to 4 days, 1 to 3 days, or 1 to 2 days. In some embodiments, the population of engineered cells is produced in less than 5 days, 4 days, 3 days, 2 days, or 1 day. In some embodiments, the population of engineered cells is produced in more than 1 day, 2 days, 3 days, 4 days, or 5 days.

In some embodiments, the cells are not exogenously activated ex vivo. In some embodiments, the cells are not cultured in the presence of an exogenous cytokine ex vivo. In some embodiments, the polycistronic vector is introduced into resting T cells (e.g., by electroporation) ex vivo. In some embodiments, the T cells express CCR7 on the cell surface and do not express a detectable level of CD45RO.

In some embodiments, the cells are cultured ex vivo for no more than 96 hours, 72 hours, 48 hours, 24 hours, 12 hours, or 6 hours, post introduction (e.g., by electroporation) of a polycistronic vector described herein. In some embodiments, the cells are cultured ex vivo for about 96 hours, about 72 hours, about 48 hours, about 24 hours, about 12 hours, or about 6 hours, post introduction (e.g., by electroporation) of a polycistronic vector described herein. In some embodiments, the cells are cultured ex vivo for about 6-96 hours, about 6-72 hours, about 6-48 hours, about 6-24 hours, about 6-12 hours, about 12-96 hours, about 12-72 hours, about 12-48 hours, about 12-24 hours, about 24-96 hours, about 24-72 hours, about 24-48 hours, about 48-96 hours, or about 48-72 hours post introduction (e.g., by electroporation) of a polycistronic vector described herein.

In some embodiments, the cells are administered to a subject in need thereof no more than 96 hours, 72 hours, 48 hours, 24 hours, 12 hours, or 6 hours, post introduction (e.g., by electroporation) of a polycistronic vector described herein. In some embodiments, the cells are administered to a subject in need thereof about 96 hours, about 72 hours, about 48 hours, about 24 hours, about 12 hours, or about 6 hours post introduction (e.g., by electroporation) of a polycistronic vector described herein. In some embodiments, the cells are administered to a subject in need thereof about 6-96 hours, about 6-72 hours, about 6-48 hours, about 6-24 hours, about 6-12 hours, about 12-96 hours, about 12-72 hours, about 12-48 hours, about 12-24 hours, about 24-96 hours, about 24-72 hours, about 24-48 hours, about 48-96 hours, or about 48-72 hours post introduction (e.g., by electroporation) of a polycistronic vector described herein.

### 5.8 Pharmaceutical Compositions

Provided herein are pharmaceutical compositions comprising a population of engineered immune effector cells disclosed herein having the desired degree of purity in a physiologically acceptable carrier, excipient or stabilizer (see, e.g., Remington's Pharmaceutical Sciences (1990) Mack Publishing Co., Easton, PA). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Pharmaceutical compositions described herein can be useful in inducing an immune response in a subject and treating a condition, such as cancer. In one embodiment, the present disclosure provides a pharmaceutical composition comprising a population of engineered immune effector cells described herein for use as a medicament. In another embodiment, the disclosure provides a pharmaceutical composition for use in a method for the treatment of cancer. In some embodiments, pharmaceutical compositions comprise a population of engineered immune effector cells disclosed herein, and optionally one or more additional prophylactic or therapeutic agents, in a pharmaceutically acceptable carrier.

A pharmaceutical composition may be formulated for any route of administration to a subject. Specific examples of routes of administration include parenteral administration (e.g., intravenous, subcutaneous, intramuscular). In some embodiments, the pharmaceutical composition is formulated for intravenous administration. Injectables can be prepared in conventional forms, either as liquid solutions or suspensions. The injectables can contain one or more excipients. Exemplary excipients include, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In some embodiments, the pharmaceutical composition is formulated for intravenous administration. Suitable carriers for intravenous administration include physiological saline or phosphate buffered saline (PBS), and solutions containing thickening and solubilizing agents, such as glucose, polyethylene glycol, and polypropylene glycol and mixtures thereof.

The compositions to be used for in vivo administration can be sterile. This is readily accomplished by filtration through, e.g., sterile filtration membranes.

Pharmaceutically acceptable carriers used in parenteral preparations include for example, aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include sodium chloride injection, Ringer's injection, isotonic dextrose injection, sterile water injection, dextrose and lactated Ringer's injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The precise dose to be employed in a pharmaceutical composition will also depend on the route of administration, and the seriousness of the condition caused by it, and should be decided according to the judgment of the practitioner and each subject's circumstances. For example, effective doses may also vary depending upon means of administration, target site, physiological state of the subject (including age, body weight, and health), other medications administered, or whether treatment is prophylactic or therapeutic. Treatment dosages are optimally titrated to optimize safety and efficacy.

### 5.9 Therapeutic Methods of Use and Applications

In another aspect, the present disclosure provides a method of inducing an immune response in a subject in need thereof comprising administering a population of engineered immune effector cells, vector, polynucleotide, or pharmaceutical composition described herein. In some embodiments, the subject has cancer. In another aspect, the instant disclosure provides a method of treating a disease or disorder, e.g., cancer or an autoimmune disease or disorder, in a subject in need thereof comprising administering a population of engineered immune effector cells, vector, polynucleotide, or pharmaceutical composition described herein. In another aspect, the instant disclosure provides a method of treating a disease or disorder, e.g., cancer or an autoimmune disease or disorder, in a subject in need thereof comprising administering a population of engineered immune effector cells, vector, polynucleotide, or pharmaceutical composition described herein.

In some embodiments, the cells are autologous to the subject being administered said population of engineered immune effector cells. In some embodiments, the cells are allogeneic to the subject being administered said population of engineered immune effector cells.

In some embodiments, the disease or disorder is cancer. In some embodiments, the cancer is associated with expression or overexpression of CD19 on the surface of cancer cells relative to non-cancerous cells. In some embodiments, the disease or disorder is a hematological cancer. In some embodiments, the hematological cancer is a leukemia or lymphoma, e.g., an acute leukemia, an acute lymphoma, a chronic leukemia, or a chronic lymphoma. Exemplary cancers include, but are not limited to, cancer associated with expression of CD19, B-cell acute lymphoid leukemia (B-ALL) (also known as B-cell acute lymphoblastic leukemia or B-cell acute lymphocytic leukemia), B lymphoblastic leukemia with t(v; 11q23.3); KMT2A rearranged, B acute lymphoblastic leukemia with t(v; 11q23.3); KMT2A rearranged, T-cell acute lymphoid leukemia (T-ALL) (also known as T-cell acute lymphoblastic leukemia or T-cell acute lymphocytic leukemia), acute lymphoid leukemia (ALL) (also known as acute lymphoblastic leukemia or acute lymphocytic leukemia), Ph-like acute lymphoid leukemia (Ph-like ALL) (also known as Ph-like acute lymphoblastic leukemia or Ph-like acute lymphocytic leukemia), chronic myelogenous leukemia (CMIL), chronic lymphoid leukemia (CLL) (also known as chronic lymphoblastic leukemia or chronic lymphocytic leukemia), chronic lymphocytic lymphoma, small lymphocytic lymphoma (SLL), B cell prolymphocytic leukemia, blastic plasmacytoid dendritic cell neoplasm, Burkitt's lymphoma, diffuse large B-cell lymphoma (DLBCL), primary mediastinal (e.g., thymic) large B-cell lymphoma (PMBCL), follicular lymphoma, hairy cell leukemia, small-cell follicular lymphoma, large-cell follicular lymphoma, MALT lymphoma, mantle cell lymphoma, marginal zone lymphoma, multiple myeloma, myelodysplasia, myelodysplastic syndrome, non-Hodgkin lymphoma (NHL), plasmablastic lymphoma, plasmacytoid dendritic cell neoplasm, Waldenstrom macroglobulinemia, and minimal residual disease.

In some embodiments, the hematological cancer is a B cell cancer. In some embodiments, the B cell cancer is a leukemia or lymphoma. In some embodiments, the hematological malignancy is B-ALL, T-ALL, ALL, CLL, SLL, NHL, DLBCL, acute biphenotypic leukemia, or minimal residual disease.

In some embodiments, the cancer is a recurrent cancer. In some embodiments, the recurrent cancer is associated with expression or overexpression of CD19 on the surface of cancer cells relative to non-cancerous cells. In some embodiments, the disease or disorder is a recurrent hematological cancer. In some embodiments, the recurrent hematological cancer is a recurrent leukemia or recurrent lymphoma. Exemplary recurrent cancers include, but are not limited to, recurrent cancer associated with expression of CD19, recurrent B-cell acute lymphoid leukemia (recurrent B-ALL) (also known as recurrent B-cell acute lymphoblastic leukemia or recurrent B-cell acute lymphocytic leukemia), recurrent B lymphoblastic leukemia with t(v; 11q23.3); KMT2A rearranged, recurrent B acute lymphoblastic leukemia with t(v; 11q23.3); KMT2A rearranged, recurrent T-cell acute lymphoid leukemia (recurrent T-ALL) (also known as recurrent T-cell acute lymphoblastic leukemia or recurrent T-cell acute lymphocytic leukemia), recurrent acute lymphoid leukemia (recurrent ALL) (also known as recurrent acute lymphoblastic leukemia or recurrent acute lymphocytic leukemia), recurrent Ph-like acute lymphoid leukemia (recurrent Ph-like ALL) (also known as recurrent Ph-like acute lymphoblastic leukemia or recurrent Ph-like acute lymphocytic leukemia), recurrent chronic myelogenous leukemia (recurrent CML), recurrent chronic lymphoid leukemia (recurrent CLL) (also known as recurrent chronic lymphoblastic leukemia or recurrent chronic lymphocytic leukemia), recurrent chronic lymphocytic lymphoma, recurrent small lymphocytic lymphoma (recurrent SLL), recurrent B cell prolymphocytic leukemia, recurrent blastic plasmacytoid dendritic cell neoplasm, recurrent Burkitt's lymphoma, recurrent diffuse large B-cell lymphoma (recurrent DLBCL), recurrent primary mediastinal (e.g., thymic) large B-cell lymphoma (recurrent PMBCL), recurrent follicular lymphoma, recurrent hairy cell leukemia, recurrent small-cell follicular lymphoma, recurrent large-cell follicular lymphoma, recurrent MALT lymphoma, recurrent mantle cell lymphoma, recurrent marginal zone lymphoma, recurrent multiple myeloma, recurrent myelodysplasia, recurrent myelodysplastic syndrome, recurrent non-Hodgkin lymphoma (NHL), recurrent plasmablastic lymphoma, recurrent plasmacytoid dendritic cell neoplasm, recurrent Waldenstrom macroglobulinemia, and recurrent minimal residual disease.

In some embodiments, the recurrent hematological cancer is a recurrent B cell cancer. In some embodiments, the recurrent hematological malignancy is recurrent B-ALL, recurrent T-ALL, recurrent ALL, recurrent CLL, recurrent SLL, recurrent NHL, recurrent DLBCL, recurrent acute biphenotypic leukemia, or recurrent minimal residual disease.

In some embodiments, the cancer is a refractory cancer, e.g., a cancer that is resistant to treatment, e.g., standard of care, or becomes resistant to treatment over time. In some embodiments, the refractory cancer is associated with expression or overexpression of CD19 on the surface of cancer cells relative to non-cancerous cells. In some embodiments, the disease or disorder is a refractory hematological cancer. In some embodiments, the refractory hematological cancer is a refractory leukemia or refractory lymphoma. Exemplary refractory cancers include, but are not limited to, refractory cancer associated with expression of CD19, refractory B-cell acute lymphoid leukemia (refractory B-ALL) (also known as refractory B-cell acute lymphoblastic leukemia or refractory B-cell acute lymphocytic leukemia), refractory B lymphoblastic leukemia with t(v; 11q23.3); KMT2A rearranged, refractory B acute lymphoblastic leukemia with t(v; 11q23.3); KMT2A rearranged, refractory T-cell acute lymphoid leukemia (refractory T-ALL) (also known as refractory T-cell acute lymphoblastic leukemia or refractory T-cell acute lymphocytic leukemia), refractory acute lymphoid leukemia (refractory ALL) (also known as refractory acute lymphoblastic leukemia or refractory acute lymphocytic leukemia), refractory Ph-like acute lymphoid leukemia (refractory Ph-like ALL) (also known as refractory Ph-like acute lymphoblastic leukemia or refractory Ph-like acute lymphocytic leukemia), refractory chronic myelogenous leukemia (refractory CML), refractory chronic lymphoid leukemia (refractory CLL) (also known as refractory chronic lymphoblastic leukemia or refractory chronic lymphocytic leukemia), refractory chronic lymphocytic lymphoma, refractory small lymphocytic lymphoma (refractory SLL), refractory B cell prolymphocytic leukemia, refractory blastic plasmacytoid dendritic cell neoplasm, refractory Burkitt's lymphoma, refractory diffuse large B-cell lymphoma (refractory DLBCL), refractory primary mediastinal (e.g., thymic) large B-cell lymphoma (refractory PMBCL), refractory follicular lymphoma, refractory hairy cell leukemia, refractory small-cell follicular lymphoma, refractory large-cell follicular lymphoma, refractory MALT lymphoma, refractory mantle cell lymphoma, refractory marginal zone lymphoma, refractory multiple myeloma, refractory myelodysplasia, refractory myelodysplastic syndrome, refractory non-Hodgkin lymphoma (NHL), refractory plasmablastic lymphoma, refractory plasmacytoid dendritic cell neoplasm, refractory Waldenstrom macroglobulinemia, and refractory minimal residual disease.

In some embodiments, the refractory hematological cancer is a refractory B cell cancer. In some embodiments, the refractory hematological malignancy is refractory B-ALL, refractory T-ALL, refractory ALL, refractory CLL, refractory SLL, refractory NHL, refractory DLBCL, refractory acute biphenotypic leukemia, or refractory minimal residual disease.

In some embodiments, the disease or disorder is an autoimmune disease or disorder, e.g., a recurrent autoimmune disease or disorder or a refractory autoimmune disease or disorder.

In some embodiments, the population of engineered cells is administered to the subject after a hematopoietic stem cell transplant.

In some embodiments, the population of engineered cells is administered to the subject in combination (e.g., before, simultaneously, or after) with one or more prophylactic or therapeutic agents. In some embodiments, the therapeutic agent is a chemotherapeutic agent, an anti-cancer agent, an anti-angiogenic agent, an anti-fibrotic agent, an immunotherapeutic agent, a therapeutic antibody, a bispecific antibody, an “antibody-like” therapeutic protein (such as DARTs®, Duobodies®, Bites®, XmAbs®, TandAbs®, Fab derivatives), an antibody-drug conjugate (ADC), a radiotherapeutic agent, an anti-neoplastic agent, an anti-proliferation agent, an oncolytic virus, a gene modifier or editor (such as CRISPR/Cas9, zinc finger nucleases or synthetic nucleases, or TALENs), a CAR T-cell immunotherapeutic agent, an engineered T cell receptor (TCR-T), or any combination thereof. In some embodiments, the therapeutic agent is an anti-cancer agent. In some embodiments, the therapeutic agent is a chemotherapeutic agent. These therapeutic agents may be in the forms of compounds, antibodies, polypeptides, or polynucleotides.

In some embodiments, the population of engineered immune effector cells, vector, polynucleotide, or pharmaceutical composition is administered to the subject after administration of a lymphodepleting preparative regimen. In some embodiments, the lymphodepleting preparative regimen comprises at least one chemotherapeutic agent. In some embodiments, the lymphodepleting preparative regimen comprises at least two different chemotherapeutic agents. In some embodiments, the lymphodepleting preparative regimen comprises cyclophosphamide. In some embodiments, the lymphodepleting preparative regimen comprises cyclophosphamide administered to a subject in an amount sufficient to reduce an immune response in the subject. In some embodiments, the lymphodepleting preparative regimen comprises fludarabine. In some embodiments, the lymphodepleting preparative regimen comprises fludarabine administered to a subject in an amount sufficient to reduce an immune response in the subject. In some embodiments, the lymphodepleting preparative regimen comprises cyclophosphamide and fludarabine. In some embodiments, the lymphodepleting preparative regimen comprises cyclophosphamide and fludarabine, each administered to a subject in an amount sufficient to reduce an immune response in the subject.

### 5.10 Kits

In one aspect, provided herein are kits comprising one or more pharmaceutical composition, population of engineered effector cells, polynucleotide, or vector described herein and instructions for use. Such kits may include, e.g., a carrier, package, or container that is compartmentalized to receive one or more containers such as vials, tubes, and the like. Suitable containers include, for example, bottles, vials, syringes, and test tubes. In one embodiment, the containers are formed from a variety of materials such as glass or plastic.

In a specific embodiment, provided herein is a pharmaceutical kit comprising one or more containers filled with one or more of the ingredients of the pharmaceutical compositions described herein, population of engineered immune effector cells, polynucleotides, or vectors provided herein. In one embodiment, the kit comprises a pharmaceutical composition comprising a population of engineered immune effector cells described herein. In one embodiment, the kit comprises a pharmaceutical composition comprising a population of immune effector cells engineered according to a method described herein. In some embodiments, the kit contains a pharmaceutical composition described herein and a prophylactic or therapeutic agent. Optionally associated with such container(s) can be a notice in the form prescribed by a governmental agency regulating the manufacture, use or sale of pharmaceuticals or biological products, which notice reflects approval by the agency of manufacture, use or sale for human administration.

### 6. EXAMPLES

The examples in this Section (i.e., Section 6) are offered by way of illustration, and not by way of limitation.

### 6.1 Example 1: Construction of Transposon Plasmids Encoding CD19CAR, mbIL15, and HER1t

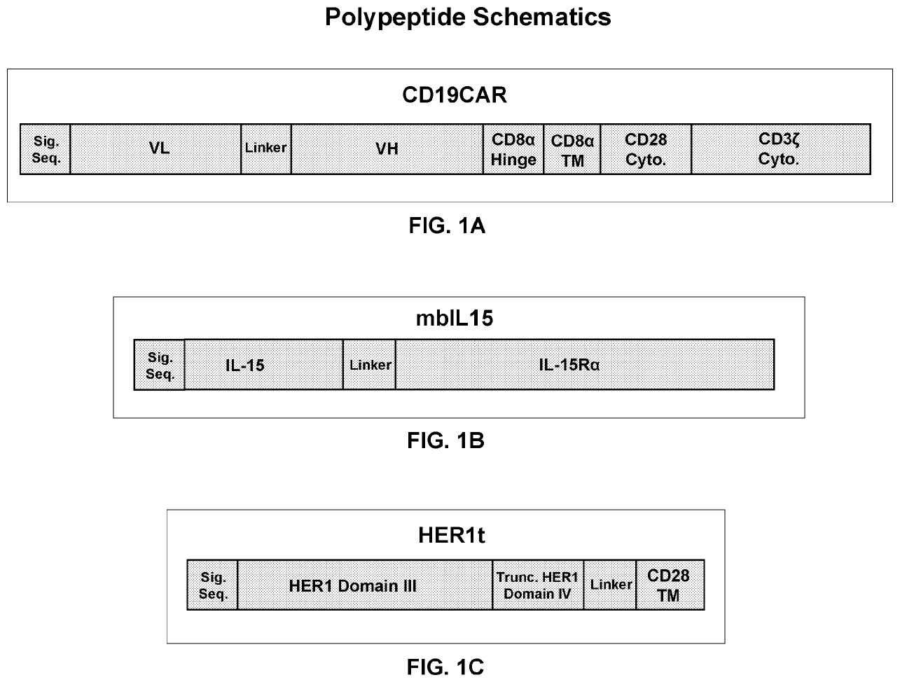

To improve homogeneity of multigene co-expression and product manufacturability, recombinant nucleic acid Sleeping Beauty transposon plasmids comprising polycistronic expression cassettes were constructed. The polycistronic expression plasmids each include a transcriptional regulatory element operably linked to a polynucleotide that encodes the anti-CD19 CAR (CD19CAR) of SEQ ID NO: 72, the membrane-bound IL-15/IL-15Rα fusion protein (mbIL15) of SEQ ID NO: 119, and the “kill switch” marker protein (HER1t) of SEQ ID NO: 96 or SEQ ID NO: 166, each separated by an F2A element or T2A element that mediates ribosome skipping to enable expression of separate polypeptide chains. Schematics of each of the encoded proteins are shown in FIGS. 1A-1C, respectively, from N terminus (left) to C terminus (right).

Briefly, CD19CAR was generated using the light chain variable region (VL) (SEQ ID NO: 1) and heavy chain variable region (VH) (SEQ ID NO: 2) of murine monoclonal antibody FMC63. The VL was placed at the mature N terminus of CD19CAR and was joined to the VH via a Whitlow linker peptide (SEQ ID NO: 9), with a human GM-CSF receptor alpha-chain signal sequence (SEQ ID NO: 10) N-terminal to the VL. The resulting scFv was joined to a human CD8α hinge domain (SEQ ID NO: 37), a human CD8α transmembrane domain (SEQ ID NO: 43), a human CD28 cytoplasmic domain (SEQ ID NO: 57), and a human CD3ζ cytoplasmic domain (SEQ ID NO: 60), in order from N terminus to C terminus. To enhance CAR expression, the amino acid sequence of the human CD28 cytoplasmic domain was modified to incorporate the amino acid sequence Gly-Gly, rather than wild-type sequence Leu-Leu, at amino acids 7-8 of SEQ ID NO: 57.

mbIL15 was constructed by joining human IL-15 (SEQ ID NO: 123) to human IL-15Ra (SEQ ID NO: 124) via a Gly-Ser-rich linker peptide (SEQ ID NO: 125), with an IgE signal sequence (SEQ ID NO: 176) N-terminal to the human IL-15.

HER1t was constructed by joining Domain III of human HER1 (SEQ ID NO: 98) to amino acids 1-21 of Domain 4 of human HER1 (SEQ ID NO: 100), with an Igκ signal sequence (SEQ ID NO: 169 or SEQ ID NO: 170) N-terminal to Domain III. The resulting sequence was joined to a human CD28 transmembrane domain (SEQ ID NO: 101) via a Gly-Ser-rich linker peptide (SEQ ID NO: 102).

To explore the effect of gene/element order on expression and function, three tricistronic polynucleotide expression cassettes, Cassettes 1-3, were generated. The 5′ to 3′ order of elements in each expression cassette is as follows: Cassette 1: CD19CAR-F2A-mbIL15-T2A-HER1t; Cassette 2: mbIL15-T2A-HER1t-F2A-CD19CAR; and Cassette 3: HER1t-T2A-mbIL15-F2A-CD19CAR. The polynucleotide sequence of each expression cassette is shown in Table 10.

The corresponding theoretical polypeptide translation product of each expression cassette, not accounting for N-terminal signal sequence cleavage or ribosomal skipping at each F2A and T2A site, is shown in Table 11.

Six recombinant nucleic acid Sleeping Beauty transposon plasmids incorporating the foregoing expression cassettes were generated. In each plasmid, one of Cassettes 1-3, as well as suitable transcriptional regulatory elements, was flanked by a pair of inverted terminal repeat sequences (ITRs) recognized by Sleeping Beauty transposase SB11. Two pairs of ITRs were evaluated for each expression cassette: ITR Pair a and ITR Pair 3. The resulting six transposon plasmids are summarized in Table 14.

TABLE 14
Tricistronic Sleeping Beauty Transposon Plasmids.

| Name | Cassette | ITR Pair | Order of Elements (5′ to 3′) |
|---|---|---|---|
| Plasmid A | 1 | α | CD19CAR-F2A-mbIL15-T2A-HER1t |
| Plasmid B | 2 | α | mbIL15-T2A-HER1t-F2A-CD19CAR |
| Plasmid C | 3 | α | HER1t-T2A-mbIL15-F2A-CD19CAR |
| Plasmid D | 4 | β | CD19CAR-F2A-mbIL15-T2A-HER1t |
| Plasmid E | 5 | β | mbIL15-T2A-HER1t-F2A-CD19CAR |
| Plasmid F | 6 | β | HER1t-T2A-mbIL15-F2A-CD19CAR |

For control purposes, two additional transposon plasmids were prepared: Plasmid DP1, which encodes CD19CAR, and Plasmid DP2, which contains an expression cassette encoding, from N terminus to C terminus, mbIL15-T2A-HER1t. Plasmid DP1 and Plasmid DP2, when combined in a 1:1 ratio, are referred to herein as “dTp Control.”

### 6.2 Example 2: Generation and Evaluation of T Cells Co-Expressing CD19CAR, mbIL15, and HER1t

This Example describes the generation and evaluation of T cells co-expressing CD19CAR, mbIL15, and HER1t from the plasmids described in Example 1.

### 6.2.1 Materials and Methods

### 6.2.1.1 Cell Lines

K562-derived activating and propagating cells (AaPC), designated as Clone 9, expressing CD64, CD86, CD137L, and truncated CD19 (as described, e.g., in Denman et al., PLoS One. 2012; 7(1):e30264, the contents of which are incorporated by reference in their entirety herein) were used in ex vivo for expansion of genetically modified T cells. Target cell lines for cytotoxicity assays were CD19⁺ (NALM-6, Daudi, CD19-EL4 and CD19neg (parental EL4) tumor cell lines and were obtained from American Type Culture Collection (Manassas, VA) (or, e.g., as described in Singh et al., PLoS One. 2013; 8(5):e64138, the contents of which are incorporated by reference in their entirety herein). Cells were routinely cultured in R10 (RPMI 1640 containing 10% heat-inactivated fetal bovine serum (FBS; Hyclone/GE Healthcare, Logan, UT) and 1% Glutamax-100 (ThermoFisher Scientific, Waltham, MA)). Cells were cultured under standard conditions of 37° C. with 5% CO₂. Cells were tested and found to be negative for mycoplasma. Identity of the cell line was confirmed by short tandem repeat DNA fingerprinting.

### 6.2.1.2 Normal Donor Human T Cells

Peripheral blood or leukapheresis product was obtained from normal donors (Key Biologics, Memphis, TN). A T-cell enriched starting product was used. The apheresis product was diluted using CliniMACS® PBS/EDTA buffer with 0.5% (v/v) HSA, and a platelet depletion step was performed via centrifugation at 400×g for 10 minutes at room temperature (RT) with subsequent resuspension in the same buffer. According to manufacturer protocol, CD4- and CD8-specific CliniMACS® microbeads (CD4 GMP MicroBeads #170-076-702, CD8 GMP MicroBeads #170-076-703; Miltenyi) were incubated with cells for 30 minutes at RT under mixing conditions that subsequently underwent paramagnetic selection on the CliniMACS Plus to enrich the starting product for T cells. Live/dead cells were enumerated on a Cellometer instrument (Nexcelom Bioscience; Lawrence, MA). Isolated T cells were cryopreserved in CryoStor CS10 and stored in the vapor phase of a liquid nitrogen tank.

### 6.2.1.3 Generation of RPM CD19CAR-mbIL15-HER1t T Cells Using SB System

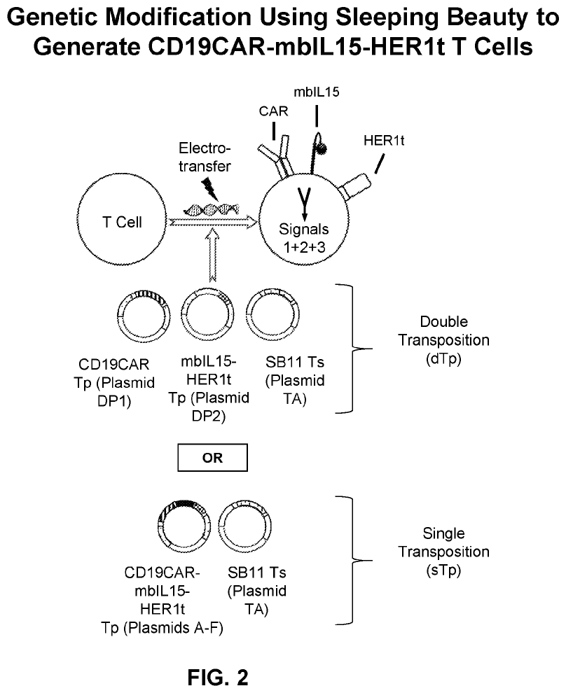

To generate the CAR-T cells described in this Example, the Nucleofector™ 2b device (Lonza; Basel, Switzerland) was used to transfer the dTp Control or Plasmids A-F, as described in Example 1, into T cell-enriched starting product. Plasmid TA, encoding the SB11 transposase, was co-transfected in each instance of transposon transfection to enable stable genetic integration of the transposon. A schematic of the gene transfer process for both double transposition (using dTp Control) and single transposition (using Plasmids A-F) is shown in FIG. 2.

The day before electroporation, cryopreserved CD3-enriched cells were thawed in R10, washed and resuspended in R10 and placed in a 37° C./5% CO₂ incubator overnight. Details for the electroporation of each test article are as follows:

Mock CD3 Cells (no DNA; also referred to herein as “Negative Control”): Rested cells were harvested, spun down, and resuspended in a device-specific Nucleofector buffer (Human T Cell Nucleofector Kit; Lonza) without any DNA plasmids.

dTp Control RPM CD19CAR-mbIL15-HER1t T Cells: Rested cells were harvested, spun down, and resuspended in Nucleofector buffer containing transposon DNA (dTp Control) and transposase DNA (Plasmid TA, encoding SB11 transposase) at a final transposon:transposase ratio of 3:1.

sTp RPM CD19CAR-mbIL15-HER1t T Cells: Rested cells were harvested, spun down, and resuspended in Nucleofector buffer containing transposon DNA (one of Plasmids A-F) and transposase DNA (Plasmid TA) at a final transposon:transposase ratio of 3:1.

Immediately following electro-transfer, the contents from each cuvette were resuspended and transferred to R10 media containing DNase for a 1-2-hour incubation in a 37° C./5% CO₂ incubator. Subsequently, a whole medium exchange was performed with R10 media, and the cells were placed overnight in a 37° C./5% CO₂ incubator. Within 24 hours (and at least 16 hours) post-electro-transfer (Day 1), the cells were harvested from culture and sampled by flow cytometry to determine cell surface expression of CD19CAR, mbIL15, and HER1t.

The Day 1 transfected T cells were stimulated with γ-irradiated (100 Gy) K562-AaPC Clone 9 at a 1:1 T cell/AaPC ratio. Additional γ-irradiated AaPC Clone 9 were added every 7-10 days at the same ratio. Soluble recombinant human IL-21 (Cat #34-8219-85, eBioscience, San Diego, CA) was added at a concentration of 30 ng/mL beginning the day after electroporation and supplemented three times per week during the 7-10-day stimulation cycles (each such stimulation cycle referred to as a “Stim”) marked by the addition of AaPC. T cells were enumerated at the end of each Stim and viable cells counted based on AOPI exclusion using Cellometer automated cell counter. Expression of T cell markers, CD19CAR, mbIL15, and HER1t was assessed using flow cytometry every 7-10 days. Expansion of unwanted NK cells in cultures was addressed by performing a depletion (positive selection using CD56 microbeads; Miltenyi) according to manufacturer's instructions. The expansion of total, CD3⁺, CD19CAR⁺, and HER1t⁺ T cells at the end of Stims 1, 2, 3, and 4 was determined.

### 6.2.1.4 Flow Cytometry

Up to 1×10⁶ cells were stained with human-specific fluorochrome conjugated antibodies. Staining for cell surface markers on samples and corresponding controls first underwent an Fc-receptor blocking step to reduce background staining by incubation with 50% mouse serum (Jackson ImmunoResearch, PA) in FACS buffer (PBS, 2% FBS, 0.1% sodium azide) for 10 minutes at 4° C. Immunostaining was performed by the addition of 100 μL of antibody master mix of combinations of antibodies listed in Table 15 that were diluted in Brilliant Stain Buffer (BD Biosciences). Briefly, CD19CAR expression was detected using Alexa Fluor® (AF) 488 conjugated anti-idiotype antibody specific for the anti-CD19 portion of the CD19CAR (clone no. 136.20.1) (as described, e.g., in Jena et al., PLoS. 2013; 8(3):e57838, the contents of which are incorporated by reference in their entirety herein). The CD19CAR anti-idiotype antibody was conjugated to the AF-488 fluorophore by Invitrogen/Thermo Fisher Scientific (Waltham, MA). The HER1t molecule was detected using fluorescently conjugated cetuximab antibody. The fluorescent-conjugated cetuximab reagent was commercially purchased Erbitux that was conjugated to AF-647 by Invitrogen/Thermo Fisher Scientific. Other fluorescently conjugated antibodies used included: CD3 (Clone SK7), IL-15 (34559), CD45 (Clone H130), and CD19-CAR idiotype (Clone 136.20.1) (Table 15).

TABLE 15
Fluorescently Conjugated Antibodies.

| Antibody Target | Clone | Fluorophore | Company |
|---|---|---|---|
| CD45 | HI30 | BV-786 | BD Biosciences |
| CD3 | SK7 | PE-Cy7 | BD Biosciences |
| CD19CAR | 136.20.1 | AF-488 | Invitrogen |
| IL-15 | 34559 | PE | R&D Systems |
| HER1t | C225 | AF-647 | Invitrogen |

The master mixes containing combinations of the antibodies in Table 15 were added in a sequential manner (CD19CAR, mbIL15, followed by the remaining antibody cocktail) and incubated up to 30 minutes at 4° C. Cells were washed with FACS buffer and then incubated with fixable viability stain-620 viability dye (1:1000 in PBS; BD Biosciences) for 10 minutes at 4° C. followed by washing with FACS buffer. Data were acquired using an LSR Fortessa (BD Biosciences) with FACSDiva software (v.8.0.1, BD Biosciences) and analyzed with FlowJo software (version 10.4.2; TreeStar, Ashland, OR). Unless described otherwise, transgene expression was assessed on gated cell events, singlets, viable events, and CD3⁺ cells.

### 6.2.1.5 Western Blot Analysis

Ex vivo expanded CD19CAR-modified T cells were centrifuged and the pellet was lysed with RIPA buffer containing protease inhibitors (Complete Mini, Roche). The lysate was incubated at 4° C. for 20 minutes and supernatants stored at −20° C. A bicinchoninic acid (BCA) assay (Thermo Fisher Scientific, 23227) was performed to determine the total protein concentration of the lysate. Western blot was performed on Wes 2010 western blot platform (ProteinSimple, Wes 2010) according to the manufacturer's instructions. For each sample, 0.1-0.2 g/mL protein lysate was mixed with 5× fluorescent master mixture (ProteinSimple, DM-002), heat denatured, cooled on ice, and loaded onto the cartridge (ProteinSimple, SM-W004). For detection of CD19CAR protein, mouse anti-human CD247 (BD Biosciences, 551033) primary antibody and HRP-goat anti-mouse (ProteinSimple, DM-002) secondary antibody were used. Jurkat cells expressing CD19 CAR were used as a positive control. For the detection of mbIL15 chimeric protein the primary antibody, goat anti-human IL-15 antibody (R&D, AF315) and secondary antibody, HRP-anti goat (ProteinSimple, 043-552-2) were used. Recombinant human IL-15 protein (R&D, 247-ILB) was loaded as a positive control. For the detection of HER1t chimeric protein the primary antibody, mouse anti-human EGFR (Sigma, AMAB90819-100 μL) and secondary antibody HRP-anti mouse antibody (ProteinSimple, DM-002) were used. Human EGFR protein (Biosystems Acro, EGR-H5252-100 μg) was used as a positive control.

### 6.2.1.6 Chromium Release Assay

Antigen specific cytotoxicity of ex vivo expanded CD19-specific T cells generated using dTp Control, Plasmid A, and Plasmid D was determined by lysis of radiolabeled (⁵¹Cr) target cells at different effector-to-target (E:T) ratios (20:1, 10:1, 5:1, 2.5:1 and 1.25:1). CD19® (NALM-6, Daudi, CD19-EL4) and CD19neg (EL4) tumor cell lines were used as targets. T cells and radiolabeled target cells were co-incubated in triplicate, and lysis was determined by measuring radioactivity in the supernatant at the end of the 4-hour incubation. Chromium release was detected using TopCount NXT (Perkin Elmer), and specific lysis was calculated as follows:

$$\% {}^{51} Cr lysis = \frac{Experimental Lysis - Background Lysis}{Maximum Lysis - Background Lysis} \times 100$$

Media and Triton-X 100-treated target cells served as controls for background and maximum lysis, respectively. Mean±SD for dTp Control (N=6), Plasmid A (N=4), and Plasmid D (N=1) lysis at each E:T ratio was calculated.

### 6.2.1.7 Antibody Dependent Cell Cytotoxicity (ADCC)

ADCC of CD19-specific T cells expressing mbIL15-HER1t was determined by a modified 4-hour chromium release assay whereby the T cells (with specific antibody treatment) served as the target cells and ex vivo activated and expanded NK cells expressing Fc receptor were used as effector cells. A range of five different effector-to-target (E:T) ratios (40:1, 20:1, 10:1, 5:1 and 2.5:1) were tested, and measurement of the amount of target lysis was established by detection of ⁵¹Cr release from the radiolabeled target T cells. Ex vivo expanded (Stim 4) CD19CAR-mbIL15-HER1t T cells were incubated with the HER1t-specific antibody Cetuximab (Imclone LLC, NDC 66733-948-23) or non-specific (irrelevant) antibody Rituximab (Biogen Inc. and Genentech USA Inc., NDC 50242-051-21) at 20 μg/mL for 20-30 minutes at RT, and these T cells were used as targets. NALM-6 and K562 cell lines were used as negative and positive controls, respectively (without antibody treatment), to assess cytolytic activity of NK cells. Target cells treated with medium alone or Triton X-100 (Sigma) were used as controls for spontaneous and maximum lysis, respectively. Percent (%)⁵¹Cr lysis was calculated as follows:

$$\% {}^{51} Cr lysis = \frac{Experimental Lysis - Background Lysis}{Maximum Lysis - Background Lysis} \times 100$$

Percent lysis data were normalized to the maximum cytolysis observed by NK cells. Mean±SD for dTp Control (N=6), Plasmid A (N=4), and Plasmid D (N=1) was calculated.

6.2.1.8 Quantitative Droplet Digital PCR (ddPCR) to Determine Transgene Copy Number

The ddPCR method was used to determine presence and quantification of CD19CAR, mbIL15, and HER1t average transgene integration events per cell of genetically modified T cells. Genomic DNA (gDNA) from ex vivo expanded (Stim 4) CD19-mbIL15-HER1t T cells transfected with the double-transposon control or test plasmids (dTp Control or Plasmids A-F, respectively), Mock transfected CD3 (no DNA negative control), CD19CAR⁺ Jurkat cells (positive control for CD19CAR), mbIL15⁺ Jurkat cells (positive control for mbIL15), and CD19CAR⁺HER1t⁺ T cells (positive control for HER1t) was isolated using a commercially available kit (Qiagen). Primer/probe sequences were designed to be specific for CD19CAR, mbIL15, and HER1t transgenes. The target primer/probes were synthesized by Bio-Rad system (Bio-Rad) with a FAM-labeled probe. All samples were duplexed with the specific human endogenous reference gene, EIF2C1, using a HEX-labeled probe (Bio-Rad). PCR droplets were generated, per manufacturer protocol, in a DG8 cartridge (Bio-Rad) using the QX-100 droplet generator, where each 20 μL PCR mixture was partitioned into approximately 20,000 nano-liter size droplets. PCR droplets were transferred into a 96-well PCR plate and sealed with foil. PCR was performed with a Bio-Rad C1000 Thermal Cycler [95° C. (10 minutes); 40 cycles of 94° C. (30 seconds), 58° C. (30 seconds), and 98° C. (10 minutes); 12° C. (indefinite)]. DNA copy number was evaluated using the QX-100 Digital Droplet PCR system (Bio-Rad). All samples were run in triplicate. After completion of the reaction in the thermocycler, the PCR plate was transferred to the QX200™ Droplet Digital™ PCR System reader to acquire the data. Data was analyzed using the QuantaSoft™ software (Version 1.7.4, Bio-Rad). To determine transgene copy number, the target (CD19CAR, mbIL15, and HER1t) to reference gene (EIF2C1) ratio was multiplied by 2, since each cell contains two copies of the reference EIF2C1 gene. The copy number variant (CNV) setting was utilized in the software program, setting the reference gene to 2 copies/cell (see, e.g., Belgrader et al., Clinical Chemistry, 2013; 59(6):991-994, and Hindson et al., Anal Chem. 2011; 83:8604-8610, the contents of each of which are incorporated by reference in their entirety herein). In the QuantaSoft™ software, the copy number is automatically determined by calculating the ratio of the target molecule concentration relative to the reference molecule concentration, multiplied by the number of copies of reference species in the genome.

### 6.2.1.9 Statistical Analysis

Statistical tests are stated with the reporting of each statistic. Post-hoc analysis was performed to compare differences between treatment groups and is reported with each statistical result. Error is reported as standard deviation (SD). GraphPad Prism (version 8) software was used to perform statistical analyses. P<0.05 was considered statistically significant.

6.2.2 Genetic Modification, Expression Characterization, and Expansion of CAR-T Cells Co-Expressing CD19CAR, mbIL15, and HER1t

Donor T cell-enriched starting product was transfected with either no transposon plasmid (Negative Control), dTp Control, or Plasmids A-F. RPM CD19CAR-mbIL15HER1t T cells were generated from three donors via electroporation using the SB system and evaluation of resultant transgenic subpopulations (CD19CAR⁺-mbIL15-HER1t⁺, CD19CAR⁺mbIL15-HER1^{neg}, CD19CAR^{neg}-mbIL15-HER1t⁺, CD19CAR^{neg}-mbIL15-HER1t^{neg}) present in the RPM T-cell products was performed one day post-transfection (Table 16).

TABLE 16
Day 1 Post-Electroporation Specifications and Transgene
Expression of RPM CD19CAR-mbIL15-HER1t T Cells.

| | | No DNA | dTp | Plasmid | Plasmid | Plasmid | Plasmid | Plasmid | Plasmid |
|---|---|---|---|---|---|---|---|---|---|
| Donor | Parameter | Control | Control | A | B | C | D | E | F |
| A | Viability (%) | 69.2 | 56.8 | 55.7 | 55.2 | 60.1 | 57.5 | 63.3 | 60.4 |
| | CD3 (%) | 95.8 | 99.0 | 98.3 | 98.6 | 98.7 | 98.5 | 97.8 | 98.5 |
| | mbIL15⁺ (%) | 0.0 | 6.1 | 13.7 | 26.2 | 10.7 | 25.3 | 24.2 | 19.9 |
| | CD19CAR (%) | 0.0 | 15.0 | 21.2 | 26.2 | 16.4 | 25.6 | 25.4 | 30.8 |
| | HER1t (%) | 0.7 | 6.4 | 22.0 | 6.8 | 22.3 | 30.6 | 6.7 | 34.4 |
| B | Viability (%) | 86.2 | 73.2 | 72.3 | 71.8 | 71.2 | 73.8 | 72.7 | 67.4 |
| | CD3 (%) | 98.1 | 98.5 | 98.4 | 98.5 | 98.6 | 98.5 | 98.7 | 98.3 |
| | mbIL15⁺ (%) | 0.3 | 18.8 | 17.9 | 31.2 | 8.2 | 20.6 | 28.0 | 13.0 |
| | CD19CAR (%) | 0.2 | 37.2 | 46.1 | 44.0 | 24.3 | 46.2 | 34.9 | 31.9 |
| | HER1t (%) | 0.1 | 27.9 | 43.0 | 15.7 | 30.7 | 43.1 | 13.3 | 38.5 |
| C | Viability (%) | 78.8 | 59.3 | 60.1 | 58.3 | 58.1 | 58.3 | 55.4 | 61.5 |
| | CD3 (%) | 94.9 | 94.2 | 94.7 | 95.1 | 93.7 | 94.0 | 95.1 | 95.1 |
| | mbIL15⁺ (%) | 0.6 | 3.3 | 7.4 | 14.4 | 1.7 | 2.7 | 7.7 | 4.6 |
| | CD19CAR (%) | 0.1 | 7.9 | 25.3 | 14.7 | 3.1 | 13.2 | 6.9 | 7.3 |
| | HER1t (%) | 0.6 | 5.6 | 25.3 | 7.9 | 16.5 | 13.1 | 3.6 | 28.9 |

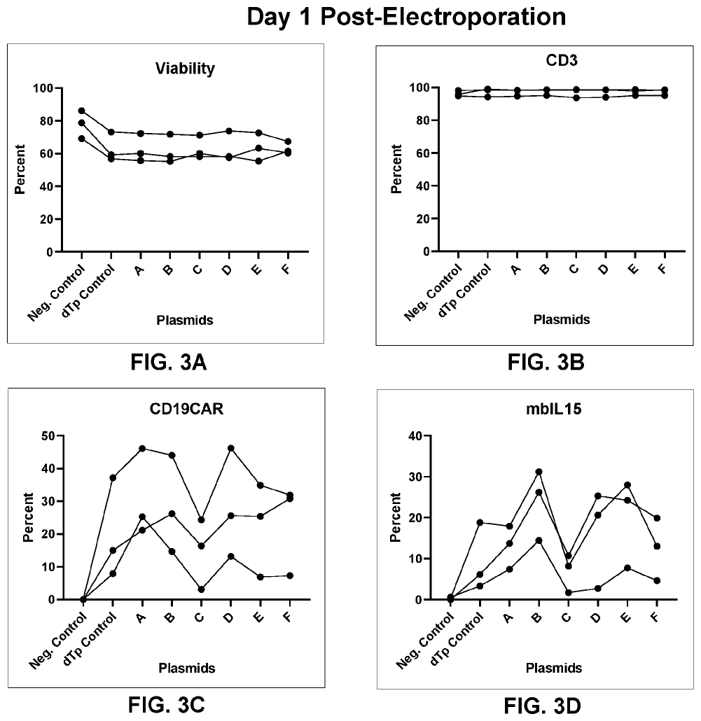

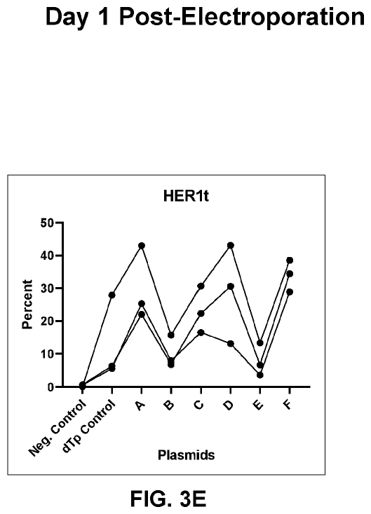

On Day 1, each of the RPM CAR-T cell groups showed comparable mean viability (620%-64%) (Table 16 and FIG. 3A) and a mean CD3⁺ frequency of 9700 (Table 16 and FIG. 3B). Regarding assessment of individual transgene expression, Plasmid A (31%±13%), Plasmid B (28%±15%), and Plasmid D (28%±17%) yielded the greatest CD19CAR expression between the sTp variants, which was ˜1.5-fold greater expression than that of the dTp Control (20%±15%) and corresponded to CD19CAR transgene in position 1 (most N-terminal) or 3 (most C-terminal) (Table 16 and FIG. 3C). Plasmid B (24%±90%) and Plasmid E (20%±110%) showed highest expression of mbIL15, followed by Plasmid A (13%±5%) and Plasmid D (16%±12%), which was higher than the observed 9%±8% expression by the dTp Control-modified T cells and corresponded to mbTL15 transgene in position 1 followed by intermediate expression when in position 2 (middle position) (Table 16 and FIG. 3D). Plasmid A, Plasmid D, and Plasmid F possessed the highest HER1t expression (30%±11%, 29%±15%, and 34%±5%, respectively), which was ˜2-fold greater expression than that of the dTp Control (13%±13%) and corresponded to HER1t in position 1 or 3 (Table 16 and FIG. 3E).

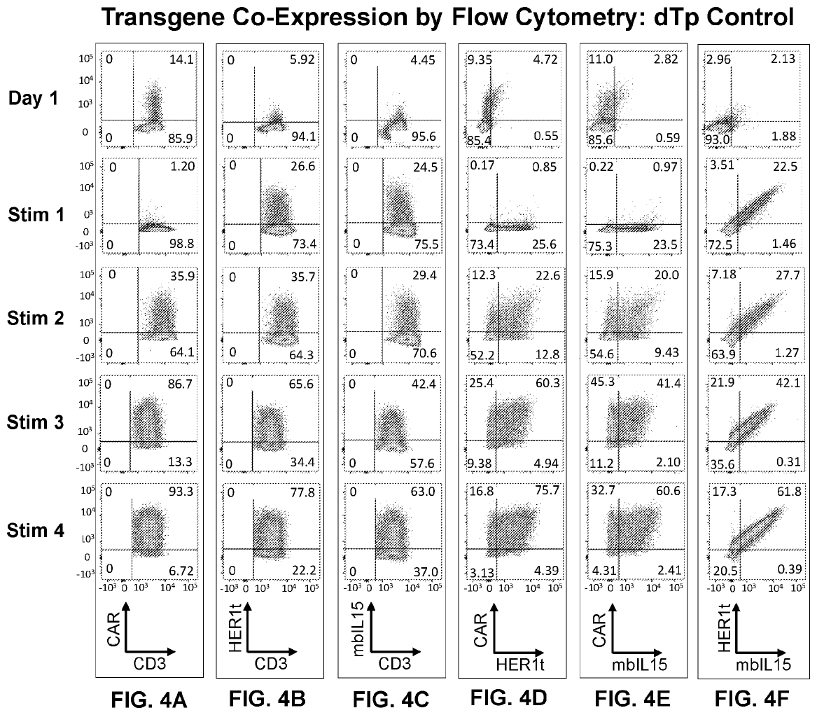

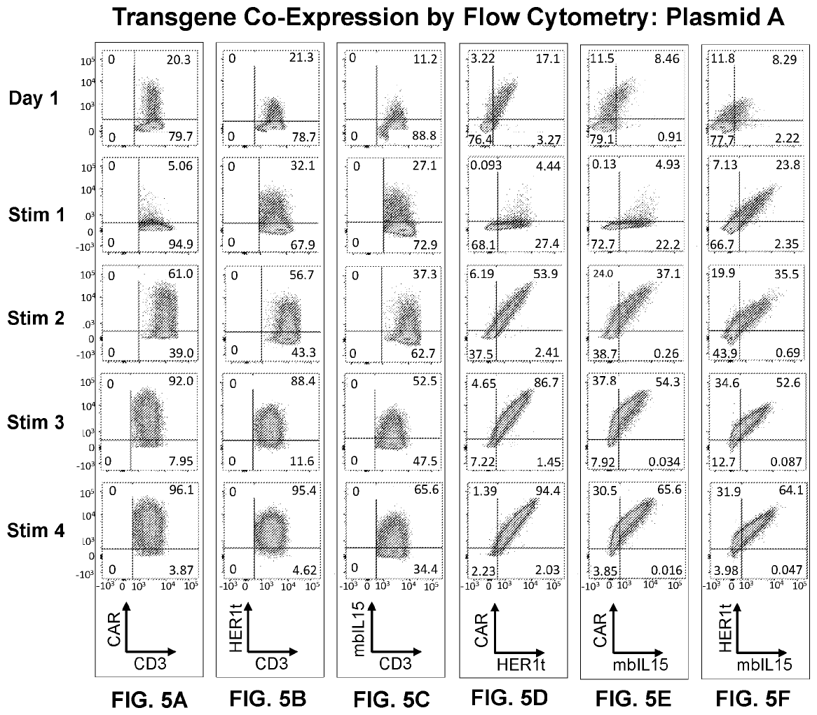

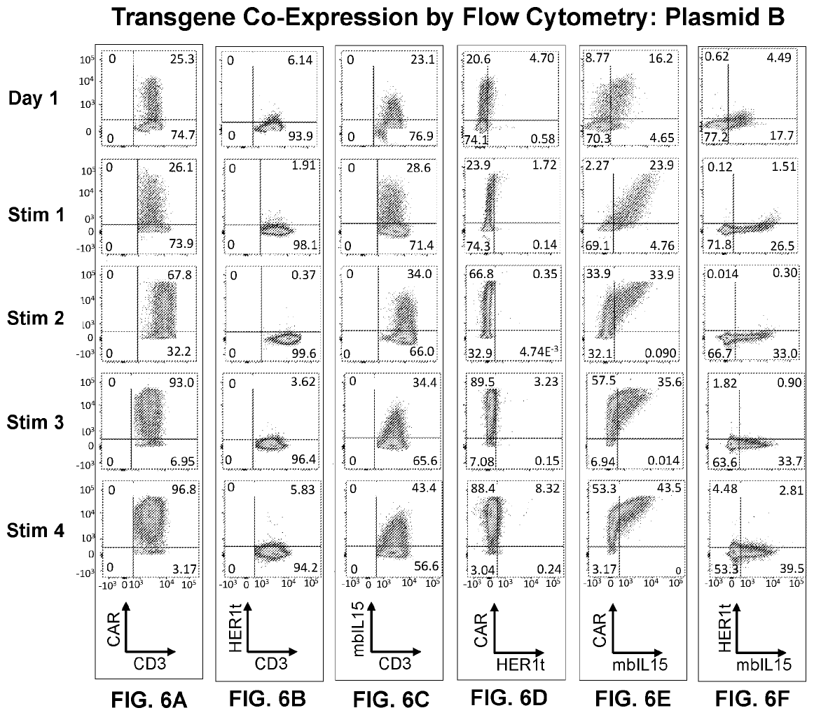

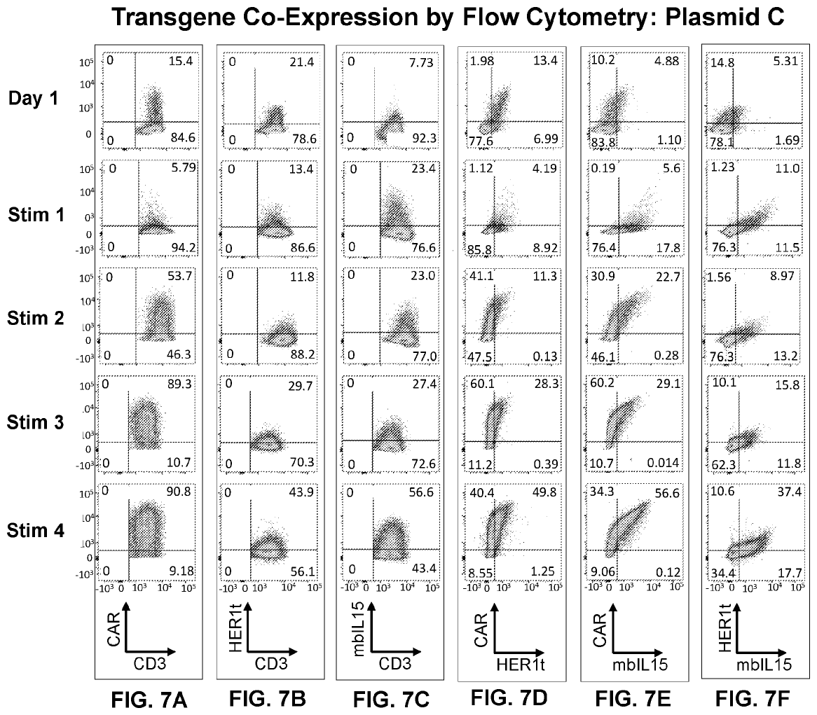

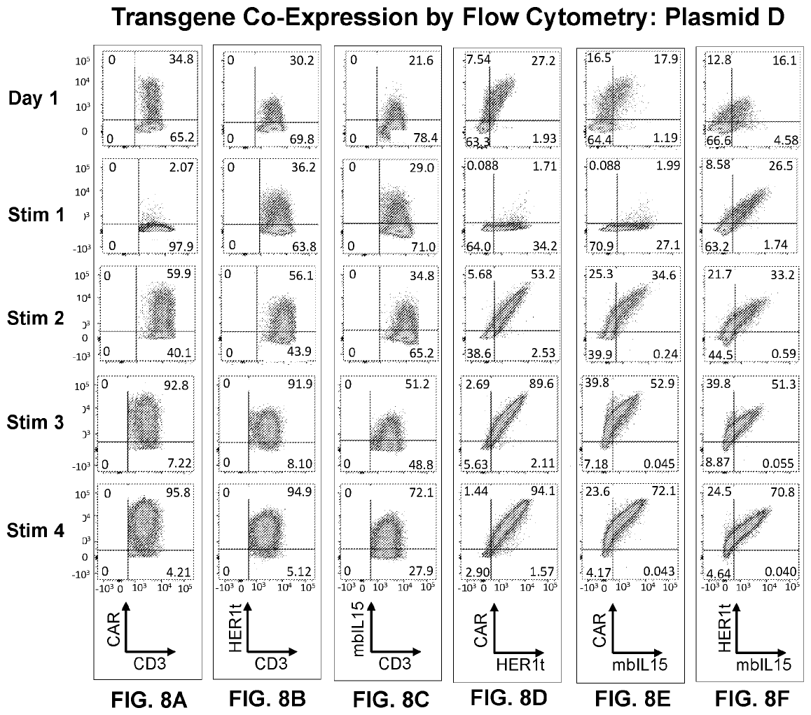

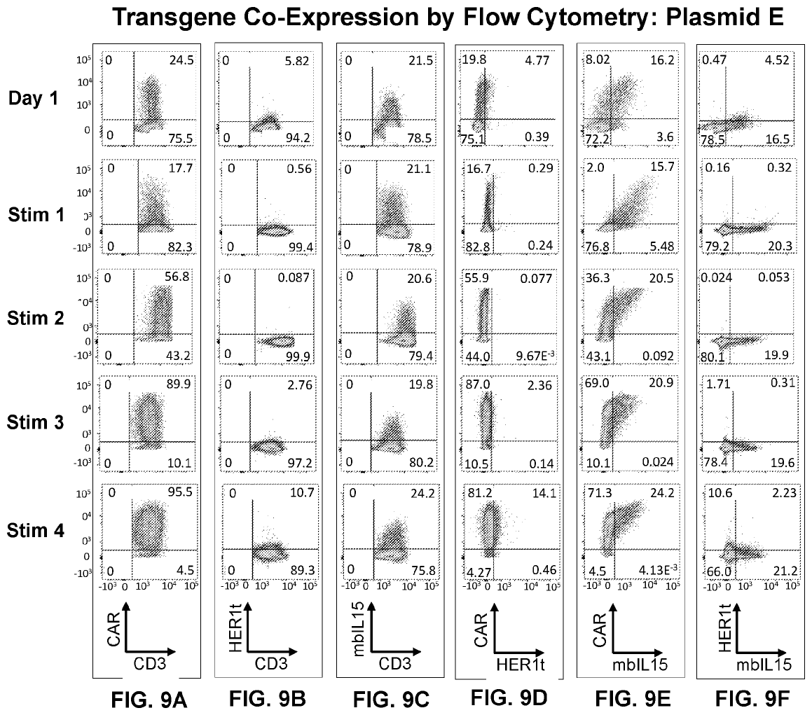

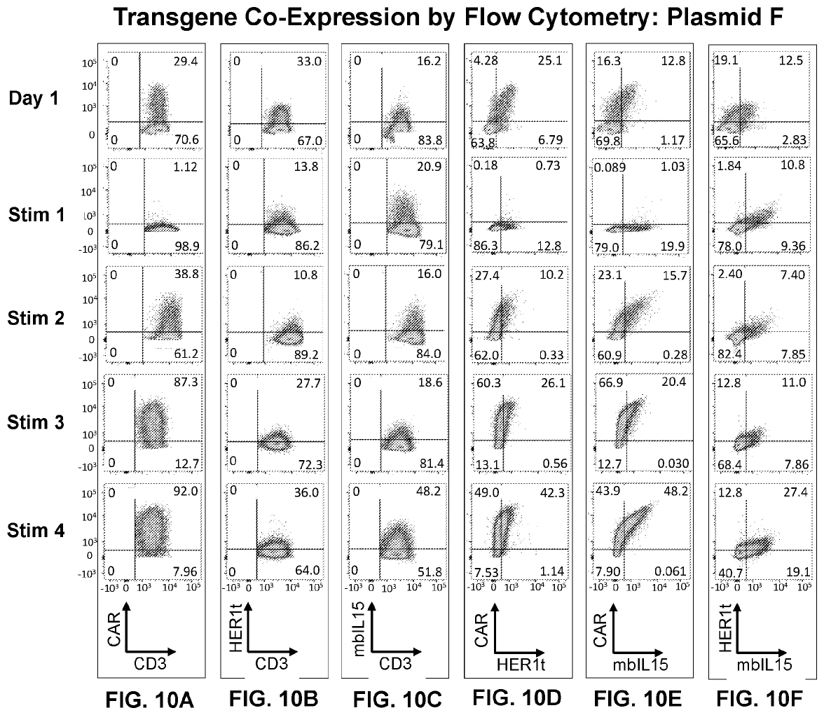

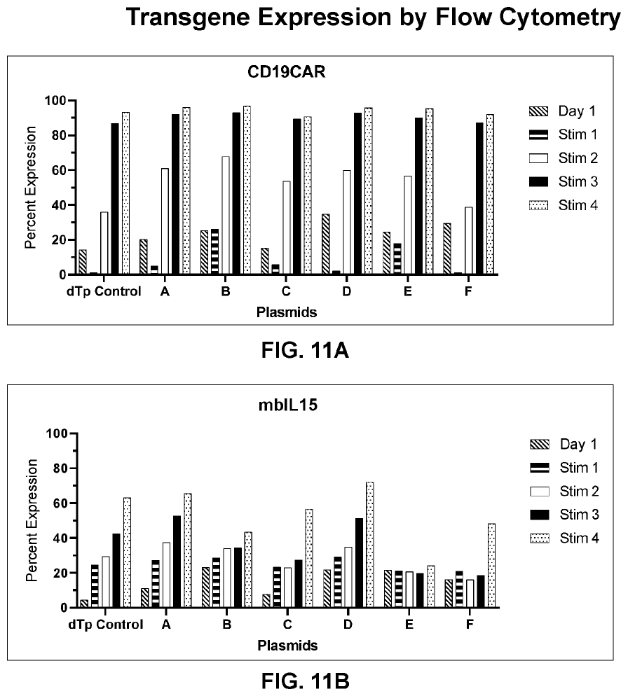

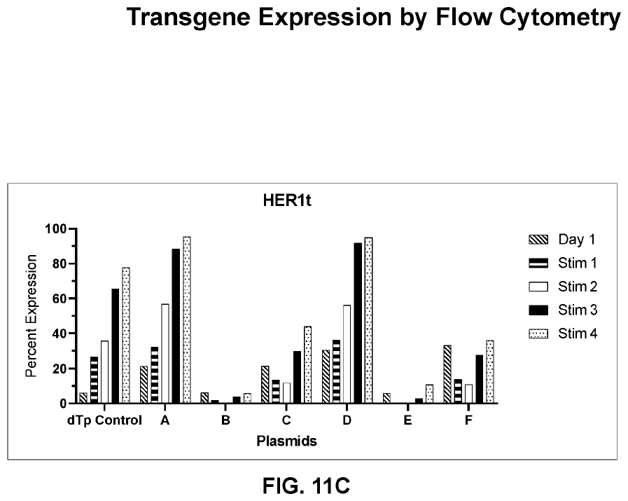

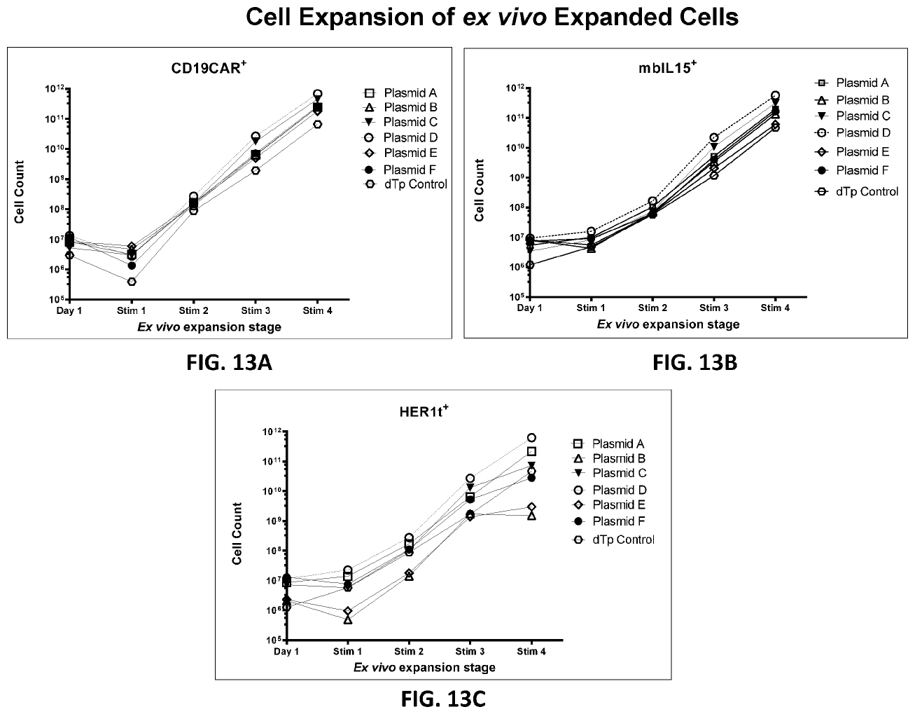

Cells from Donor A were ex vivo expanded with four recursive stimulations on K562-AaPC Clone 9. As shown in FIGS. 4A-4F, 5A-5F, 6A-6F, 7A-7F, 8A-8F, 9A-9F, and 10A-10F, transgene co-expression was assessed on Day 1 and at the end of Stims 1, 2, 3, and 4 via 2-parameter flow plots. For Day 1, it was observed that dTp Control-modified T cells exhibited the standard heterogenous transgene expression pattern of a small population of CD19CAR⁺HER1t (5%)/CD19CAR⁺mbIL15⁺ (3%) T cells (FIGS. 4D and 4E, respectively) and a ˜2-fold larger population of CD19CAR⁺HER1t^{neg} (9%) T cells (FIG. 4D). Low-level co-expression of 2% for HER1t and mbIL15 was observed (FIG. 4F). The Plasmid A-modified T cells showed co-expression of CD19CAR and HER1t (17%) as well as 8% HER1t⁺mbIL15⁺ T cells (FIGS. 5D and 5F, respectively) with 12% HER1t⁺mbIL15^{neg} (FIG. 5F), and 8% CD19CAR⁺mbIL15⁺ subset. Plasmid B-modified T cells showed poor HER1t expression (21% CD19CAR⁺HER1t^{neg} and 5% CD19CAR⁺HER1t) (FIGS. 6D and 11C), though they had improved expression of mbIL15 (16% CD19CAR⁺mbIL15⁺) (FIGS. 6E and 11B). Plasmid C-modified T cells showed co-expression of CD19CAR and HER1t (13%) (FIG. 7D) but lower HER1t⁺mbIL15⁺ (FIG. 7F) compared to Plasmid A. Plasmid D-modified T cells showed a 27% CD19CAR⁺HER1t⁺ subset and an 8% CD19CAR⁺HER1t^{neg} subset (FIG. 8D). The mbIL15 also showed good expression, with 18% CD19CAR⁺mbIL15⁺ cells detected (FIG. 8E). There was some heterogeneity in HER1t and mbIL15 co-expression with HER1t expression over mbIL15 (13% HER1t⁺mbIL15^{neg} and 16% HER1t⁺mbIL15⁺) (FIG. 8F). As with Plasmid B-modified T cells, Plasmid E-modified T cells showed poor HER1t expression (20% CD19CAR⁺HER1t^{neg} and 5% CD19CAR⁺HER1t⁺) (FIGS. 9D and 11C) but had improved expression of mbIL15 (16% CD19CAR⁺mbIL15⁺) (FIGS. 9E and 11B). Similar to Plasmid C-modified T cells, Plasmid F-modified T cells showed co-expression of CD19CAR and HER1t (25%) (FIG. 10D) and 13% HER1t⁺mbIL15⁺ expression (FIG. 10F). Overall, transgene expression patterns on Day 1 in RPM T cells showed most favorable CD19CAR/HER1t co-expression and total mbIL15 expression in Plasmids A and Plasmid D, followed by Plasmid F.

Stim 4 ex vivo expanded T cells yielded >90% CAR expression in all treatments. The greatest mbIL15 expression was observed in Plasmid A and Plasmid D-modified cells (66% and 72%, respectively) (FIGS. 5C and 8C, respectively, and FIG. 11B) compared to 63% on dTp Control-modified T cells (FIG. 4C). Additionally, the greatest total HER1t expression was only observed in Plasmid A and Plasmid D-modified cells (95% each) (FIGS. 5B, 8B, and 11C), which surpassed the dTp Control (78%) (FIGS. 4B and 11C) and was decisively better than the other sTp variants, which all showed expression below 44% (FIGS. 6B, 7B, 9B, 10B, and 11C).

Notably, not only was Stim 4 ex vivo expanded CD19CAR⁺HER1t⁺ co-expression highest in Plasmid A-modified cells (94%) and Plasmid D-modified cells (94%), but additionally the CD19CAR and HER1t expression levels were highly associated, leading to a uniform CAR⁺HER1t⁺ expression pattern (FIGS. 5D and 8D). This contrasted with the diffuse CAR⁺HER1t⁺ population exhibited by dTp Control-modified cells (FIG. 4D). Likewise, the CAR⁺mbIL15⁺ expression pattern in Plasmid A-modified cells and Plasmid D-modified cells was highly associated and uniform, in contrast to the diffuse pattern observed in dTp Control-modified cells (FIGS. 5E, 8E, and 4E, respectively).

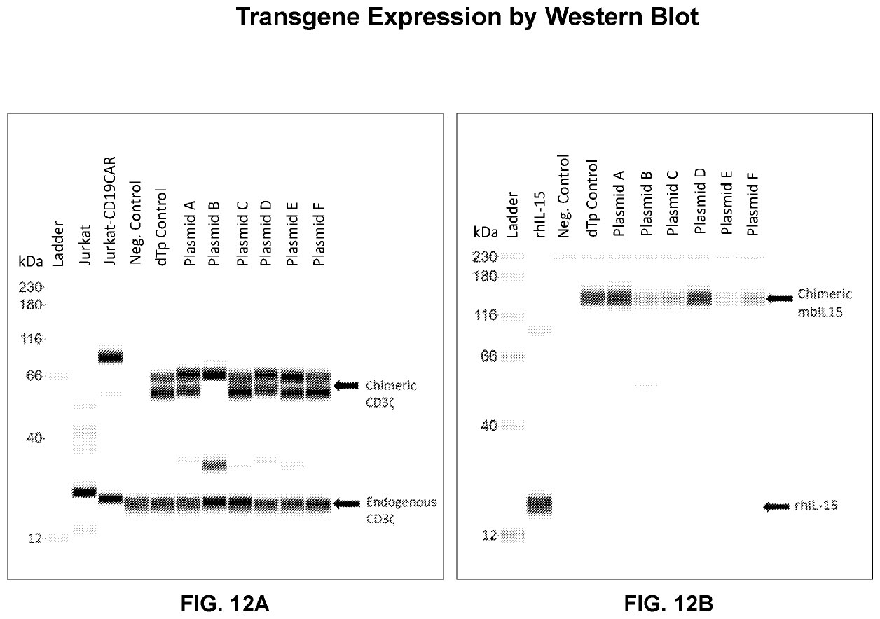

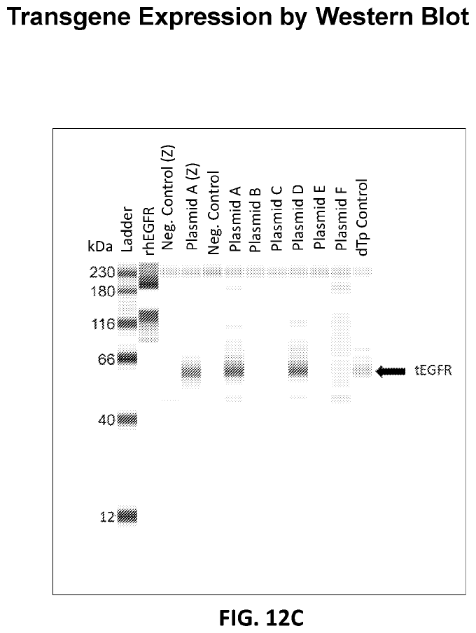

Confirmation of protein expression was performed on cell lysates from the Stim 4 ex vivo expanded CD19CAR-mbIL15-HER1t T cells by Western blot. Cells were prepared and underwent protein transfer and probing with anti-human CD247 for detection of CD19CAR protein (FIG. 12A), anti-human IL-15 for detection of mbIL15 (FIG. 12B), and anti-human EGFR for detection of HER1t (FIG. 12C) on the modified cells. Secondary HIRP antibody, with appropriate specificity, was used for detection. Jurkat cells expressing CD19CAR were used as a positive control for CD19CAR detection. Recombinant human IL-15 (rhIL-15) and No DNA (Negative Control) T cells served as positive and negative controls, respectively, for detection of the chimeric IL-15. Recombinant human EGFR was used as a positive control for detection of truncated EGFR (tEGFR).

CD19CAR expression was confirmed by Western blot analysis of CD3ζ using anti-CD3ζ antibody. As shown in FIG. 12A, detection of the endogenous CD3ζ band (˜16 kDa) was observed in all T cell samples. The ˜60 kDa band/bands represent the chimeric CD3ζ protein of the CD19-specific CAR. Detection of control rhIL-15 occurred at the expected ˜15 kDa with the chimeric mbIL15 bands observed at ˜140 kDa (FIG. 12B). HER1t (truncated EGFR, tEGFR) expression was observed in modified T cells at ˜50 kDa, with the full-length EGFR detected at ˜190 kDa in rhEGFR (FIG. 12C).

Numeric expansion was assessed in Donor A for all of the transposon variants. T-cell enriched starting product was thawed and rested overnight. Cells were electroporated using Amaxa Nucleofector solution along with dTp Control and compared with Plasmids A-F for Donor A. The cells were stimulated the next day with γ-irradiated (100 Gy) K562-AaPC Clone #9. Additional recursive stimulations (Stims) occurred every 7-10 days. The T cells were enumerated on Day 1 and at the end of each Stim and viable cells counted based on AOPI exclusion using a Cellometer automated cell counter. Overall, all cultures achieved numeric expansion. CD19CAR-specific expansion was ˜0.5-1 log greater than dTp Control for all of the sTp variants (FIG. 13A). The mbIL15-specific expansion was ˜0.5-1 log greater than dTp Control for all of the sTp variants, except for Plasmid E, which had comparable expansion to dTp Control (FIG. 13B). HER1t-specific expansion was variable: Plasmid B and Plasmid E showed lowest expansion of HER1t+ T cells, Plasmid C and Plasmid F showed comparable expansion to dTp Control, and Plasmid A and Plasmid D demonstrated the greatest numeric expansion (FIG. 13C).

This Example demonstrates that Plasmid A and Plasmid D best meet the primary objectives of genetic modification of the T cells with CD19CAR-mbIL15-HER1t tricistronic transposons plasmids, namely, redirection of antigen specificity toward CD19CAR, HER1t co-expression to enable conditional elimination of mbIL15⁺ cells, acceptable total expression of mbIL15, and efficient and uniform co-expression of all three transgenes. With these criteria considered, the Plasmid A and Plasmid D single transposon constructs, having element order CD19CAR-F2A-mbIL15-T2A-HER1t, best satisfy the desired criteria.

### 6.2.3 Functional Characterization of CAR-T Cells Co-Expressing CD19CAR, mbIL15, and HER1t

Assays were performed to evaluate functional characteristics of CAR-T cells co-expressing CD19CAR, mbIL15, and HER1t.

6.2.3.1 Specificity of CD19-Directed Cytotoxicity and Cytokine Expression with CAR-T Cells Expressing CD19CAR, mbIL15, and HER1t

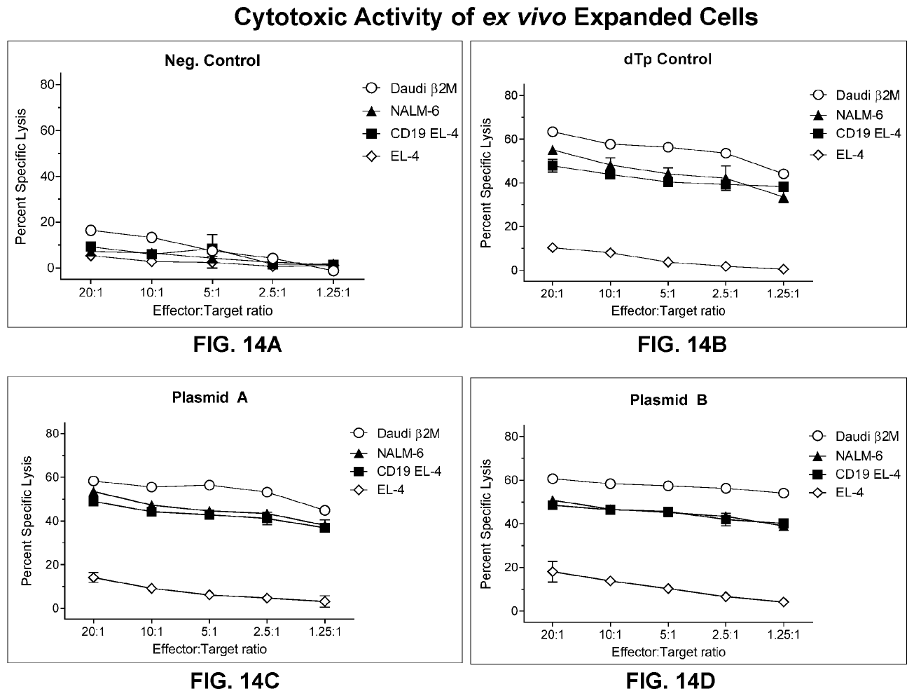

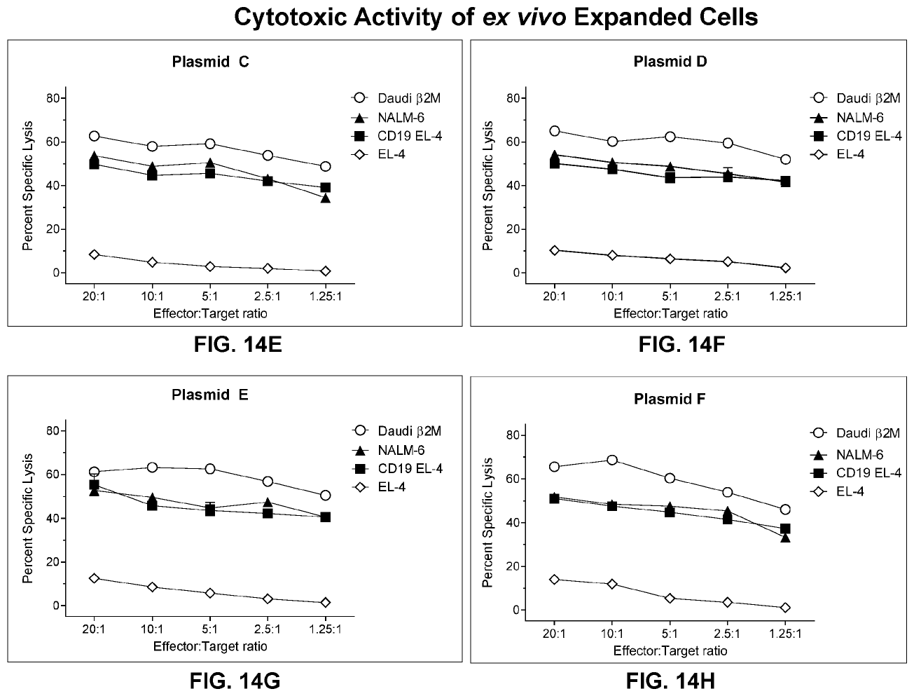

Cytotoxicity assays were performed to demonstrate the specificity of targeting the CD19⁺ tumor cells. The specificity for CD19⁺ tumor targets was demonstrated by comparing the activity of CD19-expressing tumor cell lines (NALM-6, Daudi β2M, and engineered CD19 EL-4) and the CD19^{neg} parental EL-4 cell line. The cytotoxicity assay tested E:T ratios ranging from 20:1 to 1.25:1 in a standard 4-hour chromium release assay. The CD19CAR-mbIL15-HER1t T cells transfected with Plasmids A-F demonstrated specific lysis of all CD19⁺ targets of ˜50% at the lowest E:T, and it was comparable to the dTp Control cells (FIGS. 14A-14H). Lysis of CD19^{neg} targets was minimal at low E:T. In summary, modification of T cells with Plasmids A-F, resulting in co-expression of transgenes from a single transposon, did not alter cytotoxic function of CD19CAR-mbIL15-HER1t T cells relative to cells modified with dTp Control.

### 6.2.3.2 HER1t-Mediated Depletion of CD19CAR-mbIL15-HER1t T Cells via ADCC

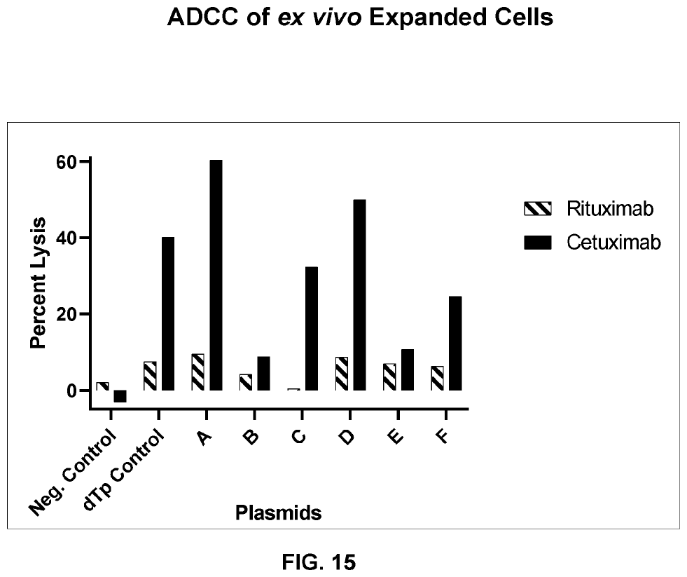

HER1t was included in the tricistronic design in order to co-express HER1t with mbIL15 and CD19CAR on the cell surface and to provide a mechanism to selectively deplete infused mbIL15⁺ T cells. HER1t-expressing cells can be eliminated by administration of cetuximab, a clinically available monoclonal antibody that binds to HER1t and mediates antibody-dependent cellular cytotoxicity (ADCC). In vitro assessment was performed to confirm the ability of cetuximab to induce ADCC against the ex vivo expanded CD19CAR-mbIL15-HER1t T cells. The genetically modified T cells served as targets in this assay, which was a standard 4-hour chromium release assay in the presence of cetuximab (anti-HER1t antibody) or rituximab (anti-CD20 antibody; negative control) using Fc receptor-expressing NK cells as effectors. As shown in FIG. 15, addition of cetuximab resulted in depletion of target HER1t-modified T cells that were generated with dTp Control, Plasmid A, Plasmid C, Plasmid D, and Plasmid F. CD19CAR-mbIL15-HER1t T cells generated with Plasmid A and Plasmid D showed the highest level of selective depletion (˜60% and ˜50%, respectively). Cetuximab failed to show lysis of Negative Control (HER1t^{neg}) cells, confirming the HER1t-specific mechanism of action.

These data support the use of cetuximab to deplete CD19CAR-mbIL15-HER1t T cells generated using Plasmid A and Plasmid D, in the event of adverse clinical effects that require a depletion strategy.

6.2.3.3 Stable Integration of CD19CAR, mbIL15, and HER1t Transgenes after Ex Vivo Expansion of SB System-Modified CD19CAR-mbIL15-HER1t T Cells

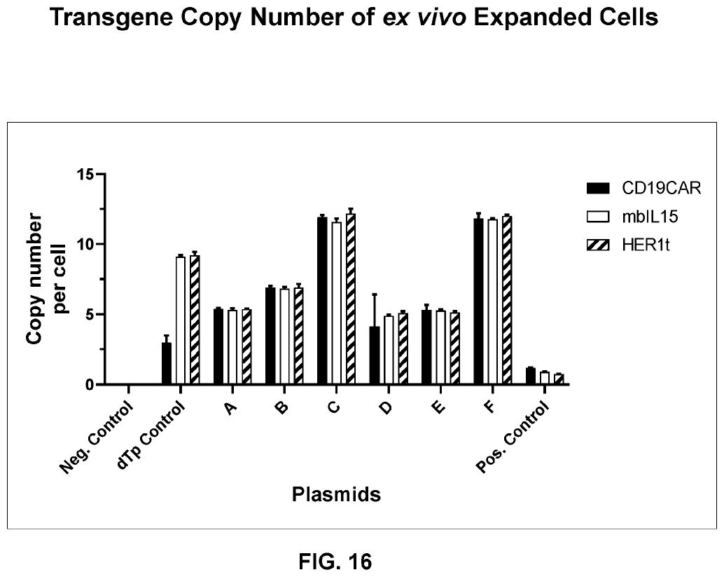

Copy numbers of the CD19CAR, mbIL15, and HER1t transgenes in ex vivo expanded CD19CAR-mbIL15-HER1t T cells was examined using ddPCR and primer/probe sets specific to CD19CAR, mbIL15, and HER1t. Results are shown in FIG. 16. The copy number was normalized to the human reference gene EIF2C1, which is known to be present in 2 copies/cell. It was observed that dTp Control had different integration levels between CD19CAR and each of mbIL15 and HER1t (˜2.5 copies per cell for CD19CAR and ˜8 copies per cell for mbIL15 and HER1t). Plasmid C- and Plasmid F-generated T cells exhibited >10 transgene copies per cell. Plasmid A-, Plasmid D-, and Plasmid E-generated cells exhibited an average copy number per cell of ˜5, while Plasmid B-generated cells exhibited an average copy number per cell of ˜7. The positive control T cells (propagated on AaPC) showed transgene insertion at an average of ˜1 copy per cell.

In summary, based on analysis of the number of transposon insertions into the primary human T cell genome of cells manufactured under RPM, such cells undergo stable integration of transgenes, and the Plasmid A-, Plasmid D-, and Plasmid E-generated CD19-mbIL15-HER1t T cells demonstrated the most favorable (low) integration values compared to the other sTp variants as well as dTp Control. In addition, all sTp variant-generated T cells demonstrated much more consistent integration values across all three transgenes than dTp Control-generated T cells.

### 6.3 Example 3: Multi-Donor Assessment of Candidate Tricistronic sTp SB DNA Plasmids

Evaluation of sTp plasmids in Example 2 identified Plasmid A and Plasmid D as candidates to proceed with further testing, based on: (i) their favorable co-expression of transgenes at Day 1 and Stim 4, as detected by flow cytometry; (ii) overall transgene expression Stim 4, as detected by Western blot; (iii) acceptable transgene-specific numeric expansion; (iv) unaffected cytotoxicity; and (v) favorable selective elimination. This Example describes continued evaluation of the candidate Plasmid A in additional donors. Plasmid D data for a single donor are included for reference and are comparable to Plasmid A, as the transgene order is the same. 6.3.1 Materials and Methods

Materials and Methods were as described in Section 6.2.1, except as indicated.

### 6.3.2 Genetic Modification, Expression Characterization, and Expansion of CAR-T Cells Co-Expressing CD19CAR, mbIL15, and HER1t

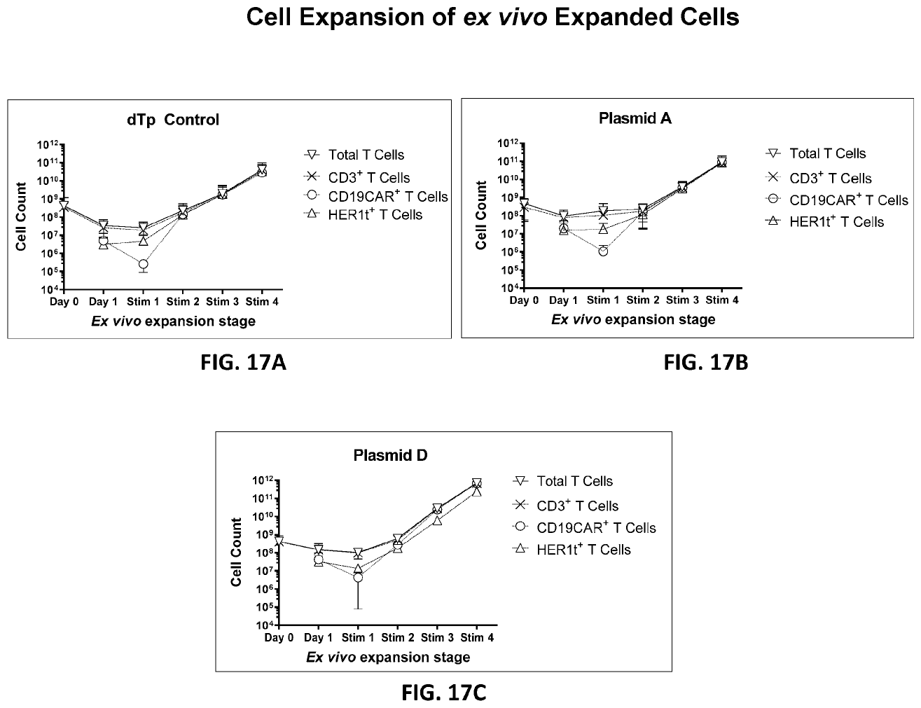

Similar to Example 2, T cell-enriched products were electroporated with dTp Control, Plasmid A, and Plasmid D and ex vivo expanded via co-culture on irradiated Clone 9 AaPCs to assess RPM T cells (Day 1) and Stim 4 propagated cells. The growth kinetics and transgene-specific expansion of T cells generated using dTp Control (n=10, Day 1; n=5, Stim 1; n=7, Stim 2; n=6, Stim 3; n=5, Stim 4; FIG. 17A), Plasmid A (n=8, Day 1; n=3, Stim 1; n=4, Stim 2; n=4, Stim 3; n=4, Stim 4; FIG. 17B), and Plasmid D (n=7, Day 1; n=2, Stim 1; n=2, Stim 2; n=1, Stim 3; n=1, Stim 4; FIG. 17C) were comparable across treatments at ˜10¹¹ cells.

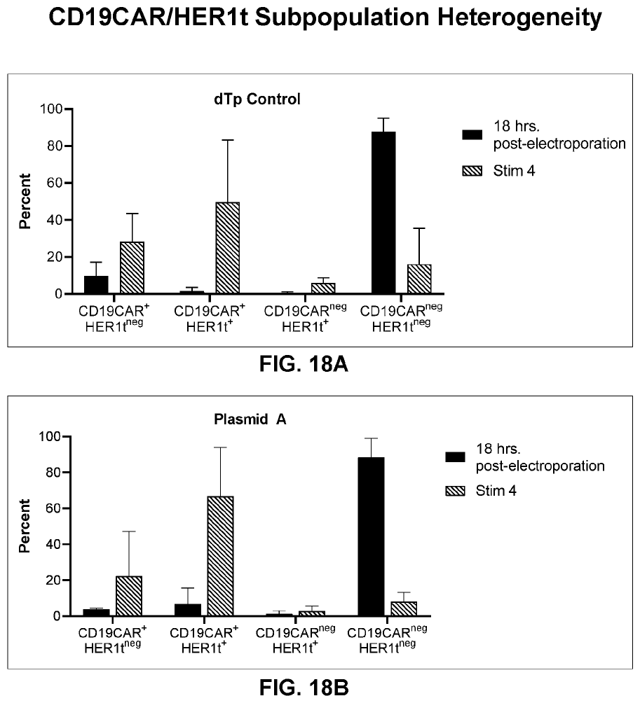

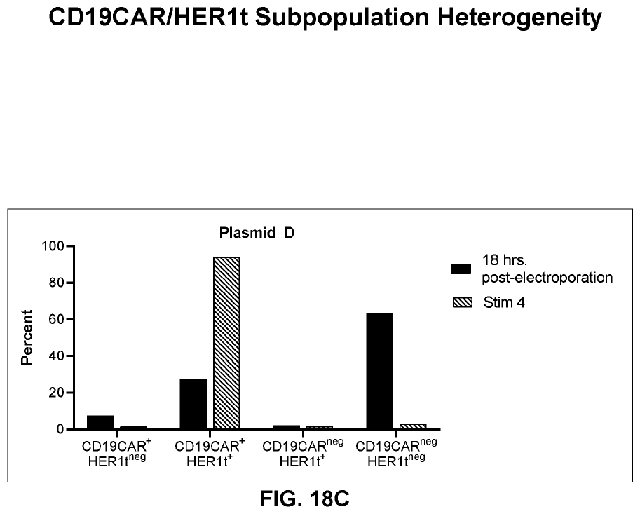

Similarly, T cell-enriched products were electroporated with dTp Control (n=6, Day 1; n=3, Stim 4) (FIG. 18A), Plasmid A (n=3, Day 1; n=3, Stim 4) (FIG. 18B), and Plasmid D (n=1, Day 1; n=1, Stim 4) (FIG. 18C) and were ex vivo expanded via co-culture on irradiated Clone 9 AaPCs. Evaluation of CD19CAR/HER1t co-expression in Day 1 RPM T cells showed that cells modified with Plasmid A or Plasmid D had higher frequencies of the target CD19CAR⁺HER1t T-cell population compared to dTp Control-modified cells (7%±9%, 27%±0%, 2%±2%, respectively). At the end of Stim 4, both Plasmid A and Plasmid D-modified cells had higher frequencies of the target CD19CAR⁺HER1t⁺ T-cell population compared to dTp Control-modified cells (67%±27%, 94%±0%, 50%±34%, respectively). The additional donor evaluations for dTp Control and Plasmid A support the observations of Example 2 that transgene co-expression is improved with the use of Plasmid A and Plasmid D (which share the same transgene order), compared to dTp Control.

### 6.3.3 Functional Characterization of CAR-T Cells Co-Expressing CD19CAR, mbIL15, and HER1t

Assays were performed to evaluate functional characteristics of CAR-T cells co-expressing CD19CAR, mbIL15, and HER1t.

6.3.3.1 Specificity of CD19-Directed Cytotoxicity and Cytokine Expression with CAR-T Cells Expressing CD19CAR, mbIL15, and HER1t

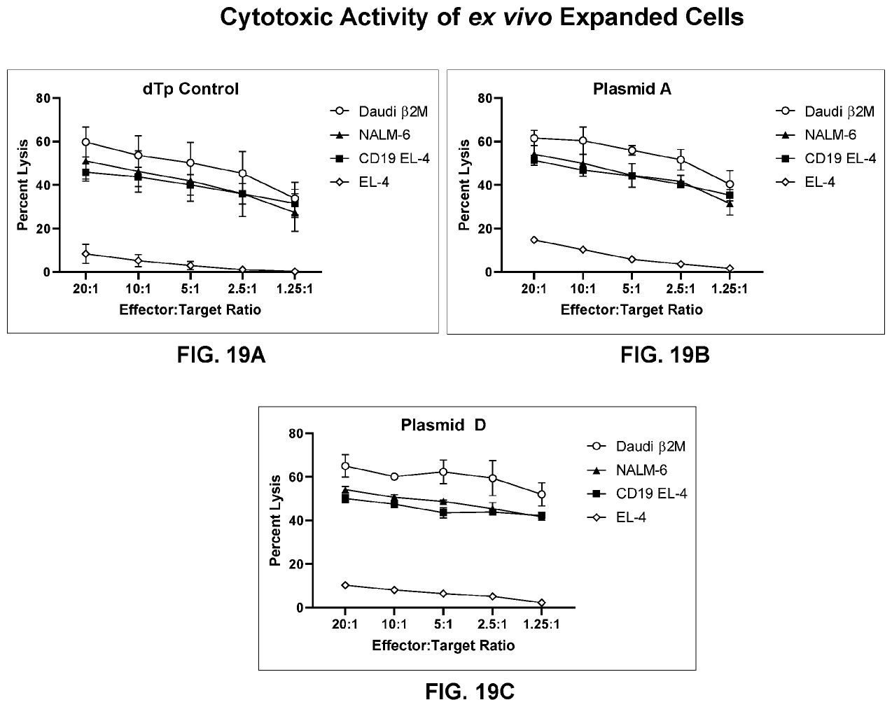

Similar to Section 6.2.3.1, cytotoxicity was evaluated in additional donors for dTp Control (n=6), Plasmid A (n=4), and Plasmid D (n=1)-generated and ex vivo expanded CD19CAR-mbIL15-HER1t T cells in a standard 4-hour chromium release assay. Cytotoxicity of CD19⁺ target cell lines was comparable across the three conditions, with specific lysis observed at ˜40% for dTp Control (FIG. 19A) and Plasmid A (FIG. 19B) and ˜50% for Plasmid D (FIG. 19C) at the 1.25:1 E:T ratio. CD19^{neg} cell lysis was negligible. In summary, these data show that Plasmid A and Plasmid D do not alter cytotoxic ability and further support the observations in Section 6.2.3.1.

### 6.3.3.2 HER1t-Mediated Depletion of CD19CAR-mbIL15-HER1t T Cells via ADCC

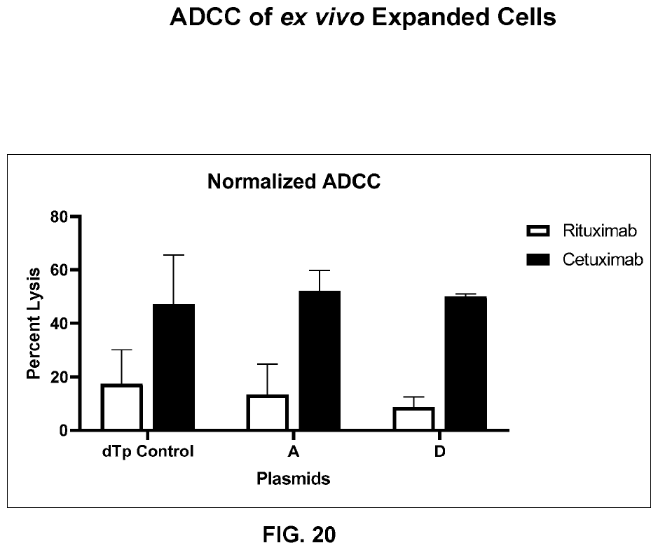

Similar to Section 6.2.3.2, selective elimination of CD19CAR-mbIL15-HER1t T cells via ADCC was evaluated in additional donors for dTp Control (n=6), Plasmid A (n=4), and Plasmid D (n=1)-generated and ex vivo expanded CD19CAR-mbIL15-HER1t T cells. For all three conditions, cetuximab treatment resulted in ˜50% lysis of target CD19CAR-mbIL15-HER1t T cells by effector NK cells (FIG. 20). These data provide additional support to data in Section 6.2.3.2, showing that Plasmid A and Plasmid D generate CD19CAR-mbIL15-HER1t T cells that may be selectively depleted via ADCC using cetuximab.

6.3.3.3 Stable Integration of CD19CAR, mbIL15, and HER1t Transgenes after Ex Vivo Expansion of SB System-Modified CD19CAR-mbIL15-HER1t T Cells

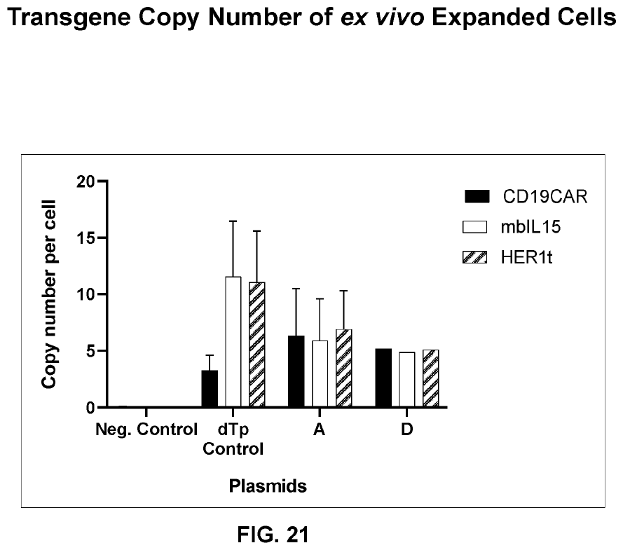

Similar to Section 6.2.3.3, ex vivo expanded Stim 4 CD19CAR-mbIL15-HER1t T cells generated using dTp Control (n=7), Plasmid A (n=5), or Plasmid D (n=1) were evaluated for transgene copy number using ddPCR and primer/probe sets specific to CD19CAR, mbIL15, and HER1t as shown in FIG. 21. The dTp Control-generated cells showed an average of ˜3 copies/cell for CD19CAR, ˜11 copies/cell for mbIL15, and ˜11 copies/cell for HER1t. Plasmid A-generated cells had an average copy/cell of ˜6 for the three transgenes, and Plasmid D-generated cells had an average copy/cell of ˜5 for the three transgenes.

In summary, these data corroborate the observations from Section 6.2.3.3, demonstrating that Plasmid A and Plasmid D each generated CD19CAR-mbIL15-HER1t T cells that had nearly identical integration numbers for all three transgenes, whereas dTp Control generated cells with substantially different integration numbers between CD19CAR on the one hand and mbIL15 and HER1t on the other, with mbIL15 and HER1t integrating at a substantially higher level.

### 6.4 Example 4: Generation and Evaluation In Vivo of RPM T Cells Co-Expressing CD19CAR, mbIL15, and HER1t

This Example describes the generation and evaluation in vivo of RPM T cells co-expressing CD19CAR, mbIL15, and HER1t from dTp Control or Plasmid A.

### 6.4.1 Materials and Methods

### 6.4.1.1 Cell Lines

The human tumor cell line, NALM-6/fLUC, was generated at MD Anderson Cancer Center (MDACC; Houston, TX) from the parental pre-B cell CD19⁺ NALM-6 cell line (American Type Culture Collection (ATCC; Manassas, VA)) (or, e.g., as described in Singh et al., Cancer Res. 2011; 71(10):3516-3527, the contents of which are incorporated by reference in their entirety herein). These tumor cells co-express firefly luciferase (fLUC) for non-invasive bioluminescent imaging (BLI) and enhanced green fluorescent protein (EGFP) for fluorescent imaging. Cells were routinely cultured in RPMI 1640 or Hyclone: R10 media containing 10% FBS (Hyclone/GE Healthcare, Logan, UT) and 1% Glutamax-100 (ThermoFisher Scientific, Waltham, MA). Cells were cultured under normal conditions of 37° C. with 5% CO₂. Cells were tested and found to be negative for mycoplasma. Identity of the cell line was confirmed by short tandem repeat DNA fingerprinting.

### 6.4.1.2 Normal Donor Human T Cells

Peripheral blood or leukapheresis product was obtained from normal donors (Key Biologics, Memphis, TN). Multiple collections from the same donor were obtained. The apheresis products were divided to allow for testing two starting cell products for the manufacture of RPM T cells.

One portion of the apheresis was processed for isolating PBMC using Sepax S-100 Cell Separation System (BioSafe, Newark, DE). Live/dead cells were enumerated on a Cellometer instrument (Nexcelom Bioscience; Lawrence, MA). Isolated PBMC were cryopreserved in CryoStor CS10 (Biolife Solutions; Bothell, WA; or equivalent) and stored in the vapor phase of a liquid nitrogen tank.

Preparation of the T cell-enriched starting product (for the CD3 treatment groups), the other portion of apheresis product, was diluted using CliniMACS® PBS/EDTA buffer with 0.5% (v/v) HSA, and a platelet depletion step was performed via centrifugation at 400×g for 10 minutes at room temperature (RT) with subsequent resuspension in the same buffer. Both CD4- and CD8-specific CliniMACS® microbeads were incubated with cells for 30 minutes at RT under mixing conditions and underwent paramagnetic selection on the CliniMACS Plus to enrich the starting product for T cells. Live/dead cells were enumerated on a Cellometer instrument (Nexcelom Bioscience; Lawrence, MA). Isolated T cells were cryopreserved in CryoStor CS10 and stored in the vapor phase of a liquid nitrogen tank.

### 6.4.1.3 Generation of RPM CD19CAR-mbIL15-HER1t T Cells Using SB System

To generate the test article groups of RPM CD19CAR-mbIL15-HER1t T cells assessed in this study, either PBMC or T cell-enriched starting product was used, and gene transfer used either dTp Control or Plasmid A, each as described in Example 1, with the Nucleofector™ 2b device (Lonza; Basel, Switzerland). Details for the generation of each test article are as follows:

Mock PBMC: The day before electroporation, cryopreserved PBMC were thawed in RPMI 1640 media (Phenol Red free media (Hyclone), 10% FBS, and 1% Glutamax-100 (R10)), washed and resuspended with R10, and placed in a 37° C./5% CO₂ incubator overnight. Rested cells were harvested, spun down, and resuspended in Nucleofector buffer (Human T Cell Nucleofector Kit; Lonza) without any transposon or transposase DNA plasmids.

Mock CD3: Cryopreserved CD3-enriched cells were thawed and processed as described above for Mock PBMC.

dTp Control (P, 5e6): Cryopreserved PBMC were thawed and rested one hour. Rested cells were harvested, spun down, and resuspended in Nucleofector buffer containing dTp Control and Plasmid TA (encoding the SB11 transposase, as described in Example 1) at a final transposon:transposase ratio of 3:1 (Table 17). “(P, 5e6)” refers to 5×10⁶ PBMC-derived cells infused.

Plasmid A (P, 5e6): Cryopreserved PBMC were thawed and rested one hour. Rested cells were harvested and resuspended in Nucleofector buffer containing Plasmid A and Plasmid TA at a final transposon:transposase ratio of 3:1 (Table 17). As with dTp Control, “(P, 5e6)” refers to 5×10⁶ PBMC-derived cells infused.

Plasmid A (T, 1e6) and Plasmid A (T, 0.5e6): Cryopreserved CD3 cells were thawed and processed as described above for Mock CD3. Rested cells were harvested and resuspended in Nucleofector buffer containing Plasmid A and Plasmid TA at a final transposon:transposase ratio of 3:1 (Table 17). “(T, 1e6)” refers to 1×10⁶ CD19CAR⁺CD3⁺ cells infused, and “(T, 0.5e6)” refers to 0.5×10⁶ CD19CAR⁺CD3⁺ cells infused.

For the PBMC-derived RPM cells, immediately following electro-transfer, the contents from each cuvette were resuspended and transferred to R10 media and rested in a 37° C./5% CO₂ incubator for 1-2 hours. Subsequently, a whole medium exchange was performed with R10 medium, and the cells were placed overnight in a 37° C./5% CO₂ incubator. Within 24 hours post-electro-transfer, the cells were harvested from culture and sampled by flow cytometry to determine cell surface expression of CD19CAR, mbIL15, and HER1t, as well as other T-cell markers, e.g., to characterize T cell memory subsets. To formulate for injection into mice, the desired cell number for each test article was resuspended in Plasmalyte A to achieve a 300 μL injection volume per mouse.

For the T cell-derived RPM cells, immediately following electro-transfer, the contents from each cuvette were resuspended and transferred to R10 media containing DNase for a 1-2 hour incubation in a 37° C./5% CO₂ incubator. Subsequently, a whole medium exchange was performed with R10 media, and the cells were placed overnight in a 37° C./5% CO₂ incubator. Within 24 hours post-electro-transfer, the cells were harvested from culture and sampled by flow cytometry to determine cell surface expression of CD19CAR, mbIL15, and HER1t, as well as other T-cell markers, e.g., to characterize T cell memory subsets. Additionally, dead cells and debris were removed from harvested cells, and the cells were enriched for viable cells. To formulate for injection into mice, the desired cell number for each test article was resuspended in Plasmalyte A to achieve a 300 μL injection volume per mouse.

TABLE 17
Test Articles.

| | Starting | | Total Cells | CD19CAR⁺CD3⁺ | |
|---|---|---|---|---|---|
| Test Article | Cell | | Infused | Cells Infused | Animal |
| (Cells) | Product | Transposon(s) | (×10⁶) | (×10⁶) | Groups |
| Tumor Only | N/A | N/A | N/A | N/A | A |
| Mock PBMC | PBMC | N/A | 5.00 | N/A | B |
| Mock CD3 | T cell- | N/A | 3.94 | N/A | C |
| | enriched | | | | |
| dTp Control | PBMC | dTp Control (as | 5.00 | N/A | D |
| (P, 5e6) | | described in | | | |
| | | Example 1) | | | |
| Plasmid A | PBMC | Plasmid A (as | 5.00 | N/A | E |
| (P, 5e6) | | described in | | | |
| | | Example 1) | | | |
| Plasmid A | T cell- | Plasmid A (as | 3.94 | 1.00 | F |
| (T, 1e6) | enriched | described in | | | |
| | | Example 1) | | | |
| Plasmid A | T cell- | Plasmid A (as | 1.97 | 0.50 | G |
| (T, 0.5e6) | enriched | described in | | | |
| | | Example 1) | | | |

### 6.4.1.4 Animals

Approximately eight-week-old female NOD/SCID/gamma mice (NOD.Cg-Prkdc^{scid} Il2^{rgtm1Wjl}/SzJ, NSG) were purchased from Jackson Laboratory (Bar Harbor, MVIE). NSG mice lack both B and T lymphocytes and NIK cells (as described, e.g., in Ali et al., PLoS ONE. 2012; 7(8):e44219, the contents of which are incorporated by reference in their entirety herein). This strain has superior engraftment of human hematopoietic cells, as well as ALL with ability to detect blasts in the peripheral blood (as described, e.g., in Agliano et al., Int J Cancer. 2008; 123:2222-2227, and Santos et al., Nat Med. 2009; 15(3):338-344, the contents of each of which are incorporated by reference in their entirety herein). The test articles were manufactured and the study was performed at MIDACC and in compliance with its Institutional Animal Care and Use Committee (IACUC) and the Guidelines for the Care and Use of Laboratory Animals (Eighth Edition, NRC, 2011, published by the National Academy Press, the contents of which are incorporated by reference in their entirety herein) and the Public Health Service Policy on Humane Care and Use of Laboratory Animals, Office of Laboratory Animal Welfare, Department of Health and Human Services (OLAW/NI, 2002, the contents of which are incorporated by reference in their entirety herein). Previous reports have shown that 6-12-week-old NSG mice engraft efficiently with 10⁷ human PBMC in the absence of host pre-conditioning and developed xGvHID consistently with accelerated weight loss and significantly faster disease development (median survival time (MST)=40 days) (as described, e.g., in Ali et al., PLoS ONE. 2012; 7(8):e44219, the contents of which are incorporated by reference in their entirety herein).

### 6.4.1.5 Study Design

On Day 1, NSG mice were injected via the tail vein with 1.5×10⁴ viable NALM-6/fLUC cells in 0.2 mL of sterile PBS. On Day 6, animals underwent bioluminescence imaging (BLI) to detect the presence of tumor. Based on these data, the animals were stratified into treatment groups, which all observed a similar mean tumor flux signal. Animals received test article treatment on Day 7 as shown in Table 18, with total cell numbers in control groups B and C matching the total cell numbers of the corresponding genetically modified T cell treatment group.

TABLE 18
Study Design of Animal Experiments.

| | | Administration | | | | Administration | | |
|---|---|---|---|---|---|---|---|---|
| | | Route (vol., conc.) | | Injection | Test | Route (vol., conc.) | | Injection |
| Groups | N | μL/mouse | cells/mL | Day | Article | μL/mouse | cells/mL | Day |
| A | 10 | 200 | 7.5 × 10⁴ | 1 | N/A | N/A | N/A | N/A |
| B | 6 | 200 | 7.5 × 10⁴ | 1 | Mock PBMC | 300 | 1.67 × 10⁷ | 7 |
| C | 5 | 200 | 7.5 × 10⁴ | 1 | Mock CD3 | 300 | 1.31 × 10⁷ | 7 |
| D | 10 | 200 | 7.5 × 10⁴ | 1 | dTp Control | 300 | 1.67 × 10⁷ | 7 |
| | | | | | (P, 5e6) | | | |
| E | 9 | 200 | 7.5 × 10⁴ | 1 | Plasmid A | 300 | 1.67 × 10⁷ | 7 |
| | | | | | (P, 5e6) | | | |
| F | 10 | 200 | 7.5 × 10⁴ | 1 | Plasmid A | 300 | 1.31 × 10⁷ | 7 |
| | | | | | (T, 1e6) | | | |
| G | 4 | 200 | 7.5 × 10⁴ | 1 | Plasmid A | 300 | 6.57 × 10⁶ | 7 |
| | | | | | (T, 0.5e6) | | | |

### 6.4.1.6 Methodology for Animal Handling and Imaging

### 6.4.1.6.1 Body Weight Measurements

Animals were weighed two to three times per week for the duration of the study.

### 6.4.1.6.2 In Vivo BLI

BLI is a high-sensitivity, low-noise, non-invasive technique used for visualizing, tracking, and monitoring specific cellular activity in an animal. Longitudinal monitoring of the luminescent signal provides quantitative assessment of tumor burden. NALM 6-derived firefly luciferase (fLUC) was used as the bioluminescence reporter with D-luciferin provided as the substrate. On Days 6, 14, 19, 22, 25, 28, 32, 35, 39, 42, 43, 46, 49, 53, 56, 60, and 62, BLI was performed using Xenogen IVIS Spectrum In Vivo Imaging System (Xenogen, Caliper LifeSciences, Hopkinton, MA). Living Image software (v.4.5; Xenogen, Caliper LifeSciences, Hopkinton, MA) was used to acquire and quantitate the bioluminescence imaging data sets. Ten minutes before the time of imaging, a single subcutaneous (s.q.) injection of 214.5 μg D-luciferin (1.43 mg/mL working stock solution; Caliper) in 150 μL PBS was administered to each mouse. Animals were maintained with 2% isoflurane and positioned within a biocontainment device (as described, e.g., in Gade et al., Cancer Res. 2005; 65(19):9080-9088, the contents of which are incorporated by reference in their entirety herein). Mice were imaged with exposure times as determined by the automated exposure, except for Day 6, on which a 4-minute exposure acquisition was also performed. Ventral images were obtained for each animal and quantified. Total flux values were determined by drawing regions of interest (ROI) of equivalent size over each mouse and presented in photons/s (p/s) (as described, e.g., in Gade et al., Cancer Res. 2005; 65(19):9080-9088, and Cooke et al., Blood. 1996; 8(8):3230-3239, the contents of each of which are incorporated by reference in their entirety herein). “Background” BLI to define mice that have no tumor (i.e., flux≤2× background) is established using NSG mice injected with luciferin, but which do not have NALM-6 (thus no fLUC activity), with capture of ventral images.

### 6.4.1.6.3 Blood Collection

Terminal bleeds were collected by retro-orbital bleeding with collection in sodium heparin-coated tubes. The presence of CAR⁺ T cells and tumor were determined by flow cytometry. Blood was collected from moribund animals as feasible. Samples were incubated in ACK lysing buffer (Thermo-Fisher) to lyse red blood cells, resuspended in PBS and 2% FBS and kept at 4° C. until immunostaining was performed (typically within 4 hours of tissue collection) to assess for the presence of CD19CAR, mbIL15, and HER1t on T cells by flow cytometry.

### 6.4.1.6.4 Clinical Observation and End Points

Hydragel was placed in the cages of animals appearing sick to aid in recovery. Mice were monitored daily for any signs of pain or other discomfort due to treatments. Any indication of animal suffering was documented. Animals experiencing the following indications were humanely euthanized by cervical dislocation, after notifying and obtaining consent from the PI as per IACUC protocol: 1) Failure to eat or drink over a 24- to 48-hour period, resulting in emaciation or dehydration; 2) consistent or rapid body weight loss reaching 20% at any time or 15% maintained for 72 hours compared with the pre-treatment weight of mice or age-matched, vehicle-treated controls; 3) persistent hypothermia; 4) bloodstained or mucopurulent discharge from any orifice; 5) labored respiration, particularly if accompanied by nasal discharge and/or cyanosis; 6) enlarged lymph nodes or spleen; 7) hind-limb paralysis or weakness; 8) significant abdominal distension or where ascites burden exceeds 10% of the bodyweight of age-matched controls; 9) urinary incontinence or diarrhea over a 48-hour period; 10) lack of response to stimuli.

### 6.4.1.6.4.1 Bioanalytical Assays

Peripheral blood (PB), spleen, and BM samples were immunophenotyped and evaluated by flow cytometry for the presence of NALM-6/fLUC tumor cells and genetically modified T cells.

### 6.4.1.6.4.2 Flow Cytometry

Up to 2×10⁶ cells were stained with human-specific (unless otherwise stated) fluorochrome conjugated antibodies. Staining for cell surface markers on samples and corresponding controls first underwent an Fc-receptor blocking step to reduce background staining by incubation with 50% mouse serum (Jackson ImmunoResearch, PA) in FACS buffer (PBS, 2% FBS, 0.1% sodium azide) for 10 minutes at 4° C. Immunostaining was performed by the addition of 100 μl of antibody master mix of combinations of antibodies listed in Table 19 that were diluted in Brilliant Stain Buffer (BD Biosciences). Briefly, CD19CAR expression was detected using Alexa Fluor® (AF) 488 conjugated anti-idiotype antibody specific for the anti-CD19 portion of CD19CAR (clone no. 136.20.1) (as described, e.g., in Jena et al., PLoS. 2013; 8(3):e57838, the contents of which are incorporated by reference in their entirety herein). The CD19CAR anti-idiotype antibody was conjugated to the AF-488 fluorophore by Invitrogen/Thermo Fisher Scientific (Waltham, MA). The HER1t molecule was detected using fluorescently conjugated cetuximab antibody. The fluorescent-conjugated cetuximab reagent was commercially purchased Erbitux that was conjugated to AF-647 by Invitrogen/Thermo Fisher Scientific. The fluorescently conjugated antibodies included: CD8 (Clone RPA-T8), CD3 (Clone SK7), CD45RO (UCHL1), IL-15 (34559), CD45 (Clone HI30), CCR7 (Clone G043H7), CD19CAR ideotype (Clone 136.20.1) and mouse CD45.1 (Clone A20) (Table 19).

TABLE 19
Antibodies.

| Antibody Target | Clone | Fluorophore | Company |
|---|---|---|---|
| CD45 | HI30 | BV-786 | BD Biosciences |
| Mouse CD45.1 | A20 | CF-594 | BD Biosciences |
| CD3 | SK7 | PE-Cy7 | BD Biosciences |
| CD8 | RPA-T8 | AF-700 | BD Biosciences |
| CD19CAR | 136.20.1 | AF-488 | Invitrogen |
| IL-15 | 34559 | PE | R&D Systems |
| CD45RO | UCHL1 | Various | BD Biosciences |
| CCR7 | G043H7 | BV-421 | BioLegend |
| CD95 | DX2 | BV-711 | BD Biosciences |
| HER1t | C225 | AF-647 | Invitrogen |

The master mix containing combinations of the antibodies in Table 19 were added in a sequential manner (CD19CAR, mbIL15, followed by the remaining antibody cocktail) and incubated up to 30 minutes at each addition at 4° C. Cells were washed with FACS buffer and then incubated with fixable viability stain-620 viability dye (1:1000 in PBS; BD Biosciences) for 10 minutes at 4° C. followed by washing with FACS buffer. Data were acquired using an LSR Fortessa (BD Biosciences) with FACSDiva software (v.8.0.1, BD Biosciences) and analyzed with FlowJo software (version 10.4.2; TreeStar, Ashland, OR).

### 6.4.1.6.5 Statistical Analysis

Statistical tests are stated with the reporting of each statistic. Post-hoc analysis was performed to compare differences between treatment groups and is reported with each statistical result. Error is reported as standard deviation (SD). GraphPad Prism (version 8) software was used to perform statistical analyses. P<0.05 was considered statistically significant. Specific handling of the total flux values for statistical analysis involved log transforming the flux values to address heteroscedasticity prior to significance testing.

### 6.4.2 Generation and Evaluation of RPM T Cells Co-Expressing CD19CAR, mbIL15, and HER1t In Vivo

6.4.2.1 Genetic Modification of T Cells with SB System to Manufacture RPM CD19CAR-mbIL15-HER1t T Cells

On cell process day 1, generation of PBMC-derived RPM T cells began with a total 3.68×10⁹ PBMC that were rested for 1 hour and electroporated. 1.12×10⁹ PBMC per group were used to manufacture test articles dTp Control (P, 5e6) and Plasmid A (P, 5e6) derived from PBMC, as described in Table 17. On Day 2 (approximately 18 hours after electro-transfer), 1.25×10⁸ to 1.29×10⁸ viable cells were recovered.

For the T-cell-derived RPM T cell study arm, 3.00×10⁹ enriched T cells were thawed, with 1.70×10⁹ cells recovered after overnight rest. 1.26×10⁹ cells were used for electro-transfer to manufacture Plasmid A (T, 1e6) and Plasmid A (T, 0.5e6). On Day 3 (approximately 18 hours after electro-transfer) 4.23×10⁸ viable cells were recovered.

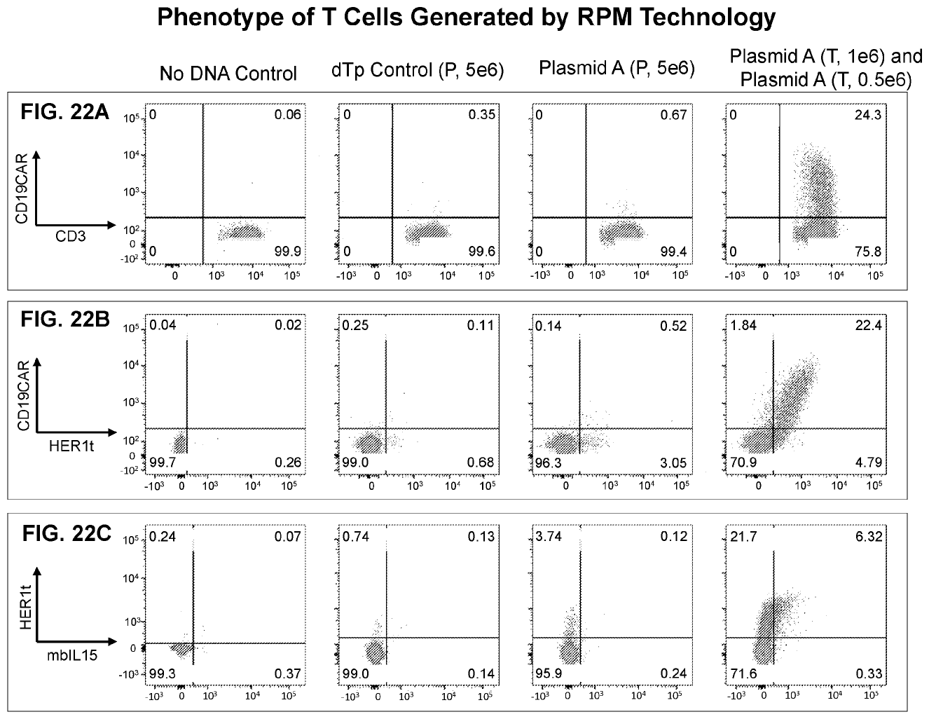

Approximately eighteen hours after electro-transfer, T cells were assessed for transgene expression by flow cytometry, as gated on singlet/live cells/CD3⁺ events (FIGS. 22A-22C). Because low transgene expression was detected for the PBMC-derived test articles, mice dosages were set to 5×10⁶ total viable cells rather than 1×10⁶ CAR⁺ cells.

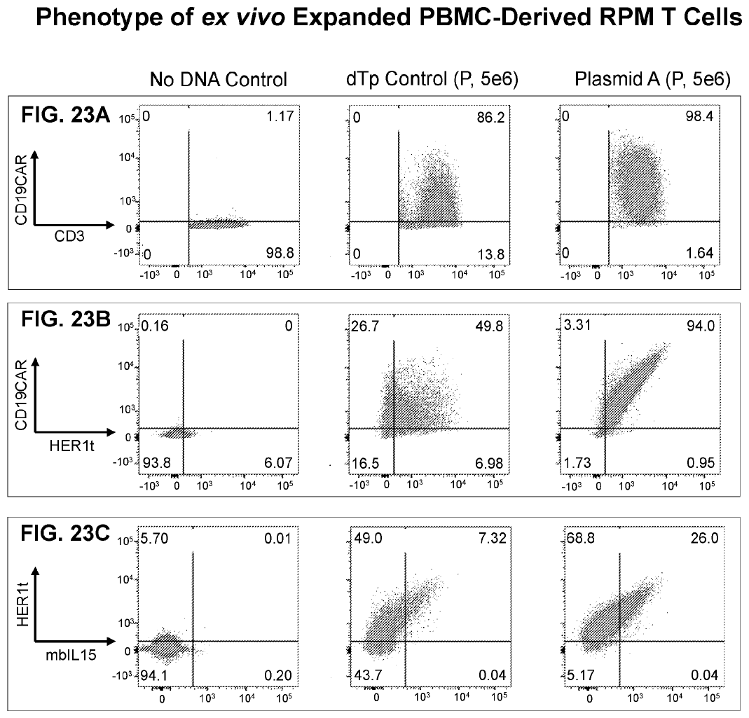

Subsequently, the remaining PBMC-derived test articles were ex vivo expanded with three recursive stimulations on activating and propagating cells (AaPC) and supplemented with IL-21 (30 ng/mL) to confirm gene transfer. These propagated cells were assessed for whether expected antigen-specific outgrowth of transgene positive T cells would occur. Despite <1% CAR⁺, <1% mbIL15⁺, and <4% HER1t expression detected at 18-hours post-electroporation, these RPM T cells showed observable and high transgene expression after numeric expansion (FIGS. 23A-23C). CD3⁺CAR⁺ events for the expanded cells were 86% and 98% for the dTp Control (P, 5e6) and Plasmid A (P, 5e6) RPM T cells, respectively. The dTp Control (P, 5e6) cells demonstrated population heterogeneity, consistent with the preceding Examples. As shown in FIG. 23B, the following percentages of CD19CAR/Her1t phenotypes were observed: CD19CAR⁺HER1t⁺ (50%), CD19CAR⁺HER1t^{neg} (27%), CD19CAR^{neg}HER1t⁺ (7%), and CD19CAR^{neg}HER1t^{neg} (16%). Likewise, as shown in FIG. 23C, the following percentages of HER1t/mbIL15 phenotypes were observed: HER1t⁺mbIL15^{neg} (49%), HER1t⁺mbIL15⁺ (7%), HER1t^{neg}mbIL15⁺ (<1%), and HER1t^{neg}mbIL15^{neg} (44%).

In contrast, as shown in FIG. 23B, uniform CAR and HER1t co-expression was observed in the Plasmid A (P, 5e6) cells: CAR⁺HER1t (94%), CAR⁺HER1t^{neg} (3%), CAR^{neg}HER1t (<1%), and CAR^{neg}HER1t^{neg} (2%). Likewise, as shown in FIG. 23C, HER1t and mbIL15 co-expression was improved: HER1t⁺mbIL15^{neg} (69%), HER1t⁺mbIL15⁺ (26%), HER1t^{neg}mbIL15⁺ (<1%), and HER1t^{neg}mbIL15^{neg} (5%).

### 6.4.2.2 Anti-Tumor Efficacy of RPM CD19CAR-mbIL15-HER1t T Cells

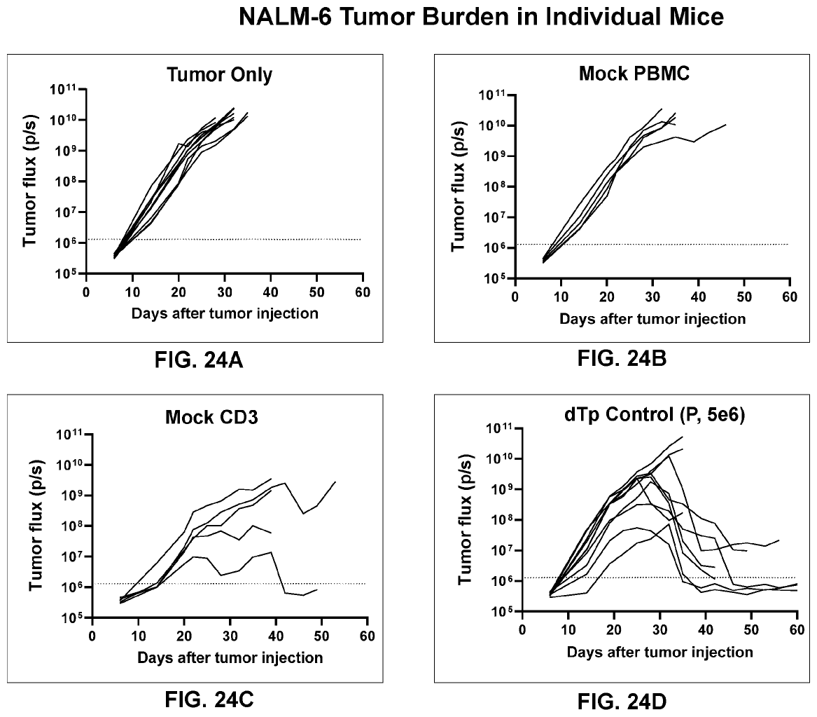

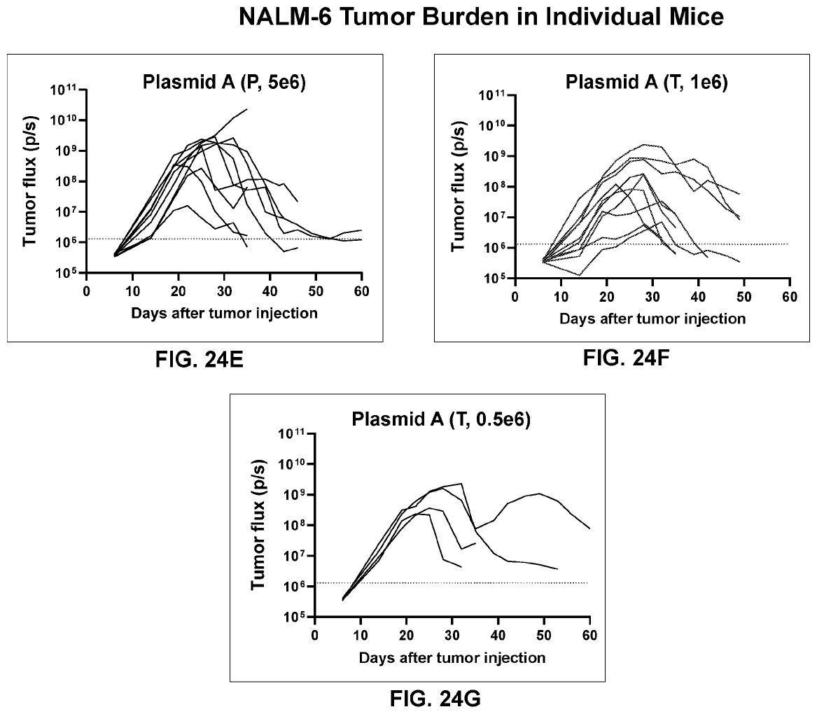

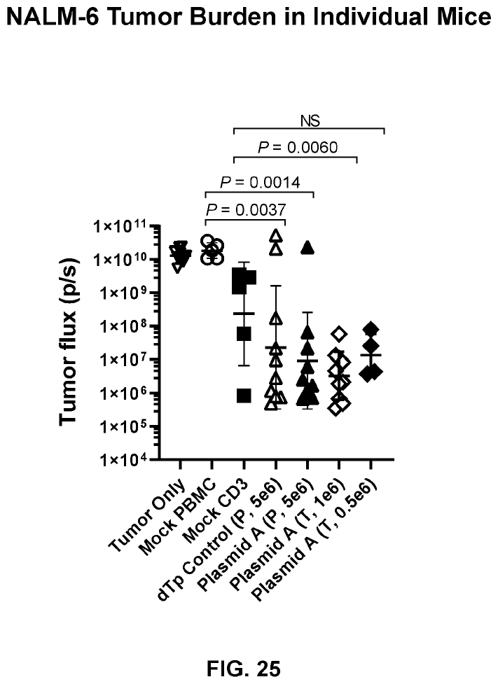

The anti-tumor effect of RPM CD19CAR-mbIL15-HER1t T cells was examined in a NALM-6 mouse xenograft model. The study design is illustrated in Table 18, and tumor burden results are shown in FIGS. 24A-24G. In particular, tumor-bearing mice not receiving treatment all succumbed to disease by Day 35 (FIG. 24A). Mice receiving Mock PBMC succumbed to disease by Day 35, except for one mouse that perished on Day 7 due to injection complication and one mouse reaching Day 46 (FIG. 24B). In the Mock CD3 treatment group, three of five mice succumbed to disease (tumor flux >1×10⁹ p/s) between Days 39 and 53. Two mice were moribund from suspected xGvHD (tumor flux <6×10⁷ p/s) at Days 39 and 49, which is an anticipated outcome in an NSG model engrafted with human lymphocytes, and one of the mice reached 2× background flux (<1.2×10⁶ p/s) (FIG. 24C). Mice treated with dTp Control (P, 5e6) generally became moribund between Days 35 and 62, with two of ten mice possessing high disease burden (>5×10⁹ p/s), and thus likely disease-related mortality. Remaining mice showed stable disease or low tumor burden (xGvHD-related mortality), and of those, 63% were below or approaching the 2× background flux threshold (FIG. 24D). Mice treated with Plasmid A (P, 5e6) exhibited mortality between Days 35 and 60, with a single mouse possessing high tumor load and the remaining eight mice with low tumor burden (<7×10⁷ p/s) and likely xGvHD-related mortality, and of those, 75% were below or approaching the 2× background flux threshold (FIG. 24E). Mice treated with Plasmid A (T, 1e6) survived to between Days 35 and 52, with nine of 10 mice exhibiting low tumor burden (<5×10⁷ p/s) with evident tumor signal decline in the days preceding the end point, and thus xGvHD-related morbidity. Of those nine mice with rapidly diminishing tumor, four mice (44%) showed tumor signal fall below the 2× background flux threshold (FIG. 24F). The mice treated with Plasmid A (T, 0.5e6) survived to between Days 32 and 60, with four of four mice exhibiting low tumor burden (<8×10⁷ p/s) at end point, and thus likely xGvHD related morbidity, and of those, 50% approached the 2× background flux threshold (FIG. 24G). In summary, all RPM CD19CAR-mbIL15-HER1t T cell test articles exhibited significant antitumor activity compared to the no treatment control (<0.0006 for all groups, n=4-10, one-way ANOVA, Dunnett post-test) and all demonstrated significantly lower tumor burden compared to the mock control cells, with the exception of the Plasmid A (T, 0.5e6) treatment, for which statistical significance was not achieved at the tested group size (FIG. 25). Kinetics of antitumor response are consistent with previous studies and are typically observed to initiate after Day 20 post-tumor injection, thus approximately two weeks after T-cell transfer.

Overall, the results clearly demonstrate potent antitumor responses by RPM CD19CAR-mbIL15-HER1t T cells in the established CD19⁺ NALM-6 xenograft model of ALL.

6.4.2.3 Overall and Disease-Free Survival in Animals Treated with RPM CD19CAR-mbIL15-HER1t T Cells

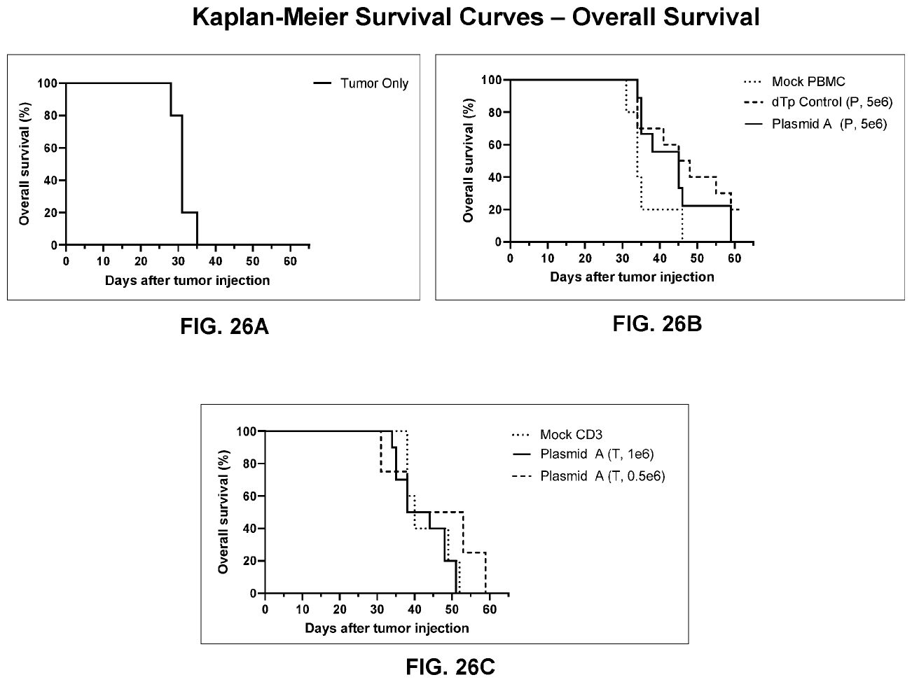

The administration of any of the RPM CD19CAR-mbIL15-HER1t T cell test articles (dTp Control (P, 5e6), Plasmid A (P, 5e6), Plasmid A (T, 1e6), or Plasmid A (T, 0.5e6), corresponding to animal Groups D-G, respectively) significantly enhanced the OS of mice when compared to the Tumor Only control group (P=0.0002, P=0.0004, P<0.0002, and P=0.0098 for Groups D-G, respectively; n=4-10; log rank, Mantel-Cox; FIGS. 26A-26C). Mortality was observed in mice with low tumor burden (total flux <1×10⁸ p/s) that was likely due to xGvHD rather than disease progression in mice, as Tumor Only mice moribund from disease showed total flux >5×10⁹ p/s.

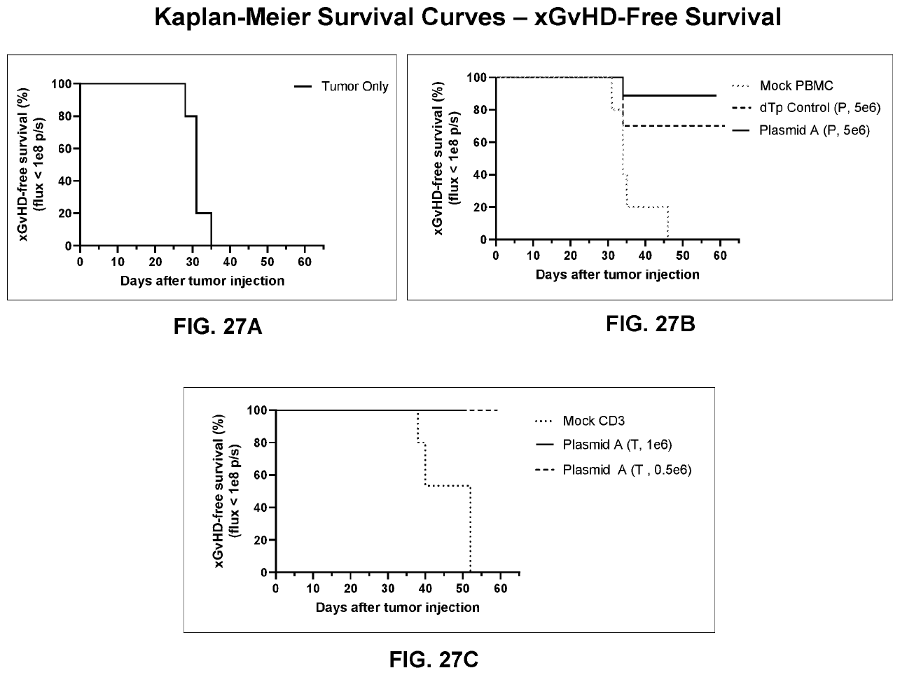

The induction of xGvHD is an anticipated process in an NSG model engrafted with human lymphocytes (as described, e.g., in Ali et al., PLoS ONE. 2012; 7(8):e44219, the contents of which are incorporated by reference in their entirety herein). In consideration of that factor, xGvHD-free survival was calculated whereby animals having total flux <1×10⁸ p/s were censored. In this analysis, survival was increased for all of the RPM CD19CAR-mbIL15-HER1t T cell test articles compared to the Tumor Only control group (P=0.0002, P<0.0001, P<0.0001, and P=0.0018 for Groups D-G, respectively; n=4-10; log rank, Mantel-Cox; FIGS. 27A-27C).

In summary, these results demonstrate that the tested RPM CD19CAR-mbIL15-HER1t T cells, both derived from PBMC and from T cell-enriched products, provide a marked increase in OS when compared to the Tumor Only control group.

6.4.2.4 Determination of xGvHD and Lack of Toxicity of RPM CD19CAR-mbIL15-HER1t T Cells in Mice

There were no RPM CD19-mbIL15-CAR-T cell test article-related changes in body weight observed prior to possible induction of xGvHD processes (i.e., within the first week after T-cell adoptive transfer), whereas the Mock PBMC and Mock CD3 treatments did show body weight decline during this time period. Additionally, over the course of the experiment, the Mock PBMC and Mock CD3 treatments caused progressive weight loss in the mice (i.e., linear regression slopes are negative and significantly different from 0; R²=0.14 and R²=0.44; slope −0.06 and −0.11; P=0.0123 and P<0.001, respectively). No significant decline in mouse body weight for the duration of the experiment was observed in Groups D-G (i.e., linear regression slopes were positive±significant difference from 0; R²<0.05; slope >0.03; P>0.02 for Groups D-G). This suggests that Groups B and C experienced xGvHD effects throughout much of the study, while Groups D-G experienced more sudden morbidity just prior to becoming moribund. Tumor Only mice exhibited weight gain until they became moribund due to tumor burden.

In summary, the intravenous administration of RPM CD19CAR-mbIL15-HER1t T cells (Groups D-G) in the mice bearing NALM-6 was well tolerated. No toxicity was observed proximal to administration of RPM test articles (Groups D-G), and body weight changes proximal to euthanasia was likely due to xGvHD.

### 6.4.2.5 Persistence, Localization, and Memory Phenotype of RPM CD19CAR-mbIL15-HER1t T Cells

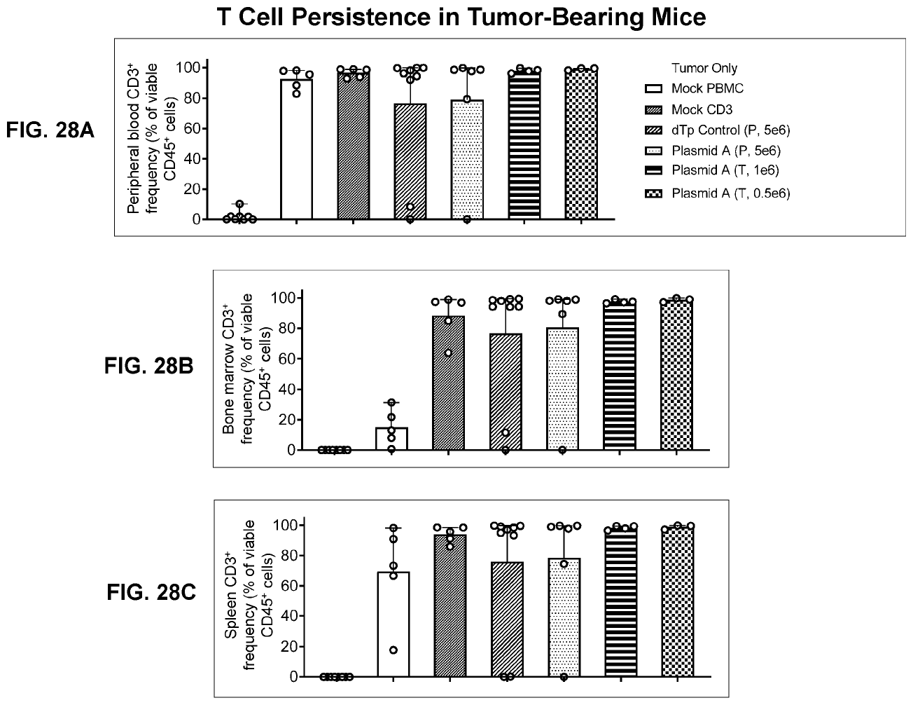

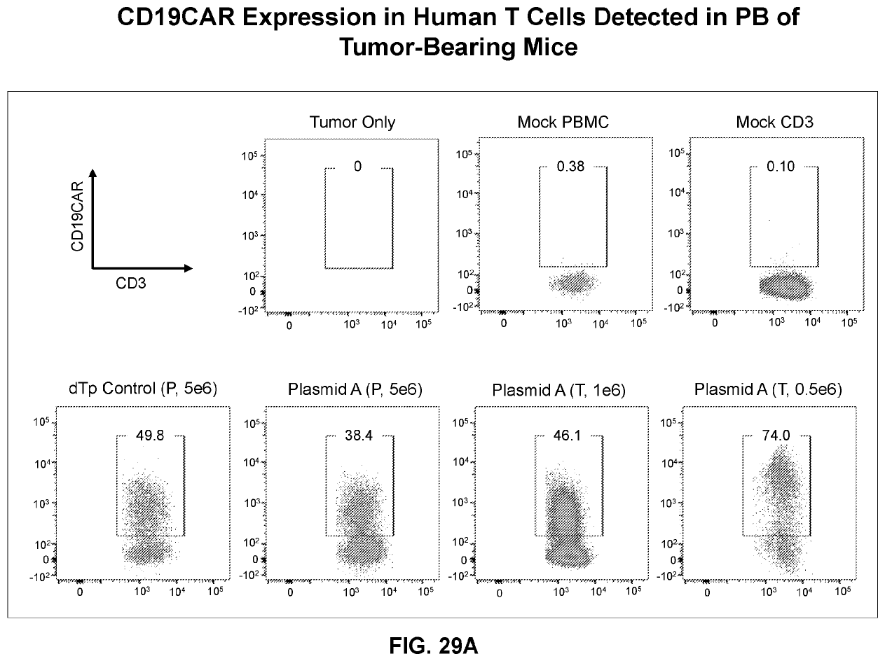

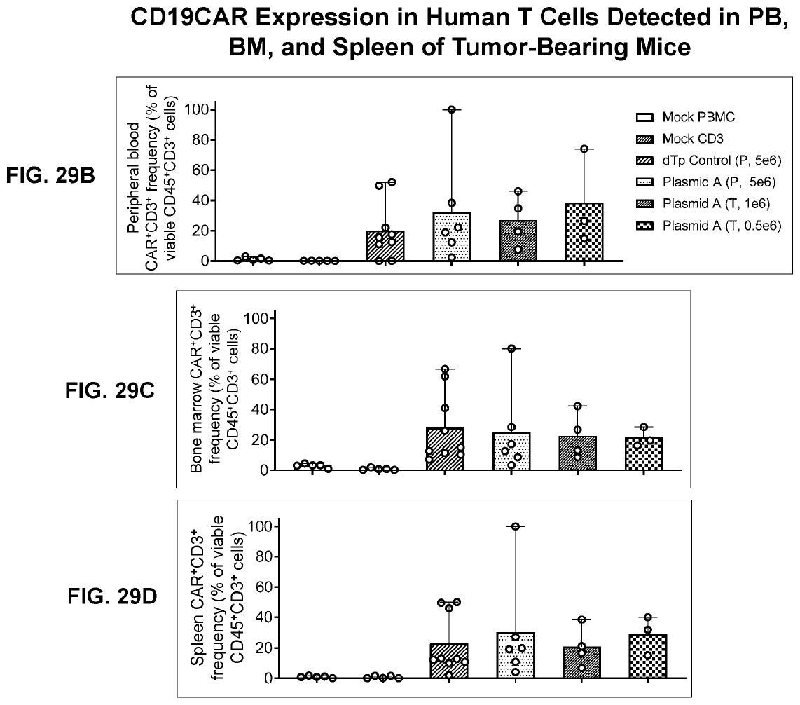

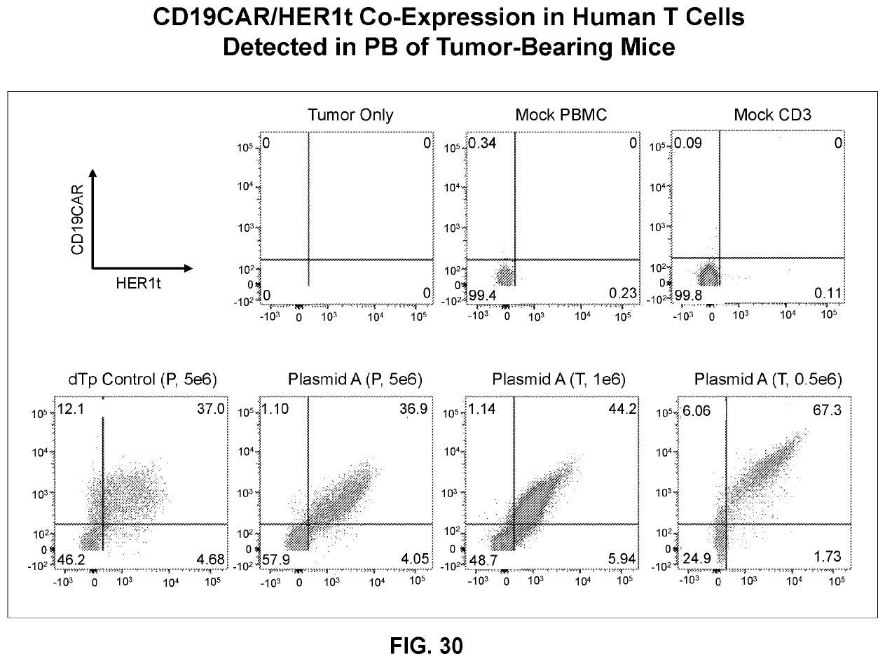

Flow cytometric analysis was performed on peripheral blood (PB), bone marrow (BM), and spleen isolated from mice to assess the persistence, localization, and memory phenotype of RPM CD19CAR-mbIL15-HER1t T cells. Samples were obtained when mice became moribund or at the end of study (study Days 32-62). T-cell engraftment was observed in all T cell-treated mice (Mock PBMC, Mock CD3, dTp Control (P, 5e6), Plasmid A (P, 5e6), Plasmid A (T, 1e6), and Plasmid A (T, 0.5e6; Groups B-G, respectively; FIGS. 28A-28C). Of the engrafted CD3⁺ cells, CAR⁺ T cells were observed to persist at conspicuous levels in the PB, BM, and spleen of mice treated with RPM CD19CAR-mbIL15-HER1t T cells (Groups D-G) (FIG. 29A) ranging from 0%-52%, 2%-100%, 8%-46%, and 15%-74%, respectively, in PB (FIG. 29B), and with no apparent CD19CAR⁺ populations detected in the Mock PBMC and Mock CD3 treatment groups. Similar frequencies of CAR⁺ T cells were observed in the BM and spleen (FIGS. 29C-29D).

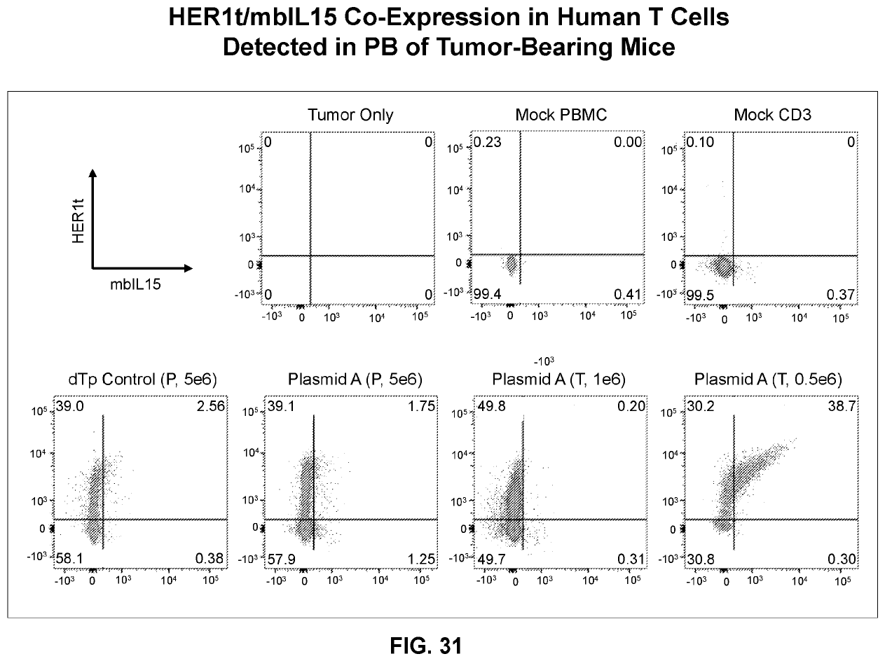

The primary aim for introducing the tricistronic Plasmid A genetic modification of T cells was to decrease the transgene population heterogeneity. This was observed in samples assessed for co-expression of CD19CAR and HER1t from cells isolated from PB. The Plasmid A test articles demonstrated improved homogeneity of expression of CAR⁺HER1t⁺ T cells compared to dTp Control (P, 5e6) (FIG. 30). The detected co-expression of HER1t and mbIL15, and thus expression of mbIL15, was more variable (FIG. 31) and was likely influenced by the cycling kinetics of mbIL15, perhaps due to mechanisms for responding cells to internalize IL-15 bound to IL-15Rα that is cleaved from the presenting cell (see, e.g., Tamzalit et al., Proc Natl Acad Sci USA. 2014; 111(23):8565-8570, the contents of which are incorporated by reference in their entirety herein). Nevertheless, there were instances of high co-expression of HER1t and mbIL15 (e.g., in the Plasmid A (T, 0.5e6) sample). Importantly, there was no significant population of HER1t^{neg}mbIL15⁺ cells present under in vivo conditions (FIG. 31).

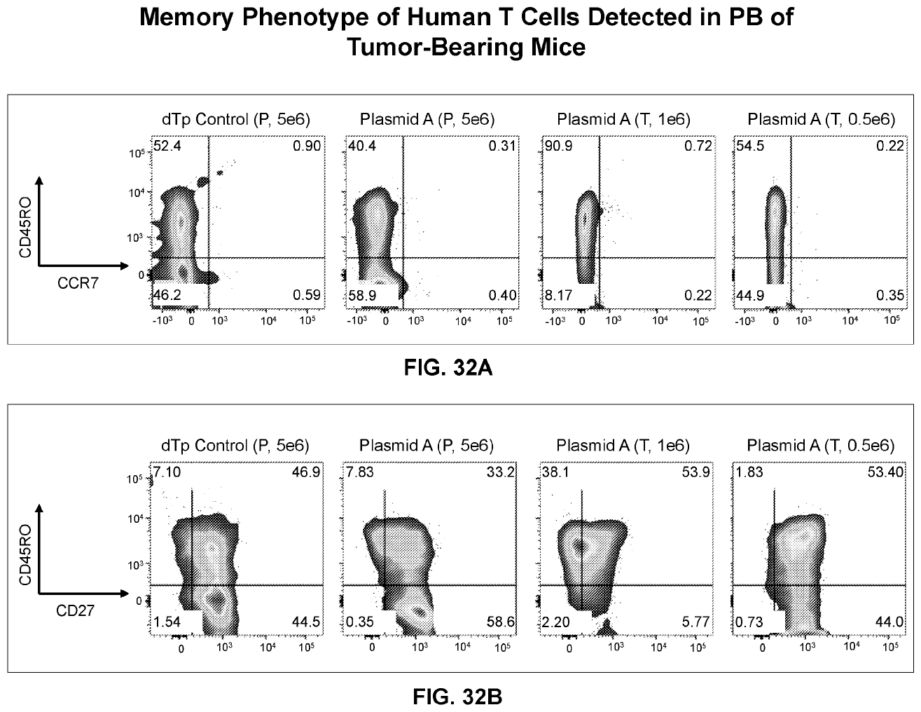

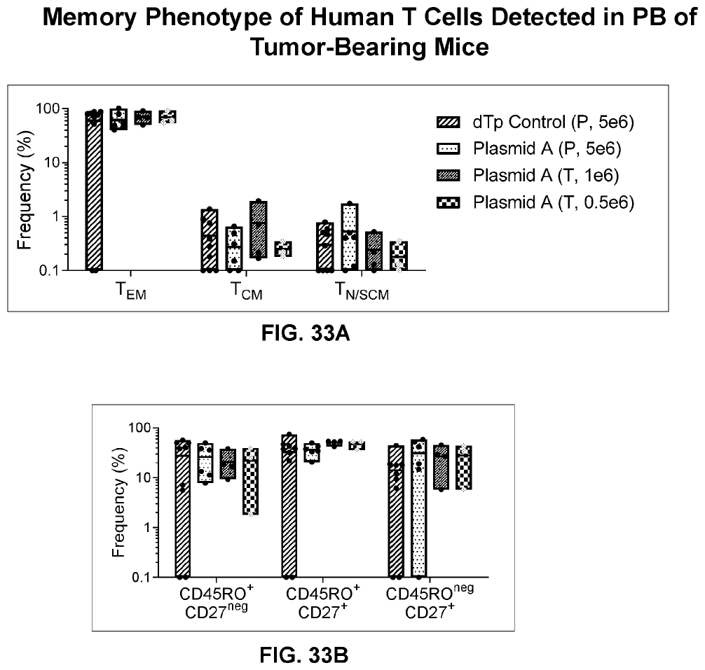

Memory phenotype was assessed for CD19CAR⁺CD3⁺ T cells persisting in the PB of moribund mice. T-cell memory subsets are defined as: CD45RO⁺CCR7⁺: central memory (T_{CM}); CD45RO^{neg} CCR7⁺: naïve/stem cell memory (T_{N/SCM}); CD45RO⁺CCR7^{neg}. effector memory (T_{EM}); and CD45RO^{neg}CCR7^{neg}. effector T (T_{Eff.}). Additionally, T cell differentiation (from low to high) may be represented as: CD45RO^{neg}CD27⁺, CD45RO⁺CD27⁺, CD45RO⁺CD27^{neg}, and CD45RO^{neg}CD27^{neg}. CD19CAR⁺CD3⁺ T cells found persisting were predominantly T_{EM} (FIG. 32A), with means ranging from 59%-70% in the RPM test articles (Groups D-G), when using CD45RO and CCR7 as classifying criteria (FIG. 33A). However, dominant CD27 expression was observed in Groups D-G (FIG. 32B), with means ranging from 33%-51% for CD45RO⁺CD27⁺CD19CAR⁺CD3⁺ T cells and 14%-31% for less differentiated CD45RO^{neg}CD27⁺CAR⁺CD3⁺ T cells (FIG. 33B). The expression of CD27 indicates a less differentiated memory phenotype that is not terminally differentiated (see, e.g., Larbi and Fulop, Cytometry A. 2014; 85(1):25-35, the contents of which are incorporated by reference in their entirety herein).

Overall, these data show that all of the evaluated RPM CD19CAR-mbIL15-HER1t T cell test articles persisted in vivo to end timepoints predominantly as T_{EM} that express CD27.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entireties and for all purposes to the same extent as if each individual reference (e.g., publication or patent or patent application) was specifically and individually indicated to be incorporated by reference in its entirety for all purposes.

Other embodiments are within the following claims.

What is claimed:

1. A recombinant vector comprising a polycistronic expression cassette, wherein said polycistronic expression cassette comprises a transcriptional regulatory element operably linked to a polynucleotide that comprises, from 5′ to 3′:
a. a first polynucleotide sequence that encodes a chimeric antigen receptor (CAR) that comprises an extracellular antigen-binding domain that specifically binds to CD19, a transmembrane domain, and a cytoplasmic domain;
b. a second polynucleotide sequence that comprises an F2A element;
c. a third polynucleotide sequence that encodes a fusion protein that comprises IL-15, or a functional fragment or functional variant thereof, and IL-15Rα, or a functional fragment or functional variant thereof,
d. a fourth polynucleotide sequence that comprises a T2A element; and
e. a fifth polynucleotide sequence that encodes a marker protein.

2. The recombinant vector of claim 1, wherein said F2A element comprises a polynucleotide sequence that encodes the amino acid sequence of SEQ ID NO: 137, or the amino acid sequence of SEQ ID NO: 137, comprising 1, 2, or 3 amino acid modifications.

3. The recombinant vector of claim 1, wherein said F2A element comprises a polynucleotide sequence at least 75%, 80%, 85%, 90%, 95%, 96%, 97%, 98%, 99%, or 100% identical to the polynucleotide sequence of SEQ ID NO: 141.

4. The recombinant vector of claim 1, wherein said F2A element comprises a polynucleotide sequence that encodes the amino acid sequence of SEQ ID NO: 138, or the amino acid sequence of SEQ ID NO: 138, comprising 1, 2, or 3 amino acid modifications.

5. The recombinant vector of claim 1, wherein said F2A element comprises a polynucleotide sequence at least 75%, 80%, 85%, 90%, 95%, 96%, 97%, 98%, 99%, or 100% identical to the polynucleotide sequence of SEQ ID NO: 142.

6. The recombinant vector of any one of claim 1-5, wherein said T2A element comprises a polynucleotide sequence that encodes the amino acid sequence of SEQ ID NO: 139, or the amino acid sequence of SEQ ID NO: 139, comprising 1, 2, or 3 amino acid modifications.

7. The recombinant vector of any one of claims 1-5, wherein said T2A element comprises a polynucleotide sequence at least 75%, 80%, 85%, 90%, 95%, 96%, 97%, 98%, 99%, or 100% identical to the polynucleotide sequence SEQ ID NO: 143.

8. The recombinant vector of any one of claims 1-5, wherein said T2A element comprises a polynucleotide sequence that encodes the amino acid sequence of SEQ ID NO: 140 or 182, or the amino acid sequence of SEQ ID NO: 140 or 182, comprising 1, 2, or 3 amino acid modifications.

9. The recombinant vector of any one of claims 1-5, wherein said T2A element comprises a polynucleotide sequence at least 75%, 80%, 85%, 90%, 95%, 96%, 97%, 98%, 99%, or 100% identical to the polynucleotide sequence of SEQ ID NO: 144, 145, or 165.

10. The recombinant vector of any one of claims 1-9, wherein said antigen-binding domain comprises: a heavy chain variable region (VH) comprising complementarity determining regions VH CDR1, VH CDR2, and VH CDR3; and a light chain variable region (VL) comprising complementarity determining regions VL CDR1, VL CDR2, and VL CDR3.

11. The recombinant vector of claim 10, wherein said antigen-binding domain comprises an scFv that comprises said VH and said VL operably linked via a first peptide linker.

12. The recombinant vector of claim 10 or 11, wherein said VH comprises the VH CDR1, VH CDR2, and VH CDR3 amino acid sequences set forth in SEQ ID NO: 2.

13. The recombinant vector of claim 10 or 11, wherein
a. said VH CDR1 comprises the amino acid sequence of SEQ ID NO: 6; or the amino acid sequence of SEQ ID NO: 6, comprising 1, 2, or 3 amino acid modifications;
b. said VH CDR2 comprises the amino acid sequence of SEQ ID NO: 7; or the amino acid sequence of SEQ ID NO: 7, comprising 1, 2, or 3 amino acid modifications; and
c. said VH CDR3 comprises the amino acid sequence of SEQ ID NO: 8; or the amino acid sequence of SEQ ID NO: 8, comprising 1, 2, or 3 amino acid modifications.

14. The recombinant vector of any one of claims 10-13, wherein said VL comprises the VL CDR1, VL CDR2, and VL CDR3 amino acid sequences set forth in SEQ ID NO: 1.

15. The recombinant vector of any one of claims 10-13, wherein
a. said VL CDR1 comprises the amino acid sequence of SEQ ID NO: 3; or the amino acid sequence of SEQ ID NO: 3, comprising 1, 2, or 3 amino acid modifications;
b. said VL CDR2 comprises the amino acid sequence of SEQ ID NO: 4; or the amino acid sequence of SEQ ID NO: 4, comprising 1, 2, or 3 amino acid modifications; and
c. said VL CDR3 comprises the amino acid sequence of SEQ ID NO: 5; or the amino acid sequence of SEQ ID NO: 5, comprising 1, 2, or 3 amino acid modifications.

16. The recombinant vector of any one of claims 10-15, wherein said VH comprises an amino acid sequence at least 95%, 96%, 97%, 98%, 99%, or 100% identical to the amino acid sequence of SEQ ID NO: 2.

17. The recombinant vector of any one of claims 10-16, wherein said VH is encoded by a polynucleotide sequence at least 75%, 80%, 85%, 90%, 95%, 96%, 97%, 98%, 99%, or 100% identical to the polynucleotide sequence of SEQ ID NO: 20.

18. The recombinant vector of any one of claims 10-17, wherein said VL comprises an amino acid sequence at least 95%, 96%, 97%, 98%, 99%, or 100% identical to the amino acid sequence of SEQ ID NO: 1.

19. The recombinant vector of any one of claims 10-18, wherein said VL is encoded by a polynucleotide sequence at least 75%, 80%, 85%, 90%, 95%, 96%, 97%, 98%, 99%, or 100% identical to the polynucleotide sequence of SEQ ID NO: 19.

20. The recombinant vector of any one of claims 11-19, wherein said first peptide linker comprises the amino acid sequence of SEQ ID NO: 9 or SEQ ID NO: 17, or the amino acid sequence of SEQ ID NO: 9 or 17, comprising 1, 2, or 3 amino acid modifications.

21. The recombinant vector of any one of claims 11-20, wherein said first peptide linker is encoded by a polynucleotide sequence at least 75%, 80%, 85%, 90%, 95%, 96%, 97%, 98%, 99%, or 100% identical to the polynucleotide sequence of SEQ ID NO: 27 or SEQ ID NO: 35.

22. The recombinant vector of claim 21, wherein said first peptide linker is encoded by a polynucleotide sequence at least 75%, 80%, 85%, 90%, 95%, 96%, 97%, 98%, 99%, or 100% identical to the polynucleotide sequence of SEQ ID NO: 27.

23. The recombinant vector of any one of claims 1-22, wherein said CAR further comprises a hinge region positioned between said antigen-binding domain and said transmembrane domain of said CAR.

24. The recombinant vector of claim 23, wherein said hinge region comprises the amino acid sequence of SEQ ID NO: 37, 38, or 39, or the amino acid sequence of SEQ ID NO: 37, 38, or 39, comprising 1, 2, or 3 amino acid modifications.

25. The recombinant vector of claim 23, wherein said hinge region is encoded by a polynucleotide sequence at least 75%, 80%, 85%, 90%, 95%, 96%, 97%, 98%, 99%, or 100% identical to the polynucleotide sequence of SEQ ID NO: 40, 41, or 42.

26. The recombinant vector of any one of claims 1-25, wherein said transmembrane domain of said CAR comprises the amino acid sequence of SEQ ID NO: 43, 44, or 45, or the amino acid sequence of SEQ ID NO: 43, 44, or 45, comprising 1, 2, or 3 amino acid modifications.

27. The recombinant vector of any one of claims 1-25, wherein said transmembrane domain of said CAR is encoded by a polynucleotide sequence at least 75%, 80%, 85%, 90%, 95%, 96%, 97%, 98%, 99%, or 100% identical to the polynucleotide sequence of SEQ ID NO: 49, 50, 51, or 52.

28. The recombinant vector of any one of claims 23-27, wherein said hinge region and said transmembrane domain together comprise the amino acid sequence of SEQ ID NO: 46, 47, or 48, or the amino acid sequence of SEQ ID NO: 46, 47, or 48, comprising 1, 2, or 3 amino acid modifications.

29. The recombinant vector of any one of claims 23-27, wherein said hinge region and said transmembrane domain together are encoded by a polynucleotide sequence at least 75%, 80%, 85%, 90%, 95%, 96%, 97%, 98%, 99%, or 100% identical to the polynucleotide sequence of SEQ ID NO: 53, 54, 55, or 56.

30. The recombinant vector of any one of claims 1-29, wherein said cytoplasmic domain comprises a primary signaling domain of human CD3ζ, or a functional fragment or functional variant thereof.

31. The recombinant vector of claim 30, wherein said cytoplasmic domain comprises an amino acid sequence at least 95%, 96%, 97%, 98%, 99%, or 100% identical to the amino acid sequence of SEQ ID NO: 60.

32. The recombinant vector of claim 30, wherein said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 67 or 68.

33. The recombinant vector of any one of claims 1-32, wherein said cytoplasmic domain comprises a co-stimulatory domain, or functional fragment or variant thereof, of a protein selected from the group consisting of CD28, 4-1BB, OX40, CD2, CD7, CD27, CD30, CD40, CDS, ICAM-1, LFA-1, B7-H3, and ICOS.

34. The recombinant vector of claim 33, wherein said protein is CD28 or 4-1BB.

35. The recombinant vector of claim 33 or 34, wherein said protein is CD28.

36. The recombinant vector of any one of claims 33-35, wherein said cytoplasmic domain comprises the amino acid sequence of SEQ ID NO: 57 or 58, or the amino acid sequence of SEQ ID NO: 57 or 58, comprising 1, 2, or 3 amino acid modifications.

37. The recombinant vector of any one of claims 33-35, wherein said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 64 or 65.

38. The recombinant vector of claim 33 or 34, wherein said protein is 4-1BB.

39. The recombinant vector of any one of claims 33, 34, or 38, wherein said cytoplasmic domain comprises the amino acid sequence of SEQ ID NO: 59, or the amino acid sequence of SEQ ID NO: 59, comprising 1, 2, or 3 amino acid modifications.

40. The recombinant vector of any one of claims 33, 34, 38, or 39, wherein said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 66.

41. The recombinant vector of any one of claims 1-40, wherein said cytoplasmic domain comprises the amino acid sequence of SEQ ID NO: 61, 62, or 63, or the amino acid sequence of SEQ ID NO: 61, 62, or 63, comprising 1, 2, or 3 amino acid modifications.

42. The recombinant vector of any one of claims 1-41, wherein said cytoplasmic domain is encoded by a polynucleotide sequence at least 75%, 80%, 85%, 90%, 95%, 96%, 97%, 98%, 99%, or 100% identical to the polynucleotide sequence of SEQ ID NO: 69, 70, or 71.

43. The recombinant vector of any one of claims 1-42, wherein said CAR comprises an amino acid sequence at least at least 95%, 96%, 97%, 98%, 99%, or 100% identical to the amino acid sequence of SEQ ID NO: 72, 74, 76, 77, 78, 79, 80, or 81.

44. The recombinant vector of any one of claims 1-43, wherein said CAR is encoded by a polynucleotide sequence at least 75%, 80%, 85%, 90%, 95%, 96%, 97%, 98%, 99%, or 100% identical to the polynucleotide sequence of SEQ ID NO: 82, 83, 86, 87, 90, 91, 92, 93, 94, or 95.

45. The recombinant vector of any one of claims 1-44, wherein said IL-15, or said functional fragment or functional variant thereof, is operably linked to said IL-15Rα, or said functional fragment or functional variant thereof, via a second peptide linker.

46. The recombinant vector of any one of claims 1-45, wherein said fusion protein comprises an amino acid sequence at least 95%, 96%, 97%, 98%, 99%, or 100% identical to the amino acid sequence of SEQ ID NO: 119, 121, or 180.

47. The recombinant vector of any one of claims 1-46, wherein said fusion protein is encoded by a polynucleotide sequence at least 75%, 80%, 85%, 90%, 95%, 96%, 97%, 98%, 99%, or 100% identical to the polynucleotide sequence of SEQ ID NO: 126, 127, 130, 131, or 181.

48. The recombinant vector of any one of claims 1-47, wherein said marker protein comprises: domain III of HER1, or a functional fragment or functional variant thereof; an N-terminal portion of domain IV of HER1; and a transmembrane domain of CD28, or a functional fragment or functional variant thereof.

49. The recombinant vector of claim 48, wherein said domain III of HER1, or a functional fragment or functional variant thereof, comprises an amino acid sequence at least 95%, 96%, 97%, 98%, 99%, or 100% identical to the amino acid sequence of SEQ ID NO: 98.

50. The recombinant vector of claim 48, wherein said domain III of HER1, or a functional fragment or functional variant thereof, is encoded by a polynucleotide sequence at least 75%, 80%, 85%, 90%, 95%, 96%, 97%, 98%, 99%, or 100% identical to the polynucleotide sequence of SEQ ID NO: 110 or 164.

51. The recombinant vector of any one of claims 48-50, wherein said N-terminal portion of domain IV of HER1 comprises amino acids 1-40, 1-39, 1-38, 1-37, 1-36, 1-35, 1-34, 1-33, 1-32, 1-31, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, or 1-10 of SEQ ID NO: 99.

52. The recombinant vector of any one of claims 48-51, wherein said N-terminal portion of domain IV of HER1 comprises amino acids 1-21 of SEQ ID NO: 99.

53. The recombinant vector of any one of claims 48-52, wherein said N-terminal portion of domain IV of HER1 comprises the amino acid sequence of SEQ ID NO: 100, or the amino acid sequence of SEQ ID NO: 100, comprising 1, 2, or 3 amino acid modifications.

54. The recombinant vector of any one of claims 48-53, wherein said N-terminal portion of domain IV of HER1 is encoded by a polynucleotide sequence at least 75%, 80%, 85%, 90%, 95%, 96%, 97%, 98%, 99%, or 100% identical to the polynucleotide sequence of SEQ ID NO: 112.

55. The recombinant vector of any one of claims 48-54, wherein said transmembrane region of CD28 comprises the amino acid sequence of SEQ ID NO: 101, or the amino acid sequence of SEQ ID NO: 101, comprising 1, 2, or 3 amino acid modifications.

56. The recombinant vector of any one of claims 48-55, wherein said transmembrane region of CD28 is encoded by a polynucleotide sequence at least 75%, 80%, 85%, 90%, 95%, 96%, 97%, 98%, 99%, or 100% identical to the polynucleotide sequence of SEQ ID NO: 113.

57. The recombinant vector of any one of claims 1-56, wherein said marker protein comprises an amino acid sequence at least 95%, 96%, 97%, 98%, 99%, or 100% identical to the amino acid sequence of SEQ ID NO: 96, 97, 166, or 167.

58. The recombinant vector of any one of claims 1-57, wherein said marker protein is encoded by a polynucleotide sequence at least 75%, 80%, 85%, 90%, 95%, 96%, 97%, 98%, 99%, or 100% identical to the polynucleotide sequence of SEQ ID NO: 107, 108, 109, 162, 173, or 174.

59. The recombinant vector of any one of claims 1-58, wherein said regulatory element comprises a promoter.

60. The recombinant vector of claim 59, wherein said promoter is a human elongation factor 1-alpha (hEF-1α) hybrid promoter.

61. The recombinant vector of claim 59 or 60, wherein said promoter comprises a polynucleotide sequence at least 75%, 80%, 85%, 90%, 95%, 96%, 97%, 98%, 99%, or 100% identical to the polynucleotide sequence of SEQ ID NO: 146.

62. The recombinant vector of any one of claims 1-61, wherein said vector further comprises a polyA sequence 3′ of said fifth polynucleotide sequence.

63. The recombinant vector of claim 62, wherein said polyA sequence comprises a polynucleotide sequence at least 75%, 80%, 85%, 90%, 95%, 96%, 97%, 98%, 99%, or 100% identical to the polynucleotide sequence of SEQ ID NO: 148.

64. A recombinant vector comprising a polycistronic expression cassette, wherein said polycistronic expression cassette comprises a transcriptional regulatory element operably linked to a polynucleotide that comprises, from 5′ to 3′:
a. a first polynucleotide sequence that encodes a CAR that comprises an amino acid sequence at least 95%, 96%, 97%, 98%, 99%, or 100% identical to the amino acid sequence of SEQ ID NO: 72 or 74;
b. a second polynucleotide sequence that comprises an F2A element;
c. a third polynucleotide sequence that encodes a fusion protein that comprises an amino acid sequence at least 95%, 96%, 97%, 98%, 99%, or 100% identical to the amino acid sequence of SEQ ID NO: 119, 121, or 180;
d. a fourth polynucleotide sequence that comprises a T2A element; and
e. a fifth polynucleotide sequence that encodes a marker protein that comprises an amino acid sequence at least 95%, 96%, 97%, 98%, 99%, or 100% identical to the amino acid sequence of SEQ ID NO: 96 or 97.

65. The recombinant vector of claim 64, wherein said F2A element comprises a polynucleotide sequence that encodes the amino acid sequence of SEQ ID NO: 137, or the amino acid sequence of SEQ ID NO: 137, comprising 1, 2, or 3 amino acid modifications.

66. The recombinant vector of claim 64, wherein said F2A element comprises a polynucleotide sequence at least 75%, 80%, 85%, 90%, 95%, 96%, 97%, 98%, 99%, or 100% identical to the polynucleotide sequence of SEQ ID NO: 141.

67. The recombinant vector of claim 64, wherein said F2A element comprises a polynucleotide sequence that encodes the amino acid sequence of SEQ ID NO: 138, or the amino acid sequence of SEQ ID NO: 138, comprising 1, 2, or 3 amino acid modifications.

68. The recombinant vector of claim 64, wherein said F2A element comprises a polynucleotide sequence at least 75%, 80%, 85%, 90%, 95%, 96%, 97%, 98%, 99%, or 100% identical to the polynucleotide sequence of SEQ ID NO: 142.

69. The recombinant vector of any one of claims 64-68, wherein said T2A element comprises a polynucleotide sequence that encodes the amino acid sequence of SEQ ID NO: 139, or the amino acid sequence of SEQ ID NO: 139, comprising 1, 2, or 3 amino acid modifications.

70. The recombinant vector of any one of claims 64-68, wherein said T2A element comprises a polynucleotide sequence at least 75%, 80%, 85%, 90%, 95%, 96%, 97%, 98%, 99%, or 100% identical to the polynucleotide sequence SEQ ID NO: 143.

71. The recombinant vector of any one of claims 64-68, wherein said T2A element comprises a polynucleotide sequence that encodes the amino acid sequence of SEQ ID NO: 140 or 182, or the amino acid sequence of SEQ ID NO: 140 or 182, comprising 1, 2, or 3 amino acid modifications.

72. The recombinant vector of any one of claims 64-68, wherein said T2A element comprises a polynucleotide sequence at least 75%, 80%, 85%, 90%, 95%, 96%, 97%, 98%, 99%, or 100% identical to the polynucleotide sequence of SEQ ID NO: 144, 145, or 165.

73. A recombinant vector comprising a polycistronic expression cassette, wherein said polycistronic expression cassette comprises a transcriptional regulatory element operably linked to a polynucleotide that comprises, from 5′ to 3′:
a. a first polynucleotide sequence at least 75%, 80%, 85%, 90%, 95%, 96%, 97%, 98%, 99%, or 100% identical to the polynucleotide sequence of SEQ ID NO: 82, 83, 86, or 87;
b. a second polynucleotide sequence that comprises an F2A element;
c. a third polynucleotide sequence at least 75%, 80%, 85%, 90%, 95%, 96%, 97%, 98%, 99%, or 100% identical to the polynucleotide sequence of SEQ ID NO: 126, 127, 130, 131, or 181;
d. a fourth polynucleotide sequence that comprises a T2A element; and
e. a fifth polynucleotide sequence at least 75%, 80%, 85%, 90%, 95%, 96%, 97%, 98%, 99%, or 100% identical to the polynucleotide sequence of SEQ ID NO: 107, 108, 109, or 162.

74. The recombinant vector of claim 73, wherein said F2A element comprises a polynucleotide sequence that encodes the amino acid sequence of SEQ ID NO: 137, or the amino acid sequence of SEQ ID NO: 137, comprising 1, 2, or 3 amino acid modifications.

75. The recombinant vector of claim 73, wherein said F2A element comprises a polynucleotide sequence at least 75%, 80%, 85%, 90%, 95%, 96%, 97%, 98%, 99%, or 100% identical to the polynucleotide sequence of SEQ ID NO: 141.

76. The recombinant vector of claim 73, wherein said F2A element comprises a polynucleotide sequence that encodes the amino acid sequence of SEQ ID NO: 138, or the amino acid sequence of SEQ ID NO: 138, comprising 1, 2, or 3 amino acid modifications.

77. The recombinant vector of claim 73, wherein said F2A element comprises a polynucleotide sequence at least 75%, 80%, 85%, 90%, 95%, 96%, 97%, 98%, 99%, or 100% identical to the polynucleotide sequence of SEQ ID NO: 142.

78. The recombinant vector of any one of claims 73-77, wherein said T2A element comprises a polynucleotide sequence that encodes the amino acid sequence of SEQ ID NO: 139, or the amino acid sequence of SEQ ID NO: 139, comprising 1, 2, or 3 amino acid modifications.

79. The recombinant vector of any one of claims 73-77, wherein said T2A element comprises a polynucleotide sequence at least 75%, 80%, 85%, 90%, 95%, 96%, 97%, 98%, 99%, or 100% identical to the polynucleotide sequence SEQ ID NO: 143.

80. The recombinant vector of any one of claims 73-77, wherein said T2A element comprises a polynucleotide sequence that encodes the amino acid sequence of SEQ ID NO: 140 or 182, or the amino acid sequence of SEQ ID NO: 140 or 182, comprising 1, 2, or 3 amino acid modifications.

81. The recombinant vector of any one of claims 73-77, wherein said T2A element comprises a polynucleotide sequence at least 75%, 80%, 85%, 90%, 95%, 96%, 97%, 98%, 99%, or 100% identical to the polynucleotide sequence of SEQ ID NO: 144, 145, or 165.

82. The recombinant vector of any one of claims 1-81, further comprising a Left inverted terminal repeat (ITR) and a Right ITR, wherein said Left ITR and said Right ITR flank said polycistronic expression cassette.

83. The recombinant vector of claim 82, which comprises, from 5′ to 3′:
a. said Left ITR;
b. said transcriptional regulatory element;
c. said first polynucleotide sequence;
d. said second polynucleotide sequence;
e. said third polynucleotide sequence;
f. said fourth polynucleotide sequence;
g. said fifth polynucleotide sequence; and
h. said Right ITR.

84. A recombinant vector comprising a polycistronic expression cassette, wherein said polycistronic expression cassette comprises a transcriptional regulatory element operably linked to a polynucleotide sequence at least 75%, 80%, 85%, 90%, 95%, 96%, 97%, 98%, 99%, or 100% identical to the polynucleotide sequence of SEQ ID NO: 149.

85. A recombinant vector comprising a polycistronic expression cassette, wherein said polycistronic expression cassette comprises a transcriptional regulatory element operably linked to a polynucleotide that encodes an amino acid sequence at least 95%, 96%, 97%, 98%, 99%, or 100% identical to the amino acid sequence of SEQ ID NO: 152.

86. The recombinant vector of claim 84 or 85, further comprising a Left inverted terminal repeat (ITR) and a Right ITR, wherein said Left ITR and said Right ITR flank said polycistronic expression cassette.

87. The recombinant vector of any one of claims 82, 83, or 86, wherein said Left ITR and said Right ITR are ITRs of a DNA transposon selected from the group consisting of a Sleeping Beauty transposon, a piggyBac transposon, TcBuster transposon, and a Tol2 transposon.

88. The recombinant vector of claim 87, wherein said DNA transposon is said Sleeping Beauty transposon.

89. The recombinant vector of any one of claims 1-88, wherein said vector is a non-viral vector.

90. The recombinant vector of claim 89, wherein said non-viral vector is a plasmid.

91. The recombinant vector of any one of claims 1-90, wherein said vector is a viral vector.

92. The recombinant vector of any one of claims 1-91, wherein said vector is a polynucleotide.

93. A polynucleotide encoding an amino acid sequence at least 95%, 96%, 97%, 98%, 99%, or 100% identical to the amino acid sequence of SEQ ID NO: 152.

94. A population of cells that comprise the vector of any one of claims 1-92.

95. The population of cells of claim 94, wherein said vector is integrated into the genome of said population of cells.

96. A population of cells that comprise the polynucleotide of claim 93.

97. The population of cells of claim 96, wherein said polynucleotide is integrated into the genome of said population of cells.

98. A population of cells that comprise a polypeptide comprising an amino acid sequence encoded by the polynucleotide of claim 93.

99. The population of cells of any one of claims 94-98, comprising a CAR comprising the amino acid sequence of SEQ ID NO: 72, 73, 74, 75, 76, 77, 78, 79, 80, or 81; a fusion protein comprising the amino acid sequence of SEQ ID NO: 119, 120, 121, 122, 180, or 183; and a marker protein comprising the amino acid sequence of SEQ ID NO: 96, 97, 166, or 167.

100. The population of cells of any one of claims 94-98, comprising a CAR comprising the amino acid sequence of SEQ ID NO: 74; a fusion protein comprising the amino acid sequence of SEQ ID NO: 121; and a marker protein comprising the amino acid sequence of SEQ ID NO: 97.

101. The population of cells of any one of claims 94-98, comprising a CAR comprising the amino acid sequence of SEQ ID NO: 75; a fusion protein comprising the amino acid sequence of SEQ ID NO: 122; and a marker protein comprising the amino acid sequence of SEQ ID NO: 97.

102. The population of cells of any one of claims 94-101, wherein said cells are immune effector cells.

103. The population of cells of claim 102, wherein said immune effector cells are selected from the group consisting of T cells, natural killer (NK) cells, B cells, mast cells, and myeloid-derived phagocytes.

104. The population of cells of claim 103, wherein said immune effector cells are T cells.

105. The population of cells of claim 104, comprising alpha/beta T cells, gamma/delta T cells, or natural killer T (NK-T) cells.

106. The population of cells of claim 104 or 105, comprising CD4⁺ T cells, CD8⁺ T cells, or both CD4⁺ T cells and CD8⁺ T cells.

107. The population of cells of any one of claims 94-106, wherein said cells are ex vivo.

108. The population of cells of any one of claims 94-107, wherein said cells are human.

109. A method of producing a population of engineered cells, comprising:
a. introducing into a population of cells the recombinant vector of any one of claims 82, 83, or 86-88, and a DNA transposase or a polynucleotide encoding a DNA transposase; and
b. culturing said population of cells under conditions wherein said transposase integrates the polycistronic expression cassette into the genome of said population of cells,
thereby producing the population of engineered cells.

110. The method of claim 109, wherein said Left ITR and said Right ITR are ITRs of a DNA transposon selected from the group consisting of a Sleeping Beauty transposon, a piggyBac transposon, a TcBuster transposon, and a Tol2 transposon.

111. The method of claim 109 or 110, wherein said DNA transposon is said Sleeping Beauty transposon.

112. The method of any one of claims 109-111, wherein said transposase is a Sleeping Beauty transposase.

113. The method of claim 112, wherein said Sleeping Beauty transposase is selected from the group consisting of SB11, SB100X, hSB110, and hSB81.

114. The method of claim 112 or 113, wherein said Sleeping Beauty transposase is SB11.

115. The method of claim 114, wherein said SB11 comprises an amino acid sequence at least 95%, 96%, 97%, 98%, 99%, or 100% identical to the amino acid sequence of SEQ ID NO: 160.

116. The method of claim 114 or 115, wherein said SB11 is encoded by a polynucleotide sequence at least at least 75%, 80%, 85%, 90%, 95%, 96%, 97%, 98%, 99%, or 100% identical to the polynucleotide sequence of SEQ ID NO: 161.

117. The method of any one of claims 109-116, wherein said polynucleotide encoding said DNA transposase is a DNA vector or an RNA vector.

118. The method of any one of claims 109-117, wherein said Left ITR comprises a polynucleotide sequence at least 75%, 80%, 85%, 90%, 95%, 96%, 97%, 98%, 99%, or 100% identical to the polynucleotide sequence of SEQ ID NO: 155 or 156; and said Right ITR comprises a polynucleotide sequence at least 75%, 80%, 85%, 90%, 95%, 96%, 97%, 98%, 99%, or 100% identical to the polynucleotide sequence of SEQ ID NO: 157, 159, or 184.

119. The method of any one of claims 109-118, wherein said recombinant vector, and said DNA transposase or polynucleotide encoding said DNA transposase, are introduced to said population of cells using electro-transfer, calcium phosphate precipitation, lipofection, particle bombardment, microinjection, mechanical deformation by passage through a microfluidic device, or a colloidal dispersion system.

120. The method of claim 119, wherein said recombinant vector, and said DNA transposase or polynucleotide encoding said DNA transposase, are introduced to said population of cells using electro-transfer.

121. The method of any one of claims 109-120, wherein said method is completed in less than two days.

122. The method of any one of claims 109-120, wherein said method is completed in 1-2 days.

123. The method of any one of claims 109-120, wherein said method is completed in more than two days.

124. The method of any one of claims 109-123, wherein said population of cells is cryopreserved and thawed before introduction of said recombinant vector and said DNA transposase or polynucleotide encoding said DNA transposase.

125. The method of any one of claims 109-124, wherein said population of cells is rested before introduction of said recombinant vector and said DNA transposase or polynucleotide encoding said DNA transposase.

126. The method of any one of claims 109-125, wherein said population of cells comprises human ex vivo cells.

127. The method of any one of claims 109-126, wherein said population of cells is not activated ex vivo.

128. The method of any one of claims 109-127, wherein said population of cells comprises T cells.

129. A method of treating cancer in a subject in need thereof comprising administering to the subject a therapeutically effective amount of the population of cells of any one of claims 94-108, thereby treating the cancer.

130. A method of treating cancer in a subject in need thereof comprising administering to the subject a therapeutically effective amount of the population of engineered cells produced by the method of any one of claims 109-128, thereby treating the cancer.

131. A method of treating an autoimmune disease or disorder in a subject in need thereof comprising administering to the subject a therapeutically effective amount of the population of cells of any one of claims 94-108, thereby treating the autoimmune disease or disorder.

132. A method of treating an autoimmune disease or disorder in a subject in need thereof comprising administering to the subject a therapeutically effective amount of the population of engineered cells produced by the method of any one of claims 109-128, thereby treating the autoimmune disease or disorder.
